(19) United States
(12) Patent Application Publication
(10) Pub. No.: US 2022/0362311 A1
(43) Pub. Date: Nov. 17, 2022

(54) OPTIMIZED BACTERIA ENGINEERED TO TREAT DISORDERS INVOLVING THE CATABOLISM OF LEUCINE, ISOLEUCINE, AND/OR VALINE

(71) Applicants: Synlogic Operating Company, Inc., Cambridge, MA (US); Ginkgo Bioworks, Inc., Boston, MA (US)

(72) Inventors: Ning Li, Winchester, MA (US); Jian-Rong Gao, Malden, MA (US); Philippa Jane Reeder Morrison, Arlington, MA (US); Christopher George Bergeron, Fitchburg, MA (US); Dylan Alexander Carlin, Jamaica Plain, MA (US); Laura K. Stone, Cambridge, MA (US); Alex C. Tucker, Dorchester, MA (US); Patrick M. Boyle, Boston, MA (US)

(21) Appl. No.: 17/621,121

(22) PCT Filed: Jun. 19, 2020
(86) PCT No.: PCT/US2020/038675

§ 371 Date: Dec. 20, 2021

Related U.S. Application Data
(60) Provisional application No. 62/864,875, filed on Jun. 21, 2019.
(60) Provisional application No. 62/865,129, filed on Jun. 21, 2019.

(51) Int. Cl. A61K 35/747; A61K 35/741; A61K 35/745; A61K 35/742; A61K 35/744; A61P 13/02; C12N 1/20; C12N 15/52; C12N 15/70

(52) U.S. Cl. CPC A61K 35/747; A61K 35/741; A61K 35/745; A61K 35/742; A61K 35/744; A61P 13/02; C12N 1/205; C12Y 101/01105; C12Y 104/01009; C12N 15/52; C12N 15/70; C12R 2001/19

(57) ABSTRACT

The present disclosure provides recombinant bacterial cells that have been engineered with genetic circuitry which allow the recombinant bacterial cells to sense a patients internal environment and respond by turning an engineered metabolic pathway on or off. When turned on, the recombinant bacterial cells complete all of the steps in a metabolic pathway to achieve a therapeutic effect in a host subject. These recombinant bacterial cells are designed to drive therapeutic effects throughout the body of a host from a point of origin of the microbiome. Specifically, the present disclosure provides recombinant bacterial cells comprising a heterologous gene encoding an improved leucine catabolism enzyme with higher activity and/or specificity for leucine over other branched chain amino acids, such as isoleucine or valine. The disclosure further provides pharmaceutical compositions comprising the recombinant bacteria, and methods for treating disorders involving the catabolism of leucine, isoleucine, and/or valine using the pharmaceutical compositions disclosed herein.

### RELATED APPLICATIONS

This application claims priority to U.S. Provisional Application No. 62/864,875, filed on Jun. 21, 2019, and U.S. Provisional Application No. 62/865,129, filed on Jun. 21, 2019. The entire contents of each of the foregoing applications are hereby incorporated by reference.

### SEQUENCE LISTING

The instant application contains a sequence Listing which has been submitted electronically in ASCII format and is hereby incorporated by reference in its entirety. Said ASCII copy, created on Jun. 18, 2020, is named 126046-04220_SL.txt and is 876,737 bytes in size.

### BACKGROUND

The branched chain amino acids (BCAAs), e.g., leucine, isoleucine, and valine, play an important role in the metabolism of living organisms. Transamination of branched chain amino acids gives rise to their corresponding branched chain α-keto acids (BCKAs) (α-keto-β-methylvalerate, α-ketoisocaproate, and α-ketoisovalerate), which undergo further oxidative decarboxylation to produce acyl-CoA derivatives that enter the TCA cycle. Branched chain amino acids provide a nonspecific carbon source of oxidation for production of energy and also act as a precursor for muscle protein synthesis (Monirujjaman and Ferdouse, Advances in Molec. Biol., 2014, Article ID 36976, 6 pages, 2014).

Enzyme deficiencies or mutations which lead to the toxic accumulation of branched chain amino acids and their corresponding alpha-keto acids in the blood, cerebrospinal fluid, and tissues result in the development of metabolic disorders associated with the abnormal catabolism of branched chain amino acids in subjects, such as maple syrup urine disease (MSUD), isovaleric acidemia, propionic acidemia, methylmalonic acidemia, and diabetes ketoacidosis. Clinical manifestations of the disease vary depending on the degree of enzyme deficiency and include neurological dysfunction, seizures and death (Homanics et al. 2009).

Branched chain amino acids, such as leucine, or their corresponding alpha-keto acids, have also been linked to mTor activation (see, for example, Harlan et al., Cell Metabolism, 17:599-606, 2013) which is, in turn, associated with diseases such as cancer, obesity, type 2 diabetes, neurodegeneration, autism, Alzheimer's disease, Lymphangioleiomyomatosis (LAM), transplant rejection, glycogen storage disease, obesity, tuberous sclerosis, hypertension, cardiovascular disease, hypothalamic activation, musculoskeletal disease, Parkinson's disease, Huntington's disease, psoriasis, rheumatoid arthritis, lupus, multiple sclerosis, Leigh's syndrome, and Friedrich's ataxia (see Laplante and Sabatini, Cell, 149(2):74-293, 2012).

Currently available treatments for disorders involving the catabolism of branched chain amino acids are inadequate for the long term management of the disorders and have severe limitations (Svkvorak, J. Inherit. Metab. Dis., 32(2):229-246, 2009). A low protein/BCAA-restricted diet, with micronutrient and vitamin supplementation, as necessary, is the widely accepted long-term disease management strategy for many such disorders (Homanics et al., BMC Med. Genet., 7:33, 2006). However, BCAA-intake restrictions can be particularly problematic since branched chain amino acids can only be acquired through diet and are necessary for metabolic activities including protein synthesis and branched-chain fatty acid synthesis (Skvorak, 2009). Thus, even with proper monitoring and patient compliance, branched chain amino acid dietary restrictions result in a high incidence of mental retardation and mortality (Skvorak, 2009; Homanics et al., 2009). A few cases of MSUD have been treated by liver transplantation (Popescu and Dima, Liver Transpl., 1:22-28, 2012). However, the limited availability of donor organs, the costs associated with the transplantation itself, and the undesirable effects associated with continued immunosuppressant therapy limit the practicality of liver transplantation for treatment of disorders involving the catabolism of a branched chain amino acid (Homanics et al., 2012; Popescu and Dima, 2012). Therefore, there is significant unmet need for effective, reliable, and/or long-term treatment for disorders involving the catabolism of branched chain amino acids.

### SUMMARY

The present disclosure relates to compositions and therapeutic methods for reducing one or more excess branched chain amino acids, and/or an accumulated metabolite(s) thereof, for example, by converting the one or more excess branched chain amino acid(s) or accumulated metabolite(s) into alternate by product(s). In certain aspects, the disclosure relates to genetically engineered microorganisms, e.g., bacteria, yeast or viruses, that have been optimized to reduce one or more excess branched chain amino acids, and/or an accumulated metabolite(s) thereof, particularly in low-oxygen conditions, such as in the mammalian gut. In certain aspects, the compositions and methods disclosed herein may be used to treat disorders associated with excess branched chain amino acids and/or an accumulated metabolite(s) thereof, e.g., MSUD.

Specifically, the present disclosure provides recombinant microorganisms that have been engineered with optimized genetic circuitry, which allows the recombinant microorganism to import and/or metabolize one or more branched chain amino acid and/or one or more metabolite(s) thereof at an improved rate. In some embodiments, the engineered microorganism is capable of sensing a patient's internal environment, e.g., the gut, and responding by turning an engineered metabolic pathway on or off. When turned on, the engineered microorganism, e.g., bacterial, yeast or virus cell, expresses one or more enzymes in a metabolic pathway to achieve a therapeutic effect in a host subject.

In certain aspects, the present disclosure provides engineered bacterial cells, pharmaceutical compositions thereof, and methods of modulating BCAA(s) and/or metabolite(s) thereof and treating diseases associated with the catabolism of branched chain amino acids. Specifically, the engineered bacteria disclosed herein have been modified to comprise optimized gene sequence(s) encoding one or more enzymes involved in branched chain amino acid catabolism, as well as other circuitry, e.g., to regulate gene expression, including, for example, sequences for one or more inducible promoter(s), sequences for ribosome binding sites, sequences for importing one or more BCAA(s) and/or metabolite(s) thereof into the bacterial cell (e.g., transporter sequence(s)), sequences for the secretion or non-secretion of BCAA(s), metabolites or by-products (e.g., exporter(s) or exporter knockouts), and circuitry to guarantee the safety and non-colonization of the subject that is administered the recombinant bacteria, such as auxotrophies, kill switches, etc. These engineered bacteria are safe and well tolerated and augment the innate activities of the subject's microbiome to achieve a therapeutic effect.

In one aspect, disclosed herein is a recombinant bacterium capable of consuming leucine at a rate of at least about 0.5 μmol/10⁹ CFU/h in vitro. In one embodiment, the bacterium is capable of consuming leucine at a rate of at least about 0.75 μmol/10⁹ CFU/h. In one embodiment, the bacterium is capable of consuming leucine at a rate of at least about 1 μmol/10⁹ CFU/h. In one embodiment, the bacterium is capable of consuming leucine at a rate of at least about 1.25 μmol/10⁹ CFU/h. In one embodiment, the bacterium is capable of consuming leucine at a rate of at least about 1.5 μmol/10⁹ CFU/h. In one embodiment, the bacterium is capable of consuming leucine at a rate of at least about 1.75 μmol/10⁹ CFU/h. In one embodiment, the bacterium is capable of consuming leucine at a rate of at least about 2 μmol/10⁹ CFU/h. In one embodiment, the bacterium is capable of consuming leucine at a rate of at least about 2.25 μmol/10⁹ CFU/h. In one embodiment, the bacterium is capable of consuming leucine at a rate of at least about 2.5 μmol/10⁹ CFU/h. In one embodiment, the bacterium is capable of consuming leucine at a rate of at least about 2.75 μmol/10⁹ CFU/h.

In one embodiment, the bacterium is capable of consuming leucine at a rate of about 0.5 to about 2.75 μmol/10⁹ CFU/h. In one embodiment, the bacterium is capable of consuming leucine at a rate of about 0.75 to about 2.75 μmol/10⁹ CFU/h. In one embodiment, the bacterium is capable of consuming leucine at a rate of about 1 to about 2.75 μmol/10⁹ CFU/h. In one embodiment, the bacterium is capable of consuming leucine at a rate of about 1.25 to about 2.75 μmol/10⁹ CFU/h. In one embodiment, the bacterium is capable of consuming leucine at a rate of about 1.75 to about 2.75 μmol/10⁹ CFU/h. In one embodiment, the bacterium is capable of consuming leucine at a rate of about 2 to about 2.75 μmol/10⁹ CFU/h. In one embodiment, the bacterium is capable of consuming leucine at a rate of about 2.25 to about 2.75 μmol/10⁹ CFU/h. In one embodiment, the bacterium is capable of consuming leucine at a rate of about 2.5 to about 2.75 μmol/10⁹ CFU/h. In one embodiment, the bacterium is capable of consuming leucine at a rate of about 0.5 to about 2 μmol/10⁹ CFU/h. In one embodiment, the bacterium is capable of consuming leucine at a rate of about 1 to about 2 μmol/10⁹ CFU/h. In one embodiment, the bacterium is capable of consuming leucine at a rate of about 0.5 to about 2.5 μmol/10⁹ CFU/h.

In one embodiment, the bacterium comprises at least one gene sequence encoding at least one leucine catabolism enzyme operably linked to at least one directly or indirectly inducible promoter that is not associated with a gene encoding the at least one leucine catabolism enzyme in nature. In one embodiment, the promoter is selected from the group consisting of an FNRS promoter, a Pₜₑₜ promoter, a P_{cmt} promoter, an FNRS promoter, a P_{cl857} promoter, and a P_{BAD} promoter. In one embodiment, the at least one gene sequence encoding the at least one leucine catabolism enzyme is leuDH, kivD, and/or adh2.

In one embodiment, the bacterium further comprises at least one gene sequence encoding at least one transporter capable of transporting leucine. In one embodiment, the at least one gene sequence encoding the at least one transporter is brnQ.

In one embodiment, the bacterium comprises an operon, wherein the operon comprises leuDH, kivD, adh2, and brnQ. In one embodiment, the operon comprises a sequence having at least 90% identity to a sequence of any one of SEQ ID NOs: 25-48. In one embodiment, the operon comprises a sequence having at least 95% identity to a sequence of any one of SEQ ID NOs: 25-48. In one embodiment, the operon comprises a sequence comprising a sequence of any one of SEQ ID NOs: 25-48. In one embodiment, the operon comprises a sequence consisting of any one of SEQ ID NOs: 25-48.

In one embodiment, the leuDH gene encodes a LeuDH protein which comprises a sequence having at least 90% identity to a sequence of any one of SEQ ID NOs: 49-72. In one embodiment, the leuDH gene encodes a LeuDH protein which comprises a sequence having at least 95% identity to a sequence of any one of SEQ ID NOs: 49-72. In one embodiment, the leuDH gene encodes a LeuDH protein which comprises a sequence of any one of SEQ ID NOs: 49-72. In one embodiment, the leuDH gene encodes a LeuDH protein which consists of a sequence of any one of SEQ ID NOs: 49-72.

In one embodiment, the kivD gene encodes a KivD protein which comprises a sequence having at least 90% identity to a sequence of any one of SEQ ID NOs: 73-96. In one embodiment, the kivD gene encodes a KivD protein which comprises a sequence having at least 95% identity to a sequence of any one of SEQ ID NOs: 73-96. In one embodiment, the kivD gene encodes a KivD protein which comprises a sequence of any one of SEQ ID NOs: 73-96. In one embodiment, the kivD gene encodes a KivD protein which consists of a sequence of any one of SEQ ID NOs: 73-96.

In one embodiment, the adh2 gene encodes an Adh2 protein which comprises a sequence having at least 90% identity to a sequence of any one of SEQ ID NOs: 97-120. In one embodiment, the adh2 gene encodes an Adh2 protein which comprises a sequence having at least 95% identity to a sequence of any one of SEQ ID NOs: 97-120. In one embodiment, the adh2 gene encodes an Adh2 protein which comprises a sequence of any one of SEQ ID NOs: 97-120. In one embodiment, the adh2 gene encodes an Adh2 protein which consists of a sequence of any one of SEQ ID NOs: 97-120.

In one embodiment, the brnQ gene encodes a BrnQ protein which comprises a sequence having at least 90% identity to a sequence of any one of SEQ ID NOs: 121-144. In one embodiment, the brnQ gene encodes a BrnQ protein which comprises a sequence having at least 95% identity to a sequence of any one of SEQ ID NOs: 121-144. In one embodiment, the brnQ gene encodes a BrnQ protein which comprises a sequence of any one of SEQ ID NOs: 121-144. In one embodiment, the brnQ gene encodes a BrnQ protein which consists of a sequence of any one of SEQ ID NOs: 121-144.

In another aspect, disclosed herein is a recombinant bacterium comprising an operon, wherein the operon comprises an optimized leuDH gene which encodes a LeuDH protein, an optimized kivD gene which encodes a KivD protein, and an optimized adh2 gene which encodes an Adh2 protein, and an optimized brnQ gene which encodes a BrnQ protein, wherein the operon comprises a sequence having at least 90% identity to a sequence of any one of SEQ ID NOs: 25-48, and wherein the operon is operably linked to an inducible promoter that is not associated with a leuDH gene, a kivD gene, a adh2 gene, or a brnQ gene in nature.

In one embodiment, the operon comprises a sequence having at least 95% identity to a sequence of any one of SEQ ID NOs: 25-48. In one embodiment, the operon comprises a sequence of any one of SEQ ID NOs: 25-48. In one embodiment, the operon consists of a sequence of any one of SEQ ID NOs: 25-48.

In one embodiment, the LeuDH protein comprises a sequence having at least 90% identity to a sequence of any one of SEQ ID NOs: 49-72. In one embodiment, the LeuDH protein comprises a sequence having at least 95% identity to a sequence of any one of SEQ ID NOs: 49-72. In one embodiment, the LeuDH protein comprises a sequence of any one of SEQ ID NOs: 49-72. In one embodiment, the LeuDH protein consists of a sequence of any one of SEQ ID NOs: 49-72.

In one embodiment, the KivD protein comprises a sequence having at least 90% identity to a sequence of any one of SEQ ID NOs: 73-96. In one embodiment, the KivD protein comprises a sequence having at least 95% identity to a sequence of any one of SEQ ID NOs: 73-96. In one embodiment, the KivD protein comprises a sequence of any one of SEQ ID NOs: 73-96. In one embodiment, the KivD protein consists of a sequence of any one of SEQ ID NOs: 73-96.

In one embodiment, the Adh2 protein comprises a sequence having at least 90% identity to a sequence of any one of SEQ ID NOs: 97-120. In one embodiment, the Adh2 protein comprises a sequence having at least 95% identity to a sequence of any one of SEQ ID NOs: 97-120. In one embodiment, the Adh2 protein comprises a sequence of any one of SEQ ID NOs: 97-120. In one embodiment, the Adh2 protein comprises a sequence consisting of any one of SEQ ID NOs: 97-120.

In one embodiment, the BrnQ protein comprises a sequence having at least 90% identity to a sequence of any one of SEQ ID NOs: 121-144. In one embodiment, the BrnQ protein comprises a sequence having at least 95% identity to a sequence of any one of SEQ ID NOs: 121-144. In one embodiment, the BrnQ protein comprises a sequence of any one of SEQ ID NOs: 121-144. In one embodiment, the BrnQ protein consists of a sequence of any one of SEQ ID NOs: 121-144.

In one embodiment, the at least one gene sequence encoding the at least one leucine catabolism enzyme and the inducible promoter, are integrated into the chromosome of the bacterium. In one embodiment, the at least one gene sequence encoding the at least one leucine catabolism enzyme and the inducible promoter, are located in a plasmid in the bacterium. In another embodiment, the operon and the inducible promoter are integrated into the chromosome of the bacterium. In another embodiment, the operon and the inducible promoter are located in a plasmid in the bacterium.

In one embodiment, the promoter is selected from the group consisting of an FNRS promoter, a Pₜₑₜ promoter, a P_{cmt} promoter, an FNRS promoter, a P_{cl857} promoter, and a P_{BAD} promoter.

In one embodiment, the bacterium further comprises a second brnQ gene which encodes a second BrnQ protein, wherein the second brnQ gene is operably linked to an inducible promoter that is not associated with a brnQ gene in nature, and wherein the second brnQ gene is inserted into the malE/K or malP/T locus on a chromosome of the bacterium.

In one embodiment, the bacterium comprises a genetic modification in leuE that reduces leucine export from the bacterium. In one embodiment, the bacterium comprises a genetic modification in ilvC that reduces endogenous biosynthesis of leucine in the bacterium. In one embodiment, the bacterium further comprises a livKHMGF gene sequence encoding at least one transporter capable of transporting leucine. In one embodiment, the livKHMGF gene sequence is operably linked to at least one promoter that is not associated with a livKHMGF gene in nature.

In one embodiment, the bacterium is capable of consuming leucine at a rate of at least about 0.5 μmol/10⁹ CFU/h in vitro. In one embodiment, the bacterium is capable of consuming leucine at a rate of at least about 1 μmol/10⁹ CFU/h in vitro. In one embodiment, the bacterium is capable of consuming leucine at a rate of at least about 1.5 μmol/10⁹ CFU/h in vitro. In one embodiment, the bacterium is capable of consuming leucine at a rate of at least about 2.0 μmol/10⁹ CFU/h in vitro. In one embodiment, the bacterium is capable of consuming leucine at a rate of at least about 2.5 μmol/10⁹ CFU/h in vitro. In one embodiment, the bacterium is capable of consuming leucine at a rate of at least about 2.75 μmol/10⁹ CFU/h in vitro.

In one embodiment, the bacterium is capable of producing isopentanol at a rate of at least about 0.2 μmol/10⁹ CFU/h in vitro. In one embodiment, the bacterium is capable of producing isopentanol at a rate of at least about 0.3 μmol/10⁹ CFU/h in vitro. In one embodiment, the bacterium is capable of producing isopentanol at a rate of at least about 0.4 μmol/10⁹ CFU/h in vitro. In one embodiment, the bacterium is capable of producing isopentanol at a rate of at least about 0.5 μmol/10⁹ CFU/h in vitro. In one embodiment, the bacterium is capable of producing isopentanol at a rate of at least about 0.2 to 0.5 μmol/10⁹ CFU/h in vitro

In one embodiment, the bacterium exhibits preferentially consumes leucine over valine and isoleucine. In one embodiment, the bacterium exhibits a leucine/valine activity ratio of at least about 1.1 to at least about 2.75. In one embodiment, the bacterium exhibits a leucine/isoleucine activity ratio of at least about 1.1 to at least about 5.

In one embodiment, the bacterium is a probiotic bacterium selected from the group consisting of Bacteroides, Bifidobacterium, Clostridium, Escherichia, Lactobacillus, and Lactococcus. In one embodiment, the bacterium is Escherichia coli strain Nissle.

In one aspect, disclosed herein is a pharmaceutically acceptable composition comprising the recombinant bacterium of disclosed herein, and a pharmaceutically acceptable carrier.

In one aspect, disclosed herein is a method of reducing the level of leucine in a subject, the method comprising a step of administering to the subject a pharmaceutically acceptable composition.

In one aspect, disclosed herein is a method of treating a disease associated with excess leucine and/or a metabolic disorder involving the abnormal catabolism of leucine in a subject, the method comprising a step of administering to the subject a pharmaceutically acceptable composition.

In one embodiment, the subject has, or is suspected of having maple syrup urine disease (MSUD).

### BRIEF DESCRIPTION OF THE DRAWINGS

FIG. 1 depicts graphs showing sequence similar networks for LeuDH (FIG. 1A), KivD (FIG. 1B), and ADH (FIG. 1C) demonstrating diversity of each enzyme metagenomic library. Each spot in FIG. 1(A-C) represents a single amino acid sequence available in sequence databases, and the distance between spots demonstrating relatedness. Each sequence similarity network has a color key with information regarding the annotation or source of the enzyme. For LeuDH enzymes, color indicates the annotation of the enzyme. For KivD and Adh, the color of each spot represents the phylogenetic clade from which the enzyme was sourced. In each network, the seed enzyme sequence from the prototype pathway (SYN1980: comprising ΔleuE, ΔilvC, lacZ:tetR-Ptet-livKHMGF, tetR-Ptet-leuDH(Bc)-kivD-adh2-brnQ-rrnB ter (in low-copy pSC101 plasmid) is indicated by a red dot.

FIG. 2 depicts a graph showing the LeuDH enzyme activity measured in high throughput. The top 220 LeuDH enzymes from the primary screen were re-screened with increased biological replication (n=4) to validate enzyme activity. Activities are reported as fold-improvement over the B. cereus LeuDH activity.

FIG. 3 depicts a graph showing the activity specificity for Leu over Val and Ile among the top ˜200 LeuDH enzymes screened for the activity on Leu, Val, and Ile. Activity of LeuDH enzymes on Leu are reported relative the B. cereus LeuDH activity. Specificity was measured as the ratio of activity on Leu relative to Val/Leu. In the left panel, enzyme activity on Leu is reported relative to the Leu/Val specificity. In the right panel, enzyme activity is reported relative to the Leu/Ile specificity. Rationally engineered active site variants are shown in green. Sourced LeuDH enzymes are shown in blue-gray. The negative control is shown in gray. Enzymes exhibited up to ˜2.7-fold preference for Leu over Val, and up to a 5-fold preference for Leu over Ile. The positive control B. cereus LeuDH, shown in red, exhibited equal preference for Leu, Val, and Ile.

FIG. 4 depicts a graph showing the specificity comparison for Leu/Ile to Leu/Val among the top ˜200 LeuDH enzymes screened for the activity on Leu, Val, and Ile. Specificity was measured as the ratio of activity on Leu relative to Val/Leu. Rationally engineered active site variants are shown in green. Sourced LeuDH enzymes are shown in blue-gray. The negative control is shown in gray. The positive control B. cereus LeuDH, shown in red, showed equal preference for Leu, Val, and Ile when measured in this assay.

FIG. 5 depicts a graph showing the KivD enzyme activity measured in high throughput. The top 55 KivD enzymes from the primary screen were re-screened for activity with increased biological replication (n=4). Activities are reported as fold-improvement over the S. aureus KivD activity (which was equated to background).

FIG. 6 depicts a graph showing the Adh enzyme activity measured in high throughput. The top 55 KivD enzymes from the primary screen were re-screened for activity with increased biological replication (n=4). Activities are reported as fold-improvement over the S. cerevisiase ADH2 activity (which was equated to background).

FIG. 7 depicts a graph showing the consumption of Leu, Ile, and Val within the reaction mixture for each LeuDH enzyme. A total of 21 LeuDH enzymes were screened in cell lysate assays similar to the HTP screen, except that the reaction mixture contained Leu, Val, and Ile at 1:1:1 molar ratio. At least 10 LeuDH enzymes showed improved preference for Leu over Val and Ile when compared to the parent B. subtilis LeuDH. For nearly all LeuDH enzymes, least preference was shown for valine.

FIG. 8 depicts a graph showing a schematic view of the optimized pathways. Each selected pathway enzyme (6 LeuDH enzymes, 3 KivD enzymes, and 3 Adh enzymes) were each paired with 3 RBSs. The RBS-enzyme pairs were combined in a partial combinatorial library, maintaining the gene order of the prototype pathway and keeping the plasmid-encoded BmQ intact. In the schematic above, unique enzyme sequences for each family are indicated by a unique color. Unique RBSs are indicated with unique grayscale shading.

FIG. 9 depicts graphs showing pathways with optimized enzymes screened for Leu consumption (top panel) and isopentanol production (bottom panel, same strain order). Strains were assayed in biological triplicate and rank-ordered based on the best performance of any single replicate. The top ˜100 leucine consuming strains are shown here. The control strains are SYN1980 (comprising ΔleuE, ΔilvC, lacZ:tetR-Ptet-livKHMGF, tetR-Ptet-leuDH(Bc)-kivD-adh2-brnQ-rrnB ter (in low-copy pSC101 plasmid)), SYN1980 ΔbrnQ, and SYN1980 ΔleuDH.

FIG. 10 depicts graphs showing targeted metabolomics of top 10 Leu consuming strains demonstrating improved Leu consumption and relieved KivD bottleneck. Strains harboring the top 10 improved pathways identified in FIG. 9 were characterized for pathway intermediates in the Leu consuming assay. Reactions were sampled at 2-hour intervals over 4 hours.

FIG. 11 depicts a graph showing the leucine consumption activities of engineered strains SYN5721-SYN5744 optimized for BCAA consumption (comprising ΔleuE, ΔilvC, lacZ:tetR-Ptet-livKHMGF, pSC101-Ptet-optimized operon-brnQ-ampR). SYN5721-SYN5744 were compared against engineered strain SYN1980 (comprising ΔleuE, ΔilvC, lacZ:tetR-Ptet-livKHMGF, tetR-Ptet-leuDH(Bc)-kivD-adh2-brnQ-rrnB ter (in low-copy pSC101 plasmid). Strains were assayed by adding 4 mM leucine to minimum media and sampling at 0, 1, or 2 hour time points after anaerobic incubation.

FIG. 12 depicts a graph showing engineered strains generated with different host strains to demonstrate the host strain effects on leucine consumption activity. See Table 1 for detailed strain description. Strains were assayed by adding 8 mM leucine to minimum media and sampling at 0, 2, or 4 hour time points after anaerobic incubation.

FIG. 13 depicts a graph showing engineered strains generated with different inducible promoters operatively linked to the optimized operon to demonstrate the promoter effects on leucine consumption activity. See Table 1 for detailed strain description. Strains were assayed by adding 8 mM leucine to minimum media and sampling at 0, 1.5, or 3 hour time points after anaerobic incubation.

FIG. 14 depicts graphs showing the ability of engineered strain SYN5941 optimized for BCAA consumption (comprising pSC101-Pₜₑₜ-optimized operon-brnQ-ampR) to consume BCAA in the in vitro gastrointestinal simulation (IVS) model system. SYN5941 was compared against engineered strain SYN1980 (comprising ΔleuE, ΔilvC, lacZ:tetR-Ptet-livKHMGF, tetR-Ptet-leuDH(Bc)-kivD-adh2-brnQ-rrnB ter (in low-copy pSC101 plasmid) and the wild-type strain SYN001 (i.e., EcN). FIG. 14A depicts a higher ability for SYN5941 than SYN1980 and SYN001 to consume leucine (left panel) and produce isopentanol (right panel) in the presence of 5 mM leucine in IVS mimicking the low oxygen tention condition of the human upper gastrointestinal tract. FIG. 14B depicts a higher depicts higher abilities for SYN5941 (left panel) than SYN1980 (right panel) to consume leucine, isoleucine and valine in the presence of a mixture of leucine, isoleucine and valine each at 5 mM.

FIGS. 15A, 15B, and 15C depict graphs showing the ability of leucine consumption of engineered strain SYN5941 in various conditions. FIG. 15A, FIG. 15B, and FIG. 15C show leucine consumption in increasing concentrations of leucine, increasing pH, and increasing oxygen. For testing the leucine consumption rate under various substrate concentration, 0, 0.63, 1.25, 2.5, 5, 10, 15 or 20 mM of leucine was added into the reaction media. For testing the leucine consumption rate under various pH values, the reaction media was pH at 3, 4, 5, 6, or 7 when performed the assay. For testing the leucine consumption rate under various oxygen concentration, the reaction was carried out at 0, 7 or 21% oxygen by having the reaction in anaerobic chamber, microaerobic chamber or ambient.

FIG. 16 depicts graphs showing the ability of engineered strain SYN5941 optimized for BCAA consumption (comprising pSC101-Pₜₑₜ-optimized operon-brnQ-ampR) to decrease plasma BCAA levels in vivo in the intermediate MSUD (iMSUD) animal model fed with a high-protein diet, wherein the leucine levels were 1647±265.8 μM in iMSUD males (left panels) and 1165±151.9 μM in iMSUD females (right panels). SYN5941 was compared against engineered strain SYN1980 (comprising ΔleuE, ΔilvC, lacZ:tetR-Ptet-livKHMGF, tetR-Ptet-leuDH(Bc)-kivD-adh2-brnQ-rrnB ter (in low-copy pSC101 plasmid). FIG. 16A, FIG. 16B, and FIG. 16C show plasma leucine, valine and isoleucine concentrations on day 5 of the study. SYN5941 lowered the levels of leucine, isoleucine, and valine more significantly in males than SYN1980. Data were collected from 11 males and 15 females and presented as mean±SEM. Statistical significance determined as p<0.05 using one-way ANOVA followed by Tukey's multiple comparison test. *p<0.05.

FIG. 17 depicts graphs showing the ability of engineered strain SYN5941 optimized for BCAA consumption (comprising pSC101-Pₜₑₜ-optimized operon-brnQ-ampR) to decrease plasma BCAA levels in vivo in the intermediate MSUD (iMSUD) animal model fed with a high-protein diet, wherein the leucine levels were 2428±485.1 μM in iMSUD males (left panels) and 1626±150.1 μM in iMSUD females (right panels). SYN5941 was compared against engineered strain SYN1980 (comprising ΔleuE, ΔilvC, lacZ:tetR-Ptet-livKHMGF, tetR-Ptet-leuDH(Bc)-kivD-adh2-brnQ-rrnB ter (in low-copy pSC101 plasmid). FIG. 17A, FIG. 17B, and FIG. 17C show plasma leucine, valine and isoleucine concentrations on day 5 of the study. Data were collected from 11 males and 15 females and presented as mean±SEM. Statistical significance determined as p<0.05 using one-way ANOVA followed by Tukey's multiple comparison test. *p<0.05.

FIG. 18 depicts graphs showing the ability of engineered strain SYN5941 optimized for BCAA consumption (comprising pSC101-Pₜₑₜ-optimized operon-brnQ-ampR) to decrease plasma BCAA levels in vivo in healthy non-human primates. SYN5941 was compared against engineered strain SYN1980 (comprising ΔleuE, ΔilvC, lacZ:tetR-Ptet-livKHMGF, tetR-Ptet-leuDH(Bc)-kivD-adh2-brnQ-rrnB ter (in low-copy pSC101 plasmid). An orally administered vehicle (15% glycerol in PBS) served as control. FIG. 18A, FIG. 18B, and FIG. 18C show plasma leucine, valine and isoleucine concentrations as a function of time. FIG. 18D, FIG. 18E, and FIG. 18F show area under the curves. SYN5941 lowered the levels of leucine more significantly than SYN1980. Data collected from n=10-12 animals and presented as mean±SEM. Statistical significance determined as p<0.05 using two-way ANOVA (top) or one-way ANOVA (bottom) followed by Tukey's multiple comparison test.

FIG. 19 depicts a schematic view of the intermediate MSUD (iMSUD) animal model design.

FIG. 20 depicts a graph showing a comparison of the rate of Leu consumption over time between top Leu consuming strains (5941, 5942 and 5943) and a prototype strain (1980). 8 mM leucine was added to minimum media and samples were taken at 0, 2, and 4 hour time points after anaerobic incubation.

FIG. 21 depicts a graph showing extracellular profiles of the isopentanol pathway intermediates for SYN5941 assayed in Ambr15 bioreactors (n=2). Error bars reflect standard deviation across the duplicate bioreactors. The “Sum” shown in black bars represents the aggregate total concentration of the intermediates shown. Leu=Leucine, Acid=2-oxoisocaproate, Aldehyde=isovaleraldehyde, Alcohol=isopentanol.

### DETAILED DESCRIPTION

The disclosure includes optimized, engineered, and programmed microorganisms, e.g., bacteria, yeast, and viruses, pharmaceutical compositions thereof, and methods of modulating and treating disorders involving the catabolism of a branched chain amino acid, such as leucine, valine, and isoleucine. In some embodiments, the microorganism, e.g., bacterium, yeast, or virus, has been engineered to comprise heterologous gene sequence(s) encoding one or more branched chain amino acid catabolism enzyme(s), such as a leucine catabolism enzyme.

In some embodiments, the engineered microorganism, e.g., engineered bacterium, comprises optimized heterologous gene sequence(s) encoding one or more branched chain amino acid catabolism enzyme(s) and is capable of reducing the level of one or more branched chain amino acids and/or corresponding alpha-keto acid(s) and/or other corresponding metabolite(s). For example, the engineered bacterium, may comprise a BCAA transporter, such as LivKHMGF and/or BmQ. In some embodiments, the engineered bacterium comprises heterologous gene sequence(s) encoding one or more branched chain amino acid catabolism enzyme(s) and is capable of metabolizing one or more branched chain amino acids and/or corresponding alpha-keto acid(s) and/or other corresponding metabolite(s). In some embodiments, the engineered bacterium comprises heterologous gene sequence(s) encoding one or more branched chain amino acid catabolism enzyme(s) and is capable of transporting one or more branched chain amino acids and/or corresponding alpha-keto acid(s) and/or other corresponding metabolite(s) into the bacterium. In some embodiments, the engineered bacterium comprises heterologous gene sequence(s) encoding one or more branched chain amino acid catabolism enzyme(s) and is capable of reducing the level of and/or metabolizing one or more branched chain amino acids and/or corresponding alpha-keto acid(s) and/or other corresponding metabolite(s) in low-oxygen environments, e.g., the gut. In some embodiments, the engineered bacteria convert the branched chain amino acid(s) and/or corresponding alpha-keto acid(s) and/or other corresponding metabolite(s) to a non-toxic or low toxicity metabolite, e.g., isovaleraldehyde, isobutyraldehyde, 2-methylbutyraldehyde, isovaleric acid, isobutyric acid, 2-methylbutyric acid, isopentanol, isobutanol, and 2-methylbutanol. In some embodiments, the engineered bacterium comprises a genetic modification that reduces export of a branched chain amino acid from the bacterial cell, for example, the bacterial cell may comprise a knockout or knock-down of a gene that encodes a BCAA exporter, such as leuE (which encodes a leucine exporter). In some embodiments, the engineered bacterium comprises gene sequence(s) or gene cassette(s) encoding one or more transporters of a branched chain amino acid, which transporters function to import one or more BCAA(s) into the bacterial cell. In some embodiments, the bacterium has been engineered to comprise a genetic modification that reduces or inhibits endogenous production of one or more branched chain amino acids and/or one or more corresponding alpha-keto acids or other metabolite(s), for example, the bacterium may comprise a knockout or knock-down of a gene that encodes a molecule required for BCAA synthesis, such as ilvC (which encodes keto acid reductoisomerase). In some embodiments, the bacterium has been engineered to comprise an auxotroph, including, for example, a BCAA auxotrophy, such as ilvC (which is required for BCAA synthesis and requires the cell to import isoleucine and valine to survive) or other auxotrophy, as provided herein and known in the art, e.g., thyA auxotrophy. In some embodiments, the bacterium has been engineered to comprise a kill-switch, such as any of the kill-switches provided herein and known in the art. In some embodiments, the bacterium has been engineered to comprise antibiotic resistance, such as any of the antibiotic resistance provided herein and known in the art. In any of these embodiments, the gene sequence(s) encoding one or more branched chain amino acid catabolism enzyme(s), transporter(s), and other molecules can be integrated into the bacterial chromosome and/or can be present on a plasmid(s) (low copy and/or high copy). In any of these embodiments, the gene sequence(s) encoding one or more branched chain amino acid catabolism enzyme(s), transporter(s), and other molecules can be under the control of an inducible or constitutive promoter. Exemplary inducible promoters described herein include oxygen level-dependent promoters (e.g., FNR-inducible promoter), promoters induced by inflammation or an inflammatory response (RNS, ROS promoters), and promoters induced by a metabolite that may or may not be naturally present (e.g., can be exogenously added) in the gut, e.g., arabinose and tetracycline.

Thus, the recombinant bacterial cells and pharmaceutical compositions comprising the bacterial cells disclosed herein may be used to catabolize branched chain amino acids, e.g., leucine, isoleucine, valine, and/or their corresponding alpha-keto acid counterparts, to modify, ameliorate, treat and/or prevent conditions associated with disorders involving the catabolism of a branched chain amino acid. In one embodiment, the disorder involving the catabolism of a branched chain amino acid is a metabolic disorder involving the abnormal catabolism of a branched chain amino acid, including but not limited to maple syrup urine disease (MSUD), isovaleric acidemia, propionic acidemia, methylmalonic acidemia, or diabetes ketoacidosis, 3-MCC Deficiency, 3-Methylglutaconyl-CoA hydratase Deficiency, HMG-CoA Lyase Deficiency, Acetyl-CoA Carboxylase Deficiency, Malonyl-CoA Decarboxylase Deficiency, short-branched chain acylCoA dehydrogenase deficiency, 2-methyl-3-hydroxybutyric acidemia, beta-ketothiolase deficiency, isobutyryl-CoA dehydrogenase deficiency, HIBCH deficiency), and 3-Hydroxyisobutyric aciduria. In another embodiment, the disorder involving the catabolism of a branched chain amino acid is a disorder caused by the activation of mTor, for example, cancer, obesity, type 2 diabetes, neurodegeneration, autism, Alzheimer's disease, Lymphangioleiomyomatosis (LAM), transplant rejection, glycogen storage disease, obesity, tuberous sclerosis, hypertension, cardiovascular disease, hypothalamic activation, musculoskeletal disease, Parkinson's disease, Huntington's disease, psoriasis, rheumatoid arthritis, lupus, multiple sclerosis, Leigh's syndrome, and Friedrich's ataxia.

### Definitions

In order that the disclosure may be more readily understood, certain terms are first defined. These definitions should be read in light of the remainder of the disclosure and as understood by a person of ordinary skill in the art. Unless defined otherwise, all technical and scientific terms used herein have the same meaning as commonly understood by a person of ordinary skill in the art. Additional definitions are set forth throughout the detailed description.

As used herein, the term “recombinant microorganism” refers to a microorganism, e.g., bacterial, yeast or viral cell, or bacteria, yeast or virus, that has been genetically modified from its native state. Thus, a “recombinant bacterial cell” or “recombinant bacteria” refers to a bacterial cell or bacteria that have been genetically modified from their native state. For instance, a recombinant bacterial cell may have nucleotide insertions, nucleotide deletions, nucleotide rearrangements, and nucleotide modifications introduced into their DNA. These genetic modifications may be present in the chromosome of the bacteria or bacterial cell, or on a plasmid in the bacteria or bacterial cell. Recombinant bacterial cells disclosed herein may comprise exogenous nucleotide sequences on plasmids. Alternatively, recombinant bacterial cells may comprise exogenous nucleotide sequences stably incorporated into their chromosome.

A “programmed or engineered microorganism” refers to a microorganism, e.g., bacterial, yeast, or viral cell, or bacteria, yeast, or virus, that has been genetically modified from its native state to perform a specific function. Thus, a “programmed or engineered bacterial cell” or “programmed or engineered bacteria” refers to a bacterial cell or bacteria that has been genetically modified from its native state to perform a specific function, e.g., to metabolize a branched chain amino acid. In certain embodiments, the programmed or engineered bacterial cell has been modified to express one or more proteins, for example, one or more proteins that have a therapeutic activity or serve a therapeutic purpose. The programmed or engineered bacterial cell may additionally have the ability to stop growing or to destroy itself once the protein(s) of interest have been expressed.

As used herein, the term “gene” refers to a nucleic acid fragment that encodes a protein or fragment thereof, optionally including regulatory sequences preceding (5′ non-coding sequences) and following (3′ non-coding sequences) the coding sequence. In one embodiment, a “gene” does not include regulatory sequences preceding and following the coding sequence. A “native gene” refers to a gene as found in nature, optionally with its own regulatory sequences preceding and following the coding sequence. A “chimeric gene” refers to any gene that is not a native gene, optionally comprising regulatory sequences preceding and following the coding sequence, wherein the coding sequences and/or the regulatory sequences, in whole or in part, are not found together in nature. Thus, a chimeric gene may comprise regulatory sequences and coding sequences that are derived from different sources, or regulatory and coding sequences that are derived from the same source, but arranged differently than is found in nature.

As used herein, the term “gene sequence” is meant to refer to a genetic sequence, e.g., a nucleic acid sequence. The gene sequence or genetic sequence is meant to include a complete gene sequence or a partial gene sequence. The gene sequence or genetic sequence is meant to include sequence that encodes a protein or polypeptide and is also meant to include genetic sequence that does not encode a protein or polypeptide, e.g., a regulatory sequence, leader sequence, signal sequence, or other non-protein coding sequence.

As used herein, a “heterologous” gene or “heterologous sequence” refers to a nucleotide sequence that is not normally found in a given cell in nature. As used herein, a heterologous sequence encompasses a nucleic acid sequence that is exogenously introduced into a given cell and can be a native sequence (naturally found or expressed in the cell) or non-native sequence (not naturally found or expressed in the cell) and can be a natural or wild-type sequence or a variant, non-natural, or synthetic sequence. “Heterologous gene” includes a native gene, or fragment thereof, that has been introduced into the host cell in a form that is different from the corresponding native gene. For example, a heterologous gene may include a native coding sequence that is a portion of a chimeric gene to include non-native regulatory regions that is reintroduced into the host cell. A heterologous gene may also include a native gene, or fragment thereof, introduced into a non-native host cell. Thus, a heterologous gene may be foreign or native to the recipient cell; a nucleic acid sequence that is naturally found in a given cell but expresses an unnatural amount of the nucleic acid and/or the polypeptide which it encodes; and/or two or more nucleic acid sequences that are not found in the same relationship to each other in nature. As used herein, the term “endogenous gene” refers to a native gene in its natural location in the genome of an organism. As used herein, the term “transgene” refers to a gene that has been introduced into the host organism, e.g., host bacterial cell, genome.

As used herein, a “non-native” nucleic acid sequence refers to a nucleic acid sequence not normally present in a microorganism, e.g., an extra copy of an endogenous sequence, or a heterologous sequence such as a sequence from a different species, strain, or substrain of bacteria, yeast, or virus, or a sequence that is modified and/or mutated as compared to the unmodified sequence from bacteria, yeast, or virus of the same subtype. In some embodiments, the non-native nucleic acid sequence is a synthetic, non-naturally occurring sequence (see, e.g., Purcell et al., 2013). The non-native nucleic acid sequence may be a regulatory region, a promoter, a gene, and/or one or more genes in gene cassette. In some embodiments, “non-native” refers to two or more nucleic acid sequences that are not found in the same relationship to each other in nature. The non-native nucleic acid sequence may be present on a plasmid or chromosome. In some embodiments, the genetically engineered microorganism of the disclosure comprises a gene that is operably linked to a promoter that is not associated with said gene in nature. For example, in some embodiments, the genetically engineered bacteria disclosed herein comprise a gene that is operably linked to a directly or indirectly inducible promoter that is not associated with said gene in nature, e.g., an FNR responsive promoter (or other promoter disclosed herein) operably linked to a gene encoding a branched chain amino acid catabolism enzyme. In some embodiments, the genetically engineered yeast or virus of the disclosure comprises a gene that is operably linked to a directly or indirectly inducible promoter that is not associated with said gene in nature, e.g., a promoter operably linked to a gene encoding a branched chain amino acid catabolism enzyme.

As used herein, the term “coding region” refers to a nucleotide sequence that codes for a specific amino acid sequence. The term “regulatory sequence” refers to a nucleotide sequence located upstream (5′ non-coding sequences), within, or downstream (3′ non-coding sequences) of a coding sequence, and which influences the transcription, RNA processing, RNA stability, or translation of the associated coding sequence. Examples of regulatory sequences include, but are not limited to, promoters, translation leader sequences, effector binding sites, signal sequences, and stem-loop structures. In one embodiment, the regulatory sequence comprises a promoter, e.g., an FNR responsive promoter or another promoter disclosed herein.

“Operably linked” refers to the association of nucleic acid sequences on a single nucleic acid fragment so that the function of one is affected by the other. A regulatory element is operably linked with a coding sequence when it is capable of affecting the expression of the gene coding sequence, regardless of the distance between the regulatory element and the coding sequence. More specifically, operably linked refers to a nucleic acid sequence, e.g., a gene encoding a branched chain amino acid catabolism enzyme, that is joined to a regulatory sequence in a manner which allows expression of the nucleic acid sequence, e.g., the gene encoding the branched chain amino acid catabolism enzyme. In other words, the regulatory sequence acts in cis. In one embodiment, a gene may be “directly linked” to a regulatory sequence in a manner which allows expression of the gene. In another embodiment, a gene may be “indirectly linked” to a regulatory sequence in a manner which allows expression of the gene. In one embodiment, two or more genes may be directly or indirectly linked to a regulatory sequence in a manner which allows expression of the two or more genes. A regulatory region or sequence is a nucleic acid that can direct transcription of a gene of interest and may comprise promoter sequences, enhancer sequences, response elements, protein recognition sites, inducible elements, promoter control elements, protein binding sequences, 5′ and 3′ untranslated regions, transcriptional start sites, termination sequences, polyadenylation sequences, and introns.

A “promoter” as used herein, refers to a nucleotide sequence that is capable of controlling the expression of a coding sequence or gene. Promoters are generally located 5′ of the sequence that they regulate. Promoters may be derived in their entirety from a native gene, or be composed of different elements derived from promoters found in nature, and/or comprise synthetic nucleotide segments. Those skilled in the art will readily ascertain that different promoters may regulate expression of a coding sequence or gene in response to a particular stimulus, e.g., in a cell- or tissue-specific manner, in response to different environmental or physiological conditions, or in response to specific compounds. Prokaryotic promoters are typically classified into two classes: inducible and constitutive. A “constitutive promoter” refers to a promoter that allows for continual transcription of the coding sequence or gene under its control.

“Constitutive promoter” refers to a promoter that is capable of facilitating continuous transcription of a coding sequence or gene under its control and/or to which it is operably linked. Constitutive promoters and variants are well known in the art and include, but are not limited to, Ptac promoter, BBa_J23100, a constitutive Escherichia coli σ^{S} promoter (e.g., an osmY promoter (International Genetically Engineered Machine (iGEM) Registry of Standard Biological Parts Name BBa_J45992; BBa_J45993)), a constitutive Escherichia coli σ³² promoter (e.g., htpG heat shock promoter (BBa_J45504)), a constitutive Escherichia coli σ⁷⁰ promoter (e.g., lacq promoter (BBa_J54200; BBa_J56015), E. coli CreABCD phosphate sensing operon promoter (BBa_J64951), GlnRS promoter (BBa_K088007), lacZ promoter (BBa_K119000; BBa_K119001); M13K07 gene I promoter (BBa_M13101); M13K07 gene II promoter (BBa_M13102), M13K07 gene III promoter (BBa_M13103), M13K07 gene IV promoter (BBa_M13104), M13K07 gene V promoter (BBa_M13105), M13K07 gene VI promoter (BBa_M13106), M13K07 gene VIII promoter (BBa_M13108), M13110 (BBa_M13110)), a constitutive Bacillus subtilis σ^{A} promoter (e.g., promoter veg (BBa_K143013), promoter 43 (BBa_K143013), P_{liaG} (BBa_K823000), P_{lepA} (BBa_K823002), P_{veg} (BBa_K823003)), a constitutive Bacillus subtilis σ^{B} promoter (e.g., promoter ctc (BBa_K143010), promoter gsiB (BBa_K143011)), a Salmonella promoter (e.g., Pspv2 from Salmonella (BBa_K112706), Pspv from Salmonella (BBa_K112707)), a bacteriophage T7 promoter (e.g., T7 promoter (BBa I712074; BBa 1719005; BBa_J34814; BBa_J64997; BBa_K113010; BBa_K113011; BBa_K113012; BBa_R0085; BBa_R0180; BBa_R0181; BBa_R0182; BBa_R0183; BBa_Z0251; BBa_Z0252; BBa_Z0253)), and a bacteriophage SP6 promoter (e.g., SP6 promoter (BBa_J64998)).

An “inducible promoter” refers to a regulatory region that is operably linked to one or more genes, wherein expression of the gene(s) is increased in the presence of an inducer of said regulatory region. An “inducible promoter” refers to a promoter that initiates increased levels of transcription of the coding sequence or gene under its control in response to a stimulus or an exogenous environmental condition. A “directly inducible promoter” refers to a regulatory region, wherein the regulatory region is operably linked to a gene encoding a protein or polypeptide, where, in the presence of an inducer of said regulatory region, the protein or polypeptide is expressed. An “indirectly inducible promoter” refers to a regulatory system comprising two or more regulatory regions, for example, a first regulatory region that is operably linked to a first gene encoding a first protein, polypeptide, or factor, e.g., a transcriptional regulator, which is capable of regulating a second regulatory region that is operably linked to a second gene, the second regulatory region may be activated or repressed, thereby activating or repressing expression of the second gene. Both a directly inducible promoter and an indirectly inducible promoter are encompassed by “inducible promoter.” Exemplary inducible promoters described herein include oxygen level-dependent promoters (e.g., FNR-inducible promoter), promoters induced by inflammation or an inflammatory response (RNS, ROS promoters), and promoters induced by a metabolite that may or may not be naturally present (e.g., can be exogenously added) in the gut, e.g., arabinose and tetracycline. Examples of inducible promoters include, but are not limited to, an FNR responsive promoter, a P_{araC} promoter, a P_{araBAD} promoter, and a P_{TetR} promoter, each of which are described in more detail herein. Examples of other inducible promoters are provided herein below.

As used herein, “stably maintained” or “stable” bacterium is used to refer to a bacterial host cell carrying non-native genetic material, e.g., a gene encoding a branched chain amino acid catabolism enzyme, which is incorporated into the host genome or propagated on a self-replicating extra-chromosomal plasmid, such that the non-native genetic material is retained, expressed, and propagated. The stable bacterium is capable of survival and/or growth in vitro, e.g., in medium, and/or in vivo, e.g., in the gut. For example, the stable bacterium may be a genetically engineered bacterium comprising a gene encoding a branched chain amino acid catabolism enzyme, in which the plasmid or chromosome carrying the gene is stably maintained in the bacterium, such that branched chain amino acid catabolism enzyme can be expressed in the bacterium, and the bacterium is capable of survival and/or growth in vitro and/or in vivo. In some embodiments, copy number affects the stability of expression of the non-native genetic material. In some embodiments, copy number affects the level of expression of the non-native genetic material.

As used herein, the term “expression” refers to the transcription and stable accumulation of sense (mRNA) or anti-sense RNA derived from a nucleic acid, and/or to translation of an mRNA into a polypeptide.

As used herein, the term “plasmid” or “vector” refers to an extrachromosomal nucleic acid, e.g., DNA, construct that is not integrated into a bacterial cell's genome. Plasmids are usually circular and capable of autonomous replication. Plasmids may be low-copy, medium-copy, or high-copy, as is well known in the art. Plasmids may optionally comprise a selectable marker, such as an antibiotic resistance gene, which helps select for bacterial cells containing the plasmid and which ensures that the plasmid is retained in the bacterial cell. A plasmid disclosed herein may comprise a nucleic acid sequence encoding a heterologous gene, e.g., a gene encoding a branched chain amino acid catabolism enzyme.

As used herein, the term “transform” or “transformation” refers to the transfer of a nucleic acid fragment into a host bacterial cell, resulting in genetically-stable inheritance. Host bacterial cells comprising the transformed nucleic acid fragment are referred to as “recombinant” or “transgenic” or “transformed” organisms.

The term “genetic modification,” as used herein, refers to any genetic change. Exemplary genetic modifications include those that increase, decrease, or abolish the expression of a gene, including, for example, modifications of native chromosomal or extrachromosomal genetic material. Exemplary genetic modifications also include the introduction of at least one plasmid, modification, mutation, base deletion, base addition, base substitution, and/or codon modification of chromosomal or extrachromosomal genetic sequence(s), gene over-expression, gene amplification, gene suppression, promoter modification or substitution, gene addition (either single or multi-copy), antisense expression or suppression, or any other change to the genetic elements of a host cell, whether the change produces a change in phenotype or not. Genetic modification can include the introduction of a plasmid, e.g., a plasmid comprising a branched chain amino acid catabolism enzyme operably linked to a promoter, into a bacterial cell. Genetic modification can also involve a targeted replacement in the chromosome, e.g., to replace a native gene promoter with an inducible promoter, regulated promoter, strong promoter, or constitutive promoter. Genetic modification can also involve gene amplification, e.g., introduction of at least one additional copy of a native gene into the chromosome of the cell. Alternatively, chromosomal genetic modification can involve a genetic mutation.

As used herein, the term “genetic mutation” refers to a change or changes in a nucleotide sequence of a gene or related regulatory region that alters the nucleotide sequence as compared to its native or wild-type sequence. Mutations include, for example, substitutions, additions, and deletions, in whole or in part, within the wild-type sequence. Such substitutions, additions, or deletions can be single nucleotide changes (e.g., one or more point mutations), or can be two or more nucleotide changes, which may result in substantial changes to the sequence. Mutations can occur within the coding region of the gene as well as within the non-coding and regulatory sequence of the gene. The term “genetic mutation” is intended to include silent and conservative mutations within a coding region as well as changes which alter the amino acid sequence of the polypeptide encoded by the gene. A genetic mutation in a gene coding sequence may, for example, increase, decrease, or otherwise alter the activity (e.g., enzymatic activity) of the gene's polypeptide product. A genetic mutation in a regulatory sequence may increase, decrease, or otherwise alter the expression of sequences operably linked to the altered regulatory sequence.

Specifically, the term “genetic modification that reduces export of a branched chain amino acid from the bacterial cell” refers to a genetic modification that reduces the rate of export or quantity of export of a branched chain amino acid from the bacterial cell, as compared to the rate of export or quantity of export of the branched chain amino acid from a bacterial cell not having said modification, e.g., a wild-type bacterial cell. In one embodiment, a recombinant bacterial cell having a genetic modification that reduces export of a branched chain amino acid from the bacterial cell comprises a genetic mutation in a native gene, e.g., a leuE gene. In another embodiment, a recombinant bacterial cell having a genetic modification that reduces export of a branched chain amino acid from the bacterial cell comprises a genetic mutation in a native promoter, e.g., a leuE promoter, which reduces or inhibits transcription of the leuE gene. In another embodiment, a recombinant bacterial cell having a genetic modification that reduces export of a branched chain amino acid from the bacterial cell comprises a genetic mutation leading to overexpression of a repressor of an exporter of a branched chain amino acid. In another embodiment, a recombinant bacterial cell having a genetic modification that reduces export of a branched chain amino acid from the bacterial cell comprises a genetic mutation which reduces or inhibits translation of the gene encoding the exporter, e.g., the leuE gene.

Moreover, the term “genetic modification that increases import of a branched chain amino acid into the bacterial cell” refers to a genetic modification that increases the uptake rate or increases the uptake quantity of a branched chain amino acid into the cytosol of the bacterial cell, as compared to the uptake rate or uptake quantity of the branched chain amino acid into the cytosol of a bacterial cell not having said modification, e.g., a wild-type bacterial cell. In some embodiments, an engineered bacterial cell having a genetic modification that increases import of a branched chain amino acid into the bacterial cell refers to a bacterial cell comprising heterologous gene sequence (native or non-native) encoding one or more importer(s) (transporter(s)) of a branched chain amino acid. In some embodiments, the genetically engineered bacteria comprising genetic modification that increases import of a branched chain amino acid into the bacterial cell comprise gene sequence(s) encoding a BCAA transporter or other amino acid transporter that transports one or more BCAA(s) into the bacterial cell, for example a transporter that is capable of transporting leucine, valine, and/or isoleucine into a bacterial cell. The transporter can be any transporter that assists or allows import of a BCAA into the cell. In certain embodiments, the BCAA transporter is a leucine transporter, e.g., a high-affinity leucine transporter, e.g., LivKHMGF. In certain embodiments, the engineered bacterial cell contains gene sequence of one or more of livK, livH, livM, livG, and livF genes. In certain embodiments, the BCAA transporter is a BCAA transporter, e.g., a low affinity BCAA transporter, e.g., BmQ. In certain embodiments, the engineered bacterial cell contains gene sequence encoding brnQ gene. In some embodiments, the engineered bacteria comprise more than one copy of gene sequence encoding a BCAA transporter. In some embodiments, the engineered bacteria comprise gene sequence(s) encoding more than one BCAA transporter, e.g., two or more different BCAA transporters.

As used herein, the term “transporter” is meant to refer to a mechanism, e.g., protein, proteins, or protein complex, for importing a molecule, e.g., amino acid, peptide (di-peptide, tri-peptide, polypeptide, etc.), toxin, metabolite, substrate, as well as other biomolecules into the microorganism from the extracellular milieu.

As used herein, the term “polypeptide of interest” or “polypeptides of interest”, “protein of interest”, “proteins of interest”, “payload”, “payloads” includes, but is not limited to, any or a plurality of any of the branched chain amino acid catabolism enzymes, and/or branched chain amino acid transporters, and/or branched chain amino acid binding proteins and/or branched chain amino acid exporters described herein. As used herein, the term “gene of interest” or “gene sequence of interest” includes any or a plurality of any of the gene(s) an/or gene sequence(s) and or gene cassette(s) encoding one or more branched chain amino acid catabolism enzymes, ranched chain amino acid transporters, branched chain amino acid binding proteins, and branched chain amino acid exporters described herein.

The term “branched chain amino acid” or “BCAA,” as used herein, refers to an amino acid which comprises a branched side chain. Leucine, isoleucine, and valine are naturally occurring amino acids comprising a branched side chain. However, non-naturally occurring, usual, and/or modified amino acids comprising a branched side chain are also encompassed by the term branched chain amino acid.

Conversion of a branched chain amino acid to its corresponding alpha-keto acid is the first step in branched chain amino acid catabolism and is reversible when catalyzed by a leucine dehydrogenase (leuDH) or a branched chain amino acid aminotransferase (ilvE), or irreversible when catalyzed by an amino acid oxidase (also referred as amino acid deaminase, e.g., L-AAD). As used herein, the terms “alpha-keto acid” or “α-keto acid” refers to the molecules which are produced after deamination of a branched chain amino acid, and include the naturally occurring alpha-keto acids: α-ketoisocaproic acid (KIC) (also known as 4-methyl-2-oxopentanoate), α-ketoisovaleric acid (KIV) (also known as 2-oxoisopentanoate), and α-keto-beta-methylvaleric acid (KMV) (also known as 3-methyl-2-oxopentanoate). However, non-naturally occurring, unusual, or modified alpha-keto acids are also encompassed by the term “alpha-keto acid.”

Conversion of a branched chain alpha-keto acid to its corresponding branched acyl-CoA derivative is the second step in branched chain amino acid catabolism and is irreversible. As used herein, the term “acyl-CoA derivative” refers to the molecules which are produced after dehydrogenation of a branched chain alpha-keto acid, and include the naturally occurring acyl-CoA derivatives, propionyl-CoA and acetyl-CoA. However, non-naturally occurring, unusual, or modified acyl-CoA derivatives are also encompassed by the term “acyl-CoA derivative.”

In an alternative BCAA catabolism pathway, known as the “Ehrlich pathway”, a branched chain alpha-keto acid is irreversibly decarboxylated to its corresponding branched chain amino acid-derived aldehyde. This irreversible catabolic conversion of a branched chain alpha-keto acid, e.g., alpha-keto acids α-ketoisocaproic acid (KIC), α-ketoisovaleric acid (KIV), or α-keto-beta-methylvaleric acid (KMV) to its corresponding branched chain amino acid-derived aldehyde, e.g., isovaleraldehyde, isobutyraldehyde, or 2-methylbutyraldehyde, is catalyzed by ketoacid decarboxylase (KivD). As used herein, the term “branched chain amino acid-derived aldehyde” refers to the molecules which are produced after decarboxylation of a branched chain alpha-keto acid, and include the naturally occurring aldehydes isovaleraldehyde, 2-methyl butyraldehyde, and isobutyraldehyde. However, non-naturally occurring, unusual, or modified aldehydes are also encompassed by the term “aldehyde.” BCAA-derived aldehydes can then be converted to alcohols (e.g., isopentanol, isobutanol, 2-methylbutanol) by an alcohol dehydrogenase, e.g., Adh2 or Ygh.D. Alternatively, BCAA-derived aldehydes can be converted to their respective carboxylic acids (e.g., isovalerate, isobutyrate, and 2-methylbutyrate) by an aldehyde dehydrogenase, e.g., PadA.

As used herein, the phrase “exogenous environmental condition” or “exogenous environment signal” refers to settings, circumstances, stimuli, or biological molecules under which a promoter described herein is directly or indirectly induced. The phrase “exogenous environmental conditions” is meant to refer to the environmental conditions external to the engineered microorganism, but endogenous or native to the host subject environment. Thus, “exogenous” and “endogenous” may be used interchangeably to refer to environmental conditions in which the environmental conditions are endogenous to a mammalian body, but external or exogenous to an intact microorganism cell. In some embodiments, the exogenous environmental conditions are specific to the gut of a mammal. In some embodiments, the exogenous environmental conditions are specific to the upper gastrointestinal tract of a mammal. In some embodiments, the exogenous environmental conditions are specific to the lower gastrointestinal tract of a mammal. In some embodiments, the exogenous environmental conditions are specific to the small intestine of a mammal. In some embodiments, the exogenous environmental conditions are low-oxygen, microaerobic, or anaerobic conditions, such as the environment of the mammalian gut. In some embodiments, exogenous environmental conditions are molecules or metabolites that are specific to the mammalian gut, e.g., propionate. In some embodiments, the exogenous environmental condition is a tissue-specific or disease-specific metabolite or molecule(s). In some embodiments, the exogenous environmental condition is specific to a branched chain amino acid catabolic enzyme disease, e.g., MSUD. In some embodiments, the exogenous environmental condition is a low-pH environment. In some embodiments, the genetically engineered microorganism of the disclosure comprises a pH-dependent promoter. In some embodiments, the genetically engineered microorganism of the disclosure comprise an oxygen level-dependent promoter. In some aspects, bacteria have evolved transcription factors that are capable of sensing oxygen levels. Different signaling pathways may be triggered by different oxygen levels and occur with different kinetics. An “oxygen level-dependent promoter” or “oxygen level-dependent regulatory region” refers to a nucleic acid sequence to which one or more oxygen level-sensing transcription factors is capable of binding, wherein the binding and/or activation of the corresponding transcription factor activates downstream gene expression.

Examples of oxygen level-dependent transcription factors include, but are not limited to, FNR (fumarate and nitrate reductase), ANR, and DNR. Corresponding FNR-responsive promoters, ANR (anaerobic nitrate respiration)-responsive promoters, and DNR (dissimilatory nitrate respiration regulator)-responsive promoters are known in the art (see, e.g., Castiglione et al., 2009; Eiglmeier et al., 1989; Galimand et al., 1991; Hasegawa et al., 1998; Hoeren et al., 1993; Salmon et al., 2003), and non-limiting examples are shown in Table 1.

In a non-limiting example, a promoter (PfnrS) was derived from the E. coli Nissle fumarate and nitrate reductase gene S (fnrS) that is known to be highly expressed under conditions of low or no environmental oxygen (Durand and Storz, 2010; Boysen et al, 2010). The PfnrS promoter is activated under anaerobic conditions by the global transcriptional regulator FNR that is naturally found in Nissle. Under anaerobic conditions, FNR forms a dimer and binds to specific sequences in the promoters of specific genes under its control, thereby activating their expression. However, under aerobic conditions, oxygen reacts with iron-sulfur clusters in FNR dimers and converts them to an inactive form. In this way, the PfnrS inducible promoter is adopted to modulate the expression of proteins or RNA. PfnrS is used interchangeably in this application as FNRS, fnrs, FNR, P-FNRS promoter and other such related designations to indicate the promoter PfnrS.

TABLE 1
Examples of transcription factors and
responsive genes and regulatory regions

| | Examples of responsive genes, promoters, |
|---|---|
| Transcription Factor | and/or regulatory regions: |
| FNR | nirB, ydfZ, pdhR, focA, ndH, hlyE, narK, |
| | narX, narG, yfiD, tdcD |
| ANR | arcDABC |
| DNR | norb, norC |

In some embodiments, the exogenous environmental conditions are the presence or absence of reactive oxygen species (ROS). In other embodiments, the exogenous environmental conditions are the presence or absence of reactive nitrogen species (RNS). In some embodiments, exogenous environmental conditions are biological molecules that are involved in the inflammatory response, for example, molecules present in an inflammatory disorder of the gut. In some embodiments, the exogenous environmental conditions or signals exist naturally or are naturally absent in the environment in which the recombinant bacterial cell resides. In some embodiments, the exogenous environmental conditions or signals are artificially created, for example, by the creation or removal of biological conditions and/or the administration or removal of biological molecules.

In some embodiments, the exogenous environmental condition(s) and/or signal(s) stimulates the activity of an inducible promoter. In some embodiments, the exogenous environmental condition(s) and/or signal(s) that serves to activate the inducible promoter is not naturally present within the gut of a mammal. In some embodiments, the inducible promoter is stimulated by a molecule or metabolite that is administered in combination with the pharmaceutical composition of the disclosure, for example, tetracycline, arabinose, or any biological molecule that serves to activate an inducible promoter. In some embodiments, the exogenous environmental condition(s) and/or signal(s) is added to culture media comprising a recombinant bacterial cell of the disclosure. In some embodiments, the exogenous environmental condition that serves to activate the inducible promoter is naturally present within the gut of a mammal (for example, low oxygen or anaerobic conditions, or biological molecules involved in an inflammatory response). In some embodiments, the loss of exposure to an exogenous environmental condition (for example, in vivo) inhibits the activity of an inducible promoter, as the exogenous environmental condition is not present to induce the promoter (for example, an aerobic environment outside the gut). “Gut” refers to the organs, glands, tracts, and systems that are responsible for the transfer and digestion of food, absorption of nutrients, and excretion of waste. In humans, the gut comprises the gastrointestinal (GI) tract, which starts at the mouth and ends at the anus, and additionally comprises the esophagus, stomach, small intestine, and large intestine. The gut also comprises accessory organs and glands, such as the spleen, liver, gallbladder, and pancreas. The upper gastrointestinal tract comprises the esophagus, stomach, and duodenum of the small intestine. The lower gastrointestinal tract comprises the remainder of the small intestine, i.e., the jejunum and ileum, and all of the large intestine, i.e., the cecum, colon, rectum, and anal canal. Bacteria can be found throughout the gut, e.g., in the gastrointestinal tract, and particularly in the intestines.

As used herein, the term “low oxygen” is meant to refer to a level, amount, or concentration of oxygen (O₂) that is lower than the level, amount, or concentration of oxygen that is present in the atmosphere (e.g., <21% O₂; <160 torr O₂)). Thus, the term “low oxygen condition or conditions” or “low oxygen environment” refers to conditions or environments containing lower levels of oxygen than are present in the atmosphere. In some embodiments, the term “low oxygen” is meant to refer to the level, amount, or concentration of oxygen (O₂) found in a mammalian gut, e.g., lumen, stomach, small intestine, duodenum, jejunum, ileum, large intestine, cecum, colon, distal sigmoid colon, rectum, and anal canal. In some embodiments, the term “low oxygen” is meant to refer to a level, amount, or concentration of O₂ that is 0-60 mmHg O₂ (0-60 torr 02) (e.g., 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, and 60 mmHg 02), including any and all incremental fraction(s) thereof (e.g., 0.2 mmHg, 0.5 mmHg O₂, 0.75 mmHg O₂, 1.25 mmHg O₂, 2.175 mmHg O₂, 3.45 mmHg O₂, 3.75 mmHg O₂, 4.5 mmHg O₂, 6.8 mmHg O₂, 11.35 mmHg 02, 46.3 mmHg O₂, 58.75 mmHg, etc., which exemplary fractions are listed here for illustrative purposes and not meant to be limiting in any way). In some embodiments, “low oxygen” refers to about 60 mmHg O₂ or less (e.g., 0 to about 60 mmHg O₂). The term “low oxygen” may also refer to a range of O₂ levels, amounts, or concentrations between 0-60 mmHg O₂ (inclusive), e.g., 0-5 mmHg O₂, <1.5 mmHg O₂, 6-10 mmHg, <8 mmHg, 47-60 mmHg, etc. which listed exemplary ranges are listed here for illustrative purposes and not meant to be limiting in any way. See, for example, Albenberg et al., Gastroenterology, 147(5): 1055-1063 (2014); Bergofsky et al., J Clin. Invest., 41(11): 1971-1980 (1962); Crompton et al., J Exp. Biol., 43: 473-478 (1965); He et al., PNAS (USA), 96: 4586-4591 (1999); McKeown, Br. J. Radiol., 87:20130676 (2014) (doi: 10.1259/brj.20130676), each of which discusses the oxygen levels found in the mammalian gut of various species and each of which are incorporated by reference herewith in their entireties. In some embodiments, the term “low oxygen” is meant to refer to the level, amount, or concentration of oxygen (O₂) found in a mammalian organ or tissue other than the gut, e.g., urogenital tract, tumor tissue, etc. in which oxygen is present at a reduced level, e.g., at a hypoxic or anoxic level. In some embodiments, “low oxygen” is meant to refer to the level, amount, or concentration of oxygen (O₂) present in partially aerobic, semi aerobic, microaerobic, nanoaerobic, microoxic, hypoxic, anoxic, and/or anaerobic conditions. For example, Table 2 summarizes the amount of oxygen present in various organs and tissues. In some embodiments, the level, amount, or concentration of oxygen (O₂) is expressed as the amount of dissolved oxygen (“DO”) which refers to the level of free, non-compound oxygen (O₂) present in liquids and is typically reported in milligrams per liter (mg/L), parts per million (ppm; 1 mg/L=1 ppm), or in micromoles (umole) (1 umole O₂=0.022391 mg/L O₂). Fondriest Environmental, Inc., “Dissolved Oxygen”, Fundamentals of Environmental Measurements, 19 Nov. 2013. In some embodiments, the term “low oxygen” is meant to refer to a level, amount, or concentration of oxygen (O₂) that is about 6.0 mg/L DO or less, e.g., 6.0 mg/L, 5.0 mg/L, 4.0 mg/L, 3.0 mg/L, 2.0 mg/L, 1.0 mg/L, or 0 mg/L, and any fraction therein, e.g., 3.25 mg/L, 2.5 mg/L, 1.75 mg/L, 1.5 mg/L, 1.25 mg/L, 0.9 mg/L, 0.8 mg/L, 0.7 mg/L, 0.6 mg/L, 0.5 mg/L, 0.4 mg/L, 0.3 mg/L, 0.2 mg/L and 0.1 mg/L DO, which exemplary fractions are listed here for illustrative purposes and not meant to be limiting in any way. The level of oxygen in a liquid or solution may also be reported as a percentage of air saturation or as a percentage of oxygen saturation (the ratio of the concentration of dissolved oxygen (O₂) in the solution to the maximum amount of oxygen that will dissolve in the solution at a certain temperature, pressure, and salinity under stable equilibrium). Well-aerated solutions (e.g., solutions subjected to mixing and/or stirring) without oxygen producers or consumers are 100% air saturated. In some embodiments, the term “low oxygen” is meant to refer to 40% air saturation or less, e.g., 40%, 39%, 38%, 37%, 36%, 35%, 34%, 33%, 32%, 31%, 30%, 29%, 28%, 27%, 26%, 25%, 24%, 23%, 22%, 21%, 20%, 19%, 18%, 17%, 16%, 15%, 14%, 13%, 12%, 11%, 10%, 9%, 8%, 7%, 6%, 5%, 4%, 3%, 2%, 1%, and 0% air saturation, including any and all incremental fraction(s) thereof (e.g., 30.25%, 22.70%, 15.5%, 7.7%, 5.0%, 2.8%, 2.0%, 1.65%, 1.0%, 0.9%, 0.8%, 0.75%, 0.68%, 0.5%. 0.44%, 0.3%, 0.25%, 0.2%, 0.1%, 0.08%, 0.075%, 0.058%, 0.04%. 0.032%, 0.025%, 0.01%, etc.) and any range of air saturation levels between 0-40%, inclusive (e.g., 0-5%, 0.05-0.1%, 0.1-0.2%, 0.1-0.5%, 0.5-2.0%, 0-10%, 5-10%, 10-15%, 15-20%, 20-25%, 25-30%, etc.). The exemplary fractions and ranges listed here are for illustrative purposes and not meant to be limiting in any way. In some embodiments, the term “low oxygen” is meant to refer to 9% O₂ saturation or less, e.g., 9%, 8%, 7%, 6%, 5%, 4%, 3%, 2%, 1%, 0%, O₂ saturation, including any and all incremental fraction(s) thereof (e.g., 6.5%, 5.0%, 2.2%, 1.7%, 1.4%, 0.9%, 0.8%, 0.75%, 0.68%, 0.5%. 0.44%, 0.3%, 0.25%, 0.2%, 0.1%, 0.08%, 0.075%, 0.058%, 0.04%. 0.032%, 0.025%, 0.01%, etc.) and any range of O₂ saturation levels between 0-9%, inclusive (e.g., 0-5%, 0.05-0.1%, 0.1-0.2%, 0.1-0.5%, 0.5-2.0%, 0-8%, 5-7%, 0.3-4.2% O₂, etc.). The exemplary fractions and ranges listed here are for illustrative purposes and not meant to be limiting in any way.

TABLE 2

| Compartment | Oxygen Tension |
|---|---|
| stomach | ~60 torr (e.g., 58 +/− 15 torr) |
| duodenum and first | ~30 torr (e.g., 32 +/− 8 torr); ~20% oxygen in |
| part of jejunum | ambient air |
| Ileum (mid- small | ~10 torr; ~6% oxygen in ambient air (e.g., 11 +/− |
| intestine) | 3 torr) |
| Distal sigmoid colon | ~3 torr (e.g., 3 +/− 1 torr) |
| colon | <2 torr |
| Lumen of cecum | <1 torr |
| tumor | <32 torr (most tumors are <15 torr) |

“Microorganism” refers to an organism or microbe of microscopic, submicroscopic, or ultramicroscopic size that typically consists of a single cell. Examples of microorganisms include bacteria, viruses, parasites, fungi, certain algae, yeast, and protozoa. In some aspects, the microorganism is engineered (“engineered microorganism”) to produce one or more therapeutic molecules, e.g., lysosomal enzyme(s). In certain embodiments, the engineered microorganism is an engineered bacterium. In certain embodiments, the engineered microorganism is an engineered yeast or virus.

“Non-pathogenic bacteria” refer to bacteria that are not capable of causing disease or harmful responses in a host. In some embodiments, non-pathogenic bacteria are Gram-negative bacteria. In some embodiments, non-pathogenic bacteria are Gram-positive bacteria. In some embodiments, non-pathogenic bacteria do not contain lipopolysaccharides (LPS). In some embodiments, non-pathogenic bacteria are commensal bacteria. Examples of non-pathogenic bacteria include, but are not limited to certain strains belonging to the genus Bacillus, Bacteroides, Bifidobacterium, Brevibacteria, Clostridium, Enterococcus, Escherichia coli, Lactobacillus, Lactococcus, Saccharomyces, and Staphylococcus, e.g., Bacillus coagulans, Bacillus subtilis, Bacteroides fragilis, Bacteroides subtilis, Bacteroides thetaiotaomicron, Bifidobacterium bifidum, Bifidobacterium infantis, Bifidobacterium lactis, Bifidobacterium longum, Clostridium butyricum, Enterococcus faecium, Escherichia coli, Escherichia coli Nissle, Lactobacillus acidophilus, Lactobacillus bulgaricus, Lactobacillus casei, Lactobacillus johnsonii, Lactobacillus paracasei, Lactobacillus plantarum, Lactobacillus reuteri, Lactobacillus rhamnosus, Lactococcus lactis and Saccharomyces boulardii (Sonnenborn et al., 2009; Dinleyici et al., 2014; U.S. Pat. Nos. 6,835,376; 6,203,797; 5,589,168; 7,731,976). Non-pathogenic bacteria also include commensal bacteria, which are present in the indigenous microbiota of the gut. In one embodiment, the disclosure further includes non-pathogenic Saccharomyces, such as Saccharomyces boulardii. Naturally pathogenic bacteria may be genetically engineered to reduce or eliminate pathogenicity.

“Probiotic” is used to refer to live, non-pathogenic microorganisms, e.g., bacteria, which can confer health benefits to a host organism that contains an appropriate amount of the microorganism. In some embodiments, the host organism is a mammal. In some embodiments, the host organism is a human. In some embodiments, the probiotic bacteria are Gram-negative bacteria. In some embodiments, the probiotic bacteria are Gram-positive bacteria. Some species, strains, and/or subtypes of non-pathogenic bacteria are currently recognized as probiotic bacteria. Examples of probiotic bacteria include, but are not limited to, certain strains belonging to the genus Bifidobacteria, Escherichia Coli, Lactobacillus, and Saccharomyces e.g., Bifidobacterium bifidum, Enterococcus faecium, Escherichia coli strain Nissle, Lactobacillus acidophilus, Lactobacillus bulgaricus, Lactobacillus paracasei, and Lactobacillus plantarum, and Saccharomyces boulardii (Dinleyici et al., 2014; U.S. Pat. Nos. 5,589,168; 6,203,797; 6,835,376). The probiotic may be a variant or a mutant strain of bacterium (Arthur et al., 2012; Cuevas-Ramos et al., 2010; Olier et al., 2012; Nougayrede et al., 2006). Non-pathogenic bacteria may be genetically engineered to enhance or improve desired biological properties, e.g., survivability. Non-pathogenic bacteria may be genetically engineered to provide probiotic properties. Probiotic bacteria may be genetically engineered to enhance or improve probiotic properties.

As used herein, the term “auxotroph” or “auxotrophic” refers to an organism that requires a specific factor, e.g., an amino acid, a sugar, or other nutrient) to support its growth. An “auxotrophic modification” is a genetic modification that causes the organism to die in the absence of an exogenously added nutrient essential for survival or growth because it is unable to produce said nutrient. As used herein, the term “essential gene” refers to a gene which is necessary to for cell growth and/or survival. Essential genes are described in more detail infra and include, but are not limited to, DNA synthesis genes (such as thyA), cell wall synthesis genes (such as dapA), and amino acid genes (such as serA and metA).

As used herein, the terms “modulate” and “treat” and their cognates refer to an amelioration of a disease, disorder, and/or condition, or at least one discernible symptom thereof. In another embodiment, “modulate” and “treat” refer to an amelioration of at least one measurable physical parameter, not necessarily discernible by the patient. In another embodiment, “modulate” and “treat” refer to inhibiting the progression of a disease, disorder, and/or condition, either physically (e.g., stabilization of a discernible symptom), physiologically (e.g., stabilization of a physical parameter), or both. In another embodiment, “modulate” and “treat” refer to slowing the progression or reversing the progression of a disease, disorder, and/or condition. As used herein, “prevent” and its cognates refer to delaying the onset or reducing the risk of acquiring a given disease, disorder and/or condition or a symptom associated with such disease, disorder, and/or condition.

Those in need of treatment may include individuals already having a particular medical disease, as well as those at risk of having, or who may ultimately acquire the disease. The need for treatment is assessed, for example, by the presence of one or more risk factors associated with the development of a disease, the presence or progression of a disease, or likely receptiveness to treatment of a subject having the disease. Diseases associated with the catabolism of a branched chain amino acid, e.g., MSUD, may be caused by inborn genetic mutations for which there are no known cures. Diseases can also be secondary to other conditions, e.g., liver diseases. Treating diseases involving the catabolism of a branched chain amino acid, such as MSUD, may encompass reducing normal levels of branched chain amino acids, reducing excess levels of branched chain amino acids, or eliminating branched chain amino acids, e.g., leucine, and does not necessarily encompass the elimination of the underlying disease.

As used herein, the term “catabolism” refers to the breakdown of a molecule into a smaller unit. As used herein, the term “branched chain amino acid catabolism” refers to the conversion of a branched chain amino acid, such as leucine, isoleucine, or valine, into a corresponding metabolite, for example a corresponding alpha keto acid, acyl-CoA derivative, aldehyde, alcohol, or other metabolite, such as any of the BCAA metabolites disclosed herein; or the conversion of a branched chain alpha keto acid into its corresponding acyl-CoA derivative, aldehydes, alcohols or other metabolites, such as any of the BCAA metabolites disclosed herein. The “branched chain amino acid catabolism” refers to both native and non-native conversion of a branched chain amino acid, such as leucine, isoleucine, or valine, into a corresponding metabolite. Thus, the term additionally covers catabolism of BCAA that may not occur in nature and is artificially induced as a result of genetic engineering. In one embodiment, “abnormal catabolism” refers to a decrease in the rate or the level of conversion of a branched chain amino acid or its corresponding alpha-keto acid to a corresponding metabolite, leading to the accumulation of the branched chain amino acid, accumulation of the branched chain alpha-keto acid, and/or accumulation of a BCAA metabolite that is toxic or that accumulates to a toxic level in a subject. In one embodiment, the branched chain amino acid, branched chain alpha-keto acid, or other metabolite thereof, accumulates to a toxic level in the blood or the brain of a subject, leading to the development of a disease or disorder associated with the abnormal catabolism of the branched chain amino acid in the subject. In one embodiment, “abnormal leucine catabolism” refers to a level of greater than 4 mg/dL of leucine in the plasma of a subject. In another embodiment, “normal leucine catabolism” refers to a level of less than 4 mg/dL of leucine in the plasma of a subject.

As used herein, the term “disorder involving the abnormal catabolism of a branched amino acid” or “disease involving the abnormal catabolism of a branched amino acid” or “branched chain amino acid disease” or “disease associated with excess branched chain amino acid” refers to a disease or disorder wherein the catabolism of a branched chain amino acid or a branched chain alpha-keto acid is abnormal. Such diseases are genetic disorders that result from deficiency in at least one of the enzymes required to catabolize a branched chain amino acid, e.g., leucine, isoleucine, or valine. As a result, individuals suffering from branched chain amino acid disease have accumulated branched chain amino acids in their cells and tissues. Examples of branched chain amino acid diseases include, but are not limited to, MSUD, isovaleric acidemia, 3-MCC deficiency, 3-methylglutaconyl-CoA hydrolase deficiency, HMG-CoA lysate deficiency, Acetyl CoA carboxylase deficiency, malonylCoA decarboxylase deficiency, short branched chain acyl-CoA dehydrogenase, 2-methyl-3-hydroxybutyric acidemia, beta-ketothiolase deficiency, isobutyl-CoA dehydrogenase deficiency, HIBCH deficiency, 3-hydroxyisobutyric aciduria, proprionic acidemis, methylmalonic acidemia, as well as those diseases resulting from mTor activation, including but not limited to cancer, obesity, type 2 diabetes, neurodegeneration, autism, Alzheimer's disease, Lymphangioleiomyomatosis (LAM), transplant rejection, glycogen storage disease, obesity, tuberous sclerosis, hypertension, cardiovascular disease, hypothalamic activation, musculoskeletal disease, Parkinson's disease, Huntington's disease, psoriasis, rheumatoid arthritis, lupus, multiple sclerosis, Leigh's syndrome, and Friedrich's ataxia. In one embodiment, a “disease or disorder involving the catabolism of a branched chain amino acid” is a metabolic disease or disorder involving the abnormal catabolism of a branched chain amino acid. In another embodiment, a “disease or disorder involving the catabolism of a branched chain amino acid” is a disease or disorder caused by the activation of mTor. In one embodiment, the activation of mTor is abnormal.

In one embodiment, “abnormal catabolism” refers to a decrease in the rate or the level of conversion of the branched chain amino acid or its alpha-keto acid counterpart, leading to the accumulation of the branched chain amino acid or alpha-keto acid in a subject. In one embodiment, accumulation of the branched chain amino acid in the blood or the brain of a subject becomes toxic and leads to the development of a disease or disorder associated with the abnormal catabolism of the branched chain amino acid in the subject.

As used herein, the term “disease caused by the activation of mTor” or “disorder caused by the activation of mTor” refers to a disease or a disorder wherein the levels of branched chain amino acid may be normal, and wherein the branched chain amino acid causes the activation of mTor at a level higher than the normal level of mTor activity. In another embodiment, the subject having a disorder caused by the activation of mTor may have higher levels of a branched chain amino acid than normal. Diseases caused by the activation of mTor are known in the art. See, for example, Laplante and Sabatini, Cell, 149(2):74-293, 2012. As used herein, the term “disease caused by the activation of mTor” includes cancer, obesity, type 2 diabetes, neurodegeneration, autism, Alzheimer's disease, Lymphangioleiomyomatosis (LAM), transplant rejection, glycogen storage disease, obesity, tuberous sclerosis, hypertension, cardiovascular disease, hypothalamic activation, musculoskeletal disease, Parkinson's disease, Huntington's disease, psoriasis, rheumatoid arthritis, lupus, multiple sclerosis, Leigh's syndrome, and Friedrich's ataxia. In one embodiment, the subject has normal levels of a branched chain amino acid, such as leucine, before administration of the engineered bacteria of the present disclosure. In another embodiment, the subject has decreased levels of the branched chain amino acid after the administration of the engineered bacteria of the present disclosure, thereby decreasing the levels of mTor or the activity of mTor, thereby treating the disorder in the subject. In one embodiment, the pharmaceutical composition disclosed herein decreases the activity of mTor by at least about 2-fold, 3-fold, 4-fold, or 5-fold in the subject.

As used herein, the term “anabolism” refers the conversion of a branched chain alpha-keto acid or an acyl-CoA derivative or other metabolite into its corresponding branched chain amino acid, such as leucine, isoleucine, or valine or alpha-keto acid, respectively.

As used herein a “pharmaceutical composition” refers to a preparation of genetically engineered microorganism of the disclosure, e.g., genetically engineered bacteria yeast or virus, with other components such as a physiologically suitable carrier and/or excipient.

The phrases “physiologically acceptable carrier” and “pharmaceutically acceptable carrier” which may be used interchangeably refer to a carrier or a diluent that does not cause significant irritation to an organism and does not abrogate the biological activity and properties of the administered bacterial or viral compound. An adjuvant is included under these phrases.

The term “excipient” refers to an inert substance added to a pharmaceutical composition to further facilitate administration of an active ingredient. Examples include, but are not limited to, calcium bicarbonate, sodium bicarbonate calcium phosphate, various sugars and types of starch, cellulose derivatives, gelatin, vegetable oils, polyethylene glycols, and surfactants, including, for example, polysorbate 20.

The terms “therapeutically effective dose” and “therapeutically effective amount” are used to refer to an amount of a compound that results in prevention, delay of onset of symptoms, or amelioration of symptoms of a condition, e.g., lysosomal storage disease (LSD). A therapeutically effective amount may, for example, be sufficient to treat, prevent, reduce the severity, delay the onset, and/or reduce the risk of occurrence of one or more symptoms of a disorder associated with lysosomal storage disease. A therapeutically effective amount, as well as a therapeutically effective frequency of administration, can be determined by methods known in the art and discussed below.

As used herein, the term “bacteriostatic” or “cytostatic” refers to a molecule or protein which is capable of arresting, retarding, or inhibiting the growth, division, multiplication or replication of recombinant bacterial cell of the disclosure.

As used herein, the term “bactericidal” refers to a molecule or protein which is capable of killing the recombinant bacterial cell of the disclosure.

As used herein, the term “toxin” refers to a protein, enzyme, or polypeptide fragment thereof, or other molecule which is capable of arresting, retarding, or inhibiting the growth, division, multiplication or replication of the recombinant bacterial cell of the disclosure, or which is capable of killing the recombinant bacterial cell of the disclosure. The term “toxin” is intended to include bacteriostatic proteins and bactericidal proteins. The term “toxin” is intended to include, but not limited to, lytic proteins, bacteriocins (e.g., microcins and colicins), gyrase inhibitors, polymerase inhibitors, transcription inhibitors, translation inhibitors, DNases, and RNases. The term “anti-toxin” or “antitoxin,” as used herein, refers to a protein or enzyme which is capable of inhibiting the activity of a toxin. The term anti-toxin is intended to include, but not limited to, immunity modulators, and inhibitors of toxin expression. Examples of toxins and antitoxins are known in the art and described in more detail infra.

As used herein, the term “branched chain amino acid catabolic or catabolism enzyme” or “BCAA catabolic or catabolism enzyme” or “branched chain or BCAA amino acid metabolic enzyme” refers to any enzyme that is capable of metabolizing a branched chain amino acid or capable of reducing accumulated branched chain amino acid or that can lessen, ameliorate, or prevent one or more branched chain amino acid diseases or disease symptoms. Examples of branched chain amino acid metabolic enzymes include, but are not limited to, leucine dehydrogenase (e.g., LeuDH), branched chain amino acid aminotransferase (e.g., IlvE), branched chain α-ketoacid dehydrogenase (e.g., KivD), L-Amino acid deaminase (e.g., L-AAD), alcohol dehydrogenase (e.g., Adh2, YqhD)), and aldehyde dehydrogenase (e.g., PadA), and any other enzymes that catabolizes BCAA. Functional deficiencies in these proteins result in the accumulation of BCAA or its corresponding α-keto acid in cells and tissues. BCAA metabolic enzymes of the present disclosure include both wild-type or modified BCAA metabolic enzymes and can be produced using recombinant and synthetic methods or purified from nature sources. BCAA metabolic enzymes include full-length polypeptides and functional fragments thereof, as well as homologs and variants thereof. BCAA metabolic enzymes include polypeptides that have been modified from the wild-type sequence, including, for example, polypeptides having one or more amino acid deletions, insertions, and/or substitutions and may include, for example, fusion polypeptides and polypeptides having additional sequence, e.g., regulatory peptide sequence, linker peptide sequence, and other peptide sequence.

As used herein, “payload” refers to one or more molecules of interest to be produced by a genetically engineered microorganism, such as a bacterium, yeast, or a virus. In some embodiments, the payload is a therapeutic payload, e.g., a branched chain amino acid catabolic enzyme or a BCAA transporter polypeptide. In some embodiments, the payload is a regulatory molecule, e.g., a transcriptional regulator such as FNR. In some embodiments, the payload comprises a regulatory element, such as a promoter or a repressor. In some embodiments, the payload comprises an inducible promoter, such as from FNRS. In some embodiments, the payload comprises a repressor element, such as a kill switch. In some embodiments, the payload comprises an antibiotic resistance gene or genes. In some embodiments, the payload is encoded by a gene, multiple genes, gene cassette, or an operon. In alternate embodiments, the payload is produced by a biosynthetic or biochemical pathway, wherein the biosynthetic or biochemical pathway may optionally be endogenous to the microorganism. In alternate embodiments, the payload is produced by a biosynthetic or biochemical pathway, wherein the biosynthetic or biochemical pathway is not endogenous to the microorganism. In some embodiments, the genetically engineered microorganism comprises two or more payloads.

As used herein, the term “conventional branched chain amino acid or BCAA disease treatment” or “conventional branched chain amino acid or BCAA disease therapy” refers to treatment or therapy that is currently accepted, considered current standard of care, and/or used by most healthcare professionals for treating a disease or disorder associated with BCAA. It is different from alternative or complementary therapies, which are not as widely used.

As used herein, the term “polypeptide” includes “polypeptide” as well as “polypeptides,” and refers to a molecule composed of amino acid monomers linearly linked by amide bonds (i.e., peptide bonds). The term “polypeptide” refers to any chain or chains of two or more amino acids, and does not refer to a specific length of the product. Thus, “peptides,” “dipeptides,” “tripeptides, “oligopeptides,” “protein,” “amino acid chain,” or any other term used to refer to a chain or chains of two or more amino acids, are included within the definition of “polypeptide,” and the term “polypeptide” may be used instead of, or interchangeably with any of these terms. The term “polypeptide” is also intended to refer to the products of post-expression modifications of the polypeptide, including but not limited to glycosylation, acetylation, phosphorylation, amidation, derivatization, proteolytic cleavage, or modification by non-naturally occurring amino acids. A polypeptide may be derived from a natural biological source or produced by recombinant technology. In other embodiments, the polypeptide is produced by the genetically engineered bacteria, yeast, or virus of the current invention. A polypeptide of the invention may be of a size of about 3 or more, 5 or more, 10 or more, 20 or more, 25 or more, 50 or more, 75 or more, 100 or more, 200 or more, 500 or more, 1,000 or more, or 2,000 or more amino acids. Polypeptides may have a defined three-dimensional structure, although they do not necessarily have such structure. Polypeptides with a defined three-dimensional structure are referred to as folded, and polypeptides, which do not possess a defined three-dimensional structure, but rather can adopt a large number of different conformations, are referred to as unfolded. The term “peptide” or “polypeptide” may refer to an amino acid sequence that corresponds to a protein or a portion of a protein or may refer to an amino acid sequence that corresponds with non-protein sequence, e.g., a sequence selected from a regulatory peptide sequence, leader peptide sequence, signal peptide sequence, linker peptide sequence, and other peptide sequence.

An “isolated” polypeptide or a fragment, variant, or derivative thereof refers to a polypeptide that is not in its natural milieu. No particular level of purification is required. Recombinantly produced polypeptides and proteins expressed in host cells, including but not limited to bacterial or mammalian cells, are considered isolated for purposed of the invention, as are native or recombinant polypeptides which have been separated, fractionated, or partially or substantially purified by any suitable technique. Recombinant peptides, polypeptides or proteins refer to peptides, polypeptides or proteins produced by recombinant DNA techniques, i.e. produced from cells, microbial or mammalian, transformed by an exogenous recombinant DNA expression construct encoding the polypeptide. Proteins or peptides expressed in most bacterial cultures will typically be free of glycan. Fragments, derivatives, analogs or variants of the foregoing polypeptides, and any combination thereof are also included as polypeptides. The terms “fragment,” “variant,” “derivative” and “analog” include polypeptides having an amino acid sequence sufficiently similar to the amino acid sequence of the original peptide and include any polypeptides, which retain at least one or more properties of the corresponding original polypeptide. Fragments of polypeptides of the present invention include proteolytic fragments, as well as deletion fragments. Fragments also include specific antibody or bioactive fragments or immunologically active fragments derived from any polypeptides described herein. Variants may occur naturally or be non-naturally occurring. Non-naturally occurring variants may be produced using mutagenesis methods known in the art. Variant polypeptides may comprise conservative or non-conservative amino acid substitutions, deletions or additions.

Polypeptides also include fusion proteins. As used herein, the term “variant” includes a fusion protein, which comprises a sequence of the original peptide or sufficiently similar to the original peptide. As used herein, the term “fusion protein” refers to a chimeric protein comprising amino acid sequences of two or more different proteins. Typically, fusion proteins result from well known in vitro recombination techniques. Fusion proteins may have a similar structural function (but not necessarily to the same extent), and/or similar regulatory function (but not necessarily to the same extent), and/or similar biochemical function (but not necessarily to the same extent) and/or immunological activity (but not necessarily to the same extent) as the individual original proteins which are the components of the fusion proteins. “Derivatives” include but are not limited to peptides, which contain one or more naturally occurring amino acid derivatives of the twenty standard amino acids. “Similarity” between two peptides is determined by comparing the amino acid sequence of one peptide to the sequence of a second peptide. An amino acid of one peptide is similar to the corresponding amino acid of a second peptide if it is identical or a conservative amino acid substitution. Conservative substitutions include those described in Dayhoff, M. O., ed., The Atlas of Protein Sequence and Structure 5, National Biomedical Research Foundation, Washington, D.C. (1978), and in Argos, EMBO J. 8 (1989), 779-785. For example, amino acids belonging to one of the following groups represent conservative changes or substitutions: Ala, Pro, Gly, Gln, Asn, Ser, Thr, Cys, Ser, Tyr, Thr, Val, Ile, Leu, Met, Ala, Phe, Lys, Arg, His, Phe, Tyr, Trp, His, Asp, and Glu.

As used herein, the term “sufficiently similar” means a first amino acid sequence that contains a sufficient or minimum number of identical or equivalent amino acid residues relative to a second amino acid sequence such that the first and second amino acid sequences have a common structural domain and/or common functional activity. For example, amino acid sequences that comprise a common structural domain that is at least about 45%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or at least about 100%, identical are defined herein as sufficiently similar. Preferably, variants will be sufficiently similar to the amino acid sequence of the peptides of the invention. Such variants generally retain the functional activity of the peptides of the present invention. Variants include peptides that differ in amino acid sequence from the native and wild-type peptide, respectively, by way of one or more amino acid deletion(s), addition(s), and/or substitution(s). These may be naturally occurring variants as well as artificially designed ones.

As used herein the term “linker”, “linker peptide” or “peptide linkers” or “linker” refers to synthetic or non-native or non-naturally-occurring amino acid sequences that connect or link two polypeptide sequences, e.g., that link two polypeptide domains. As used herein the term “synthetic” refers to amino acid sequences that are not naturally occurring. Exemplary linkers are described herein. Additional exemplary linkers are provided in US 20140079701, the contents of which are herein incorporated by reference in its entirety.

As used herein the term “codon-optimized” refers to the modification of codons in the gene or coding regions of a nucleic acid molecule to reflect the typical codon usage of the host organism without altering the polypeptide encoded by the nucleic acid molecule. Such optimization includes replacing at least one, or more than one, or a significant number, of codons with one or more codons that are more frequently used in the genes of the host organism. A “codon-optimized sequence” refers to a sequence, which was modified from an existing coding sequence, or designed, for example, to improve translation in an expression host cell or organism of a transcript RNA molecule transcribed from the coding sequence, or to improve transcription of a coding sequence. Codon optimization includes, but is not limited to, processes including selecting codons for the coding sequence to suit the codon preference of the expression host organism. Many organisms display a bias or preference for use of particular codons to code for insertion of a particular amino acid in a growing polypeptide chain. Codon preference or codon bias, differences in codon usage between organisms, is allowed by the degeneracy of the genetic code, and is well documented among many organisms. Codon bias often correlates with the efficiency of translation of messenger RNA (mRNA), which is in turn believed to be dependent on, inter alia, the properties of the codons being translated and the availability of particular transfer RNA (tRNA) molecules. The predominance of selected tRNAs in a cell is generally a reflection of the codons used most frequently in peptide synthesis. Accordingly, genes can be tailored for optimal gene expression in a given organism based on codon optimization.

As used herein, the terms “secretion system” or “secretion protein” refers to a native or non-native secretion mechanism capable of secreting or exporting a biomolecule, e.g., polypeptide from the microbial, e.g., bacterial cytoplasm. The secretion system may comprise a single protein or may comprise two or more proteins assembled in a complex e.g. HlyBD. Non-limiting examples of secretion systems for gram negative bacteria include the modified type III flagellar, type I (e.g., hemolysin secretion system), type II, type IV, type V, type VI, and type VII secretion systems, resistance-nodulation-division (RND) multi-drug efflux pumps, various single membrane secretion systems. Non-liming examples of secretion systems for gram positive bacteria include Sec and TAT secretion systems. In some embodiments, the polypeptide to be secreted include a “secretion tag” of either RNA or peptide origin to direct the polypeptide to specific secretion systems. In some embodiments, the secretion system is able to remove this tag before secreting the polypeptide from the engineered bacteria. For example, in Type V auto-secretion-mediated secretion the N-terminal peptide secretion tag is removed upon translocation of the “passenger” peptide from the cytoplasm into the periplasmic compartment by the native Sec system. Further, once the auto-secretor is translocated across the outer membrane the C-terminal secretion tag can be removed by either an autocatalytic or protease-catalyzed e.g., OmpT cleavage thereby releasing the lysosomal enzyme(s) into the extracellular milieu. In some embodiments, the secretion system involves the generation of a “leaky” or de-stabilized outer membrane, which may be accomplished by deleting or mutagenizing genes responsible for tethering the outer membrane to the rigid peptidoglycan skeleton, including for example, lpp, ompC, ompA, ompF, tolA, tolB, pal, degS, degP, and nlpl. Lpp functions as the primary ‘staple’ of the bacterial cell wall to the peptidoglycan. TolA-PAL and OmpA complexes function similarly to Lpp and are other deletion targets to generate a leaky phenotype. Additionally, leaky phenotypes have been observed when periplasmic proteases, such as degS, degP or nipI, are deactivated. Thus, in some embodiments, the engineered bacteria have one or more deleted or mutated membrane genes, e.g., selected from lpp, ompA, ompA, ompF, tolA, tolB, and pal genes. In some embodiments, the engineered bacteria have one or more deleted or mutated periplasmic protease genes, e.g., selected from degS, degP, and nlpl. In some embodiments, the engineered bacteria have one or more deleted or mutated gene(s), selected from lpp, ompA, ompA, ompF, tolA, tolB, pal, degS, degP, and nlpl genes.

The articles “a” and “an,” as used herein, should be understood to mean “at least one,” unless clearly indicated to the contrary.

The phrase “and/or,” when used between elements in a list, is intended to mean either (1) that only a single listed element is present, or (2) that more than one element of the list is present. For example, “A, B, and/or C” indicates that the selection may be A alone; B alone; C alone; A and B; A and C; B and C; or A, B, and C. The phrase “and/or” may be used interchangeably with “at least one of” or “one or more of” the elements in a list.

Ranges provided herein are understood to be shorthand for all of the values within the range. For example, a range of 1 to 50 is understood to include any number, combination of numbers, or sub-range from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50.

Recombinant Bacteria

The genetically engineered microorganisms, or programmed microorganisms, such as genetically engineered bacteria, of the disclosure are capable of producing one or more enzymes for metabolizing a branched amino acid and/or a metabolite thereof. In some aspects, the disclosure provides a bacterial cell that comprises one or more optimized heterologous gene sequence(s) encoding a branched chain amino acid catabolic enzyme or other protein that results in a decrease in BCAA levels, e.g., leucine levels.

In certain embodiments, the genetically engineered bacteria are obligate anaerobic bacteria. In certain embodiments, the genetically engineered bacteria are facultative anaerobic bacteria. In certain embodiments, the genetically engineered bacteria are aerobic bacteria. In some embodiments, the genetically engineered bacteria are Gram-positive bacteria. In some embodiments, the genetically engineered bacteria are Gram-positive bacteria and lack LPS. In some embodiments, the genetically engineered bacteria are Gram-negative bacteria. In some embodiments, the genetically engineered bacteria are Gram-positive and obligate anaerobic bacteria. In some embodiments, the genetically engineered bacteria are Gram-positive and facultative anaerobic bacteria. In some embodiments, the genetically engineered bacteria are non-pathogenic bacteria. In some embodiments, the genetically engineered bacteria are commensal bacteria. In some embodiments, the genetically engineered bacteria are probiotic bacteria. In some embodiments, the genetically engineered bacteria are naturally pathogenic bacteria that are modified or mutated to reduce or eliminate pathogenicity. Exemplary bacteria include, but are not limited to, Bacillus, Bacteroides, Bifidobacterium, Brevibacteria, Caulobacter, Clostridium, Enterococcus, Escherichia coli, Lactobacillus, Lactococcus, Listeria, Mycobacterium, Saccharomyces, Salmonella, Staphylococcus, Streptococcus, Vibrio, Bacillus coagulans, Bacillus subtilis, Bacteroides fragilis, Bacteroides subtilis, Bacteroides thetaiotaomicron, Bifidobacterium adolescentis, Bifidobacterium bifidum, Bifidobacterium breve UCC2003, Bifidobacterium infantis, Bifidobacterium lactis, Bifidobacterium longum, Clostridium acetobutylicum, Clostridium butyricum, Clostridium butyricum M-55, Clostridium cochlearum, Clostridium felsineum, Clostridium histolyticum, Clostridium multifermentans, Clostridium novyi-NT, Clostridium paraputrificum, Clostridium pasteureanum, Clostridium pectinovorum, Clostridium perfringens, Clostridium roseum, Clostridium sporogenes, Clostridium tertium, Clostridium tetani, Clostridium tyrobutyricum, Corynebacterium parvum, Escherichia coli MG1655, Escherichia coli Nissle 1917, Listeria monocytogenes, Mycobacterium bovis, Salmonella choleraesuis, Salmonella typhimurium, and Vibrio cholera. In certain embodiments, the genetically engineered bacteria are selected from the group consisting of Enterococcus faecium, Lactobacillus acidophilus, Lactobacillus bulgaricus, Lactobacillus casei, Lactobacillus johnsonii, Lactobacillus paracasei, Lactobacillus plantarum, Lactobacillus reuteri, Lactobacillus rhamnosus, Lactococcus lactis, and Saccharomyces boulardii. In certain embodiments, the genetically engineered bacteria are selected from Bacteroides fragilis, Bacteroides thetaiotaomicron, Bacteroides subtilis, Bifidobacterium bifidum, Bifidobacterium infantis, Bifidobacterium lactis, Clostridium butyricum, Escherichia coli, Escherichia coli Nissle, Lactobacillus acidophilus, Lactobacillus plantarum, Lactobacillus reuteri, and Lactococcus lactis bacterial cell. In one embodiment, the bacterial cell is a Bacteroides fragilis bacterial cell. In one embodiment, the bacterial cell is a Bacteroides thetaiotaomicron bacterial cell. In one embodiment, the bacterial cell is a Bacteroides subtilis bacterial cell. In one embodiment, the bacterial cell is a Bifidobacterium bifidum bacterial cell. In one embodiment, the bacterial cell is a Bifidobacterium infantis bacterial cell. In one embodiment, the bacterial cell is a Bifidobacterium lactis bacterial cell. In one embodiment, the bacterial cell is a Clostridium butyricum bacterial cell. In one embodiment, the bacterial cell is an Escherichia coli bacterial cell. In one embodiment, the bacterial cell is a Lactobacillus acidophilus bacterial cell. In one embodiment, the bacterial cell is a Lactobacillus plantarum bacterial cell. In one embodiment, the bacterial cell is a Lactobacillus reuteri bacterial cell. In one embodiment, the bacterial cell is a Lactococcus lactis bacterial cell.

In some embodiments, the genetically engineered bacteria are Escherichia coli strain Nissle 1917 (E. coli Nissle), a Gram-negative bacterium of the Enterobacteriaceae family that has evolved into one of the best characterized probiotics (Ukena et al., 2007). The strain is characterized by its complete harmlessness (Schultz, 2008), and has GRAS (generally recognized as safe) status (Reister et al., 2014, emphasis added). Genomic sequencing confirmed that E. coli Nissle lacks prominent virulence factors (e.g., E. coli α-hemolysin, P-fimbrial adhesins) (Schultz, 2008). In addition, it has been shown that E. coli Nissle does not carry pathogenic adhesion factors, does not produce any enterotoxins or cytotoxins, is not invasive, and not uropathogenic (Sonnenborn et al., 2009). As early as in 1917, E. coli Nissle was packaged into medicinal capsules, called Mutaflor, for therapeutic use. E. coli Nissle has since been used to treat ulcerative colitis in humans in vivo (Rembacken et al., 1999), to treat inflammatory bowel disease, Crohn's disease, and pouchitis in humans in vivo (Schultz, 2008), and to inhibit enteroinvasive Salmonella, Legionella, Yersinia, and Shigella in vitro (Altenhoefer et al., 2004). It is commonly accepted that E. coli Nissle's therapeutic efficacy and safety have convincingly been proven (Ukena et al., 2007).

One of ordinary skill in the art would appreciate that the genetic modifications disclosed herein may be adapted for other species, strains, and subtypes of bacteria. Furthermore, genes from one or more different species can be introduced into one another, e.g., the kivD gene from Lactococcus lactis can be expressed in Escherichia coli. In one embodiment, the recombinant bacterial cell does not colonize the subject having the disorder. Unmodified E. coli Nissle and the genetically engineered bacteria of the invention may be destroyed, e.g., by defense factors in the gut or blood serum (Sonnenborn et al., 2009). In some embodiments, the residence time is calculated for a human subject. In some embodiments, residence time in vivo is calculated for the genetically engineered bacteria of the invention.

In some embodiments, the bacterial cell is a genetically engineered bacterial cell. In another embodiment, the bacterial cell is a recombinant bacterial cell. In some embodiments, the disclosure comprises a colony of bacterial cells disclosed herein.

In another aspect, the disclosure provides a recombinant bacterial culture which comprises bacterial cells disclosed herein. In one aspect, the disclosure provides a recombinant bacterial culture which reduces levels of a branched chain amino acid, e.g., leucine, in the media of the culture. In one embodiment, the levels of the branched chain amino acid, e.g., leucine, are reduced by about 50%, about 75%, or about 100% in the media of the cell culture. In another embodiment, the levels of the branched chain amino acid, e.g., leucine, are reduced by about two-fold, three-fold, four-fold, five-fold, six-fold, seven-fold, eight-fold, nine-fold, or ten-fold in the media of the cell culture. In one embodiment, the levels of the branched chain amino acid, e.g., leucine, are reduced below the limit of detection in the media of the cell culture.

In some embodiments of the above described genetically engineered bacteria, the gene encoding a branched chain amino acid catabolism enzyme is present on a plasmid in the bacterium and operatively linked on the plasmid to a promoter that is induced under low-oxygen or anaerobic conditions, such as any of the promoters disclosed herein. In other embodiments, the gene encoding a branched chain amino acid catabolism enzyme is present in the bacterial chromosome and is operatively linked in the chromosome to the promoter that is induced under low-oxygen or anaerobic conditions, such as any of the promoters disclosed herein. In some embodiments of the above described genetically engineered bacteria, the gene encoding a branched chain amino acid catabolic enzyme is present on a plasmid in the bacterium and operatively linked on the plasmid to the promoter that is induced under inflammatory conditions, such as any of the promoters disclosed herein. In other embodiments, the gene encoding a branched chain amino acid catabolic enzyme is present in the bacterial chromosome and is operatively linked in the chromosome to the promoter that is induced under inflammatory conditions, such as any of the promoters disclosed herein.

In some embodiments, the genetically engineered bacteria comprising gene sequence encoding a branched chain amino acid catabolic enzyme is an auxotroph. In one embodiment, the genetically engineered bacteria is an auxotroph selected from a cysE, glnA, ilvD, leuB, lysA, serA, metA, glyA, hisB, ilvA, pheA, proA, thrC, trpC, tyrA, thyA, uraA, dapA, dapB, dapD, dapE, dapF, flhD, metB, metC, proAB, and thi1 auxotroph. In some embodiments, the engineered bacteria have more than one auxotrophy, for example, they may be a ΔthyA and ΔdapA auxotroph. In some embodiments, the genetically engineered bacteria comprising gene sequence encoding a branched chain amino acid catabolic enzyme lacks functional ilvC gene sequence, e.g., is a ilvC auxotroph. IlvC encodes keto acid reductoisomerase, which enzyme is required for BCAA synthesis. Knock out of ilvC creates an auxotroph and requires the bacterial cell to import isoleucine and valine to survive.

In some embodiments, the genetically engineered bacteria comprising gene sequence encoding a branched chain amino acid catabolism enzyme further comprise gene sequence(s) encoding a BCAA transporter or other amino acid transporter that transports one or more BCAA(s) into the bacterial cell, for example a transporter that is capable of transporting leucine, valine, and/or isoleucine into a bacterial cell. In certain embodiments, the BCAA transporter is a leucine transporter, e.g., a high-affinity leucine transporter. In certain embodiments, the bacterial cell contains livK, livH, livM, livG, and livF genes. In certain embodiments, the BCAA transporter is a BCAA transporter, e.g., a low affinity BCAA transporter. In certain embodiments, the bacterial cell contains gene sequence encoding brnQ gene.

In some embodiments, the genetically engineered bacteria comprising gene sequence encoding a branched chain amino acid catabolism enzyme further comprise gene sequence(s) encoding a secretion protein or protein complex for secreting a biomolecule, such as any of the secretion systems disclosed herein.

In some embodiments, the genetically engineered bacteria comprising gene sequence encoding a branched chain amino acid catabolism enzyme further comprise gene sequence(s) encoding one or more antibiotic gene(s), such as any of the antibiotic genes disclosed herein.

In some embodiments, the genetically engineered bacteria comprising a branched chain amino acid catabolism enzyme further comprise a kill-switch circuit, such as any of the kill-switch circuits provided herein. For example, in some embodiments, the genetically engineered bacteria further comprise one or more genes encoding one or more recombinase(s) under the control of an inducible promoter, and an inverted toxin sequence. In some embodiments, the genetically engineered bacteria further comprise one or more genes encoding an antitoxin. In some embodiments, the engineered bacteria further comprise one or more genes encoding one or more recombinase(s) under the control of an inducible promoter and one or more inverted excision genes, wherein the excision gene(s) encode an enzyme that deletes an essential gene. In some embodiments, the genetically engineered bacteria further comprise one or more genes encoding an antitoxin. In some embodiments, the engineered bacteria further comprise one or more genes encoding a toxin under the control of a promoter having a TetR repressor binding site and a gene encoding the TetR under the control of an inducible promoter that is induced by arabinose, such as P_{araBAD}. In some embodiments, the genetically engineered bacteria further comprise one or more genes encoding an antitoxin.

In some embodiments, the genetically engineered bacteria are an auxotroph comprising gene sequence encoding a branched chain amino acid catabolism enzyme and further comprises a kill-switch circuit, such as any of the kill-switch circuits described herein.

In some embodiments of the above described genetically engineered bacteria, the gene encoding a branched chain amino acid catabolism enzyme is present on a plasmid in the bacterium. In some embodiments, the gene encoding a branched chain amino acid catabolism enzyme is present in the bacterial chromosome. In some embodiments, the gene sequence(s) encoding a BCAA transporter or other amino acid transporter that transports one or more BCAA(s) into the bacterial cell, for example a transporter that is capable of transporting leucine, valine, and/or isoleucine into a bacterial cell, is present on a plasmid in the bacterium. In some embodiments, the gene sequence(s) encoding a BCAA transporter or other amino acid transporter that transports one or more BCAA(s) into the bacterial cell, for example a transporter that is capable of transporting leucine, valine, and/or isoleucine into a bacterial cell, is present in the bacterial chromosome. In some embodiments, the gene sequence encoding a secretion protein or protein complex for secreting a biomolecule, such as any of the secretion systems disclosed herein, is present on a plasmid in the bacterium. In some embodiments, the gene sequence encoding a secretion protein or protein complex for secreting a biomolecule, such as any of the secretion systems disclosed herein, is present in the bacterial chromosome. In some embodiments, the gene sequence(s) encoding an antibiotic resistance gene is present on a plasmid in the bacterium. In some embodiments, the gene sequence(s) encoding an antibiotic resistance gene is present in the bacterial chromosome.

Branched Chain Amino Acid Catabolism Enzymes

As used herein, the term “branched chain amino acid catabolic or catabolism enzyme” refers to an enzyme involved in the catabolism of a branched chain amino acid to its corresponding α-keto acid counterpart; or the catabolism of an alpha-keto acid to its corresponding aldehyde, acyl-CoA, alcohol, carboxylic acid, or other metabolite counterpart. In some embodiments, the present disclosure provides an engineered bacterium comprising gene sequence(s) encoding one or more branched chain amino acid catabolism enzyme(s). In some embodiments, the branched chain amino acid catabolism enzyme is used to convert a branched chain amino acid, e.g., leucine, valine, isoleucine, to its corresponding α-keto-acid, e.g., α-ketoisocaproate, α-keto-β-methylvalerate, and α-ketoisovalerate. In some embodiments, wherein a branched chain amino acid catabolism enzyme is used to convert a branched chain amino acid, e.g., leucine, valine, isoleucine, to its corresponding α-keto-acid, the engineered bacteria further comprise one or more branched chain amino acid catabolism enzyme(s) to convert an α-keto-acid to its corresponding acetyl CoA, e.g., isovaleryl-CoA, α-methylbutyryl-CoA, and isobutyryl-CoA. In some embodiments, wherein a branched chain amino acid catabolism enzyme is used to convert a branched chain amino acid, e.g., leucine, valine, isoleucine, to its corresponding α-keto-acid, the engineered bacteria further comprise one or more branched chain amino acid catabolism enzyme(s) to convert an α-keto-acid to its corresponding aldehyde, e.g., isovaleraldehyde, isobutyraldehyde, and 2-methylbutyraldehyde. In some embodiments, the engineered bacteria may further comprise an alcohol dehydrogenase enzyme in order to convert the branched chain amino acid-derived aldehyde (e.g., isovaleraldehyde, isobutyraldehyde, 2-methylbutyraldehyde) to its respective alcohol. In some embodiments, the engineered bacteria may further comprise an aldehyde dehydrogenase enzyme in order to convert the branched chain amino acid-derived aldehyde (e.g., isovaleraldehyde, isobutyraldehyde, 2-methylbutyraldehyde) to its respective carboxylic acid.

Enzymes involved in the catabolism of branched chain amino acids are well known to those of skill in the art. For example, in bacteria, leucine dehydrogenase (LeuDH), branched chain amino acid transferase (IlvE), amino acid oxidase (also known as amino acid deaminase) (L-AAD), as well as other known enzymes, can be used to convert a BCAA to its corresponding α-keto acid, e.g., ketoisocaproate (KIC), ketoisovalerate (KIV), and ketomethylvalerate (KMV). Leucine dehydrogenases, branched chain amino acid transamination enzymes (EC 2.6.1.42), and L-amino acid deaminases (L-AAD), which oxidatively deaminate branched chain amino acids into their respective alpha-keto acid, are known (Baker et al., Structure, 3(7):693-705, 1995; Peng et al., J. Bact., 139(2):339-45, 1979; and Kline et al., J. Bact., 130(2):951-3, 1977). In bacteria, branched chain keto acid dehydrogenases (“BCKDs”) are enzyme complexes that oxidatively decarboxylate all three branched chain keto acids into their respective acyl-CoA derivatives. Thus, in one embodiment, the branched chain amino acid catabolism enzyme is a branched chain keto acid dehydrogenase (BCKD). Moreover, in mammals, dehydrogenases specific for 2-ketoisovalerate (EC 1.2.4.4) and 2-keto-3-methylvalerate and 2-keto-isocaproate (EC 1.2.4.3) have been identified (see, for example, Massey et al., Bacteriol Rev., 40(1):42-54, 1976). In bacteria, branched chain keto acid dehydrogenases (“BCKDs”) are enzyme complexes that oxidatively decarboxylate all three branched chain keto acids into their respective acyl-CoA derivatives. Also, for example, in bacteria, α-ketoisovalerate decarboxylase (KivD) enzymes are capable of converting α-keto acids into aldehydes (e.g., isovaleraldehyde, isobutyraldehyde, 2-methylbutyraldehyde). Specifically, the α-ketoisovalerate decarboxylase enzyme KivD is capable of metabolizing valine by converting α-ketoisovalerate to isobutyraldehyde (see, for example, de la Plaza et al., FEMS Microbiol. Lett. 2004, 238(2):367-374). KivD is capable of metabolizing leucine by converting α-ketoisocaproate (KIC) to isovaleraldehyde. KivD is also capable of metabolizing isoleucine by converting α-ketomethylvalerate (KMV) to 2-methylbutyraldehyde. In addition, enzymes for converting isovaleraldehyde, isobutyraldehyde, and 2-methylbutyraldehyde to their respective alcohols or carboxylic acids are known and available. For example, alcohol dehydrogenases (e.g., Adh2, YqhD) can convert isovaleraldehyde, isobutyraldehyde, 2-methylbutyraldehyde to isopentanol, isobutanol, and 2-methylbutanol, respectively. Aldehyde dehydrogenases (e.g., PadA) can convert isovaleraldehyde, isobutyraldehyde, 2-methylbutyraldehyde to isovalerate, isobutyrate, and 2-methylbutyrate, respectively.

In some embodiments, the branched chain amino acid catabolism enzyme increases the rate of branched chain amino acid catabolism. In some embodiments, the branched chain amino acid catabolism enzyme decreases the level of one or more branched chain amino acids, e.g., leucine, isoleucine, and/or valine, in a cell, tissue, or organism. In some embodiments, the branched chain amino acid catabolism enzyme decreases the level of alpha-keto acid derived from BCAA in a cell, tissue, or organism. In some embodiments, the branched chain amino acid catabolism enzyme decreases the level of branched chain amino acid as compared to the level of its corresponding alpha-keto acid in a cell, tissue, or organism. In other embodiments, the branched chain amino acid catabolism enzyme increases the level of alpha-keto acid as compared to the level of its corresponding branched chain amino acid in a cell, tissue, or organism. In some embodiments, the branched chain amino acid catabolism enzyme decreases the level of the branched chain amino acid as compared to the level of its corresponding Acyl-CoA derivative in a cell, tissue, or organism. In some embodiments, the branched chain amino acid catabolism enzyme increases the level of the Acyl-CoA derivative as compared to the level of the branched chain amino acid in a cell, tissue, or organism. In some embodiments, the branched chain amino acid catabolism enzyme decreases the level of alpha-keto aldehyde derived from BCAA, e.g., isovaleraldehyde, isobutyraldehyde, and 2-methylbutyraldehyde, in a cell, tissue, or organism. In some embodiments, the branched chain amino acid catabolism enzyme decreases the level of branched chain amino acid as compared to the level of its corresponding alpha-keto aldehyde, e.g., isovaleraldehyde, isobutyraldehyde, and 2-methylbutyraldehyde, in a cell, tissue, or organism. In other embodiments, the branched chain amino acid catabolism enzyme increases the level of alpha-keto aldehyde, e.g., isovaleraldehyde, isobutyraldehyde, and 2-methylbutyraldehyde, as compared to the level of its corresponding branched chain amino acid in a cell, tissue, or organism. In some embodiments, the branched chain amino acid catabolism enzyme decreases the level of a corresponding downstream metabolite, e.g., isovalerate, isobutyrate, 2-methylbutyrate, isopentanol, isobutanol, and 2-methylbutanol, in a cell, tissue, or organism. In some embodiments, the branched chain amino acid catabolism enzyme decreases the level of branched chain amino acid as compared to the level of a corresponding downstream metabolite, e.g., isovalerate, isobutyrate, 2-methylbutyrate, isopentanol, isobutanol, and 2-methylbutanol, in a cell, tissue, or organism. In other embodiments, the branched chain amino acid catabolism enzyme increases the level of a downstream metabolite, e.g., isovalerate, isobutyrate, 2-methylbutyrate, isopentanol, isobutanol, and 2-methylbutanol, as compared to the level of its corresponding branched chain amino acid in a cell, tissue, or organism.

In some embodiments, the branched chain amino acid catabolism enzyme is a leucine catabolism enzyme. In other embodiments, the branched chain amino acid catabolism enzyme is an isoleucine catabolism enzyme. In other embodiments, the branched chain amino acid catabolism enzyme is a valine catabolism enzyme. In some embodiments, the branched chain amino acid catabolism enzyme is involved in the catabolism of leucine, isoleucine, and valine. In another embodiment, the branched chain amino acid catabolism enzyme is involved in the catabolism of leucine and valine, isoleucine and valine, or leucine and isoleucine. In some embodiments, the branched chain amino acid catabolism enzyme converts leucine, isoleucine, and/or valine into its corresponding α-keto acid. In certain specific embodiments, the present disclosure provides an engineered bacterium comprising gene sequence(s) encoding one or more catabolism enzymes selected from leucine dehydrogenase (LeuDH), BCAA aminotransferase (IlvE), and/or amino acid oxidase (L-AAD).

In some embodiments, the branched chain amino acid catabolism enzyme is an alpha-ketoisocaproic acid (KIC) catabolism enzyme. In other embodiments, the branched chain amino acid catabolism enzyme is an alpha-ketoisovaleric acid (KIV) catabolism enzyme. In other embodiments, the branched chain amino acid catabolism enzyme is an alpha-keto-beta-methylvaleric acid (KMV) catabolism enzyme. In other embodiments, the branched chain amino acid catabolism enzyme is involved in the catabolism of alpha-ketoisocaproic acid (KIC), alpha-ketoisovaleric acid (KIV), and alpha-keto-beta-methylvaleric acid (KMV). In other embodiments, the branched chain amino acid catabolism enzyme is involved in the catabolism of KIC and KIV, KIC and KMV, or KIV and KMV. In some embodiments, the branched chain amino acid catabolism enzyme converts alpha-ketoisocaproic acid (KIC), alpha-ketoisovaleric acid (KIV), and/or alpha-keto-beta-methylvaleric acid (KMV) into its corresponding aldehyde, e.g., isovaleraldehyde, isobutyraldehyde, and/or 2-methylbutyraldehyde. In some embodiments, the present disclosure provides an engineered bacterium comprising gene sequence(s) encoding KivD.

In one embodiment, the branched chain amino acid catabolism enzyme is an isovaleraldehyde catabolism enzyme. In another embodiment, the branched chain amino acid catabolism enzyme is an isobutyraldehyde catabolism enzyme. In another embodiment, the branched chain amino acid catabolism enzyme is 2-methylbutyraldehyde catabolism enzyme. In another embodiment, the branched chain amino acid catabolism enzyme is involved in the catabolism of isovaleraldehyde, isobutyraldehyde, and 2-methylbutyraldehyde. In another embodiment, the branched chain amino acid catabolism enzyme is involved in the catabolism of isovaleraldehyde and isobutyraldehyde, isovaleraldehyde and 2-methylbutyraldehyde, or isobutyraldehyde and 2-methylbutyraldehyde. In some embodiments, the present disclosure provides an engineered bacterium comprising gene sequence(s) encoding one or more alcohol dehydrogenase(s), e.g., Ahd2.

In some embodiments, the present disclosure provides an engineered bacterium comprising gene sequence(s) encoding one or more branched chain amino acid catabolism enzyme(s) involved in the catabolism of leucine, isoleucine, and/or valine, and further comprises gene sequence(s) encoding one or more branched chain amino acid catabolism enzyme(s) involved in the catabolism of KIC, KIV, and/or KMV. In some embodiments, the present disclosure provides an engineered bacteria comprising gene sequence(s) encoding one or more branched chain amino acid catabolism enzyme(s) involved in the catabolism of leucine, isoleucine, and/or valine, further comprises gene sequence(s) encoding one or more branched chain amino acid catabolism enzyme(s) involved in the catabolism of KIC, KIV, and/or KMV, and further comprises gene sequence(s) encoding one or more branched chain amino acid catabolism enzyme(s) involved in the catabolism of. isovaleraldehyde, isobutyraldehyde, and/or 2-methylbutyraldehyde. In some embodiments, the present disclosure provides an engineered bacterium comprising gene sequence(s) encoding one or more branched chain amino acid catabolism enzyme(s) selected from LeuDH, KivD, and Adh2.

In some embodiments, the present disclosure provides an engineered bacterium comprising gene sequence(s) encoding one or more branched chain amino acid catabolism enzyme(s) involved in the conversion of leucine, isoleucine, and/or valine to KIC, KIV, and/or KMV, respectively. In some embodiments, the present disclosure provides an engineered bacterium comprising gene sequence(s) encoding one or more branched chain amino acid catabolism enzyme(s) involved in the conversion of KIC, KIV, and/or KMV to isovaleraldehyde, isobutyraldehyde, and/or 2-methylbutyraldehyde, respectively. In some embodiments, the present disclosure provides an engineered bacterium comprising gene sequence(s) encoding one or more branched chain amino acid catabolism enzyme(s) involved in the conversion of isovaleraldehyde, isobutyraldehyde, and/or 2-methylbutyraldehyde to isovalerate, isobutyrate, and/or 2-methylbutyrate, respectively. In some embodiments, the present disclosure provides an engineered bacterium comprising gene sequence(s) encoding one or more branched chain amino acid catabolism enzyme(s) involved in the conversion of isovaleraldehyde, isobutyraldehyde, and/or 2-methylbutyraldehyde to isopentanol, isobutanol, and/or 2-methylbutanol respectively.

In some embodiments, the present disclosure provides an engineered bacteria comprising gene sequence(s) encoding one or more branched chain amino acid catabolism enzyme(s) involved in the conversion of leucine, isoleucine, and/or valine to KIC, KIV, and/or KMV, respectively, and further comprises gene sequence(s) encoding one or more branched chain amino acid catabolism enzyme(s) involved in the conversion of KIC, KIV, and/or KMV to isovaleraldehyde, isobutyraldehyde, and/or 2-methylbutyraldehyde, respectively. In some embodiments, the present disclosure provides an engineered bacterium comprising gene sequence(s) encoding one or more branched chain amino acid catabolism enzyme(s) involved in the conversion of KIC, KIV, and/or KMV to isovaleraldehyde, isobutyraldehyde, and/or 2-methylbutyraldehyde, respectively, and further comprises gene sequence(s) encoding one or more branched chain amino acid catabolism enzyme(s) involved in the conversion of. isovaleraldehyde, isobutyraldehyde, and/or 2-methylbutyraldehyde to isovalerate, isobutyrate, and/or 2-methylbutyrate, respectively. In some embodiments, the present disclosure provides an engineered bacteria comprising gene sequence(s) encoding one or more branched chain amino acid catabolism enzyme(s) involved in the conversion of KIC, KIV, and/or KMV to isovaleraldehyde, isobutyraldehyde, and/or 2-methylbutyraldehyde, respectively, and further comprises gene sequence(s) encoding one or more branched chain amino acid catabolism enzyme(s) involved in the conversion of isovaleraldehyde, isobutyraldehyde, and/or 2-methylbutyraldehyde to isopentanol, isobutanol, and/or 2-methylbutanol respectively.

In some embodiments, the present disclosure provides an engineered bacteria comprising gene sequence(s) encoding one or more branched chain amino acid catabolism enzyme(s) involved in the conversion of leucine, isoleucine, and/or valine to KIC, KIV, and/or KMV, respectively, further comprises gene sequence(s) encoding one or more branched chain amino acid catabolism enzyme(s) involved in the conversion of KIC, KIV, and/or KMV to isovaleraldehyde, isobutyraldehyde, and/or 2-methylbutyraldehyde, respectively, and further comprises gene sequence(s) encoding one or more branched chain amino acid catabolism enzyme(s) involved in the conversion of isovaleraldehyde, isobutyraldehyde, and/or 2-methylbutyraldehyde to isovalerate, isobutyrate, and/or 2-methylbutyrate, respectively. In some embodiments, the present disclosure provides an engineered bacteria comprising gene sequence(s) encoding one or more branched chain amino acid catabolism enzyme(s) involved in the conversion of leucine, isoleucine, and/or valine to KIC, KIV, and/or KMV, respectively, further comprises gene sequence(s) encoding one or more branched chain amino acid catabolism enzyme(s) involved in the conversion of KIC, KIV, and/or KMV to isovaleraldehyde, isobutyraldehyde, and/or 2-methylbutyraldehyde, respectively, and further comprises gene sequence(s) encoding one or more branched chain amino acid catabolism enzyme(s) involved in the conversion of. isovaleraldehyde, isobutyraldehyde, and/or 2-methylbutyraldehyde to isopentanol, isobutanol, and/or 2-methylbutanol, respectively. In some embodiments, the present disclosure provides an engineered bacterium comprising gene sequence(s) encoding one or more branched chain amino acid catabolism enzyme(s) selected from LeuDH, KivD, and Adh2.

Enzymes involved in the catabolism of a branched chain amino acid may be expressed or modified in the bacteria disclosed herein to enhance catabolism of a branched chain amino acid, e.g., leucine. Specifically, when a branched chain amino acid catabolism enzyme is expressed in the engineered bacteria disclosed herein, the engineered bacteria are able to convert (deaminate) more branched chain amino acids (e.g., leucine, valine, isoleucine) into their respective alpha-keto acids (KIC, KIV, KMV) and/or convert more BCAA alpha-keto acids (e.g., KIC, KIV, KMV) into respective BCAA-derived aldehydes (e.g., isovaleraldehyde, isobutyraldehyde, 2-methylbutyraldehyde) and/or convert more BCAA-derived aldehydes into respective alcohols (e.g., isopentanol, isobutanol, 2-methylbutanol) and/or convert more BCAA-derived aldehydes into respective carboxylic acids (isovalerate, isobutyrate, 2-methylbutyrate), and/or convert (decarboxylate) more branched chain alpha-keto acids into their respective acyl-CoA derivatives when the catabolism enzyme(s) is expressed, in comparison with unmodified bacteria of the same bacterial subtype under the same conditions. Thus, for example, the genetically engineered bacteria comprising gene sequence encoding a branched chain amino acid catabolism enzyme can catabolize the branched chain amino acid, e.g., leucine, and/or its corresponding alpha-keto acid, e.g., alpha-ketoisocaproate, to treat diseases associated with catabolism of branched chain amino acids, such as Maple Syrup Urine Disease (MSUD) and others described herein.

In some embodiments, the engineered bacteria comprise gene sequence(s) encoding one or more branched chain amino acid catabolism enzyme(s) and gene sequence(s) encoding one or more transporter(s) capable of importing a BCAA or metabolite thereof. In some embodiments, the engineered bacteria comprise gene sequence(s) encoding at least one branched chain amino acid catabolism enzyme and gene sequence(s) encoding two or more copies of a transporter capable of importing a BCAA or metabolite thereof. In some embodiments, the engineered bacteria comprise gene sequence(s) encoding at least one branched chain amino acid catabolism enzyme and gene sequence(s) encoding two or more different transporter(s) capable of importing a BCAA or metabolite thereof. In certain embodiments, the transporter is a leucine transporter. In certain embodiments, the transporter is a valine transporter. In certain embodiments, the transporter is an isoleucine transporter. In certain embodiments, the transporter is a branched chain amino acid transporter, e.g., capable of importing leucine, isoleucine, and valine. In certain specific embodiments, the transporter is selected from LivKHMGF and BmQ.

In some embodiments, the engineered bacteria comprise gene sequence(s) encoding at least one branched chain amino acid catabolism enzyme and gene sequence(s) encoding one or more BCAA binding proteins, e.g., a BCAA binding protein that assists in bringing BCAA(s) into the bacterial cell. In some embodiments, the engineered bacteria comprise gene sequence(s) encoding at least one branched chain amino acid catabolism enzyme, gene sequence(s) encoding one or more transporter(s) capable of importing one or more BCAAs, and gene sequence(s) encoding one or more BCAA binding proteins, e.g., a BCAA binding protein that assists in bringing BCAA(s) into the bacterial cell. In any of these embodiments, the engineered bacteria comprise gene sequence(s) encoding two or more copies of a BCAA binding protein. In any of these embodiments, the engineered bacteria comprise gene sequence(s) encoding two or more different BCAA binding proteins. In certain embodiments, the BCAA binding protein is LivJ.

In any of the embodiments described above and herein, the engineered bacteria may further comprise one or more genetic modification(s) that reduces export of a branched chain amino acid from the bacteria, e.g., a deletion or mutation in at least one gene associated with the export of a BCAA, e.g., deletion or mutation in leuE gene and/or its promoter (which reduces or eliminates the export of leucine). In some embodiments, the engineered bacteria comprise gene sequence(s) encoding at least one branched chain amino acid catabolism enzyme and at least one genetic modification that reduces export of a branched chain amino acid. In certain specific embodiments, the engineered bacteria comprise gene sequence(s) encoding at least one branched chain amino acid catabolism enzyme, gene sequence(s) encoding one or more transporter(s) capable of importing a BCAA, and at least one genetic modification that reduces export of a branched chain amino acid. In certain specific embodiments, the engineered bacteria comprise gene sequence(s) encoding at least one branched chain amino acid catabolism enzyme, gene sequence(s) encoding one or more transporter(s) capable of importing a BCAA, gene sequence(s) encoding one or more BCAA binding proteins, and at least one genetic modification that reduces export of a branched chain amino acid. In some embodiments, the engineered bacteria comprise gene sequence(s) encoding at least one branched chain amino acid catabolism enzyme, gene sequence(s) encoding one or more BCAA binding proteins, and at least one genetic modification that reduces export of a branched chain amino acid. In any of these embodiments, the genetic modification may be a deletion or mutation in one or more gene(s) that allow or assist in the export of a BCAA. In any of these embodiment, the genetic modification may be a deletion or mutation in a leuE gene and/or its promoter.

In any of the embodiments described above and herein, the engineered bacteria comprise gene sequence(s) encoding one or more branched chain amino acid catabolism enzyme(s), and at least one genetic modification that reduces endogenous biosynthesis of a branched chain amino acid, for example, a deletion or mutation in at least one gene required for BCAA synthesis, e.g., deletion or mutation in ilvC gene and/or its promoter, which gene is required for BCAA synthesis and whose absence creates an auxotroph requiring the bacterial cell to import leucine. In some embodiments, the engineered bacteria comprise gene sequence(s) encoding one or more branched chain amino acid catabolism enzyme(s), gene sequence(s) encoding one or more transporter(s) capable of importing a BCAA, and at least one genetic modification that reduces endogenous biosynthesis of a branched chain amino acid, for example, a deletion or mutation in at least one gene required for BCAA synthesis. In some embodiments, the engineered bacteria comprise gene sequence(s) encoding at least one branched chain amino acid catabolism enzyme, gene sequence(s) encoding one or more BCAA binding proteins, and at least one genetic modification that reduces endogenous biosynthesis of a branched chain amino acid. In some embodiments, the engineered bacteria comprise gene sequence(s) encoding at least one branched chain amino acid catabolism enzyme, at least one genetic modification that reduces export of a branched chain amino acid, and at least one genetic modification that reduces endogenous biosynthesis of a branched chain amino acid. In some embodiments, the engineered bacteria comprise gene sequence(s) encoding at least one branched chain amino acid catabolism enzyme, gene sequence(s) encoding one or more transporter(s) capable of importing a BCAA, gene sequence(s) encoding one or more BCAA binding proteins, and at least one genetic modification that reduces endogenous biosynthesis of a branched chain amino acid. In some embodiments, the engineered bacteria comprise gene sequence(s) encoding at least one branched chain amino acid catabolism enzyme, gene sequence(s) encoding one or more transporter(s) capable of importing a BCAA, at least one genetic modification that reduces export of a branched chain amino acid, and at least one genetic modification that reduces endogenous biosynthesis of a branched chain amino acid. In some embodiments, the engineered bacteria comprise gene sequence(s) encoding at least one branched chain amino acid catabolism enzyme, gene sequence(s) encoding one or more BCAA binding proteins, at least one genetic modification that reduces export of a branched chain amino acid, and at least one genetic modification that reduces endogenous biosynthesis of a branched chain amino acid. In some embodiments, the engineered bacteria comprise gene sequence(s) encoding at least one branched chain amino acid catabolism enzyme, gene sequence(s) encoding one or more transporter(s) capable of importing a BCAA, gene sequence(s) encoding one or more BCAA binding proteins, at least one genetic modification that reduces export of a branched chain amino acid, and at least one genetic modification that reduces endogenous biosynthesis of a branched chain amino acid. In any of these embodiments, the at least one genetic modification that reduces endogenous biosynthesis of a branched chain amino acid can be a deletion or mutation in at least one gene required for BCAA synthesis, e.g., deletion or mutation in ilvC gene and/or its promoter.

In any of the embodiments described above and herein, the gene sequence(s) encoding at least one branched chain amino acid catabolism enzyme, and/or gene sequence(s) encoding one or more transporter(s) capable of importing a BCAA, and/or gene sequence(s) encoding one or more BCAA binding proteins, and/or other sequence can be present in the bacterial chromosome. In any of the embodiments described above and herein, the gene sequence(s) encoding at least one branched chain amino acid catabolism enzyme, and/or gene sequence(s) encoding one or more transporter(s) capable of importing a BCAA, and/or gene sequence(s) encoding one or more BCAA binding proteins, and/or other sequence can be present in one or more plasmids.

In some embodiments, the engineered bacteria comprise gene sequence(s) encoding one or more branched chain amino acid catabolism enzyme(s) in which the one or more enzymes are from a different organism, e.g., a different species of bacteria. In some embodiments, the engineered bacteria comprise gene sequence(s) encoding one or more branched chain amino acid catabolism enzyme(s) in which the one or more enzymes are native to the bacterium. In some embodiments, the engineered bacteria comprise gene sequence(s) encoding one or more branched chain amino acid catabolism enzyme(s) in which one or more of the enzymes are native and one or more of the enzymes are from a different organism, e.g., a different species of bacteria. In other embodiments, the bacterial cell comprises more than one copy of a native gene encoding a branched chain amino acid catabolism enzyme. In other embodiments, the bacterial cell comprises more than one copy of a non-native gene encoding a branched chain amino acid catabolism enzyme. In other embodiments, the bacterial cell comprises at least one, two, three, four, five, six or more copies of a gene encoding a branched chain amino acid catabolism enzyme, which can be native or non-native. In other embodiments, the bacterial cell comprises multiple copies of a gene encoding a branched chain amino acid catabolism enzyme. In some embodiments, the bacterial cell comprises gene sequence(s) encoding multiple copies of two or more different branched chain amino acid catabolism enzymes.

In some embodiments, the engineered bacteria comprise gene sequence(s) encoding one or more branched chain amino acid transporters in which the one or more transporters are from a different organism, e.g., a different species of bacteria. In some embodiments, the engineered bacteria comprise gene sequence(s) encoding one or more branched chain amino acid transporter(s) in which the one or more transporters are native to the bacterium. In some embodiments, the engineered bacteria comprise gene sequence(s) encoding one or more branched chain amino acid transporters(s) in which one or more of the transporters are native and one or more of the transporters are from a different organism, e.g., a different species of bacteria. In other embodiments, the bacterial cell comprises more than one copy of a native gene encoding a branched chain amino acid transporter. In other embodiments, the bacterial cell comprises more than one copy of a non-native gene encoding a branched chain amino acid transporter. In other embodiments, the bacterial cell comprises at least one, two, three, four, five, six or more copies of a gene encoding a branched chain amino acid transporter, which can be native or non-native. In other embodiments, the bacterial cell comprises multiple copies of a gene encoding a branched chain amino acid transporters. In some embodiments, the bacterial cell comprises gene sequence(s) encoding multiple copies of two or more different branched chain amino acid transporters.

In some embodiments, the engineered bacteria comprise gene sequence(s) encoding one or more branched chain amino acid binding protein(s) in which the one or more binding protein(s) are from a different organism, e.g., a different species of bacteria. In some embodiments, the engineered bacteria comprise gene sequence(s) encoding one or more branched chain amino acid binding protein(s) in which the one or more binding protein(s) are native to the bacterium. In some embodiments, the engineered bacteria comprise gene sequence(s) encoding one or more branched chain amino acid binding protein(s) in which one or more of the binding protein(s) are native and one or more of the binding protein(s) are from a different organism, e.g., a different species of bacteria. In other embodiments, the bacterial cell comprises more than one copy of a native gene encoding a branched chain amino acid binding protein. In other embodiments, the bacterial cell comprises more than one copy of a non-native gene encoding a branched chain amino acid binding protein. In other embodiments, the bacterial cell comprises at least one, two, three, four, five, six or more copies of a gene encoding a branched chain amino acid binding protein, which can be native or non-native. In other embodiments, the bacterial cell comprises multiple copies of a gene encoding a branched chain amino acid binding protein. In some embodiments, the bacterial cell comprises gene sequence(s) encoding multiple copies of two or more different branched chain amino acid binding proteins.

Branched chain amino acid catabolism enzymes are known in the art. In some embodiments, the branched chain amino acid catabolism enzyme is encoded by a gene encoding a branched chain amino acid catabolism enzyme derived from a bacterial species. In some embodiments, a branched chain amino acid catabolism enzyme is encoded by a gene encoding a branched chain amino acid catabolism enzyme derived from a non-bacterial species. In some embodiments, a branched chain amino acid catabolism enzyme is encoded by a gene derived from a eukaryotic species, e.g., a yeast species or a plant species. In one embodiment, a branched chain amino acid catabolism enzyme is encoded by a gene derived from a mammalian species, e.g., a human. In some embodiments, the engineered bacteria comprise gene sequence(s) encoding at least one branched chain amino acid catabolism enzyme derived from a bacterial species and at least one branched chain amino acid catabolism enzyme derived from a non-bacterial species. In one embodiment, the gene encoding the branched chain amino acid catabolism enzyme is derived from an organism of the genus or species that includes, but is not limited to, Acetinobacter, Azospirillum, Bacillus, Bacteroides, Bifidobacterium, Brevibacteria, Burkholderia, Citrobacter, Clostridium, Corynebacterium, Cronobacter, Enterobacter, Enterococcus, Erwinia, Helicobacter, Klebsiella, Lactobacillus, Lactococcus, Leishmania, Listeria, Macrococcus, Mycobacterium, Nakamurella, Nasonia, Nostoc, Pantoea, Pectobacterium, Pseudomonas, Psychrobacter, Ralstonia, Saccharomyces, Salmonella, Sarcina, Serratia, Staphylococcus, and Yersinia, e.g., Acetinobacter radioresistens, Acetinobacter baumannii, Acetinobacter calcoaceticus, Azospirillum brasilense, Bacillus anthracis, Bacillus cereus, Bacillus coagulans, Bacillus megaterium, Bacillus subtilis, Bacillus thuringiensis, Bacteroides fragilis, Bacteroides subtilis, Bacteroides thetaiotaomicron, Bifdobacterium bifidum, Bifdobacterium infantis, Bifidobacterium lactis, Bifdobacterium longum, Burkholderia xenovorans, Citrobacter youngae, Citrobacter koseri, Citrobacter rodentium, Clostridium acetobutylicum, Clostridium butyricum, Corynebacterium aurimucosum, Corynebacterium kroppenstedtii, Corynebacterium striatum, Cronobacter sakazakii, Cronobacter turicensis, Enterobacter cloacae, Enterobacter cancerogenus, Enterococcus faecium, Erwinia amylovara, Erwinia pyrifoliae, Erwinia tasmaniensis, Helicobacter mustelae, Klebsiella pneumonia, Klebsiella variicola, Lactobacillus acidophilus, Lactobacillus bulgaricus, Lactobacillus casei, Lactobacillus johnsonii, Lactobacillus paracasei, Lactobacillus plantarum, Lactobacillus reuteri, Lactobacillus rhamnosus, Lactococcus lactis, Leishmania infantum, Leishmania major, Leishmania brazilensis, Listeria grayi, Macrococcus caseolyticus, Mycobacterium avium, Mycobacterium intracellulare, Mycobacterium kansasii, Mycobacterium leprae, Mycobacterium marinum, Mycobacterium smegmatis, Mycobacterium tuberculosis, Mycobacterium ulcerans, Nakamurella multipartita, Nasonia vitipennis, Nostoc punctiforme, Pantoea ananatis, Pantoea agglomerans, Pectobacterium atrosepticum, Pectobacterium carotovorum, Pseudomonas aeruginosa, Psychrobacter articus, Psychrobacter cryohalolentis, Ralstonia eutropha, Saccharomyces boulardii, Salmonella enterica, Sarcina ventriculi, Serratia odorifera, Serratia proteamaculans, Staphylococcus aerus, Staphylococcus capitis, Staphylococcys carnosus, Staphylococcus epidermidis, Staphylococcus hominis, Staphylococcus haemolyticus, Staphylococcus lugdunensis, Staphylococcus saprophyticus, Staphylococcus warneri, Yersinia enterocolitica, Yersinia mollaretii, Yersinia kristensenii, Yersinia rohdei, and Yersinia aldovae.

In some embodiments, the gene encoding a branched chain amino acid catabolism enzyme, BCAA transporter, BCAA binding protein, and/or other sequence has been codon-optimized for use in the host organism. In one embodiment, the gene encoding a branched chain amino acid catabolism enzyme has been codon-optimized for use in Escherichia coli.

In some embodiments, the branched chain amino acid catabolism enzyme converts a branched chain amino acid to its corresponding alpha-keto acid. Such enzymes include, for example, LeuDH. In other embodiments, the branched chain amino acid catabolism enzyme converts a branched chain keto acid to its corresponding aldehyde. For example, in some embodiments, the branched chain amino acid catabolism enzyme is an α-ketoisovalerate decarboxylase (KivD).

In other embodiments, the branched chain amino acid catabolism enzyme converts an aldehyde into its corresponding alcohol. For example, the branched chain amino acid catabolism enzyme may be an alcohol dehydrogenase. In one embodiment, the alcohol dehydrogenase is Adh2.

In some embodiments, the engineered bacteria comprise gene sequence(s) encoding one or more transporter(s) capable of importing a BCAA or metabolite thereof. In certain embodiments, the transporter is a leucine transporter. In certain embodiments, the transporter is a valine transporter. In certain embodiments, the transporter is an isoleucine transporter. In certain embodiments, the transporter is a branched chain amino acid transporter, e.g., capable of importing leucine, isoleucine, and valine. The term “BCAA transporter” is meant to refer to a transporter that specifically transports leucine, isoleucine, or valine, and also to a transporter that is able to transport any BCAA, including for example, the ability to transport leucine, isoleucine, and valine. For example, in some embodiments, the transporter is LivKHMGF. In some embodiments, the transporter is BmQ.

The present disclosure further comprises genes encoding functional fragments of a branched chain amino acid catabolism enzyme, BCAA transporter, BCAA binding protein, and/or other sequence or functional variants of a branched chain amino acid catabolism enzyme, BCAA transporter, BCAA binding protein, and/or other sequence. As used herein, the term “functional fragment thereof” or “functional variant thereof” of a branched chain amino acid catabolism enzyme, BCAA transporter, BCAA binding protein, and/or other sequence refers to fragment or variant sequence having qualitative biological activity in common with the wild-type branched chain amino acid catabolism enzyme, BCAA transporter, BCAA binding protein, and/or other sequence from which the fragment or variant was derived. For example, a functional fragment or a functional variant of a mutated branched chain amino acid catabolism enzyme is one which retains essentially the same ability to catabolize a branched chain amino acid and/or its corresponding alpha-keto acid or aldehyde or other metabolite as the branched chain amino acid catabolism enzyme from which the functional fragment or functional variant was derived. For example, a polypeptide having branched chain amino acid catabolism enzyme activity may be truncated at the N-terminus or C-terminus and the retention of branched chain amino acid catabolism enzyme activity assessed using assays known to those of skill in the art, including the exemplary assays provided herein. In one embodiment, the recombinant bacterial cell disclosed herein comprises a heterologous gene encoding a branched chain amino acid catabolism enzyme functional variant. In another embodiment, the recombinant bacterial cell disclosed herein comprises a heterologous gene encoding a branched chain amino acid catabolism enzyme functional fragment.

The present disclosure encompasses genes encoding a branched chain amino acid catabolism enzyme, BCAA transporter, BCAA binding protein, and/or other sequence comprising amino acids in its sequence that are substantially the same as an amino acid sequence described herein. Amino acid sequences that are substantially the same as the sequences described herein include sequences comprising conservative amino acid substitutions, as well as amino acid deletions and/or insertions. A conservative amino acid substitution refers to the replacement of a first amino acid by a second amino acid that has chemical and/or physical properties (e.g., charge, structure, polarity, hydrophobicity/hydrophilicity) that are similar to those of the first amino acid. Conservative substitutions include replacement of one amino acid by another within the following groups: lysine (K), arginine (R) and histidine (H); aspartate (D) and glutamate (E); asparagine (N), glutamine (Q), serine (S), threonine (T), tyrosine (Y), K, R, H, D and E; alanine (A), valine (V), leucine (L), isoleucine (I), proline (P), phenylalanine (F), tryptophan (W), methionine (M), cysteine (C) and glycine (G); F, W and Y; C, S and T. Similarly contemplated is replacing a basic amino acid with another basic amino acid (e.g., replacement among Lys, Arg, His), replacing an acidic amino acid with another acidic amino acid (e.g., replacement among Asp and Glu), replacing a neutral amino acid with another neutral amino acid (e.g., replacement among Ala, Gly, Ser, Met, Thr, Leu, Ile, Asn, Gln, Phe, Cys, Pro, Trp, Tyr, Val).

The present disclosure encompasses branched chain amino acid catabolism enzymes, BCAA transporters, BCAA binding proteins, and/or other sequences which have a certain percent identity to a gene or protein sequence described herein. For example, the disclosure encompasses branched chain amino acid catabolism enzymes, BCAA transporters, BCAA binding proteins, and/or other sequences having at least 60%, 65%, 70%, 75%, 80%, 85%, 90%, 91%, 92%, 93%, 94%, 95%, 96%, 97%, 98%, 99%, or 100% identity to a nucleic acid sequence or amino acid sequence disclosed herein. As used herein, the term “percent (%) sequence identity” or “percent (%) identity,” also including “homology,” is defined as the percentage of amino acid residues or nucleotides in a candidate sequence that are identical with the amino acid residues or nucleotides in the reference sequences after aligning the sequences and introducing gaps, if necessary, to achieve the maximum percent sequence identity, and not considering any conservative substitutions as part of the sequence identity. Optimal alignment of the sequences for comparison may be produced, besides manually, by means of the local homology algorithm of Smith and Waterman, 1981, Ads App. Math. 2, 482, by means of the local homology algorithm of Neddleman and Wunsch, 1970, J. Mol. Biol. 48, 443, by means of the similarity search method of Pearson and Lipman, 1988, Proc. Natl. Acad. Sci. USA 85, 2444, or by means of computer programs which use these algorithms (GAP, BESTFIT, FASTA, BLAST P, BLAST N and TFASTA in Wisconsin Genetics Software Package, Genetics Computer Group, 575 Science Drive, Madison, Wis.).

Assays for testing the activity of a branched chain amino acid catabolism enzyme, BCAA transporter, BCAA binding protein, and/or other sequence, or a functional variant, or a functional fragment thereof are well known to one of ordinary skill in the art. For example, branched chain amino acid catabolism, BCAA transporter, BCAA binding protein, and/or other sequence can be assessed by expressing the protein, functional variant, or fragment thereof, in a recombinant bacterial cell that lacks endogenous branched chain amino acid catabolism enzyme activity. Branched chain amino acid catabolism can be assessed using the coupled enzymatic assay method as described by Zhang et al. (see, for example, Zhang et al., Proc. Natl. Acad. Sci., 105(52):20653-58, 2008). Furthermore, catabolism of branched chain amino acids can also be assessed in vitro by measuring the disappearance of alpha-ketoisovalerate as described by de la Plaza (see, for example, de la Plaza et al., FEMS Microbiol. Letters, 2004, 238(2):367-374). BCAA as well as the branched chain keto acid can be quantified by liquid chromatography tandem mass spectrometry (LC-MS/MS), as described herein.

In some embodiments, the gene encoding a branched chain amino acid catabolism enzyme, BCAA transporter, BCAA binding protein, and/or other sequence is mutagenized; mutants exhibiting increased activity are selected; and the mutagenized gene encoding the branched chain amino acid catabolism enzyme, BCAA transporter, BCAA binding protein, and/or other sequence is isolated and inserted into the bacterial cell. In some embodiments, the gene encoding an α-ketoisovalerate decarboxylase, e.g., kivD, is mutagenized; mutants exhibiting decreased activity are selected; and the mutagenized gene encoding the α-ketoisovalerate decarboxylase, e.g., kivD, is isolated and inserted into the bacterial cell. The gene comprising the modifications described herein may be present on a plasmid or chromosome.

In some embodiments, the gene encoding the branched chain amino acid catabolism enzyme, BCAA transporter, BCAA binding protein, and/or other sequence is directly operably linked to a first promoter. In other embodiments, the gene encoding the branched chain amino acid catabolism enzyme, BCAA transporter, BCAA binding protein, and/or other sequence is indirectly operably linked to a first promoter. In some embodiment, the gene encoding the branched chain amino acid catabolism enzyme, BCAA transporter, BCAA binding protein, and/or other sequence is operably linked to a promoter that is not its native promoter.

In some embodiments, the gene encoding the branched chain amino acid catabolism enzyme, BCAA transporter, BCAA binding protein, and/or other sequence is expressed under the control of a constitutive promote. In other embodiments, the gene encoding the branched chain amino acid catabolism enzyme, BCAA transporter, BCAA binding protein, and/or other sequence is expressed under the control of an inducible promoter. In some embodiments, the gene encoding the branched chain amino acid catabolism enzyme, BCAA transporter, BCAA binding protein, and/or other sequence is expressed under the control of a promoter that is directly or indirectly induced by exogenous environmental conditions. In some embodiments, the gene encoding the branched chain amino acid catabolism enzyme, BCAA transporter, BCAA binding protein, and/or other sequence is expressed under the control of a promoter that is directly or indirectly induced by low-oxygen or anaerobic conditions, wherein expression of the gene encoding the branched chain amino acid catabolism enzyme is activated under low-oxygen or anaerobic environments, such as the environment of the mammalian gut. In some embodiments, the gene encoding the branched chain amino acid catabolism enzyme, BCAA transporter, BCAA binding protein, and/or other sequence is expressed under the control of a promoter that is directly or indirectly induced by inflammatory conditions. Exemplary inducible promoters described herein include oxygen level-dependent promoters (e.g., FNR-inducible promoter), promoters induced by inflammation or an inflammatory response (RNS, ROS promoters), and promoters induced by a metabolite that may or may not be naturally present (e.g., can be exogenously added) in the gut, e.g., arabinose and tetracycline. Examples of inducible promoters include, but are not limited to, an FNR responsive promoter, a P_{araC} promoter, a P_{araBAD} promoter, and a P_{TetR} promoter, each of which are described in more detail herein. Examples of other inducible promoters are provided herein below.

The gene encoding the branched chain amino acid catabolism enzyme, BCAA transporter, BCAA binding protein, and/or other sequence may be present on a plasmid or chromosome in the bacterial cell. In some embodiments, the gene encoding the branched chain amino acid catabolism enzyme, BCAA transporter, BCAA binding protein, and/or other sequence is located on a plasmid in the bacterial cell. In other embodiments, the gene encoding the branched chain amino acid catabolism enzyme, BCAA transporter, BCAA binding protein, and/or other sequence is located in the chromosome of the bacterial cell. In other embodiments, a native copy of the gene encoding the branched chain amino acid catabolism enzyme, BCAA transporter, BCAA binding protein, and/or other sequence is located in the chromosome of the bacterial cell, and a gene encoding a branched chain amino acid catabolism enzyme, BCAA transporter, BCAA binding protein, and/or other sequence from the same or a different species of bacteria is located on a plasmid in the bacterial cell. In other embodiments, a native copy of the gene encoding the branched chain amino acid catabolism enzyme, BCAA transporter, BCAA binding protein, and/or other sequence is located on a plasmid in the bacterial cell, and a gene encoding the branched chain amino acid catabolism enzyme, BCAA transporter, BCAA binding protein, and/or other sequence from a different species of bacteria is located on a plasmid in the bacterial cell. In other embodiments, a native copy of the gene encoding the branched chain amino acid catabolism enzyme, BCAA transporter, BCAA binding protein, and/or other sequence is located in the chromosome of the bacterial cell, and a gene encoding the branched chain amino acid catabolism enzyme, BCAA transporter, BCAA binding protein, and/or other sequence from a different species of bacteria is located in the chromosome of the bacterial cell.

In some embodiments, the gene encoding the branched chain amino acid catabolism enzyme, BCAA transporter, BCAA binding protein, and/or other sequence is expressed on a low-copy plasmid. In some embodiments, the gene encoding the branched chain amino acid catabolism enzyme, BCAA transporter, BCAA binding protein, and/or other sequence is expressed on a high-copy plasmid. In some embodiments, the high-copy plasmid may be useful for increasing expression of the branched chain amino acid catabolism enzyme, BCAA transporter, BCAA binding protein, and/or other sequence, thereby increasing the catabolism of the branched chain amino acid, e.g., leucine.

In some embodiments, the engineered bacteria convert the branched chain amino acid(s) and/or corresponding alpha-keto acid(s) and/or other corresponding metabolite(s) to a non-toxic or low toxicity metabolite, e.g., isovaleraldehyde, isobutyraldehyde, 2-methylbutyraldehyde, isovaleric acid, isobutyric acid, 2-methylbutyric acid, isopentanol, isobutanol, and 2-methylbutanol. Table 3 chart showing that the products of BCAA degradation by the engineered bacteria have very low oral toxicity.

TABLE 3
Toxicity of BCAA degradation products

| | Oral LD50 | Oral NOAEL* |
|---|---|---|
| Compound | (rat) (mg/kg) | (rat)(mg/kg/d) |
| Isovaleraldehyde | 5740 | N/D |
| Isolbutyraldehyde | 3730 | N/D |
| 2-methylbutyraldehyde | 6884 | N/D |
| Isovaleric acid | 2500 | N/D |
| Isobutyric acid | 2230 | N/D |
| 2-metylbutyric acid | 1750 | N/D |
| Isopentanol | >5000 | 1250 |
| Isobutanol | 3350 | >1450 |
| 2-methylbutanol | 4170 | N/D |

*No-Observed-Adverse-Effect

A. Optimized Branched Chain Ketoacid Decarboxylase

In one embodiment, the branched chain amino acid catabolism enzyme is an optimized branched chain ketoacid decarboxylase, including but not limited to, KivD. In a non-limiting example, KivD is from Lactococcus lactis. Thus, in some embodiments, the engineered bacteria comprise gene sequence(s) encoding one or more copies of a branched chain ketoacid decarboxylase. In some embodiments, the engineered bacteria comprise gene sequence(s) encoding one, two, three, four, five, six, or more copies of a branched chain ketoacid decarboxylase. The one or more copies of a branched chain ketoacid decarboxylase can be one or more copies of the same gene or can be different genes encoding α-ketoisovalerate decarboxylase, e.g., gene(s) from a different species or otherwise having a different gene sequence. The one or more copies of a branched chain ketoacid decarboxylase can be present in the bacterial chromosome or can be present in one or more plasmids.

As used herein “α-ketoisovalerate decarboxylase” or “alpha-ketoisovalerate decarboxylase” or “branched-chain α-keto acid decarboxylase” or “α-ketoacid decarboxylase” or “branched chain ketoacid decarboxylase” or “2-ketoisovalerate decarboxylase” (referred to herein also as KivD or ketoisovalerate decarboxylase) refers to any polypeptide having enzymatic activity that catalyzes the conversion of a branched chain alpha-keto acid (BCKA), such as α-ketoisovalerate (2-oxoisopentanoate), α-ketomethylvalerate (3-methyl-2-oxopentanoate), or α-ketoisocaproate 4-methyl-2-oxopentanoate), to its corresponding aldehyde, such as isobutyraldehyde, 2-methylbutyraldehyde, or isovaleraldehyde, and carbon dioxide. Branched chain ketoacid decarboxylase enzymes are available from many microorganism sources, including those disclosed herein. Branched chain ketoacid decarboxylase employs the co-factor thiamine diphosphate (also known as thiamine pyrophosphate or “TPP” or “TDP”). Thiamine is the vitamin form of the co-factor which, when transported into a cell, is converted to thiamine diphosphate. Alpha-ketoisovalerate decarboxylase also employs Mg²⁺. Branched chain ketoacid decarboxylase may be a homotetramer.

The bacterial cells disclosed herein may comprise a heterologous gene encoding a branched chain ketoacid decarboxylase enzyme and are capable of converting α-keto acids into aldehydes. For example, the branched chain ketoacid decarboxylase enzyme KivD is capable of metabolizing leucine (see, for example, de la Plaza et al., FEMS Microbiol. Lett. 2004, 238(2):367-374), and a cytosolically active KivD should generally exhibit the ability to convert ketoisovalerate, ketomethylvalerate, and ketoisocaproate to isobutyraldehyde, 2-methylbutyraldehyde, and isovaleraldehyde.

Multiple distinct a branched chain ketoacid decarboxylase proteins are known in the art (see, e.g., US Pat. Appl. Publ. No. 2013/0203138, the entire contents of which are incorporated herein by reference). In some embodiments, branched chain ketoacid decarboxylase is encoded by a branched chain ketoacid decarboxylase gene derived from a bacterial species. In some embodiments, a branched chain ketoacid decarboxylase is encoded by a branched chain ketoacid decarboxylase gene derived from a non-bacterial species. In some embodiments, a branched chain ketoacid decarboxylase is encoded by a branched chain ketoacid decarboxylase gene derived from a eukaryotic species, e.g., a yeast species or a plant species. In some embodiments, a branched chain ketoacid decarboxylase is encoded by a branched chain ketoacid decarboxylase gene derived from a mammalian species. In one embodiment, the a branched chain ketoacid decarboxylase gene is derived from an organism of the genus or species that includes, but is not limited to, Acetinobacter, Azospirillum, Bacillus, Bacteroides, Bifidobacterium, Brevibacteria, Burkholderia, Citrobacter, Clostridium, Corynebacterium, Cronobacter, Enterobacter, Enterococcus, Erwinia, Helicobacter, Klebsiella, Lactobacillus, Lactococcus, Leishmania, Listeria, Macrococcus, Mycobacterium, Nakamurella, Nasonia, Nostoc, Pantoea, Pectobacterium, Psychrobacter, Ralstonia, Saccharomyces, Salmonella, Sarcina, Serratia, Staphylococcus, and Yersinia, e.g., Acetinobacter radioresistens, Acetinobacter baumannii, Acetinobacter calcoaceticus, Azospirillum brasilense, Bacillus anthracis, Bacillus cereus, Bacillus coagulans, Bacillus megaterium, Bacillus subtilis, Bacillus thuringiensis, Bacteroides fragilis, Bacteroides subtilis, Bacteroides thetaiotaomicron, Bifidobacterium bifdum, Bifidobacterium infantis, Bifidobacterium lactis, Bifidobacterium longum, Burkholderia xenovorans, Citrobacter youngae, Citrobacter koseri, Citrobacter rodentium, Clostridium acetobutylicum, Clostridium butyricum, Corynebacterium aurimucosum, Corynebacterium kroppenstedtii, Corynebacterium striatum, Cronobacter sakazakii, Cronobacter turicensis, Enterobacter cloacae, Enterobacter cancerogenus, Enterococcus faecium, Erwinia amylovara, Erwinia pyrifoliae, Erwinia tasmaniensis, Helicobacter mustelae, Klebsiella pneumonia, Klebsiella variicola, Lactobacillus acidophilus, Lactobacillus bulgaricus, Lactobacillus casei, Lactobacillus johnsonii, Lactobacillus paracasei, Lactobacillus plantarum, Lactobacillus reuteri, Lactobacillus rhamnosus, Lactococcus lactis, Leishmania infantum, Leishmania major, Leishmania brazilensis, Listeria grayi, Macrococcus caseolyticus, Mycobacterium avium, Mycobacterium intracellulare, Mycobacterium kansasii, Mycobacterium leprae, Mycobacterium marinum, Mycobacterium smegmatis, Mycobacterium tuberculosis, Mycobacterium ulcerans, Nakamurella multipartita, Nasonia vitipennis, Nostoc punctiforme, Pantoea ananatis, Pantoea agglomerans, Pectobacterium atrosepticum, Pectobacterium carotovorum, Psychrobacter articus, Psychrobacter cryohalolentis, Ralstonia eutropha, Saccharomyces boulardii, Salmonella enterica, Sarcina ventriculi, Serratia odorifera, Serratia proteamaculans, Staphylococcus aerus, Staphylococcus capitis, Staphylococcys carnosus, Staphylococcus epidermidis, Staphylococcus hominis, Staphylococcus haemolyticus, Staphylococcus lugdunensis, Staphylococcus saprophyticus, Staphylococcus warneri, Yersinia enterocolitica, Yersinia mollaretii, Yersinia kristensenii, Yersinia rohdei, and Yersinia aldovae. In some embodiments, the branched chain ketoacid decarboxylase is encoded by an a branched chain ketoacid decarboxylase gene derived from Lactococcus lactis, e.g., IFPL730. In another embodiment, the branched chain ketoacid decarboxylase, e.g., kivD gene, is derived from Enterobacter cloacae (Accession No. P23234.1), Mycobacterium smegmatis (Accession No. A0R480.1), Mycobacterium tuberculosis (Accession NO. 053865.1), Mycobacterium avium (Accession No. Q742Q2.1), Azospirillum brasilense (Accession No. P51852.1), or Bacillus subtilis (see Oku et al., J Biol. Chem. 263: 18386-96, 1988).

In one embodiment, the branched chain ketoacid decarboxylase gene has been codon-optimized for use in the recombinant bacterial cell. In one embodiment, the branched chain ketoacid decarboxylase gene has been codon-optimized for use in Escherichia coli.

In one embodiment, the branched chain ketoacid decarboxylase gene is a kivD gene. In another embodiment, the kivD gene is a Lactococcus lactis kivD gene or kivD gene derived from Lactococcus lactis. When a branched chain ketoacid decarboxylase is expressed in the recombinant bacterial cells disclosed herein, the bacterial cells catabolize more branched chain amino acid, e.g., leucine, than unmodified bacteria of the same bacterial subtype under the same conditions (e.g., culture or environmental conditions). Thus, the genetically engineered bacteria comprising a heterologous gene encoding a branched chain ketoacid decarboxylase may be used to catabolize excess branched chain amino acids, e.g., leucine, to treat a disease associated with the catabolism of a branched chain amino acid, including Maple Syrup Urine Disease (MSUD).

The present disclosure further comprises genes encoding functional fragments of a branched chain ketoacid decarboxylase or functional variants of a branched chain ketoacid decarboxylase gene. As used herein, the term “functional fragment thereof” or “functional variant thereof” of a branched chain ketoacid decarboxylase gene relates to a sequence having qualitative biological activity in common with the wild-type branched chain ketoacid decarboxylase from which the fragment or variant was derived. For example, a functional fragment or a functional variant of a mutated branched chain ketoacid decarboxylase protein is one which retains essentially the same ability to catabolize BCKAs as a branched chain ketoacid decarboxylase protein from which the functional fragment or functional variant was derived. For example, a polypeptide having branched chain ketoacid decarboxylase activity may be truncated at the N-terminus or C-terminus and the retention of branched chain ketoacid decarboxylase activity assessed using assays known to those of skill in the art, including the exemplary assays provided herein. In one embodiment, the recombinant bacterial cell disclosed herein comprises a heterologous gene encoding a branched chain ketoacid decarboxylase functional variant. In another embodiment, the recombinant bacterial cell disclosed herein comprises a heterologous gene encoding a branched chain ketoacid decarboxylase functional fragment.

Assays for testing the activity of a branched chain ketoacid decarboxylase, a branched chain ketoacid decarboxylase functional variant, or a branched chain ketoacid decarboxylase functional fragment are well known to one of ordinary skill in the art. For example, branched chain ketoacid decarboxylase activity can be assessed by expressing the protein, functional variant, or fragment thereof, in a recombinant bacterial cell that lacks endogenous branched chain ketoacid decarboxylase activity. Also, branched chain ketoacid decarboxylase activity can be assessed using the coupled enzymatic assay method as described by Zhang et al. (see, for example, Zhang et al., Proc. Natl. Acad. Sci., 105(52):20653-58, 2008). Alpha-ketoisovalerate decarboxylase activity can also be assessed in vitro by measuring the disappearance of alpha-ketoisovalerate as described by de la Plaza (see, for example, de la Plaza et al., FEMS Microbiol. Letters, 2004, 238(2):367-374).

In some embodiments, the gene encoding a branched chain ketoacid decarboxylase, e.g., kivD, is mutagenized; mutants exhibiting increased activity are selected; and the mutagenized gene encoding the α-ketoisovalerate decarboxylase, e.g., kivD, is isolated and inserted into the bacterial cell. The gene comprising the modifications described herein may be present on a plasmid or chromosome.

Accordingly, in some embodiments, the kivD gene has at least about 80% identity with the entire sequence of any one of SEQ ID NOs: 73-96. Accordingly, in one embodiment, the kivD gene has at least about 90% identity with the entire sequence of any one of SEQ ID NOs: 73-96. Accordingly, in one embodiment, the kivD gene has at least about 95% identity with the entire sequence of any one of SEQ ID NOs: 73-96. Accordingly, in one embodiment, the kivD gene has at least about 85%, 86%, 87%, 88%, 89%, 90%, 91%, 92%, 93%, 94%, 95%, 96%, 97%, 98%, or 99% identity with the entire sequence of any one of SEQ ID NOs: 73-96. In another embodiment, the kivD gene comprises the sequence of any one of SEQ ID NOs: 73-96. In yet another embodiment, the kivD gene consists of any one of SEQ ID NOs: 73-96.

In other embodiments, the engineered bacteria comprise gene sequence(s) encoding at least one branched chain ketoacid decarboxylase enzyme and further comprise gene sequence encoding one or more polypeptides selected from other branched chain amino acid catabolism enzyme(s), BCAA transporter(s), and BCAA binding protein(s). Thus, in some embodiments, the at least one branched chain ketoacid decarboxylase enzyme is coexpressed with an additional branched chain amino acid catabolism enzyme, e.g., a branched chain amino acid dehydrogenase, amino acid oxidase (also known as amino acid deaminase), and/or aminotransferase. In some embodiments, the at least one α-ketoisovalerate decarboxylase gene is coexpressed with a leucine dehydrogenase, e.g., (leuDH or ldh), described in more detail below. In other embodiments, the at least one branched chain ketoacid decarboxylase gene is coexpressed with a branched chain amino acid aminotransferase, e.g., ilvE, described in more detail below. In other embodiments, the at least one branched chain ketoacid decarboxylase gene is coexpressed with an amino acid deaminase, e.g., L-AAD, described in more detail below. In some embodiments, the engineered bacteria comprise gene sequence(s) encoding at least one branched chain ketoacid decarboxylase enzyme and gene sequence(s) encoding one or more branched chain amino acid dehydrogenase(s) (e.g., leuDH). In some embodiments, the engineered bacteria comprise gene sequence(s) encoding at least one branched chain ketoacid decarboxylase enzyme and gene sequence(s) encoding one or more aminotransferase(s) (e.g., ilvE).

In some embodiments, the at least one branched chain ketoacid decarboxylase enzyme is coexpressed with an alcohol dehydrogenase, e.g., adh2, described in more detail below. In some embodiments, the engineered bacteria comprise gene sequence(s) encoding at least one branched chain ketoacid decarboxylase enzyme and gene sequence(s) encoding one or more alcohol dehydrogenase(s) (e.g., adh2).

In some embodiments, the at least one α-ketoisovalerate decarboxylase gene is coexpressed with a leucine dehydrogenase, e.g., leuDH, and an alcohol dehydrogenase, e.g., adh2.

In some embodiments, the at least one branched chain ketoacid decarboxylase enzyme is coexpressed with one or more BCAA transporter(s), for example, a high affinity leucine transporter, e.g., LivKHMGF and/or low affinity BCAA transporter, e.g., BrnQ. In some embodiments, the engineered bacteria comprise gene sequence(s) encoding at least one branched chain ketoacid decarboxylase enzyme and gene sequence(s) encoding one or more BCAA transporter(s) (e.g., livKHMGF, brnQ).

In some embodiments, the engineered bacteria comprise gene sequence(s) encoding at least one branched chain ketoacid decarboxylase enzyme and genetic modification that reduces export of a branched chain amino acid, e.g., a genetic mutation in a leuE gene or promoter thereof. In one embodiment, the engineered bacteria comprise gene sequence(s) encoding at least one branched chain ketoacid decarboxylase enzyme and a genetic modification that reduces or eliminates branched chain amino acid synthesis, e.g., a genetic mutation in a ilvC gene or promoter thereof.

In some embodiments, the gene sequence(s) encoding the one or more branched chain ketoacid decarboxylase enzyme(s) is expressed under the control of a constitutive promoter. In some embodiments, the gene sequence(s) encoding the one or more branched chain ketoacid decarboxylase enzyme(s) is expressed under the control of an inducible promoter. In some embodiments, the gene sequence(s) encoding the one or more branched chain ketoacid decarboxylase enzyme(s) is expressed under the control of a promoter that is directly or indirectly induced by exogenous environmental conditions. In some embodiments, the gene sequence(s) encoding the one or more branched chain ketoacid decarboxylase enzyme(s) is expressed under the control of a promoter that is directly or indirectly induced by low-oxygen or anaerobic conditions, wherein expression of the gene encoding the branched chain ketoacid decarboxylase enzyme is activated under low-oxygen or anaerobic environments, such as the environment of the mammalian gut. In some embodiments, the gene sequence(s) encoding the one or more branched chain ketoacid decarboxylase enzyme(s) is expressed under the control of a promoter that is directly or indirectly induced by inflammatory conditions. Exemplary inducible promoters described herein include oxygen level-dependent promoters (e.g., FNR-inducible promoter), promoters induced by inflammation or an inflammatory response (RNS, ROS promoters), and promoters induced by a metabolite that may or may not be naturally present (e.g., can be exogenously added) in the gut, e.g., arabinose and tetracycline. Examples of inducible promoters include, but are not limited to, an FNR responsive promoter, a P_{araC} promoter, a P_{araBAD} promoter, and a P_{TetR} promoter, each of which are described in more detail herein.

B. Optimized Branched Chain Amino Acid Deamination Enzymes

In one embodiment, the branched chain amino acid catabolism enzyme is a branched chain amino acid deamination enzyme. Thus, in some embodiments, the engineered bacteria comprise gene sequence(s) encoding one or more copies of a branched chain amino acid deamination enzyme. In some embodiments, the engineered bacteria comprise gene sequence(s) encoding one, two, three, four, five, six, or more copies of a branched chain amino acid deamination enzyme. The one or more copies of branched chain amino acid deamination enzyme can be one or more copies of the same gene or can be different genes encoding branched chain amino acid deamination enzyme, e.g., gene(s) from a different species or otherwise having a different gene sequence. The one or more copies of branched chain amino acid deamination enzyme can be present in the bacterial chromosome or can be present in one or more plasmids. As used herein, the term “branched chain amino acid deamination enzyme” refers to an enzyme involved in the deamination, or the removal of an amine group, of a branched chain amino acid, which produces a corresponding branched chain alpha-keto acid (e.g., α-ketoisocaproate, α-keto-βmethylvalerate, α-ketoisovalerate). Enzymes involved in the deamination of branched chain amino acids are well known to those of skill in the art. For example, in bacteria, leucine dehydrogenase (LeuDH, leuDH), e.g., derived from Pseudomonas aeruginosa PA01, is capable of catalyzing the reversible deamination of branched chain amino acids, such as leucine, into their corresponding keto-acid counterpart (Baker et al., Structure, 3(7):693-705, 1995).

In one embodiment, the branched chain amino acid deamination enzyme increases the rate of branched chain amino acid deamination in the cell. In one embodiment, the branched chain amino acid deamination enzyme decreases the level of branched chain amino acid in the cell as compared to the level of its corresponding alpha-keto acid in the cell. In another embodiment, the branched chain amino acid deamination enzyme increases the level of alpha-keto acid in the cell as compared to the level of its corresponding branched chain amino acid in the cell.

In one embodiment, the branched chain amino acid deamination enzyme is a leucine deamination enzyme. In another embodiment, the branched chain amino acid deamination enzyme is an isoleucine deamination enzyme. In another embodiment, the branched chain amino acid deamination enzyme is a valine deamination enzyme. In another embodiment, the branched chain amino acid deamination enzyme is involved in the deamination of leucine, isoleucine, and valine. In another embodiment, the branched chain amino acid deamination enzyme is involved in the deamination of leucine and valine, isoleucine and valine, or leucine and isoleucine. In some embodiments, the branched chain amino acid deamination enzyme is encoded by a branched chain amino acid deamination enzyme gene derived from a bacterial species. In some embodiments, the branched chain amino acid deamination enzyme is encoded by a branched chain amino acid deamination enzyme gene derived from a non-bacterial species. In some embodiments, the branched chain amino acid deamination enzyme is encoded by a branched chain amino acid deamination enzyme gene derived from a eukaryotic species, e.g., a yeast species or a plant species. In another embodiment, the branched chain amino acid deamination enzyme is encoded by a branched chain amino acid deamination enzyme gene derived from a mammalian species, e.g., human.

In some embodiments, the branched chain amino acid deamination enzyme is a branched chain amino acid dehydrogenase. Thus, in some embodiments, the engineered bacteria comprise gene sequence(s) encoding one or more copies of a branched chain amino acid dehydrogenase. In some embodiments, the engineered bacteria comprise gene sequence(s) encoding one, two, three, four, five, six, or more copies of a branched chain amino acid dehydrogenase. The one or more copies of branched chain amino acid dehydrogenase can be one or more copies of the same gene or can be different genes encoding branched chain amino acid dehydrogenase, e.g., gene(s) from a different species or otherwise having a different gene sequence. The one or more copies of branched chain amino acid dehydrogenase can be present in the bacterial chromosome or can be present in one or more plasmids.

In some embodiments, the branched chain amino acid deamination enzyme is leucine dehydrogenase (“leuDH” or “leuDH”). As used herein “leucine dehydrogenase” refers to any polypeptide having enzymatic activity that deaminates leucine to its corresponding ketoacid, alpha-ketoisocaproate (KIC), deaminates valine to its corresponding ketoacid, ketoisovalerate (KIV), and deaminates isoleucine to its corresponding ketoacid, ketomethylvalerate (KMV). In some embodiments, the bacterial cells disclosed herein comprise a heterologous gene encoding a leucine dehydrogenase enzyme and are capable of converting leucine, valine, and/or isoleucine to their respective α-keto acids. For example, the leucine dehydrogenase enzyme LeuDH is capable of metabolizing leucine and a cytosolically active LeuDH should generally exhibit the ability to convert valine, isoleucine, and leucine to ketoisovalerate, ketomethylvalerate, and ketoisocaproate, respectively. Leucine dehydrogenase employs the co-factor NAD+. In some embodiments, leuDH encodes an octamer.

Multiple distinct leucine dehydrogenases (EC 1.4.1.9) are known in the art and are available from many microorganism sources, including those disclosed herein, as well as from eukaryotic sources (see, for example, Baker et al., Structure, 3(7):693-705, 1995). In some embodiments, the branched chain amino acid deamination enzyme is encoded by at least one gene encoding a branched chain amino acid deamination enzyme derived from a bacterial species. In some embodiments, the branched chain amino acid deamination enzyme is encoded by at least one gene encoding a branched chain amino acid deamination enzyme derived from a non-bacterial species. In some embodiments, the branched chain amino acid deamination enzyme is encoded by at least one gene derived from a eukaryotic species, e.g., a yeast species or a plant species. In another embodiment, the branched chain amino acid deamination enzyme is encoded by at least one gene derived from a mammalian species, e.g., human.

In one embodiment, the engineered bacteria comprise gene sequence(s) encoding one or more branched chain amino acid dehydrogenase(s), e.g., leucine dehydrogenase enzyme(s). In some embodiments, the branched chain amino acid dehydrogenase, e.g., leucine dehydrogenase enzyme is derived from an organism of the genus or species that includes, but is not limited to, Bacillus, Brevibacillus, Geobacillus, Lysinibacillus, Moorella, Natrialba, Pseudomonas, Sporosarcinia, and Thermoactinomyces. In some embodiments, the leuDH gene is encoded by a gene derived from Bacillus caldolyticus, Bacillus cereus, Bacillus licheniformis, Bacillus megaterium, Bacillus mycoides, Bacillus niger, Bacillus pumilus, Bacillus subtilis, Brevibacillus brevis, Geobacillus stearothermophilus, Lysinibacillus sphaeriscus, Moorella Thermoacetica, Natrialba magadii, Sporosarcina psychorophila, Thermoactinomyces intermedius, Pseudomonas aeruginosa, or Pseudomonas resinovorans. In some embodiments, the branched chain amino acid dehydrogenase enzyme, e.g., leucine dehydrogenase, is encoded by at least one gene derived from Pseudomonas aeruginosa PA01. In some embodiments, the branched chain amino acid dehydrogenase enzyme, e.g., leucine dehydrogenase, is encoded by at least one gene derived from Bacillus cereus. In some embodiments, the at least one gene encoding the branched chain amino acid dehydrogenase enzyme, e.g., leucine dehydrogenase, has been codon-optimized for use in the recombinant bacterial cell. In one embodiment, the at least one gene encoding the branched chain amino acid dehydrogenase enzyme, e.g., leucine dehydrogenase, has been codon-optimized for use in Escherichia coli.

When a branched chain amino acid dehydrogenase enzyme, e.g., leucine dehydrogenase, is expressed in the recombinant bacterial cells disclosed herein, the bacterial cells catabolize more branched chain amino acid, e.g., leucine, isoleucine, and valine, than unmodified bacteria of the same bacterial subtype under the same conditions (e.g., culture or environmental conditions). Thus, the genetically engineered bacteria comprising at least one heterologous gene encoding a branched chain amino acid dehydrogenase enzyme, e.g., leucine dehydrogenase, may be used to catabolize excess branched chain amino acids, e.g., leucine, valine, and isoleucine, to treat a disease associated with the deamination of a branched chain amino acid, including Maple Syrup Urine Disease (MSUD) as well as other disease provided herein.

The present disclosure further comprises genes encoding functional fragments of a branched chain amino acid dehydrogenase enzyme, e.g., leucine dehydrogenase, or functional variants of branched chain amino acid dehydrogenase enzyme, e.g., leucine dehydrogenase. The present disclosure encompasses genes encoding a branched chain amino acid dehydrogenase enzyme, e.g., leucine dehydrogenase, comprising amino acids in its sequence that are substantially the same as an amino acid sequence described herein. As used herein, the term “functional fragment thereof” or “functional variant thereof” of a branched chain amino acid dehydrogenase enzyme, e.g., a leucine dehydrogenase, gene relates to a sequence having qualitative biological activity in common with the wild-type branched chain amino acid dehydrogenase, e.g., a leucine dehydrogenase, from which the fragment or variant was derived. For example, a functional fragment or a functional variant of a mutated branched chain amino acid dehydrogenase protein, e.g., a leucine dehydrogenase, is one which retains essentially the same ability to catabolize BCAAs as the branched chain amino acid dehydrogenase protein, e.g., a leucine dehydrogenase, from which the functional fragment or functional variant was derived. For example, a polypeptide having branched chain amino acid dehydrogenase enzyme, e.g., a leucine dehydrogenase, activity may be truncated at the N-terminus or C-terminus and the retention of branched chain amino acid dehydrogenase enzyme, e.g., a leucine dehydrogenase, activity assessed using assays known to those of skill in the art, including the exemplary assays provided herein. In one embodiment, the recombinant bacterial cell disclosed herein comprises a heterologous gene encoding an a branched chain amino acid dehydrogenase enzyme, e.g., a leucine dehydrogenase, functional variant. In another embodiment, the recombinant bacterial cell disclosed herein comprises a heterologous gene encoding a branched chain amino acid dehydrogenase enzyme, e.g., a leucine dehydrogenase, functional fragment.

Assays for testing the activity of a branched chain amino acid dehydrogenase enzyme functional variant or functional fragment, e.g., a leucine dehydrogenase functional variant or a leucine dehydrogenase functional fragment are well known to one of ordinary skill in the art. For example, leucine dehydrogenase activity can be assessed by expressing the protein, functional variant, or fragment thereof, in a recombinant bacterial cell that lacks endogenous leucine dehydrogenase activity. Also, activity can be assessed using the enzymatic assay methods as described by Soda et al. (Biochem. Biophys. Res. Commun., 44:931, 1971), and Ohshima et al. (J. Biol. Chem., 253:5719, 1978), the entire contents of each of which are expressly incorporated herein by reference.

In some embodiments, the at least one gene encoding a branched chain amino acid dehydrogenase enzyme, e.g., leucine dehydrogenase is mutagenized, mutants exhibiting increased activity are selected, and the mutagenized gene(s) encoding the branched chain amino acid dehydrogenase enzyme, e.g., leucine dehydrogenase, are isolated and inserted into the bacterial cell. In some embodiments, the at least one gene encoding a branched chain amino acid dehydrogenase enzyme, e.g., leucine dehydrogenase is mutagenized, mutants exhibiting decreased activity are selected, and the mutagenized gene(s) encoding the branched chain amino acid dehydrogenase enzyme, e.g., leucine dehydrogenase, are isolated and inserted into the bacterial cell. The gene comprising the modifications described herein may be present on a plasmid or chromosome.

In some embodiments, the engineered bacteria comprise gene sequence(s) encoding at least one branched chain amino acid dehydrogenase enzyme, e.g., leucine dehydrogenase, and further comprise gene sequence encoding one or more polypeptides selected from other branched chain amino acid catabolism enzyme(s), BCAA transporter(s), and BCAA binding protein(s). Thus, in some embodiments, the at least one branched chain amino acid dehydrogenase enzyme, e.g., leucine dehydrogenase, is coexpressed with an additional branched chain amino acid deamination enzyme, e.g., branched chain amino acid dehydrogenase, a branched chain aminotransferase, and/or amino acid oxidase (also known as amino acid deaminase). For example, in some embodiments, the branched chain amino acid dehydrogenase enzyme, e.g., leucine dehydrogenase, is coexpressed with one or more other branched chain amino acid dehydrogenase enzyme(s).

In some embodiments, the branched chain amino acid dehydrogenase enzyme, e.g., leucine dehydrogenase, is coexpressed with one or more other branched chain amino acid catabolism enzyme(s), for example, a ketoacid decarboxylase, such as kivD. In some embodiments, the branched chain amino acid dehydrogenase enzyme, e.g., leucine dehydrogenase, is coexpressed with one or more other branched chain amino acid catabolism enzyme(s), for example, a branched chain alcohol dehydrogenase, such as adh2. In other embodiments, the branched chain amino acid dehydrogenase enzyme, e.g., leucine dehydrogenase, is coexpressed with a second branched chain amino acid catabolism enzyme, for example, kivD, and a branched chain alcohol dehydrogenase, for example, adh2. In some embodiments, the engineered bacteria comprise gene sequence(s) encoding at least one branched chain amino acid dehydrogenase enzyme, e.g., leucine dehydrogenase enzyme, and gene sequence(s) encoding one or more keto-acid decarboxylase(s) (e.g., kivD). In some embodiments, the engineered bacteria comprise gene sequence(s) encoding at least one branched chain amino acid dehydrogenase enzyme, e.g., leucine dehydrogenase enzyme, and gene sequence(s) encoding one or more alcohol dehydrogenase(s) (e.g., adh2).

In some embodiments, the at least one branched chain amino acid dehydrogenase enzyme, e.g., leucine dehydrogenase enzyme is coexpressed with one or more BCAA transporter(s), for example, a high affinity leucine transporter, e.g., LivKHMGF and/or the low affinity BCAA transporter BmQ. In some embodiments, the engineered bacteria comprise gene sequence(s) encoding at least one branched chain amino acid dehydrogenase enzyme, e.g., leucine dehydrogenase enzyme and gene sequence(s) encoding one or more BCAA transporter(s) (e.g., livKHMGF and/or brnQ).

In some embodiments, the engineered bacteria comprise gene sequence(s) encoding at least one branched chain amino acid dehydrogenase enzyme, e.g., leucine dehydrogenase enzyme, and genetic modification that reduces export of a branched chain amino acid, e.g., a genetic mutation in a leuE gene or promoter thereof. In one embodiment, the engineered bacteria comprise gene sequence(s) encoding at least one branched chain amino acid dehydrogenase enzyme, e.g., leucine dehydrogenase enzyme, and a genetic modification that reduces or eliminates branched chain amino acid synthesis, e.g., a genetic mutation in a ilvC gene or promoter thereof.

In some embodiments, the gene sequence(s) encoding the one or more branched chain amino acid dehydrogenase enzyme(s), e.g., leucine dehydrogenase enzyme(s) is expressed under the control of a constitutive promoter. In some embodiments, the gene sequence(s) encoding the one or more branched chain amino acid dehydrogenase enzyme(s), e.g., leucine dehydrogenase enzyme(s) is expressed under the control of an inducible promoter. In some embodiments, the gene sequence(s) encoding the one or more branched chain amino acid dehydrogenase enzyme(s), e.g., leucine dehydrogenase enzyme(s) is expressed under the control of a promoter that is directly or indirectly induced by exogenous environmental conditions. In some embodiments, the gene sequence(s) encoding the one or more branched chain amino acid dehydrogenase enzyme(s), e.g., leucine dehydrogenase enzyme(s) is expressed under the control of a promoter that is directly or indirectly induced by low-oxygen or anaerobic conditions, wherein expression of the gene encoding the branched chain amino acid dehydrogenase enzyme, e.g., leucine dehydrogenase enzyme is activated under low-oxygen or anaerobic environments, such as the environment of the mammalian gut. In some embodiments, the gene sequence(s) encoding the one or more branched chain amino acid dehydrogenase enzyme(s), e.g., leucine dehydrogenase enzyme(s) is expressed under the control of a promoter that is directly or indirectly induced by inflammatory conditions. Exemplary inducible promoters described herein include oxygen level-dependent promoters (e.g., FNR-inducible promoter), promoters induced by inflammation or an inflammatory response (RNS, ROS promoters), and promoters induced by a metabolite that may or may not be naturally present (e.g., can be exogenously added) in the gut, e.g., arabinose and tetracycline. Examples of inducible promoters include, but are not limited to, an FNR responsive promoter, a P_{araC} promoter, a P_{araBAD} promoter, and a P_{TetR} promoter, each of which are described in more detail herein.

In some embodiments, the branched chain amino acid dehydrogenase is leucine dehydrogenase. The present disclosure further comprises genes encoding functional fragments of leucine dehydrogenase, or functional variants of leucine dehydrogenase. The present disclosure encompasses genes encoding leucine dehydrogenase, comprising amino acids in its sequence that are substantially the same as an amino acid sequence described herein.

In some embodiments, the at least one leuDH gene has at least about 80% identity with any one of SEQ ID NOs:49-72. Accordingly, in one embodiment, the at least one leuDH gene has at least about 90% identity with any one of SEQ ID NOs:49-72. Accordingly, in one embodiment, the at least one leuDH gene has at least about 95% identity with any one of SEQ ID NOs:49-72. Accordingly, in one embodiment, the at least one leuDH gene has at least about 85%, 86%, 87%, 88%, 89%, 90%, 91%, 92%, 93%, 94%, 95%, 96%, 97%, 98%, or 99% identity with any one of SEQ ID NOs:49-72. In another embodiment, the at least one leuDH gene comprises any one of SEQ ID NOs:49-72. In yet another embodiment the at least one leuDH gene consists of any one of SEQ ID NOs:49-72.

C. Optimized Alcohol Dehydrogenase Enzymes

In some embodiments, wherein a branched chain amino acid catabolism enzyme is used to convert a ketoacid to its corresponding aldehyde, the recombinant bacterial cells may further comprise an alcohol dehydrogenase enzyme in order to convert the branched chain amino acid-derived aldehyde to its respective alcohol. In some embodiments, the engineered bacteria comprise gene sequence(s) encoding one or more copies of an alcohol dehydrogenase. In some embodiments, the engineered bacteria comprise gene sequence(s) encoding one, two, three, four, five, six, or more copies of an alcohol dehydrogenase. The one or more copies of an alcohol dehydrogenase can be one or more copies of the same gene or can be different genes encoding alcohol dehydrogenase, e.g., gene(s) from a different species or otherwise having a different gene sequence. The one or more copies of alcohol dehydrogenase can be present in the bacterial chromosome or can be present in one or more plasmids. As used herein, “alcohol dehydrogenase” refers to any polypeptide having enzymatic activity that catalyzes the conversion of a branched chain amino acid-derived aldehyde, e.g., isovaleraldehyde, isobutyraldehyde, and 2-methylbutyraldehyde, into its respective alcohol, e.g., isopentanol, isobutanol, and 2-methylbutanol.

In general, alcohol dehydrogenases (EC 1.1.1.1) belong to a group of dehydrogenase enzymes that facilitate the interconversion between alcohols and aldehydes or ketones with the reduction of nicotinamide adenine dinucleotide (NAD+ to NADH). Multiple distinct alcohol dehydrogenases are known in the art and are available from many microorganism sources, including those disclosed herein, as well as eukaryotic and plant sources (see, for example, Bennetzen et al., J. Biol. Chem., 257(6):3018-25, 1982 and Teng et al., Human Genetics, 53(1):87-90, 1979, the entire contents of each of which are expressly incorporated herein by reference).

In some embodiments, the alcohol dehydrogenase is encoded by at least one gene derived from a bacterial species. In some embodiments, the alcohol dehydrogenase is encoded by at least one gene derived from a non-bacterial species. In some embodiments, the alcohol dehydrogenase is encoded by at least one gene derived from a eukaryotic species, e.g., a yeast species or a plant species. In another embodiment, the alcohol dehydrogenase is encoded by at least one gene derived from a mammalian species, e.g., human.

In one embodiment, the at least one gene encoding the alcohol dehydrogenase is derived from an organism of the genus or species that includes, but is not limited to, Acetinobacter, Azospirillum, Bacillus, Bacteroides, Bifidobacterium, Brevibacteria, Burkholderia, Citrobacter, Clostridium, Corynebacterium, Cronobacter, Enterobacter, Enterococcus, Erwinia, Helicobacter, Klebsiella, Lactobacillus, Lactococcus, Leishmania, Listeria, Macrococcus, Mycobacterium, Nakamurella, Nasonia, Nostoc, Pantoea, Pectobacterium, Proteus, Pseudomonas, Psychrobacter, Ralstonia, Saccharomyces, Salmonella, Sarcina, Serratia, Staphylococcus, Streptococcus, and Yersinia, e.g., Acetinobacter radioresistens, Acetinobacter baumannii, Acetinobacter calcoaceticus, Azospirillum brasilense, Bacillus anthracis, Bacillus cereus, Bacillus coagulans, Bacillus megaterium, Bacillus subtilis, Bacillus thuringiensis, Bacteroides fragilis, Bacteroides subtilis, Bacteroides thetaiotaomicron, Bifidobacterium bifdum, Bifidobacterium infantis, Bifdobacterium lactis, Bifidobacterium longum, Burkholderia xenovorans, Citrobacter youngae, Citrobacter koseri, Citrobacter rodentium, Clostridium acetobutylicum, Clostridium butyricum, Corynebacterium aurimucosum, Corynebacterium kroppenstedtii, Corynebacterium striatum, Cronobacter sakazakii, Cronobacter turicensis, Enterobacter cloacae, Enterobacter cancerogenus, Enterococcus faecium, Enterococcus faecalis, Erwinia amylovara, Erwinia pyrifoliae, Erwinia tasmaniensis, Helicobacter mustelae, Klebsiella pneumonia, Klebsiella variicola, Lactobacillus acidophilus, Lactobacillus bulgaricus, Lactobacillus casei, Lactobacillus johnsonii, Lactobacillus paracasei, Lactobacillus plantarum, Lactobacillus reuteri, Lactobacillus rhamnosus, Lactococcus lactis, Leishmania infantum, Leishmania major, Leishmania brazilensis, Listeria grayi, Macrococcus caseolyticus, Mycobacterium avium, Mycobacterium intracellulare, Mycobacterium kansasii, Mycobacterium leprae, Mycobacterium marinum, Mycobacterium smegmatis, Mycobacterium tuberculosis, Mycobacterium ulcerans, Nakamurella multipartita, Nasonia vitipennis, Nostoc punctiforme, Pantoea ananatis, Pantoea agglomerans, Pectobacterium atrosepticum, Pectobacterium carotovorum, Pseudomonas putida, Pseudomonas aeruginosa, Psychrobacter articus, Proteus vulgaris, Psychrobacter cryohalolentis, Ralstonia eutropha, Saccharomyces boulardii, Salmonella enterica, Sarcina ventriculi, Serratia odorifera, Serratia proteamaculans, Staphylococcus aerus, Staphylococcus capitis, Staphylococcys carnosus, Staphylococcus epidermidis, Staphylococcus hominis, Staphylococcus haemolyticus, Staphylococcus lugdunensis, Staphylococcus saprophyticus, Staphylococcus warneri, Streptococcus faecalis, Yersinia enterocolitica, Yersinia mollaretii, Yersinia kristensenii, Yersinia rohdei, and Yersinia aldovae. In some embodiments, the alcohol dehydrogenase is adh2. In some embodiments, the alcohol dehydrogenase is encoded by at least one gene derived from Saccharomyces cerevisiae. In another embodiment, the alcohol dehydrogenase is encoded by at least one gene derived from E. coli. In another embodiment, the alcohol dehydrogenase is encoded by at least one gene derived from Oryza sativa. In another embodiment, the alcohol dehydrogenase is encoded by at least one gene derived from Penicillium brasilianum. In another embodiment, the alcohol dehydrogenase is encoded by at least one gene derived from Bifidobacterium longum.

Accordingly, in some embodiments, the adh2 gene has at least about 80% identity with the entire sequence of any one of SEQ ID NOs: 97-120. Accordingly, in one embodiment, the adh2 gene has at least about 90% identity with the entire sequence of any one of SEQ ID NOs: 97-120. Accordingly, in one embodiment, the adh2 gene has at least about 95% identity with the entire sequence of any one of SEQ ID NOs: 97-120. Accordingly, in one embodiment, the adh2 gene has at least about 85%, 86%, 87%, 88%, 89%, 90%, 91%, 92%, 93%, 94%, 95%, 96%, 97%, 98%, or 99% identity with the entire sequence of any one of SEQ ID NOs: 97-120. In another embodiment, the adh2 gene comprises the sequence of any one of SEQ ID NOs: 97-120. In yet another embodiment, the adh2 gene consists of any one of SEQ ID NOs: 97-120.

In some embodiments, the at least one gene encoding the alcohol dehydrogenase has been codon-optimized for use in the recombinant bacterial cell. In some embodiments, the at least one gene encoding the alcohol dehydrogenase has been codon-optimized for use in Escherichia coli.

When an alcohol dehydrogenase is expressed in the recombinant bacterial cells disclosed herein, the bacterial cells convert more branched chain amino acid-derived aldehydes to their respective alcohols than unmodified bacteria of the same bacterial subtype under the same conditions (e.g., culture or environmental conditions). Thus, the genetically engineered bacteria comprising at least one heterologous gene encoding an alcohol dehydrogenase may be used to catabolize excess branched chain amino acid-derived aldehydes, e.g., isovaleraldehyde, to treat a disease associated with a branched chain amino acid, including Maple Syrup Urine Disease (MSUD).

The present disclosure encompasses genes encoding an alcohol dehydrogenase comprising amino acids in its sequence that are substantially the same as an amino acid sequence described herein. The present disclosure further comprises genes encoding functional fragments of an alcohol dehydrogenase or functional variants of an alcohol dehydrogenase. As used herein, the term “functional fragment thereof” or “functional variant thereof” of an alcohol dehydrogenase gene relates to a sequence having qualitative biological activity in common with the wild-type alcohol dehydrogenase, from which the fragment or variant was derived. For example, a functional fragment or a functional variant of a mutated alcohol dehydrogenase is one which retains essentially the same ability to catabolize BCAAs as alcohol dehydrogenase from which the functional fragment or functional variant was derived. For example, a polypeptide having alcohol dehydrogenase activity may be truncated at the N-terminus or C-terminus and the retention alcohol dehydrogenase activity assessed using assays known to those of skill in the art, including the exemplary assays provided herein. In one embodiment, the recombinant bacterial cell disclosed herein comprises a heterologous gene encoding an alcohol dehydrogenase functional variant. In another embodiment, the recombinant bacterial cell disclosed herein comprises a heterologous gene encoding alcohol dehydrogenase functional fragment.

In some embodiments, the at least one gene encoding an alcohol dehydrogenase is mutagenized, mutants exhibiting increased activity are selected, and the mutagenized gene(s) encoding the alcohol dehydrogenase are isolated and inserted into the bacterial cell. In some embodiments, the at least one gene encoding the alcohol dehydrogenase is mutagenized, mutants exhibiting decreased activity are selected, and the mutagenized gene(s) encoding the alcohol dehydrogenase are isolated and inserted into the bacterial cell. The gene comprising the modifications described herein may be present on a plasmid or chromosome.

Assays for testing the activity of an alcohol dehydrogenase, an alcohol dehydrogenase functional variant, or an alcohol dehydrogenase functional fragment are well known to one of ordinary skill in the art. For example, alcohol dehydrogenase activity can be assessed by expressing the protein, functional variant, or fragment thereof, in a recombinant bacterial cell that lacks endogenous alcohol dehydrogenase activity. Also, activity can be assessed using the enzymatic assay methods as described by Kagi et al. (J. Biol. Chem., 235:3188-92, 1960), and Walker (Biochem. Educ., 20(1):42-43, 1992).

In some embodiments, the alcohol dehydrogenase is co-expressed with an additional branched chain amino acid catabolism enzyme. In some embodiments, the alcohol dehydrogenase is co-expressed with one or more keto-acid decarboxylase enzyme(s), e.g., kivD. In some embodiments, the alcohol dehydrogenase is co-expressed with one or more keto-acid decarboxylase enzymes, e.g., kivD and one or more other alcohol dehydrogenase enzymes.

In some embodiments, the at least one alcohol dehydrogenase enzyme is coexpressed with one or more BCAA transporter(s), for example, a high affinity leucine transporter, e.g., LivKHMGF and/or a low affinity BCAA transporter, e.g., BrnQ.

In some embodiments, the engineered bacteria comprise gene sequence(s) encoding at least one alcohol dehydrogenase enzyme and genetic modification that reduces export of a branched chain amino acid, e.g., a genetic mutation in a leuE gene or promoter thereof. In one embodiment, the engineered bacteria comprise gene sequence(s) encoding at least one alcohol dehydrogenase enzyme and a genetic modification that reduces or eliminates branched chain amino acid synthesis, e.g., a genetic mutation in a ilvC gene or promoter thereof.

In some embodiments, the engineered bacteria comprise gene sequence(s) encoding at least one alcohol dehydrogenase enzyme and further comprise gene sequence encoding one or more polypeptides selected from other branched chain amino acid catabolism enzyme(s), BCAA transporter(s), and BCAA binding protein(s). In some embodiments, the engineered bacteria comprise gene sequence(s) encoding at least one alcohol dehydrogenase and gene sequence(s) encoding one or more branched chain amino acid dehydrogenase(s) (e.g., leuDH). In some embodiments, the engineered bacteria comprise gene sequence(s) encoding at least one alcohol dehydrogenase and gene sequence(s) encoding one or more keto-acid decarboxylase enzyme(s), e.g., kivD. In some embodiments, the engineered bacteria comprise gene sequence(s) encoding at least one alcohol dehydrogenase and gene sequence(s) encoding one or more other alcohol dehydrogenase(s) (e.g., adh2).

In some embodiments, the engineered bacteria comprise gene sequence(s) encoding at least one alcohol dehydrogenase and genetic modification that reduces export of a branched chain amino acid, e.g., a genetic mutation in a leuE gene or promoter thereof. In one embodiment, the engineered bacteria comprise gene sequence(s) encoding at least one alcohol dehydrogenase and a genetic modification that reduces or eliminates branched chain amino acid synthesis, e.g., a genetic mutation in a ilvC gene or promoter thereof.

In one embodiment, the at least one gene encoding the branched chain amino acid alcohol dehydrogenase comprises the adh2 gene. In one embodiment, the adh2 gene has at least about 80% identity with the sequence of any one of SEQ ID NOs:97-120. In one embodiment, the adh2 gene has at least about 90% identity with the sequence of any one of SEQ ID NOs:97-120. In one embodiment, the adh2 gene has at least about 95% identity with the sequence of any one of SEQ ID NOs:97-120. In one embodiment, the adh2 gene has at least about 85%, 86%, 87%, 88%, 89%, 90%, 91%, 92%, 93%, 94%, 95%, 96%, 97%, 98%, or 99% identity with the sequence of any one of SEQ ID NOs:97-120. In another embodiment, the adh2 gene comprises the sequence of any one of SEQ ID NOs:97-120. In yet another embodiment, the adh2 gene consists of the sequence of any one of SEQ ID NOs:97-120.

In any of these embodiments, the alcohol dehydrogenase is coexpressed with one or more branched chain amino acid catabolism enzymes, e.g., leuDH, and or kivD, each of which are described in more detail herein. In other embodiments, the alcohol dehydrogenase is further coexpressed with a transporter of a branched chain amino acid, e.g., brnQ, and/or a binding protein of a BCAA.

In some embodiments, the gene sequence(s) encoding the one or more alcohol dehydrogenase is expressed under the control of a constitutive promoter. In some embodiments, the gene sequence(s) encoding the one or more b alcohol dehydrogenase is expressed under the control of an inducible promoter. In some embodiments, the gene sequence(s) encoding the one or more alcohol dehydrogenase is expressed under the control of a promoter that is directly or indirectly induced by exogenous environmental conditions. In some embodiments, the gene sequence(s) encoding the one or more alcohol dehydrogenase is expressed under the control of a promoter that is directly or indirectly induced by low-oxygen or anaerobic conditions, wherein expression of the gene encoding the alcohol dehydrogenase is activated under low-oxygen or anaerobic environments, such as the environment of the mammalian gut. In some embodiments, the gene sequence(s) encoding the one or more alcohol dehydrogenase is expressed under the control of a promoter that is directly or indirectly induced by inflammatory conditions. Exemplary inducible promoters described herein include oxygen level-dependent promoters (e.g., FNR-inducible promoter), promoters induced by inflammation or an inflammatory response (RNS, ROS promoters), and promoters induced by a metabolite that may or may not be naturally present (e.g., can be exogenously added) in the gut, e.g., arabinose and tetracycline. Examples of inducible promoters include, but are not limited to, an FNR responsive promoter, a P_{araC} promoter, a P_{araBAD} promoter, and a P_{TetR} promoter, each of which are described in more detail herein.

Transporter (Importer) of a Branched Chain Amino Acid

In some embodiments, a recombinant bacterial cell disclosed herein comprising gene sequence(s) encoding at least one branched chain amino acid catabolism enzyme (e.g., in some embodiments expressed on a high-copy plasmid) does not increase branched chain amino acid catabolism or decrease branched chain amino acid levels in the absence of a heterologous transporter (importer) of the branched chain amino acid, e.g., leucine, and additional copies of a native importer of the branched chain amino acid, e.g., livKHMGF. It has been surprisingly discovered that in some embodiments, the rate-limiting step of branched chain amino acid catabolism, e.g., leucine catabolism, is not expression of a branched chain amino acid catabolism enzyme, but rather availability of the branched chain amino acid, e.g., leucine. Thus, in some embodiments, it may be advantageous to increase branched chain amino acid transport, e.g., leucine transport, into the cell, thereby enhancing branched chain amino acid catabolism. Surprisingly, in conjunction with overexpression of a transporter of a branched chain amino acid, e.g., LivKHMGF, even low copy number plasmids comprising a gene encoding at least one branched chain amino acid catabolism enzyme are capable of almost completely eliminating a branched chain amino acid, e.g., leucine, from a sample. Furthermore, in some embodiments that incorporate a transporter of a branched chain amino acid into the recombinant bacterial cell, there may be additional advantages to using a low-copy plasmid comprising the gene encoding the branched chain amino acid catabolism enzyme in conjunction in order to enhance the stability of expression of the branched chain amino acid catabolism enzyme, while maintaining high branched chain amino acid catabolism and to reduce negative selection pressure on the transformed bacterium. In alternate embodiments, the branched chain amino acid transporter is used in conjunction with a high-copy plasmid. In alternate embodiments, the gene(s) at least one BCAA catabolism enzyme is integrated in the bacterial chromosome.

In some embodiments, in which the engineered bacterial cell comprises gene sequence encoding a branched amino acid transporter, the bacterial cell comprises gene sequence encoding one branched chain amino acid catabolism enzyme. In other embodiments, in which the engineered bacterial cell comprises gene sequence encoding a branched amino acid transporter, the bacterial cell comprises gene sequence(s) encoding two branched chain amino acid catabolism enzymes. In other embodiments, in which the engineered bacterial cell comprises gene sequence encoding a branched amino acid transporter, the bacterial cell comprises gene sequence(s) encoding three or more branched chain amino acid catabolism enzymes. In other embodiments, in which the engineered bacterial cell comprises gene sequence encoding a branched amino acid transporter, the bacterial cell comprises gene sequence(s) encoding four, five, six or more branched chain amino acid catabolism enzymes.

In some embodiments, the branched chain amino acid catabolism enzyme converts a branched chain amino acid, e.g., leucine, valine, isoleucine, into its corresponding branched chain alpha-keto acid counterpart. In other embodiments, the branched chain amino acid catabolism enzyme converts a branched chain alpha-keto acid, e.g., alpha-ketoisocaproate, alpha-keto-beta-methylvalerate, alpha-ketoisovalerate into its corresponding aldehyde. In other embodiments, the branched chain amino acid catabolism enzyme converts a branched chain alpha-keto acid, e.g., alpha-ketoisocaproate, alpha keto-beta-methylvalerate, alpha-ketoisovalerate into its corresponding acetyl-CoA, e.g., isovaleryl-CoA, alpha-methylbutyryl-CoA, isobutyl-CoA. In other embodiments, the branched chain amino acid catabolism enzyme converts a branched chain aldehyde to its corresponding alcohol. In another embodiment, the branched chain amino acid catabolism enzyme converts a branched chain aldehyde to its corresponding carboxylic acid.

In some embodiments, the engineered bacteria comprising gene sequence(s) encoding one or more BCAA transporter(s), further comprise gene sequence(s) encoding one or more BCAA catabolism enzymes, e.g., leuDH. In some embodiments, the engineered bacteria comprising gene sequence(s) encoding one or more BCAA transporter(s), further comprise gene sequence(s) encoding one or more BCAA catabolism enzymes, e.g., leuDH, and gene sequence(s) encoding one or more keto acid decarboxylase enzyme(s), e.g., kivD. In any of these embodiments, the engineered bacteria also comprise a genetic modification that reduces export of a branched chain amino acid, e.g., a genetic mutation in a leuE gene or promoter thereof. In any of these embodiments, the bacteria also comprise a genetic modification that reduces or eliminates branched chain amino acid synthesis, e.g., a genetic mutation in a ilvC gene or promoter thereof.

In one embodiment, the bacterial cell comprises gene sequence encoding one or more transporter(s) of branched chain amino acids, gene sequence(s) encoding one or more branched chain amino acid catabolism enzyme(s), and at least one genetic modification that reduces export of a branched chain amino acid, e.g., genetic modification in a leuE gene or promoter thereof. In one embodiment, the bacterial cell comprises gene sequence encoding one or more transporter(s) of branched chain amino acids, gene sequence(s) encoding one or more branched chain amino acid catabolism enzyme(s), and at least one genetic modification that reduces or eliminates branched chain amino acid synthesis, e.g., a genetic mutation in a ilvC gene or promoter thereof. In one embodiment, the bacterial cell comprises gene sequence encoding one or more transporter(s) of branched chain amino acids, gene sequence(s) encoding one or more branched chain amino acid catabolism enzyme(s), at least one genetic modification that reduces export of a branched chain amino acid., and at least one genetic modification that reduces or eliminates branched chain amino acid synthesis.

The uptake of branched chain amino acids into bacterial cells is mediated by proteins well known to those of skill in the art. For example, two well characterized BCAA transport systems have been characterized in several bacteria, including Escherichia coli. BCAAs are transported by two systems into bacterial cells (i.e., imported), the osmotic-shock-sensitive systems designated LIV-I and LS (leucine-specific), and by an osmotic-shock resistant system, BrnQ, formerly known as LIV-II (see Adams et al., J. Biol. Chem. 265:11436-43 (1990); Anderson and Oxender, J. Bacteriol. 130:384-92 (1977); Anderson and Oxender, J. Bacteriol. 136:168-74 (1978); Haney et al., J. Bacteriol. 174:108-15 (1992); Landick and Oxender, J. Biol. Chem. 260:8257-61 (1985); Nazos et al., J. Bacteriol. 166:565-73 (1986); Nazos et al., J. Bacteriol. 163:1196-202 (1985); Oxender et al., Proc. Natl. Acad. Sci. USA 77:1412-16 (1980); Quay et al., J. Bacteriol. 129:1257-65 (1977); Rahmanian et al., J. Bacteriol. 116:1258-66 (1973); Wood, J. Biol. Chem. 250:4477-85 (1975); Guardiola et al., J. Bacteriol. 117:393-405 (1974); Guardiola and Iaccarino, J. Bacteriol. 108:1034-44 (1971); Ohnishi et al., Jpn. J. Genet. 63:343-57)(1988); Yamato and Anraku, J. Bacteriol. 144:36-44 (1980); and Yamato et al., J. Bacteriol. 138:24-32 (1979)). Transport by the BrnQ system is mediated by a single membrane protein. Transport mediated by the LIV-I system is dependent on the substrate binding protein LivJ (also known as LIV-BP), while transport mediated by LS system is mediated by the substrate binding protein LivK (also known as LS-BP). LivJ is encoded by the livJ gene, and binds isoleucine, leucine and valine with K_{d} values of ˜10⁻⁶ and ˜10⁻⁷ M, while LivK is encoded by the livK gene, and binds leucine with a K_{d} value of ˜10⁻⁶ M (See Landick and Oxender, J. Biol. Chem. 260:8257-61 (1985)). Both LivJ and LivK interact with the inner membrane components LivHMGF to enable ATP-hydrolysis-coupled transport of their substrates into the cell, forming the LIV-I and LS transport systems, respectively. The LIV-I system transports leucine, isoleucine and valine, and to a lesser extent serine threonine and alanine, whereas the LS system only transports leucine. The six genes encoding the E. coli LIV-I and LS systems are organized into two transcriptional units, with livKHMGF transcribed as a single operon, and livJ transcribed separately. LivKHMGF is an ABC transporter comprised of five subunits, including LivK, which is a periplasmic amino acid binding protein, LivHM, which are membrane subunits, and LivGF, which are ATP-binding subunits. The Escherichia coli liv genes can be grouped according to protein function, with the livJ and livK genes encoding periplasmic binding proteins with the binding affinities described above, the livH and livM genes encoding inner membrane permeases, and the livG and livF genes encoding cytoplasmic ATPases.

BrnQ is a branched chain amino acid transporter highly similar to the Salmonella typhimurium BrnQ branched chain amino acid transporter (Ohnishi et al., Cloning and nucleotide sequence of the brnQ gene, the structural gene for a membrane-associated component of the LIV-II transport system for branched-chain amino acids in Salmonella typhimurium. Jpn J Genet. 1988 August63(4):343-57) and corresponds to the Liv-II branched chain amino acid transport system in E. coli, which has been shown to transport leucine, valine, and isoleucine (Guardiola et al., Mutations affecting the different transport systems for isoleucine, leucine, and valine in Escherichia coli K-12. J Bacteriol. 1974 February; 117(2):393-405), Guardiola and Oxender, Genetic separation of high- and low-affinity transport systems for branched-chain amino acids in Escherichia coli K-12. J Bacteriol. 1978 October; 136(1):168-74., Anderson and Oxender, Genetic separation of high- and low-affinity transport systems for branched-chain amino acids in Escherichia coli K-12 J Bacteriol. 1978 October; 136(1):168-74.78). BrnQ is a member of the LIVCS family of branched chain amino acid transporters and likely functions as a sodium/branched chain amino acid symporter.

Branched chain amino acid transporters, e.g., leucine importers, may be expressed or modified in the bacteria disclosed herein in order to enhance branched chain amino acid, e.g., leucine, transport into the cell. For example, the gene sequence(s) for endogenous transporter(s) may be modified (e.g., codon-optimized and/or expressed by a strong promoter) to overexpress the transporter and/or additional copies of the transporter may be added. Alternatively, or additionally, gene sequence(s) for one or more non-endogenous or non-native transporters may be expressed in the bacterial cell. Specifically, when the transporter of a branched chain amino acid is expressed in the recombinant bacterial cells disclosed herein, the bacterial cells import more branched chain amino acids into the cell when the transporter is expressed than unmodified bacteria of the same bacterial subtype under the same conditions (not expressing the transporter). Thus, in some embodiments, the engineered bacteria comprise gene sequence(s) encoding one or more transporter(s) of a branched chain amino acid, e.g., leucine, which may be used to import branched chain amino acids, e.g., leucine, into the bacteria so that any gene encoding a branched chain amino acid catabolism enzyme expressed in the bacteria catabolize the branched chain amino acid, e.g., leucine, to treat diseases associated with the catabolism of branched chain amino acids, such as Maple Syrup Urine Disease (MSUD). In one embodiment, the bacterial cell comprises gene sequence(s) encoding one or more transporter(s) of branched chain amino acids. In one embodiment, the bacterial cell comprises gene sequence(s) encoding one or more transporter(s) of branched chain amino acids and a gene sequence(s) encoding one or more branched chain amino acid catabolism enzyme(s). In one embodiment, the bacterial cell comprises gene sequence(s) encoding a one or more transporter(s) of branched chain amino acids and a genetic modification that reduces export of a branched chain amino acid, e.g., a genetic mutation in a leuE gene or promoter thereof. In one embodiment, the bacterial cell comprises gene sequence(s) encoding one or more transporter(s) of branched chain amino acids and a genetic modification that reduces or eliminates branched chain amino acid synthesis, e.g., a genetic mutation in a ilvC gene or promoter thereof. In one embodiment, the bacterial cell comprises gene sequence encoding one or more transporter(s) of branched chain amino acids, gene sequence(s) encoding one or more branched chain amino acid catabolism enzyme(s), and at least one genetic modification that reduces export of a branched chain amino acid. In one embodiment, the bacterial cell comprises gene sequence encoding one or more transporter(s) of branched chain amino acids, gene sequence(s) encoding one or more branched chain amino acid catabolism enzyme(s), and at least one genetic modification that reduces or eliminates branched chain amino acid synthesis. In one embodiment, the bacterial cell comprises gene sequence encoding one or more transporter(s) of branched chain amino acids, gene sequence(s) encoding one or more branched chain amino acid catabolism enzyme(s), at least one genetic modification that reduces export of a branched chain amino acid., and at least one genetic modification that reduces or eliminates branched chain amino acid synthesis. In any of these embodiments, the engineered bacterial cell may further comprise gene sequence encoding livJ, which brings BCAA into the bacterial cell. In any of these embodiments, the transporter may be a native transporter, e.g., the bacteria may comprise additional copies of the native transporter. In any of these embodiments, the transporter may be a non-native transporter. In any of these embodiments, the transporter may be LivKHMGF. In any of these embodiments, the transporter may be BrnQ. In any of these embodiments, the bacterial cell may comprise gene sequence(s) encoding LivKHMGF and BrnQ.

In some embodiments, the engineered bacteria comprise gene sequence(s) encoding one or more BCAA catabolism enzyme(s) and gene sequence(s) encoding one or more BCAA transporters, in which the gene sequence(s) encoding one or more BCAA catabolism enzyme(s) and the gene sequence(s) encoding one or more transporter(s) are operably linked to different copies of the same promoter. In some embodiments, the engineered bacteria comprise gene sequence(s) encoding one or more BCAA catabolism enzyme(s) and gene sequence(s) encoding one or more BCAA transporters, in which the gene sequence(s) encoding one or more BCAA catabolism enzyme(s) and the gene sequence(s) encoding one or more transporter(s) are operably linked to different promoters. Thus, in some embodiments, the disclosure provides a bacterial cell that comprises gene sequence(s) encoding one or more branched chain amino acid catabolism enzyme(s) operably linked to a first promoter and gene sequence encoding one or more transporter(s) of a branched chain amino acid, e.g., leucine. In some embodiments, the disclosure provides a bacterial cell that comprises gene sequence(s) encoding one or more transporters of a branched chain amino acid operably linked to the first promoter. In other embodiments, the disclosure provides a bacterial cell impressing gene sequence(s) encoding one or more branched chain amino acid catabolism enzyme(s) operably linked to a first promoter and gene sequence(s) encoding one or more transporter(s) of a branched chain amino acid operably linked to a second promoter. In one embodiment, the first promoter and the second promoter are separate copies of the same promoter. In another embodiment, the first promoter and the second promoter are different promoters. In one embodiment, the first promoter and the second promoter are inducible promoters. In another embodiment, the first promoter is an inducible promoter and the second promoter is a constitutive promoter. In some embodiments, the gene sequence(s) encoding the one or more BCAA catabolism enzymes and the gene sequence(s) encoding the one or more transporters is expressed under the control of a constitutive promoter. In some embodiments, the gene sequence(s) encoding the one or more BCAA catabolism enzymes and the gene sequence(s) encoding the one or more BCAA transporters is expressed under the control of an inducible promoter. In some embodiments, the gene sequence(s) encoding the one or more BCAA transporters is expressed under the control of an inducible promoter that is directly or indirectly induced by exogenous environmental conditions. In some embodiments, the gene sequence(s) encoding the one or more BCAA catabolism enzymes and the gene sequence(s) encoding the one or more BCAA transporters is expressed under the control of an inducible promoter that is directly or indirectly induced by exogenous environmental conditions. In some embodiments, the gene sequence(s) encoding the one or more BCAA transporters is expressed under the control of an inducible promoter that is directly or indirectly induced by low-oxygen or anaerobic conditions, wherein expression of the gene encoding the one or more transporters is activated under low-oxygen or anaerobic environments, such as the environment of the mammalian gut. In some embodiments, the gene sequence(s) encoding the one or more BCAA catabolism enzymes and the gene sequence(s) encoding the one or more BCAA transporters is expressed under the control of an inducible promoter that is directly or indirectly induced by low-oxygen or anaerobic conditions, wherein expression of the gene(s) encoding the one or more BCAA catabolism enzymes and expression of the gene(s) encoding the one or more BCAA transporters is activated under low-oxygen or anaerobic environments, such as the environment of the mammalian gut. In some embodiments, the gene sequence(s) encoding the one or more BCAA transporters is expressed under the control of an inducible promoter that is directly or indirectly induced by inflammatory conditions. In some embodiments, the gene sequence(s) encoding the one or more BCAA catabolism enzymes and the gene sequence(s) encoding the one or more BCAA transporters is expressed under the control of an inducible promoter that is directly or indirectly induced by inflammatory conditions. Exemplary inducible promoters described herein include oxygen level-dependent promoters (e.g., FNR-inducible promoter), promoters induced by inflammation or an inflammatory response (RNS, ROS promoters), and promoters induced by a metabolite that may or may not be naturally present (e.g., can be exogenously added) in the gut, e.g., arabinose and tetracycline. Examples of inducible promoters include, but are not limited to, an FNR responsive promoter, a P_{araC} promoter, a P_{araBAD} promoter, and a P_{TetR} promoter, each of which are described in more detail herein.

In one embodiment, the bacterial cell comprises at least one gene encoding a transporter of a branched chain amino acid from a different organism, e.g., a different species of bacteria. In one embodiment, the bacterial cell comprises at least one native gene encoding a transporter of a branched chain amino acid. In some embodiments, the at least one native gene encoding a transporter of a branched chain amino acid is not modified. In another embodiment, the bacterial cell comprises more than one copy of at least one native gene encoding a transporter of a branched chain amino acid. In yet another embodiment, the bacterial cell comprises a copy of at least one gene encoding a native transporter of a branched chain amino acid, as well as at least one copy of at least one heterologous gene encoding a transporter of a branched chain amino acid. The heterologous gene sequence may encode an additional copy or copies of the native transporter, may encode one or more copies of a non-native transporter, and/or may encode one or more copies of a homologous or different transporter from a different bacterial species. In one embodiment, the bacterial cell comprises at least one, two, three, four, five, or six copies of the at least one heterologous gene encoding a transporter of a branched chain amino acid. In one embodiment, the bacterial cell comprises multiple copies of the at least one heterologous gene encoding a transporter of a branched chain amino acid. In one embodiment, the bacterial cell comprises gene sequence(s) encoding two or more different transporters of a branched chain amino acid. In one embodiment, the gene sequence(s) encoding two or more different transporters of a branched chain amino acid is under the control of one or more inducible promoters. In one embodiment, the gene sequence(s) encoding two or more different transporters of a branched chain amino acid is under the control of one or more constitutive promoters. In one embodiment, the gene sequence(s) encoding two or more different transporters of a branched chain amino acid is under the control of at least one inducible promoter and at least one constitutive promoter. In any of these embodiments, the gene sequence(s) encoding the one or more BCAA transporter(s) may be present on one or more plasmids. In any of these embodiments, the gene sequence(s) encoding the one or more BCAA transporter(s) may be present in the bacterial chromosome.

In one embodiment, the transporter of a branched chain amino acid imports a branched chain amino acid into the bacterial cell. In one embodiment, the transporter of a branched chain amino acid imports leucine into the bacterial cell. In one embodiment, the transporter of a branched chain amino acid imports isoleucine into the bacterial cell. In one embodiment, the transporter of a branched chain amino acid imports valine into the bacterial cell. In one embodiment, the transporter of a branched chain amino acid imports one or more of leucine, isoleucine, and valine into the bacterial cell.

In some embodiments, the transporter of a branched chain amino acid is encoded by a transporter of a branched chain amino acid gene derived from a bacterial genus or species, including but not limited to, Bacillus, Campylobacter, Clostridium, Escherichia, Lactobacillus, Pseudomonas, Salmonella, Staphylococcus, Bacillus subtilis, Campylobacter jejuni, Clostridium perfringens, Escherichia coli, Lactobacillus delbrueckii, Pseudomonas aeruginosa, Salmonella typhimurium, or Staphylococcus aureus. In some embodiments, the bacterial species is Escherichia coli. In some embodiments, the bacterial species is Escherichia coli strain Nissle.

Multiple distinct transporters of branched chain amino acids are known in the art. In one embodiment, the at least one gene encoding a transporter of a branched chain amino acid is the brnQ gene. In one embodiment, the at least one gene encoding a transporter of a branched chain amino acid is the livKHMGF operon. In another embodiment, the livKHMGF operon is an Escherichia coli livKHMGF operon. In any of these embodiments, the bacterial cell may comprise more than one copy of any of one or more of these gene sequences. In any of these embodiments, the bacterial cell may over-express any one or more of these gene sequences. In any of these embodiments, the bacterial cell may further comprise gene sequence(s) encoding one or more additional BCAA transporters, e.g., BmQ transporter.

The present disclosure further provides genes encoding functional fragments of a transporter of a branched chain amino acid or functional variants of an importer of a branched chain amino acid. As used herein, the term “functional fragment thereof” or “functional variant thereof” of a transporter of a branched chain amino acid relates to an element having qualitative biological activity in common with the wild-type transporter of a branched chain amino acid from which the fragment or variant was derived. For example, a functional fragment or a functional variant of a mutated transporter of a branched chain amino acid protein is one which retains essentially the same ability to import leucine into the bacterial cell as does the importer protein from which the functional fragment or functional variant was derived. In one embodiment, the recombinant bacterial cell disclosed herein comprises at least one heterologous gene encoding a functional fragment of a transporter of branched chain amino acid. In another embodiment, the recombinant bacterial cell disclosed herein comprises a heterologous gene encoding a functional variant of a transporter of branched chain amino acid.

Assays for testing the activity of an importer of a branched chain amino acid, a functional variant of a transporter of a branched chain amino acid, or a functional fragment of a transporter of a branched chain amino acid are well known to one of ordinary skill in the art. For example, import of a branched chain amino acid may be determined using the methods as described in Haney et al., J. Bact., 174(1):108-15, 1992; Rahmanian et al., J. Bact., 116(3):1258-66, 1973; and Ribardo and Hendrixson, J. Bact., 173(22):6233-43, 2011, the entire contents of each of which are expressly incorporated by reference herein.

In one embodiment, the genes encoding the transporter of a branched chain amino acid have been codon-optimized for use in the host organism. In one embodiment, the genes encoding the importer of a branched chain amino acid have been codon-optimized for use in Escherichia coli.

The present disclosure also encompasses genes encoding a transporter of a branched chain amino acid, e.g., a transporter of leucine, comprising amino acids in its sequence that are substantially the same as an amino acid sequence described herein. Amino acid sequences that are substantially the same as the sequences described herein include sequences comprising conservative amino acid substitutions, as well as amino acid deletions and/or insertions.

In some embodiments, the at least one gene encoding a transporter of a branched chain amino acid, e.g., livKHMGF, is mutagenized; mutants exhibiting increased branched chain amino acid, e.g., leucine, transport are selected; and the mutagenized at least one gene encoding a transporter of a branched chain amino acid, e.g., livKHMGF, is isolated and inserted into the bacterial cell. In some embodiments, the at least one gene encoding a transporter of a branched chain amino acid, e.g., livKHMGF, is mutagenized; mutants exhibiting decreased branched chain amino acid, e.g., leucine, transport are selected; and the mutagenized at least one gene encoding a transporter of a branched chain amino acid, e.g., livKHMGF, is isolated and inserted into the bacterial cell. The importer modifications described herein may be present on a plasmid or chromosome.

In one embodiment, the livKHMGF operon has at least about 80% identity with the uppercase sequence of SEQ ID NO: 145. Accordingly, in one embodiment, the livKHMGF operon has at least about 90% identity with the uppercase sequence of SEQ ID NO: 145. Accordingly, in one embodiment, the livKHMGF operon has at least about 95% identity with the uppercase sequence of SEQ ID NO: 145. Accordingly, in one embodiment, the livKHMGF operon has at least about 85%, 86%, 87%, 88%, 89%, 90%, 91%, 92%, 93%, 94%, 95%, 96%, 97%, 98%, or 99% identity with the uppercase sequence of SEQ ID NO: 145. In another embodiment, the livKHMGF operon comprises the uppercase sequence of SEQ ID NO:145. In yet another embodiment the livKHMGF operon consists of the uppercase sequence of SEQ ID NO: 145.

In some embodiments, the brnQ gene has at least about 80% identity with the entire sequence of any one of SEQ ID NOs: 121-144. Accordingly, in one embodiment, the brnQ gene has at least about 90% identity with the entire sequence of any one of SEQ ID NOs: 121-144. Accordingly, in one embodiment, the brnQ gene has at least about 95% identity with the entire sequence of any one of SEQ ID NOs: 121-144. Accordingly, in one embodiment, the brnQ gene has at least about 85%, 86%, 87%, 88%, 89%, 90%, 91%, 92%, 93%, 94%, 95%, 96%, 97%, 98%, or 99% identity with the entire sequence of any one of SEQ ID NOs: 121-144. In another embodiment, the brnQ gene comprises the sequence of any one of SEQ ID NOs: 121-144. In yet another embodiment, the brnQ gene consists of any one of SEQ ID NOs: 121-144.

In some embodiments, the bacterial cell comprises a heterologous gene encoding a branched chain amino acid catabolism enzyme operably linked to a first promoter and at least one heterologous gene encoding a transporter of a branched chain amino acid. In some embodiments, the at least one heterologous gene encoding a transporter of a branched chain amino acid is operably linked to the first promoter. In other embodiments, the at least one heterologous gene encoding a transporter of a branched chain amino acid is operably linked to a second promoter. In one embodiment, the at least one gene encoding a transporter of a branched chain amino acid is directly operably linked to the second promoter. In another embodiment, the at least one gene encoding a transporter of a branched chain amino acid is indirectly operably linked to the second promoter.

In some embodiments, expression of at least one gene encoding a transporter of a branched chain amino acid, e.g., livKHMGF and/or brnQ, is controlled by a different promoter than the promoter that controls expression of the gene encoding the branched chain amino acid catabolism enzyme. In some embodiments, expression of the at least one gene encoding a transporter of a branched chain amino acid, e.g., livKHMGF and/or brnQ, is controlled by the same promoter that controls expression of the branched chain amino acid catabolism enzyme. In some embodiments, at least one gene encoding a transporter of a branched chain amino acid, e.g., livKHMGF and/or brnQ, and the branched chain amino acid catabolism enzyme are divergently transcribed from a promoter region. In some embodiments, expression of each of genes encoding the at least one gene encoding a transporter of a branched chain amino acid, e.g., livKHMGF and/or brnQ, and the gene encoding the branched chain amino acid catabolism enzyme is controlled by different promoters.

In one embodiment, the at least one gene encoding a transporter of a branched chain amino acid is not operably linked to its native promoter. In some embodiments, the at least one gene encoding the transporter of a branched chain amino acid, e.g., livKHMGF, is controlled by its native promoter. In some embodiments, the at least one gene encoding a transporter of a branched chain amino acid, e.g., livKHMGF and/or brnQ, is controlled by an inducible promoter. In some embodiments, the at least one gene encoding a transporter of a branched chain amino acid, e.g., livKHMGF and/or brnQ, is controlled by a promoter that is stronger than its native promoter. In some embodiments, the at least one gene encoding a transporter of a branched chain amino acid, e.g., livKHMGF, is controlled by a constitutive promoter.

In another embodiment, the promoter is an inducible promoter. Inducible promoters are described in more detail infra.

In one embodiment, the at least one gene encoding a transporter of a branched chain amino acid is located on a plasmid in the bacterial cell. In another embodiment, the at least one gene encoding a transporter of a branched chain amino acid is in the chromosome of the bacterial cell. In yet another embodiment, a native copy of the at least one gene encoding a transporter of a branched chain amino acid is located in the chromosome of the bacterial cell, and a copy of at least one gene encoding a transporter of a branched chain amino acid from a different species of bacteria is located on a plasmid in the bacterial cell. In yet another embodiment, a native copy of the at least one gene encoding a transporter of a branched chain amino acid is located on a plasmid in the bacterial cell, and a copy of at least one gene encoding a transporter of a branched chain amino acid from a different species of bacteria is located on a plasmid in the bacterial cell. In yet another embodiment, a native copy of the at least one gene encoding a transporter of a branched chain amino acid is located in the chromosome of the bacterial cell, and a copy of the at least one gene encoding an importer of a branched chain amino acid from a different species of bacteria is located in the chromosome of the bacterial cell.

In some embodiments, the at least one native gene encoding a transporter, e.g., livKHMGF and/or brnQ, in the bacterial cell is not modified, and one or more additional copies of the native transporter, e.g., livKHMGF and/or brnQ, are inserted into the genome. In some embodiments, the at least one native gene encoding a transporter, e.g., livKHMGF and/or brnQ, in the bacterial cell is not modified, and one or more additional copies of the native transporter, e.g., livKHMGF and/or brnQ, are present on a plasmid, e.g., a high copy or low copy plasmid. In one embodiment, the one or more additional copies of the native a transporter, e.g., livKHMGF and/or brnQ, that is inserted into the genome are under the control of the same inducible promoter that controls expression of the gene encoding the branched chain amino acid catabolism enzyme, e.g., the FNR responsive promoter, or a different inducible promoter than the one that controls expression of the branched chain amino acid catabolism enzyme, or a constitutive promoter. In alternate embodiments, the at least one native gene encoding the transporter, e.g., livKHMGF and/or brnQ, is not modified, and one or more additional copies of the transporter, e.g., livKHMGF, from a different bacterial species is inserted into the genome of the bacterial cell. In one embodiment, the one or more additional copies of the transporter inserted into the genome of the bacterial cell are under the control of the same inducible promoter that controls expression of the gene encoding the branched chain amino acid catabolism enzyme, e.g., the FNR responsive promoter, or a different inducible promoter than the one that controls expression of the gene encoding the branched chain amino acid catabolism enzyme, or a constitutive promoter.

In some embodiments, at least one native gene encoding the transporter, e.g., livKHMGF and/or brnQ, in the genetically modified bacteria is not modified, and one or more additional copies of at least one native gene encoding the transporter, e.g., livKHMGF and or brnQ, are present in the bacterial cell on a plasmid. In one embodiment, the at least one native gene encoding the transporter e.g., livKHMGF and/or brnQ, present in the bacterial cell on a plasmid is under the control of the same inducible promoter that controls expression of the gene encoding the branched chain amino acid catabolism enzyme, e.g., the FNR responsive promoter, or a different inducible promoter than the one that controls expression of the gene encoding the branched chain amino acid catabolism enzyme, or a constitutive promoter. In alternate embodiments, the at least one native gene encoding the transporter, e.g., livKHMGF and/or brnQ, is not modified, and a copy of at least one gene encoding the transporter, e.g., livKHMGF and/or brnQ, from a different bacterial species is present in the bacteria on a plasmid. In one embodiment, the copy of at least one gene encoding the transporter, e.g., livKHMGF and/or brnQ, from a different bacterial species is under the control of the same inducible promoter that controls expression of the gene encoding the branched chain amino acid catabolism enzyme, e.g., the FNR responsive promoter, or a different inducible promoter than the one that controls expression of the gene encoding the branched chain amino acid catabolism enzyme, or a constitutive promoter.

In some embodiments, the bacterium is E. coli Nissle, and the at least one native gene encoding the transporter, e.g., livKHMGF and/or brnQ, in E. coli Nissle is not modified; one or more additional copies at least one native gene encoding the transporter, e.g., livKHMGF and/or brnQ, from E. coli Nissle is inserted into the E. coli Nissle genome under the control of the same inducible promoter that controls expression of the gene encoding the branched chain amino acid catabolism enzyme, e.g., the FNR responsive promoter, or a different inducible promoter than the one that controls expression of the gene encoding the branched chain amino acid catabolism enzyme, or a constitutive promoter. In an alternate embodiment, the at least one native gene encoding the a transporter, e.g., livKHMGF and/or brnQ in E. coli Nissle is not modified, and a copy of at least one gene encoding the transporter, e.g., livKHMGF and/or brnQ, from a different bacterial species is inserted into the E. coli Nissle genome under the control of the same inducible promoter that controls expression of the gene encoding the branched chain amino acid catabolism enzyme, e.g., the FNR responsive promoter, or a different inducible promoter than the one that controls expression of the gene encoding the branched chain amino acid catabolism enzyme, or a constitutive promoter.

In some embodiments, the bacterial cell is E. coli Nissle, and the at least one native gene encoding the transporter, e.g., livKHMGF and/or brnQ, in E. coli Nissle is not modified; one or more additional copies the at least one native gene encoding the transporter, e.g., livKHMGF and/or brnQ, E. coli Nissle is present in the bacterium on a plasmid and under the control of the same inducible promoter that controls expression of the gene encoding the branched chain amino acid catabolism enzyme, e.g., the FNR responsive promoter, or a different inducible promoter than the one that controls expression of the gene encoding the branched chain amino acid catabolism enzyme, or a constitutive promoter. In an alternate embodiment, the at least one native gene encoding the transporter, e.g., livKHMGF, in E. coli Nissle is not modified, and a copy of at least one native gene encoding the transporter, e.g., livKHMGF and/or brnQ, from a different bacterial species of are present in the bacterium on a plasmid and under the control of the same inducible promoter that controls expression of the gene encoding the branched chain amino acid catabolism enzyme, e.g., the FNR responsive promoter, or a different inducible promoter than the one that controls expression of the gene encoding the branched chain amino acid catabolism enzyme, or a constitutive promoter.

In one embodiment, when the transporter of a branched chain amino acid is expressed in the recombinant bacterial cells disclosed herein, the bacterial cells import 10% more branched chain amino acids, e.g., leucine, into the bacterial cell when the transporter is expressed as compared to unmodified bacteria of the same bacterial subtype under the same conditions. In another embodiment, when the transporter of a branched chain amino acid is expressed in the recombinant bacterial cells disclosed herein, the bacterial cells import 20%, 30%, 40%, 50%, 60%, 70%, 80%, 90% or 100% more branched chain amino acids, e.g., leucine, into the bacterial cell when the transporter is expressed as compared with unmodified bacteria of the same bacterial subtype under the same conditions. In yet another embodiment, when the transporter of a branched chain amino acid is expressed in the recombinant bacterial cells disclosed herein, the bacterial cells import two-fold more branched chain amino acids, e.g., leucine, into the cell when the transporter is expressed than unmodified bacteria of the same bacterial subtype under the same conditions. In yet another embodiment, when the transporter of a branched chain amino acid is expressed in the recombinant bacterial cells disclosed herein, the bacterial cells import three-fold, four-fold, five-fold, six-fold, seven-fold, eight-fold, nine-fold, or ten-fold more branched chain amino acids, e.g., leucine, into the cell when a transporter is expressed as compared with unmodified bacteria of the same bacterial subtype under the same conditions.

Exporter of a Branched Chain Amino Acid

The bacterial cells disclosed herein may comprise a genetic modification that inhibits or decreases the export of a branched chain amino acid and/or its corresponding alpha keto acid or other metabolite from the bacterial cell. Knocking-out or reducing export of one or more branched chain amino acids from a bacterial cell allows the bacterial cell to more efficiently retain and catabolize exogenous branched chain amino acids and/or their alpha-keto acid counterparts or other metabolite counterparts in order to treat the diseases and disorders described herein. Any of the bacterial cells disclosed herein comprising gene sequence encoding one or more BCAA catabolism enzymes and/or one or more BCAA transporters may further a genetic modification that inhibits or decreases the export of a branched chain amino acid and/or its corresponding alpha keto acid or other metabolite from the bacterial cell.

The export of branched chain amino acids from bacterial cells is mediated by proteins well known to those of skill in the art. For example, one branched chain amino acid exporter, the leucine exporter LeuE has been characterized in Escherichia coli (Kutukova et al., FEBS Letters 579:4629-34 (2005); incorporated herein by reference). LeuE is encoded by the leuE gene in Escherichia coli (also known as yeaS). Additionally, a two-gene encoded exporter of the branched chain amino acids isoleucine, valine and leucine, denominated BmFE was identified in the bacteria Corynebacterium glutamicum (Kennerknecht et al., J. Bacteriol. 184:3947-56 (2002); incorporated herein by reference). The BmFE system is encoded by the Corynebacterium glutamicum genes brnF and brnE, and homologues of said genes have been identified in several organisms, including Agrobacterium tumefaciens, Achaeoglobus fulgidus, Bacillus subtilis, Deinococcus radiodurans, Escherichia coli, Haemophilus influenzae, Helicobacter pylori, Lactococcus lactis, Streptococcus pneumoniae, and Vibrio cholerae (see Kennerknecht et al., 2002).

The bacterial cells disclosed herein comprise a genetic modification that reduces export of a branched chain amino acid from the bacterial cell. Multiple distinct exporters of branched chain amino acids, e.g., leucine, are known in the art. In one embodiment, the recombinant bacterial cell disclosed herein comprises a genetic modification that reduces export of a branched chain amino acid from the bacterial cell, wherein the endogenous gene encoding an exporter of a branched chain amino acid is a leuE gene.

In one embodiment, the recombinant bacterial cell disclosed herein comprises a genetic modification that reduces export of a branched chain amino acid from the bacterial cell and a heterologous gene encoding a branched chain amino acid catabolism enzyme. When the recombinant bacterial cells disclosed herein comprise a genetic modification that reduces export of a branched chain amino acid, the bacterial cells retain more branched chain amino acids, e.g., leucine, in the bacterial cell than unmodified bacteria of the same bacterial subtype under the same conditions. Thus, the recombinant bacteria comprising a genetic modification that reduces export of a branched chain amino acid may be used to retain more branched chain amino acids in the bacterial cell so that any branched chain amino acid catabolism enzyme expressed in the organism, e.g., co-expressed α-ketoisovalerate decarboxylase or co-expressed branched chain keto dehydrogenase, can catabolize the branched chain amino acids, e.g., leucine, to treat diseases associated with the catabolism of branched chain amino acids, including Maple Syrup Urine Disease (MSUD). In one embodiment, the recombinant bacteria further comprise a heterologous gene encoding an importer of a branched chain amino acid, e.g., a livKHMGF and/or brnQ gene.

In one embodiment, the genetic modification reduces export of a branched chain amino acid, e.g., leucine, from the bacterial cell. In one embodiment, the bacterial cell is from a bacterial genus or species that includes but is not limited to, Bacillus, Bacteroides, Bifdobacterium, Brevibacteria, Clostridium, Enterococcus, Escherichia, Lactobacillus, Lactococcus, and Staphylococcus, e.g., Bacillus coagulans, Bacillus subtilis, Bacteroides fragilis, Bacteroides subtilis, Bacteroides thetaiotaomicron, Bifidobacterium bifdum, Bifidobacterium infantis, Bifdobacterium lactis, Bifidobacterium longum, Clostridium butyricum, Enterococcus faecium, Escherichia coli, Lactobacillus acidophilus, Lactobacillus bulgaricus, Lactobacillus casei, Lactobacillus johnsonii, Lactobacillus paracasei, Lactobacillus plantarum, Lactobacillus reuteri, Lactobacillus rhamnosus, Lactococcus lactis. In another embodiment, the bacterial cell is an Escherichia coli bacterial cell. In another embodiment, the bacterial cell is an Escherichia coli strain Nissle bacterial cell.

In one embodiment, the genetic modification is a mutation in an endogenous gene encoding an exporter of a branched chain amino acid. In one embodiment, the genetic mutation is a deletion of the endogenous gene encoding an exporter, e.g., leuE, of a branched chain amino acid. In another embodiment, the genetic mutation results in an exporter having reduced activity as compared to a wild-type exporter protein. In one embodiment, the activity of the exporter is reduced at least 50%, at least 75%, or at least 100%. In another embodiment, the activity of the exporter is reduced at least two-fold, three-fold, four-fold, or five-fold. In another embodiment, the genetic mutation results in an exporter, e.g., LeuE, having no activity, i.e., results in an exporter, e.g., LeuE, which cannot export a branched chain amino acid, e.g., lysine, from the bacterial cell.

It is routine for one of ordinary skill in the art to make mutations in a gene of interest. Mutations include substitutions, insertions, deletions, and/or truncations of one or more specific amino acid residues or of one or more specific nucleotides or codons in the polypeptide or polynucleotide of the exporter of a branched chain amino acid. Mutagenesis and directed evolution methods are well known in the art for creating variants. See, e.g., U.S. Pat. Nos. 7,783,428; 6,586,182; 6,117,679; and Ling, et al., 1999, “Approaches to DNA mutagenesis: an overview,” Anal. Biochem., 254(2):157-78; Smith, 1985, “In vitro mutagenesis,” Ann. Rev. Genet., 19:423-462; Carter, 1986, “Site-directed mutagenesis,” Biochem. J, 237:1-7; and Minshull, et al., 1999, “Protein evolution by molecular breeding,” Current Opinion in Chemical Biology, 3:284-290. For example, the lambda red system can be used to knock-out genes in E. coli (see, for example, Datta et al., Gene, 379:109-115 (2006)).

The term “inactivated” as applied to a gene refers to any genetic modification that decreases or eliminates the expression of the gene and/or the functional activity of the corresponding gene product (mRNA and/or protein). The term “inactivated” encompasses complete or partial inactivation, suppression, deletion, interruption, blockage, promoter alterations, antisense RNA, dsRNA, or down-regulation of a gene. This can be accomplished, for example, by gene “knockout,” inactivation, mutation (e.g., insertion, deletion, point, or frameshift mutations that disrupt the expression or activity of the gene product), or by use of inhibitory RNAs (e.g., sense, antisense, or RNAi technology). A deletion may encompass all or part of a gene's coding sequence. The term “knockout” refers to the deletion of most (at least about 95%, at least about 96%, at least about 97%, at least about 98%, or at least about 99%) or all (100%) of the coding sequence of a gene. In some embodiments, any number of nucleotides can be deleted, from a single base to an entire piece of a chromosome.

Assays for testing the activity of an exporter of a branched chain amino acid, e.g., leucine, are well known to one of ordinary skill in the art. For example, export of a branched chain amino acid, such as leucine, may be determined using the methods described by Haney et al., J. Bact., 174(1):108-15, 1992; Rahmanian et al., J. Bact., 116(3):1258-66, 1973; and Ribardo and Hendrixson, J. Bact., 173(22):6233-43, 2011, the entire contents of which are expressly incorporated herein by reference.

In another embodiment, the genetic modification is a mutation in a promoter of an endogenous gene encoding an exporter of a branched chain amino acid. In one embodiment, the genetic mutation results in decreased expression of the leuE gene. In one embodiment, leuE gene expression is reduced by about 50%, 75%, or 100%. In another embodiment, leuE gene expression is reduced about two-fold, three-fold, four-fold, or five-fold. In another embodiment, the genetic mutation completely inhibits expression of the leuE gene.

Assays for testing the level of expression of a gene, such as an exporter of a branched chain amino acid, e.g., leuE, are well known to one of ordinary skill in the art. For example, reverse-transcriptase polymerase chain reaction may be used to detect the level of mRNA expression of a gene. Alternatively, Western blots using antibodies directed against a protein may be used to determine the level of expression of the protein.

In another embodiment, the genetic modification is an overexpression of a repressor of an exporter of a branched chain amino acid. In one embodiment, the overexpression of the repressor of the exporter is caused by a mutation which renders the promoter of the repressor constitutively active. In another embodiment, the overexpression of the repressor of the exporter is caused by the insertion of an inducible promoter in front of the repressor so that the expression of the repressor can be induced. Inducible promoters are described in more detail herein.

Reduction of Endogenous Bacterial Branched Chain Amino Acid Production

The bacterial cells disclosed herein may comprise a genetic modification that inhibits or decreases the biosynthesis of a branched chain amino acid and/or its corresponding alpha keto acid or other metabolite in the bacterial cell. Knocking-out or reducing production of endogenous branched chain amino acids in a bacterial cell allows the bacterial cell to more efficiently take up and catabolize exogenous branched chain amino acids and/or their alpha-keto acid counterparts or other metabolite counterparts in order to treat the diseases and disorders described herein. Knock-out or knock down of a gene encoding an enzyme required for branched chain amino acid biosynthesis creates an auxotroph, which requires the cell to import the branched chain amino acid or a metabolite to survive. Any of the bacterial cells disclosed herein comprising gene sequence encoding one or more BCAA catabolism enzymes and/or one or more BCAA transporters may further a genetic modification that inhibits or decreases the biosynthesis of a branched chain amino acid and/or its corresponding alpha keto acid or other metabolite in the bacterial cell.

As used herein, the term “branched chain amino acid biosynthesis” enzyme refers to an enzyme involved in the biosynthesis of a branched chain amino acid and/or its corresponding alpha-keto acid or other metabolite. Multiple distinct genes involved in biosynthetic pathways of branched chain amino acids, e.g., isoleucine, leucine, and valine, are known in the art. For example, the ilvC gene encodes a keto-acid reductoisomerase enzyme that catalyzes the conversion of acetohydroxy acids into dihydroxy valerates, which leads to the synthesis of the essential branched side chain amino acids valine and isoleucine (EC 1.1.1.86) and has been characterized in Escherichia coli (Wek and Hatfield, J. Biol. Chem. 261:2441-50 (1986), the entire contents of which are expressly incorporated herein by reference). Additionally, homologues of ilvC have been identified in several organisms, including Candida albicans, Oryza sativa, Saccharomyces cerevisiae, Pseudomonas aeruginosa, Corynebacterium glutamicum, and Spinacia oleracea.

In one embodiment, the genetic modification is a mutation in an endogenous gene encoding a protein that is involved in the biosynthesis of a branched chain amino acid or an alpha keto acid, e.g., ilvC. ilvC is an acetohydroxy acid isomeroreductase that is required for branched chain amino acid synthesis. In one embodiment, the genetic mutation is a deletion of an endogenous gene encoding a protein that is involved in the biosynthesis of a branched chain amino acid or an alpha-keto acid, or other BCAA metabolite, e.g., ilvC. In another embodiment, the genetic mutation results in an enzyme having reduced activity as compared to a wild-type enzyme. In one embodiment, the activity of the enzyme is reduced at least 50%, at least 75%, or at least 100%. In another embodiment, the activity of the enzyme is reduced at least two-fold, three-fold, four-fold, or five-fold. In another embodiment, the genetic mutation results in an enzyme, e.g., IlvC, having no activity, i.e., results in an enzyme, e.g., IlvC, which cannot catalyze the conversion of acetohydroxy acids into dihydroxy valerates, thereby inhibiting the endogenous synthesis of the branched chain amino acids valine and isoleucine in the recombinant bacterial cell.

It is routine for one of ordinary skill in the art to make mutations in a gene of interest. Mutations include substitutions, insertions, deletions, and/or truncations of one or more specific amino acid residues or of one or more specific nucleotides or codons in the polypeptide or polynucleotide of the exporter of a branched chain amino acid. Mutagenesis and directed evolution methods are well known in the art for creating variants. See, e.g., U.S. Pat. Nos. 7,783,428; 6,586,182; 6,117,679; and Ling, et al., 1999, “Approaches to DNA mutagenesis: an overview,” Anal. Biochem., 254(2):157-78; Smith, 1985, “In vitro mutagenesis,” Ann. Rev. Genet., 19:423-462; Carter, 1986, “Site-directed mutagenesis,” Biochem. J., 237:1-7; and Minshull, et al., 1999, “Protein evolution by molecular breeding,” Current Opinion in Chemical Biology, 3:284-290. For example, the lambda red system can be used to knock-out genes in E. coli (see, for example, Datta et al., Gene, 379:109-115 (2006)).

The term “inactivated,” as applied to a gene, refers to any genetic modification that decreases or eliminates the expression of the gene and/or the functional activity of the corresponding gene product (mRNA and/or protein). The term “inactivated” encompasses complete or partial inactivation, suppression, deletion, interruption, blockage, promoter alterations, antisense RNA, dsRNA, or down-regulation of a gene. This can be accomplished, for example, by gene “knockout,” inactivation, mutation (e.g., insertion, deletion, point, or frameshift mutations that disrupt the expression or activity of the gene product), or by use of inhibitory RNAs (e.g., sense, antisense, or RNAi technology). A deletion may encompass all or part of a gene's coding sequence. The term “knockout” refers to the deletion of most (at least about 95%, at least about 96%, at least about 97%, at least about 98%, or at least about 99%) or all (100%) of the coding sequence of a gene. In some embodiments, any number of nucleotides can be deleted, from a single base to an entire piece of a chromosome.

Assays for testing the activity of enzymes involved in the biosynthesis of branched chain amino acids and/or alpha-keto acids, and/or other BCAA metabolite e.g., ilvC, are well known to one of ordinary skill in the art. For example, the activity of a ketol-acid reductoisomerase enzyme may be determined using the methods described by Durner et al., Plant Physiol., 103:903-910, 1993, the entire contents of which are expressly incorporated herein by reference.

In another embodiment, the genetic modification is a mutation in a promoter of an endogenous gene encoding the branched chain amino acid biosynthesis enzyme. In one embodiment, the genetic mutation results in decreased expression of the branched chain amino acid biosynthesis enzyme gene. In one embodiment, gene expression is reduced by about 50%, 75%, or 100%. In another embodiment, gene expression is reduced about two-fold, three-fold, four-fold, or five-fold. In another embodiment, the genetic mutation completely inhibits expression of the gene.

Assays for testing the level of expression of a gene, such as ilvC, are well known to one of ordinary skill in the art. For example, reverse-transcriptase polymerase chain reaction may be used to detect the level of mRNA expression of a gene. Alternatively, Western blots using antibodies directed against a protein may be used to determine the level of expression of the protein.

In another embodiment, the genetic modification is an overexpression of a repressor of a branched chain amino acid biosynthesis gene. In one embodiment, the overexpression of the repressor of the gene is caused by a mutation which renders the promoter of the repressor constitutively active. In another embodiment, the overexpression of the repressor of the branched chain amino acid biosynthesis gene is caused by the insertion of an inducible promoter in front of the repressor so that the expression of the repressor can be induced. Inducible promoters are described in more detail herein.

Inducible Promoters

In some embodiments, the bacterial cell comprises a stably maintained plasmid or chromosome carrying the one or more optimized genes, e.g., kivD, leuDH, adh2, and brnQ, such that the optimized enzymes can be expressed in the host cell, and the host cell is capable of survival and/or growth in vitro, e.g., in medium, and/or in vivo, e.g., in the gut.

In some embodiments, the gene encoding the branched chain amino acid catabolism enzyme is present on a plasmid and operably linked to a directly or indirectly inducible promoter. In some embodiments, the gene encoding the branched chain amino acid catabolism enzyme is present on a plasmid and operably linked to a promoter that is induced under low-oxygen or anaerobic conditions. In some embodiments, the gene encoding the branched chain amino acid catabolism enzyme is present on a chromosome and operably linked to a directly or indirectly inducible promoter. In some embodiments, the gene encoding the branched chain amino acid catabolism enzyme is present in the chromosome and operably linked to a promoter that is induced under low-oxygen or anaerobic conditions. In some embodiments, the gene encoding the branched chain amino acid catabolism enzyme is present on a plasmid and operably linked to a promoter that is induced by exposure to tetracycline or arabinose.

In some embodiments, the bacterial cell comprises a stably maintained plasmid or chromosome carrying the at least one gene encoding a transporter of a branched chain amino acid, e.g., livKHMGF and/or brnQ, such that the transporter, e.g., LivKHMGF and/or BrnQ, can be expressed in the host cell, and the host cell is capable of survival and/or growth in vitro, e.g., in medium, and/or in vivo, e.g., in the gut. In some embodiments, bacterial cell comprises two or more distinct copies of the at least one gene encoding a transporter of a branched chain amino acid, e.g., livKHMGF and/or brnQ. In some embodiments, the genetically engineered bacteria comprise multiple copies of the same at least one gene encoding a transporter of a branched chain amino acid, e.g., livKHMGF and/or brnQ. In some embodiments, the at least one gene encoding a transporter of a branched chain amino acid, e.g., livKHMGF and/or brnQ, is present on a plasmid and operably linked to a directly or indirectly inducible promoter. In some embodiments, the at least one gene encoding a transporter of a branched chain amino acid, e.g., livKHMGF and/or brnQ, is present on a plasmid and operably linked to a promoter that is induced under low-oxygen or anaerobic conditions. In some embodiments, the at least one gene encoding a transporter of a branched chain amino acid, e.g., livKHMGF and/or brnQ, is present on a chromosome and operably linked to a directly or indirectly inducible promoter. In some embodiments, the at least one gene encoding a transporter of a branched chain amino acid, e.g., livKHMGF and/or brnQ, is present in the chromosome and operably linked to a promoter that is induced under low-oxygen or anaerobic conditions. In some embodiments, the at least one gene encoding a transporter of a branched chain amino acid, e.g., livKHMGF and/or brnQ, is present on a plasmid and operably linked to a promoter that is induced by exposure to tetracycline.

In some embodiments, the promoter that is operably linked to the gene encoding the branched chain amino acid catabolism enzyme and the promoter that is operably linked to the gene encoding the transporter of a branched chain amino acid, e.g., livKHMGF and/or brnQ, is directly induced by exogenous environmental conditions. In some embodiments, the promoter that is operably linked to the gene encoding the branched chain amino acid catabolism enzyme and the promoter that is operably linked to the gene encoding the transporter of a branched chain amino acid, e.g., livKHMGF and/or brnQ, is indirectly induced by exogenous environmental conditions. In some embodiments, the promoter is directly or indirectly induced by exogenous environmental conditions specific to the gut of a mammal. In some embodiments, the promoter is directly or indirectly induced by exogenous environmental conditions specific to the small intestine of a mammal. In some embodiments, the promoter is directly or indirectly induced by low-oxygen or anaerobic conditions such as the environment of the mammalian gut. In some embodiments, the promoter is directly or indirectly induced by molecules or metabolites that are specific to the gut of a mammal, e.g., propionate. In some embodiments, the promoter is directly or indirectly induced by a molecule that is co-administered with the bacterial cell.

In certain embodiments, the bacterial cell comprises optimized genes, e.g., kivD, leuDH, adh2, and brnQ, which are expressed under the control of the fumarate and nitrate reductase regulator (FNR) promoter. In certain embodiments, the bacterial cell comprises at least one gene encoding a transporter of a branched chain amino acid, e.g., livKHMGF, is expressed under the control of the fumarate and nitrate reductase regulator (FNR) promoter. In E. coli, FNR is a major transcriptional activator that controls the switch from aerobic to anaerobic metabolism (Unden et al., 1997). In the anaerobic state, FNR dimerizes into an active DNA binding protein that activates hundreds of genes responsible for adapting to anaerobic growth. In the aerobic state, FNR is prevented from dimerizing by oxygen and is inactive.

FNR responsive promoters include, but are not limited to, the FNR responsive promoters listed in the chart, below. Underlined sequences are predicted ribosome binding sites, and bolded sequences are restriction sites used for cloning. FNR responsive promoters are set forth as SEQ ID NO: 146-162.

In some embodiments, multiple distinct FNR nucleic acid sequences are inserted in the genetically engineered bacteria. In alternate embodiments, the genetically engineered bacteria comprise a gene encoding a branched chain amino acid catabolism enzyme, or other enzyme disclosed herein, is expressed under the control of an alternate oxygen level-dependent promoter, e.g., DNR (Trunk et al., 2010) or ANR (Ray et al., 1997). In alternate embodiments, the genetically engineered bacteria comprise at least one gene encoding a transporter of a branched chain amino acid, e.g., livKHMGF and/or brnQ, is expressed under the control of an alternate oxygen level-dependent promoter, e.g., DNR (Trunk et al., 2010) or ANR (Ray et al., 1997). In these embodiments, catabolism of the branched chain amino acid, e.g., leucine, is particularly activated in a low-oxygen or anaerobic environment, such as in the gut. In some embodiments, gene expression is further optimized by methods known in the art, e.g., by optimizing ribosomal binding sites and/or increasing mRNA stability. In one embodiment, the mammalian gut is a human mammalian gut.

In some embodiments, the bacterial cell comprises an oxygen-level dependent transcriptional regulator, e.g., FNR, ANR, or DNR, and corresponding promoter from a different bacterial species. The heterologous oxygen-level dependent transcriptional regulator and promoter increase the transcription of genes operably linked to said promoter, e.g., the gene encoding the branched chain amino acid catabolism enzyme, and/or the at least one gene encoding a transporter of a branched chain amino acid, e.g., livKHMGF and/or brnQ, and/or the at least one gene encoding a binding protein of a branched chain amino acid in a low-oxygen or anaerobic environment, as compared to the native gene(s) and promoter in the bacteria under the same conditions. In certain embodiments, the non-native oxygen-level dependent transcriptional regulator is an FNR protein from N. gonorrhoeae (see, e.g., Isabella et al., 2011). In some embodiments, the corresponding wild-type transcriptional regulator is left intact and retains wild-type activity. In alternate embodiments, the corresponding wild-type transcriptional regulator is deleted or mutated to reduce or eliminate wild-type activity.

In some embodiments, the genetically engineered bacteria comprise a wild-type oxygen-level dependent transcriptional regulator, e.g., FNR, ANR, or DNR, and corresponding promoter that is mutated relative to the wild-type promoter from bacteria of the same subtype. The mutated promoter enhances binding to the wild-type transcriptional regulator and increases the transcription of genes operably linked to said promoter, e.g., the gene encoding the branched chain amino acid catabolism enzyme, and/or the at least one gene encoding a transporter of a branched chain amino acid, e.g., livKHMGF and/or brnQ, and/or the at least one gene encoding a binding protein of a branched chain amino acid in a low-oxygen or anaerobic environment, as compared to the wild-type promoter under the same conditions. In some embodiments, the genetically engineered bacteria comprise a wild-type oxygen-level dependent promoter, e.g., FNR, ANR, or DNR promoter, and corresponding transcriptional regulator that is mutated relative to the wild-type transcriptional regulator from bacteria of the same subtype. The mutated transcriptional regulator enhances binding to the wild-type promoter and increases the transcription of genes operably linked to said promoter, e.g., the gene encoding the branched chain amino acid catabolism enzyme, and/or the at least one gene encoding a transporter of a branched chain amino acid, e.g., livKHMGF and/or brnQ, and/or the at least one gene encoding a binding protein of a branched chain amino acid in a low-oxygen or anaerobic environment, as compared to the wild-type transcriptional regulator under the same conditions. In certain embodiments, the mutant oxygen-level dependent transcriptional regulator is an FNR protein comprising amino acid substitutions that enhance dimerization and FNR activity (see, e.g., Moore et al., 2006).

In some embodiments, the bacterial cells disclosed herein comprise multiple copies of the endogenous gene encoding the oxygen level-sensing transcriptional regulator, e.g., the FNR gene. In some embodiments, the gene encoding the oxygen level-sensing transcriptional regulator is present on a plasmid. In some embodiments, the gene encoding the oxygen level-sensing transcriptional regulator and the gene encoding the branched chain amino acid catabolism enzyme are present on different plasmids. In some embodiments, the gene encoding the oxygen level-sensing transcriptional regulator and the gene encoding the branched chain amino acid catabolism enzyme and/or the at least one gene encoding a transporter of a branched chain amino acid and/or the at least one gene encoding a binding protein of a branched chain amino acid are present on different plasmids. In some embodiments, the gene encoding the oxygen level-sensing transcriptional regulator and the gene encoding the branched chain amino acid catabolism enzyme and/or the at least one gene encoding a transporter of a branched chain amino acid and/or the at least one gene encoding a binding protein of a branched chain amino acid are present on the same plasmid.

In some embodiments, the gene encoding the oxygen level-sensing transcriptional regulator is present on a chromosome. In some embodiments, the gene encoding the oxygen level-sensing transcriptional regulator and the gene encoding the gene encoding the branched chain amino acid catabolism enzyme and/or the at least one gene encoding a transporter of a branched chain amino acid and/or the at least one gene encoding a binding protein of a branched chain amino acid are present on different chromosomes. In some embodiments, the gene encoding the oxygen level-sensing transcriptional regulator and the gene encoding the branched chain amino acid catabolism enzyme and/or the at least one gene encoding a transporter of a branched chain amino acid and/or the at least one gene encoding a binding protein of a branched chain amino acid are present on the same chromosome. In some instances, it may be advantageous to express the oxygen level-sensing transcriptional regulator under the control of an inducible promoter in order to enhance expression stability. In some embodiments, expression of the transcriptional regulator is controlled by a different promoter than the promoter that controls expression of the gene encoding the branched chain amino acid catabolism enzyme and/or BCAA transporter and/or BCAA binding protein. In some embodiments, expression of the transcriptional regulator is controlled by the same promoter that controls expression of the branched chain amino acid catabolism enzyme and/or BCAA transporter and/or BCAA binding protein. In some embodiments, the transcriptional regulator and the branched chain amino acid catabolism enzyme are divergently transcribed from a promoter region.

RNS-Dependent Regulation

In some embodiments, the genetically engineered bacteria comprise a gene encoding a branched chain amino acid catabolism enzyme that is expressed under the control of an inducible promoter. In some embodiments, the genetically engineered bacterium that expresses a branched chain amino acid catabolism enzyme and/or BCAA transporter and/or BCAA binding protein is under the control of a promoter that is activated by inflammatory conditions. In one embodiment, the gene for producing the branched chain amino acid catabolism enzyme and/or BCAA transporter and/or BCAA binding protein is expressed under the control of an inflammatory-dependent promoter that is activated in inflammatory environments, e.g., a reactive nitrogen species or RNS promoter.

As used herein, “reactive nitrogen species” and “RNS” are used interchangeably to refer to highly active molecules, ions, and/or radicals derived from molecular nitrogen. RNS can cause deleterious cellular effects such as nitrosative stress. RNS includes, but is not limited to, nitric oxide (NO.), peroxynitrite or peroxynitrite anion (ONOO—), nitrogen dioxide (.NO2), dinitrogen trioxide (N2O3), peroxynitrous acid (ONOOH), and nitroperoxycarbonate (ONOOCO2-) (unpaired electrons denoted by ⋅). Bacteria have evolved transcription factors that are capable of sensing RNS levels. Different RNS signaling pathways are triggered by different RNS levels and occur with different kinetics.

As used herein, “RNS-inducible regulatory region” refers to a nucleic acid sequence to which one or more RNS-sensing transcription factors is capable of binding, wherein the binding and/or activation of the corresponding transcription factor activates downstream gene expression; in the presence of RNS, the transcription factor binds to and/or activates the regulatory region. In some embodiments, the RNS-inducible regulatory region comprises a promoter sequence. In some embodiments, the transcription factor senses RNS and subsequently binds to the RNS-inducible regulatory region, thereby activating downstream gene expression. In alternate embodiments, the transcription factor is bound to the RNS-inducible regulatory region in the absence of RNS; in the presence of RNS, the transcription factor undergoes a conformational change, thereby activating downstream gene expression. The RNS-inducible regulatory region may be operatively linked to a gene or genes, e.g., a branched chain amino acid catabolism enzymegene sequence(s), e.g., any of the amino acid catabolism enzymes described herein. For example, in the presence of RNS, a transcription factor senses RNS and activates a corresponding RNS-inducible regulatory region, thereby driving expression of an operatively linked gene sequence. Thus, RNS induces expression of the gene or gene sequences.

As used herein, “RNS-derepressible regulatory region” refers to a nucleic acid sequence to which one or more RNS-sensing transcription factors is capable of binding, wherein the binding of the corresponding transcription factor represses downstream gene expression; in the presence of RNS, the transcription factor does not bind to and does not repress the regulatory region. In some embodiments, the RNS-derepressible regulatory region comprises a promoter sequence. The RNS-derepressible regulatory region may be operatively linked to a gene or genes, e.g., a branched chain amino acid catabolism enzymegene sequence(s), BCAA transporter sequence(s), BCAA binding protein(s). For example, in the presence of RNS, a transcription factor senses RNS and no longer binds to and/or represses the regulatory region, thereby derepressing an operatively linked gene sequence or gene cassette. Thus, RNS derepresses expression of the gene or genes.

As used herein, “RNS-repressible regulatory region” refers to a nucleic acid sequence to which one or more RNS-sensing transcription factors is capable of binding, wherein the binding of the corresponding transcription factor represses downstream gene expression; in the presence of RNS, the transcription factor binds to and represses the regulatory region. In some embodiments, the RNS-repressible regulatory region comprises a promoter sequence. In some embodiments, the transcription factor that senses RNS is capable of binding to a regulatory region that overlaps with part of the promoter sequence. In alternate embodiments, the transcription factor that senses RNS is capable of binding to a regulatory region that is upstream or downstream of the promoter sequence. The RNS-repressible regulatory region may be operatively linked to a gene sequence or gene cassette. For example, in the presence of RNS, a transcription factor senses RNS and binds to a corresponding RNS-repressible regulatory region, thereby blocking expression of an operatively linked gene sequence or gene sequences. Thus, RNS represses expression of the gene or gene sequences.

As used herein, a “RNS-responsive regulatory region” refers to a RNS-inducible regulatory region, a RNS-repressible regulatory region, and/or a RNS-derepressible regulatory region. In some embodiments, the RNS-responsive regulatory region comprises a promoter sequence. Each regulatory region is capable of binding at least one corresponding RNS-sensing transcription factor. Examples of transcription factors that sense RNS and their corresponding RNS-responsive genes, promoters, and/or regulatory regions include, but are not limited to, those shown in Table 4.

TABLE 4
Examples of RNS-sensing transcription
factors and RNS-responsive genes

| RNS-sensing | Primarily | |
|---|---|---|
| transcription | capable of | Examples of responsive genes, |
| factor: | sensing: | promoters, and/or regulatory regions: |
| NsrR | NO | norB, aniA, nsrR, hmpA, ytfE, |
| | | ygbA, hcp, hcr, nrfA, aox |
| NorR | NO | norVW, norR |
| DNR | NO | norCB, nir, nor, nos |

In some embodiments, the genetically engineered bacteria of the invention comprise a tunable regulatory region that is directly or indirectly controlled by a transcription factor that is capable of sensing at least one reactive nitrogen species. The tunable regulatory region is operatively linked to a gene or genes capable of directly or indirectly driving the expression of an amino acid catabolism enzyme, BCAA transporter, and/or BCAA binding protein, thus controlling expression of the branched chain amino acid catabolism enzyme, BCAA transporter, and/or BCAA binding protein relative to RNS levels. For example, the tunable regulatory region is a RNS-inducible regulatory region, and the payload is an amino acid catabolism enzyme, BCAA transporter, and/or BCAA binding protein, such as any of the amino acid catabolism enzymes, BCAA transporters, and BCAA binding proteins provided herein; when RNS is present, e.g., in an inflamed tissue, a RNS-sensing transcription factor binds to and/or activates the regulatory region and drives expression of the branched chain amino acid catabolism enzyme, BCAA transporter, and/or BCAA binding protein gene or genes. Subsequently, when inflammation is ameliorated, RNS levels are reduced, and production of the branched chain amino acid catabolism enzyme, BCAA transporter, and/or BCAA binding protein is decreased or eliminated.

In some embodiments, the tunable regulatory region is a RNS-inducible regulatory region; in the presence of RNS, a transcription factor senses RNS and activates the RNS-inducible regulatory region, thereby driving expression of an operatively linked gene or genes. In some embodiments, the transcription factor senses RNS and subsequently binds to the RNS-inducible regulatory region, thereby activating downstream gene expression. In alternate embodiments, the transcription factor is bound to the RNS-inducible regulatory region in the absence of RNS; when the transcription factor senses RNS, it undergoes a conformational change, thereby inducing downstream gene expression.

In some embodiments, the tunable regulatory region is a RNS-inducible regulatory region, and the transcription factor that senses RNS is NorR. NorR “is an NO-responsive transcriptional activator that regulates expression of the norVW genes encoding flavorubredoxin and an associated flavoprotein, which reduce NO to nitrous oxide” (Spiro 2006). The genetically engineered bacteria of the invention may comprise any suitable RNS-responsive regulatory region from a gene that is activated by NorR. Genes that are capable of being activated by NorR are known in the art (see, e.g., Spiro 2006; Vine et al., 2011; Karlinsey et al., 2012). In certain embodiments, the genetically engineered bacteria of the invention comprise a RNS-inducible regulatory region from norVW that is operatively linked to a gene or genes, e.g., one or more branched chain amino acid catabolism enzyme, BCAA transporter, and/or BCAA binding protein gene sequence(s). In the presence of RNS, a NorR transcription factor senses RNS and activates to the norVW regulatory region, thereby driving expression of the operatively linked gene(s) and producing the amino acid catabolism enzyme, BCAA transporter, and/or BCAA binding protein.

In some embodiments, the tunable regulatory region is a RNS-inducible regulatory region, and the transcription factor that senses RNS is DNR. DNR (dissimilatory nitrate respiration regulator) “promotes the expression of the nir, the nor and the nos genes” in the presence of nitric oxide (Castiglione et al., 2009). The genetically engineered bacteria of the invention may comprise any suitable RNS-responsive regulatory region from a gene that is activated by DNR. Genes that are capable of being activated by DNR are known in the art (see, e.g., Castiglione et al., 2009; Giardina et al., 2008; Table 1). In certain embodiments, the genetically engineered bacteria of the invention comprise a RNS-inducible regulatory region from norCB that is operatively linked to a gene or gene cassette, e.g., a butyrogenic gene cassette. In the presence of RNS, a DNR transcription factor senses RNS and activates to the norCB regulatory region, thereby driving expression of the operatively linked gene or genes and producing one or more amino acid catabolism enzymes. In some embodiments, the DNR is Pseudomonas aeruginosa DNR.

In some embodiments, the tunable regulatory region is a RNS-derepressible regulatory region, and binding of a corresponding transcription factor represses downstream gene expression; in the presence of RNS, the transcription factor no longer binds to the regulatory region, thereby derepressing the operatively linked gene or gene cassette.

In some embodiments, the tunable regulatory region is a RNS-derepressible regulatory region, and the transcription factor that senses RNS is NsrR. NsrR is “an Rrf2-type transcriptional repressor [that] can sense NO and control the expression of genes responsible for NO metabolism” (Isabella et al., 2009). The genetically engineered bacteria of the invention may comprise any suitable RNS-responsive regulatory region from a gene that is repressed by NsrR. In some embodiments, the NsrR is Neisseria gonorrhoeae NsrR. Genes that are capable of being repressed by NsrR are known in the art (see, e.g., Isabella et al., 2009; Dunn et al., 2010). In certain embodiments, the genetically engineered bacteria of the invention comprise a RNS-derepressible regulatory region from norB that is operatively linked to a gene or genes, e.g., a branched chain amino acid catabolism enzyme gene or genes. In the presence of RNS, an NsrR transcription factor senses RNS and no longer binds to the norB regulatory region, thereby derepressing the operatively linked branched chain amino acid catabolism enzyme, BCAA transporter, and/or BCAA binding protein gene or genes and producing the encoding an amino acid catabolism enzyme(s).

In some embodiments, it is advantageous for the genetically engineered bacteria to express a RNS-sensing transcription factor that does not regulate the expression of a significant number of native genes in the bacteria. In some embodiments, the genetically engineered bacterium of the invention expresses a RNS-sensing transcription factor from a different species, strain, or substrain of bacteria, wherein the transcription factor does not bind to regulatory sequences in the genetically engineered bacterium of the invention. In some embodiments, the genetically engineered bacterium of the invention is Escherichia coli, and the RNS-sensing transcription factor is NsrR, e.g., from is Neisseria gonorrhoeae, wherein the Escherichia coli does not comprise binding sites for said NsrR. In some embodiments, the heterologous transcription factor minimizes or eliminates off-target effects on endogenous regulatory regions and genes in the genetically engineered bacteria.

In some embodiments, the tunable regulatory region is a RNS-repressible regulatory region, and binding of a corresponding transcription factor represses downstream gene expression; in the presence of RNS, the transcription factor senses RNS and binds to the RNS-repressible regulatory region, thereby repressing expression of the operatively linked gene or gene cassette. In some embodiments, the RNS-sensing transcription factor is capable of binding to a regulatory region that overlaps with part of the promoter sequence. In alternate embodiments, the RNS-sensing transcription factor is capable of binding to a regulatory region that is upstream or downstream of the promoter sequence.

In these embodiments, the genetically engineered bacteria may comprise a two repressor activation regulatory circuit, which is used to express an amino acid catabolism enzyme. The two repressor activation regulatory circuit comprises a first RNS-sensing repressor and a second repressor, which is operatively linked to a gene or gene cassette, e.g., encoding an amino acid catabolism enzyme. In one aspect of these embodiments, the RNS-sensing repressor inhibits transcription of the second repressor, which inhibits the transcription of the gene or gene cassette. Examples of second repressors useful in these embodiments include, but are not limited to, TetR, C1, and LexA. In the absence of binding by the first repressor (which occurs in the absence of RNS), the second repressor is transcribed, which represses expression of the gene or genes. In the presence of binding by the first repressor (which occurs in the presence of RNS), expression of the second repressor is repressed, and the gene or genes, e.g., a branched chain amino acid catabolism enzyme, BCAA transporter, and/or BCAA binding protein gene or genes is expressed.

A RNS-responsive transcription factor may induce, derepress, or repress gene expression depending upon the regulatory region sequence used in the genetically engineered bacteria. One or more types of RNS-sensing transcription factors and corresponding regulatory region sequences may be present in genetically engineered bacteria. In some embodiments, the genetically engineered bacteria comprise one type of RNS-sensing transcription factor, e.g., NsrR, and one corresponding regulatory region sequence, e.g., from norB. In some embodiments, the genetically engineered bacteria comprise one type of RNS-sensing transcription factor, e.g., NsrR, and two or more different corresponding regulatory region sequences, e.g., from norB and aniA. In some embodiments, the genetically engineered bacteria comprise two or more types of RNS-sensing transcription factors, e.g., NsrR and NorR, and two or more corresponding regulatory region sequences, e.g., from norB and norR, respectively. One RNS-responsive regulatory region may be capable of binding more than one transcription factor. In some embodiments, the genetically engineered bacteria comprise two or more types of RNS-sensing transcription factors and one corresponding regulatory region sequence. Nucleic acid sequences of several RNS-regulated regulatory regions are known in the art (see, e.g., Spiro 2006; Isabella et al., 2009; Dunn et al., 2010; Vine et al., 2011; Karlinsey et al., 2012).

In some embodiments, the genetically engineered bacteria of the invention comprise a gene encoding a RNS-sensing transcription factor, e.g., the nsrR gene, that is controlled by its native promoter, an inducible promoter, a promoter that is stronger than the native promoter, e.g., the GlnRS promoter or the P(Bla) promoter, or a constitutive promoter. In some instances, it may be advantageous to express the RNS-sensing transcription factor under the control of an inducible promoter in order to enhance expression stability. In some embodiments, expression of the RNS-sensing transcription factor is controlled by a different promoter than the promoter that controls expression of the therapeutic molecule. In some embodiments, expression of the RNS-sensing transcription factor is controlled by the same promoter that controls expression of the therapeutic molecule. In some embodiments, the RNS-sensing transcription factor and therapeutic molecule are divergently transcribed from a promoter region.

In some embodiments, the genetically engineered bacteria of the invention comprise a gene for a RNS-sensing transcription factor from a different species, strain, or substrain of bacteria. In some embodiments, the genetically engineered bacteria comprise a RNS-responsive regulatory region from a different species, strain, or substrain of bacteria. In some embodiments, the genetically engineered bacteria comprise a RNS-sensing transcription factor and corresponding RNS-responsive regulatory region from a different species, strain, or substrain of bacteria. The heterologous RNS-sensing transcription factor and regulatory region may increase the transcription of genes operatively linked to said regulatory region in the presence of RNS, as compared to the native transcription factor and regulatory region from bacteria of the same subtype under the same conditions.

In some embodiments, the genetically engineered bacteria comprise a RNS-sensing transcription factor, NsrR, and corresponding regulatory region, nsrR, from Neisseria gonorrhoeae. In some embodiments, the native RNS-sensing transcription factor, e.g., NsrR, is left intact and retains wild-type activity. In alternate embodiments, the native RNS-sensing transcription factor, e.g., NsrR, is deleted or mutated to reduce or eliminate wild-type activity.

In some embodiments, the genetically engineered bacteria of the invention comprise multiple copies of the endogenous gene encoding the RNS-sensing transcription factor, e.g., the nsrR gene. In some embodiments, the gene encoding the RNS-sensing transcription factor is present on a plasmid. In some embodiments, the gene encoding the RNS-sensing transcription factor and the gene or gene cassette for producing the therapeutic molecule are present on different plasmids. In some embodiments, the gene encoding the RNS-sensing transcription factor and the gene or gene cassette for producing the therapeutic molecule are present on the same plasmid. In some embodiments, the gene encoding the RNS-sensing transcription factor is present on a chromosome. In some embodiments, the gene encoding the RNS-sensing transcription factor and the gene or gene cassette for producing the therapeutic molecule are present on different chromosomes. In some embodiments, the gene encoding the RNS-sensing transcription factor and the gene or gene cassette for producing the therapeutic molecule are present on the same chromosome.

In some embodiments, the genetically engineered bacteria comprise a wild-type gene encoding a RNS-sensing transcription factor, e.g., the NsrR gene, and a corresponding regulatory region, e.g., a norB regulatory region, that is mutated relative to the wild-type regulatory region from bacteria of the same subtype. The mutated regulatory region increases the expression of the branched chain amino acid catabolism enzymein the presence of RNS, as compared to the wild-type regulatory region under the same conditions. In some embodiments, the genetically engineered bacteria comprise a wild-type RNS-responsive regulatory region, e.g., the norB regulatory region, and a corresponding transcription factor, e.g., NsrR, that is mutated relative to the wild-type transcription factor from bacteria of the same subtype. The mutant transcription factor increases the expression of the branched chain amino acid catabolism enzymein the presence of RNS, as compared to the wild-type transcription factor under the same conditions. In some embodiments, both the RNS-sensing transcription factor and corresponding regulatory region are mutated relative to the wild-type sequences from bacteria of the same subtype in order to increase expression of the branched chain amino acid catabolism enzyme, BCAA transporter, and/or BCAA binding protein in the presence of RNS.

In some embodiments, the gene or gene cassette for producing the anti-inflammation and/or gut barrier function enhancer molecule is present on a plasmid and operably linked to a promoter that is induced by RNS. In some embodiments, expression is further optimized by methods known in the art, e.g., by optimizing ribosomal binding sites, manipulating transcriptional regulators, and/or increasing mRNA stability.

In some embodiments, any of the gene(s) of the present disclosure may be integrated into the bacterial chromosome at one or more integration sites. For example, one or more copies of a branched chain amino acid catabolism enzyme, BCAA transporter, and/or BCAA binding protein gene(s) may be integrated into the bacterial chromosome. Having multiple copies of the gene or gen(s) integrated into the chromosome allows for greater production of the amino acid catabolism enzyme(s) and also permits fine-tuning of the level of expression. Alternatively, different circuits described herein, such as any of the secretion or exporter circuits, in addition to the therapeutic gene(s) or gene cassette(s) could be integrated into the bacterial chromosome at one or more different integration sites to perform multiple different functions.

ROS-Dependent Regulation

In some embodiments, the genetically engineered bacteria comprise a gene for producing an branched chain amino acid catabolism enzyme, BCAA transporter, and/or BCAA binding protein that is expressed under the control of an inducible promoter. In some embodiments, the genetically engineered bacterium that expresses a branched chain amino acid catabolism enzyme, BCAA transporter, and/or BCAA binding protein under the control of a promoter that is activated by conditions of cellular damage. In one embodiment, the gene for producing the branched chain amino acid catabolism enzyme is expressed under the control of a cellular damaged-dependent promoter that is activated in environments in which there is cellular or tissue damage, e.g., a reactive oxygen species or ROS promoter.

As used herein, “reactive oxygen species” and “ROS” are used interchangeably to refer to highly active molecules, ions, and/or radicals derived from molecular oxygen. ROS can be produced as byproducts of aerobic respiration or metal-catalyzed oxidation and may cause deleterious cellular effects such as oxidative damage. ROS includes, but is not limited to, hydrogen peroxide (H2O2), organic peroxide (ROOH), hydroxyl ion (OH—), hydroxyl radical (.OH), superoxide or superoxide anion (.O2-), singlet oxygen (1O2), ozone (O3), carbonate radical, peroxide or peroxyl radical (.O2-2), hypochlorous acid (HOCl), hypochlorite ion (OCl—), sodium hypochlorite (NaOCl), nitric oxide (NO.), and peroxynitrite or peroxynitrite anion (ONOO—) (unpaired electrons denoted by ⋅). Bacteria have evolved transcription factors that are capable of sensing ROS levels. Different ROS signaling pathways are triggered by different ROS levels and occur with different kinetics (Marinho et al., 2014).

As used herein, “ROS-inducible regulatory region” refers to a nucleic acid sequence to which one or more ROS-sensing transcription factors is capable of binding, wherein the binding and/or activation of the corresponding transcription factor activates downstream gene expression; in the presence of ROS, the transcription factor binds to and/or activates the regulatory region. In some embodiments, the ROS-inducible regulatory region comprises a promoter sequence. In some embodiments, the transcription factor senses ROS and subsequently binds to the ROS-inducible regulatory region, thereby activating downstream gene expression. In alternate embodiments, the transcription factor is bound to the ROS-inducible regulatory region in the absence of ROS; in the presence of ROS, the transcription factor undergoes a conformational change, thereby activating downstream gene expression. The ROS-inducible regulatory region may be operatively linked to a gene sequence or gene sequence, e.g., a sequence or sequences encoding one or more amino acid catabolism enzyme(s). For example, in the presence of ROS, a transcription factor, e.g., OxyR, senses ROS and activates a corresponding ROS-inducible regulatory region, thereby driving expression of an operatively linked gene sequence or gene sequences. Thus, ROS induces expression of the gene or genes.

As used herein, “ROS-derepressible regulatory region” refers to a nucleic acid sequence to which one or more ROS-sensing transcription factors is capable of binding, wherein the binding of the corresponding transcription factor represses downstream gene expression; in the presence of ROS, the transcription factor does not bind to and does not repress the regulatory region. In some embodiments, the ROS-derepressible regulatory region comprises a promoter sequence. The ROS-derepressible regulatory region may be operatively linked to a gene or genes, e.g., one or more genes encoding one or more amino acid catabolism enzyme(s). For example, in the presence of ROS, a transcription factor, e.g., OhrR, senses ROS and no longer binds to and/or represses the regulatory region, thereby derepressing an operatively linked gene sequence or gene cassette. Thus, ROS derepresses expression of the gene or gene cassette.

As used herein, “ROS-repressible regulatory region” refers to a nucleic acid sequence to which one or more ROS-sensing transcription factors is capable of binding, wherein the binding of the corresponding transcription factor represses downstream gene expression; in the presence of ROS, the transcription factor binds to and represses the regulatory region. In some embodiments, the ROS-repressible regulatory region comprises a promoter sequence. In some embodiments, the transcription factor that senses ROS is capable of binding to a regulatory region that overlaps with part of the promoter sequence. In alternate embodiments, the transcription factor that senses ROS is capable of binding to a regulatory region that is upstream or downstream of the promoter sequence. The ROS-repressible regulatory region may be operatively linked to a gene sequence or gene sequences. For example, in the presence of ROS, a transcription factor, e.g., PerR, senses ROS and binds to a corresponding ROS-repressible regulatory region, thereby blocking expression of an operatively linked gene sequence or gene sequences. Thus, ROS represses expression of the gene or genes.

As used herein, a “ROS-responsive regulatory region” refers to a ROS-inducible regulatory region, a ROS-repressible regulatory region, and/or a ROS-derepressible regulatory region. In some embodiments, the ROS-responsive regulatory region comprises a promoter sequence. Each regulatory region is capable of binding at least one corresponding ROS-sensing transcription factor. Examples of transcription factors that sense ROS and their corresponding ROS-responsive genes, promoters, and/or regulatory regions include, but are not limited to, those shown in Table 5.

TABLE 5
Examples of ROS-sensing transcription
factors and ROS-responsive genes

| ROS-sensing | Primarily | |
|---|---|---|
| transcription | capable of | Examples of responsive genes, |
| factor: | sensing: | promoters, and/or regulatory regions: |
| OxyR | H₂O₂ | ahpC; ahpF; dps; dsbG; fhuF; flu; fur; gor; |
| | | grxA; hemH; katG; oxyS; sufA; sufB; sufC; |
| | | sufD; sufE; sufS; trxC; uxuA; yaaA; yaeH; |
| | | yaiA; ybjM; ydcH; ydeN; ygaQ; yljA; ytfK |
| PerR | H₂O₂ | katA; ahpCF; mrgA; zoaA; fur; |
| | | hemAXCDBL; srfA |
| OhrR | Organic | ohrA |
| | peroxides | |
| | NaOCl | |
| SoxR | •O₂⁻ | soxS |
| | NO• | |
| | (also capable | |
| | of sensing | |
| | H₂O₂) | |
| RosR | H₂O₂ | rbtT; tnp16a; rluC1; tnp5a; mscL; tnp2d; |
| | | phoD; tnp15b; pstA; tnp5b; xylC; gabD1; |
| | | rluC2; cgtS9; azlC; narKGHJI; rosR |

In some embodiments, the genetically engineered bacteria comprise a tunable regulatory region that is directly or indirectly controlled by a transcription factor that is capable of sensing at least one reactive oxygen species. The tunable regulatory region is operatively linked to a gene or gene cassette capable of directly or indirectly driving the expression of an amino acid catabolism enzyme, thus controlling expression of the branched chain amino acid catabolism enzyme relative to ROS levels. For example, the tunable regulatory region is a ROS-inducible regulatory region, and the molecule is an amino acid catabolism enzyme; when ROS is present, e.g., in an inflamed tissue, a ROS-sensing transcription factor binds to and/or activates the regulatory region and drives expression of the gene sequence for the amino acid catabolism enzyme, BCAA transporter, and/or BCAA binding protein thereby producing the amino acid catabolism enzyme, BCAA transporter, and/or BCAA binding protein. Subsequently, when inflammation is ameliorated, ROS levels are reduced, and production of the branched chain amino acid catabolism enzyme, BCAA transporter, and/or BCAA binding protein is decreased or eliminated.

In some embodiments, the tunable regulatory region is a ROS-inducible regulatory region; in the presence of ROS, a transcription factor senses ROS and activates the ROS-inducible regulatory region, thereby driving expression of an operatively linked gene or gene cassette. In some embodiments, the transcription factor senses ROS and subsequently binds to the ROS-inducible regulatory region, thereby activating downstream gene expression. In alternate embodiments, the transcription factor is bound to the ROS-inducible regulatory region in the absence of ROS; when the transcription factor senses ROS, it undergoes a conformational change, thereby inducing downstream gene expression.

In some embodiments, the tunable regulatory region is a ROS-inducible regulatory region, and the transcription factor that senses ROS is OxyR. OxyR “functions primarily as a global regulator of the peroxide stress response” and is capable of regulating dozens of genes, e.g., “genes involved in H2O2 detoxification (katE, ahpCF), heme biosynthesis (hemH), reductant supply (grxA, gor, trxC), thiol-disulfide isomerization (dsbG), Fe-S center repair (sufA-E, sufS), iron binding (yaaA), repression of iron import systems (fur)” and “OxyS, a small regulatory RNA” (Dubbs et al., 2012). The genetically engineered bacteria may comprise any suitable ROS-responsive regulatory region from a gene that is activated by OxyR. Genes that are capable of being activated by OxyR are known in the art (see, e.g., Zheng et al., 2001; Dubbs et al., 2012; Table 1). In certain embodiments, the genetically engineered bacteria of the invention comprise a ROS-inducible regulatory region from oxyS that is operatively linked to a gene, e.g., a branched chain amino acid catabolism enzyme, BCAA transporter, and/or BCAA binding protein gene. In the presence of ROS, e.g., H2O2, an OxyR transcription factor senses ROS and activates to the oxyS regulatory region, thereby driving expression of the operatively linked branched chain amino acid catabolism enzyme, BCAA transporter, and/or BCAA binding protein gene and producing the amino acid catabolism enzyme, BCAA transporter, and/or BCAA binding protein. In some embodiments, OxyR is encoded by an E. coli oxyR gene. In some embodiments, the oxyS regulatory region is an E. coli oxyS regulatory region. In some embodiments, the ROS-inducible regulatory region is selected from the regulatory region of katG, dps, and ahpC.

In alternate embodiments, the tunable regulatory region is a ROS-inducible regulatory region, and the corresponding transcription factor that senses ROS is SoxR. When SoxR is “activated by oxidation of its [2Fe-2S] cluster, it increases the synthesis of SoxS, which then activates its target gene expression” (Koo et al., 2003). “SoxR is known to respond primarily to superoxide and nitric oxide” (Koo et al., 2003), and is also capable of responding to H₂O₂. The genetically engineered bacteria of the invention may comprise any suitable ROS-responsive regulatory region from a gene that is activated by SoxR. Genes that are capable of being activated by SoxR are known in the art (see, e.g., Koo et al., 2003; Table 1). In certain embodiments, the genetically engineered bacteria of the invention comprise a ROS-inducible regulatory region from soxS that is operatively linked to a gene, e.g., an amino acid catabolism enzyme. In the presence of ROS, the SoxR transcription factor senses ROS and activates the soxS regulatory region, thereby driving expression of the operatively linked branched chain amino acid catabolism enzyme, BCAA transporter, and/or BCAA binding protein gene and producing an amino acid catabolism enzyme, BCAA transporter, and/or BCAA binding protein.

In some embodiments, the tunable regulatory region is a ROS-derepressible regulatory region, and binding of a corresponding transcription factor represses downstream gene expression; in the presence of ROS, the transcription factor no longer binds to the regulatory region, thereby derepressing the operatively linked gene or gene cassette.

In some embodiments, the tunable regulatory region is a ROS-derepressible regulatory region, and the transcription factor that senses ROS is OhrR. OhrR “binds to a pair of inverted repeat DNA sequences overlapping the ohrA promoter site and thereby represses the transcription event,” but oxidized OhrR is “unable to bind its DNA target” (Duarte et al., 2010). OhrR is a “transcriptional repressor [that] . . . senses both organic peroxides and NaOCl” (Dubbs et al., 2012) and is “weakly activated by H2O2 but it shows much higher reactivity for organic hydroperoxides” (Duarte et al., 2010). The genetically engineered bacteria of the invention may comprise any suitable ROS-responsive regulatory region from a gene that is repressed by OhrR. Genes that are capable of being repressed by OhrR are known in the art (see, e.g., Dubbs et al., 2012; Table 1). In certain embodiments, the genetically engineered bacteria of the invention comprise a ROS-derepressible regulatory region from ohrA that is operatively linked to a gene or gene cassette, e.g., a branched chain amino acid catabolism enzyme, BCAA transporter, and/or BCAA binding protein gene. In the presence of ROS, e.g., NaOCl, an OhrR transcription factor senses ROS and no longer binds to the ohrA regulatory region, thereby derepressing the operatively linked branched chain amino acid catabolism enzyme, BCAA transporter, and/or BCAA binding protein gene and producing an amino acid catabolism enzyme, BCAA transporter, and/or BCAA binding protein.

OhrR is a member of the MarR family of ROS-responsive regulators. “Most members of the MarR family are transcriptional repressors and often bind to the −10 or −35 region in the promoter causing a steric inhibition of RNA polymerase binding” (Bussmann et al., 2010). Other members of this family are known in the art and include, but are not limited to, OspR, MgrA, RosR, and SarZ. In some embodiments, the transcription factor that senses ROS is OspR, MgRA, RosR, and/or SarZ, and the genetically engineered bacteria of the invention comprises one or more corresponding regulatory region sequences from a gene that is repressed by OspR, MgRA, RosR, and/or SarZ. Genes that are capable of being repressed by OspR, MgRA, RosR, and/or SarZ are known in the art (see, e.g., Dubbs et al., 2012).

In some embodiments, the tunable regulatory region is a ROS-derepressible regulatory region, and the corresponding transcription factor that senses ROS is RosR. RosR is “a MarR-type transcriptional regulator” that binds to an “18-bp inverted repeat with the consensus sequence TTGTTGAYRYRTCAACWA (SEQ ID NO: 163)” and is “reversibly inhibited by the oxidant H2O2” (Bussmann et al., 2010). RosR is capable of repressing numerous genes and putative genes, including but not limited to “a putative polyisoprenoid-binding protein (cg1322, gene upstream of and divergent from rosR), a sensory histidine kinase (cgtS9), a putative transcriptional regulator of the Crp/FNR family (cg3291), a protein of the glutathione S-transferase family (cg1426), two putative FMN reductases (cg1150 and cg1850), and four putative monooxygenases (cg0823, cg1848, cg2329, and cg3084)” (Bussmann et al., 2010). The genetically engineered bacteria of the invention may comprise any suitable ROS-responsive regulatory region from a gene that is repressed by RosR. Genes that are capable of being repressed by RosR are known in the art (see, e.g., Bussmann et al., 2010; Table 1). In certain embodiments, the genetically engineered bacteria of the invention comprise a ROS-derepressible regulatory region from cgtS9 that is operatively linked to a gene or gene cassette, e.g., an amino acid catabolism enzyme, BCAA transporter, and/or BCAA binding protein. In the presence of ROS, e.g., H2O2, a RosR transcription factor senses ROS and no longer binds to the cgtS9 regulatory region, thereby derepressing the operatively linked branched chain amino acid catabolism enzyme, BCAA transporter, and/or BCAA binding protein gene and producing the amino acid catabolism enzyme, BCAA transporter, and/or BCAA binding protein.

In some embodiments, it is advantageous for the genetically engineered bacteria to express a ROS-sensing transcription factor that does not regulate the expression of a significant number of native genes in the bacteria. In some embodiments, the genetically engineered bacterium of the invention expresses a ROS-sensing transcription factor from a different species, strain, or substrain of bacteria, wherein the transcription factor does not bind to regulatory sequences in the genetically engineered bacterium of the invention. In some embodiments, the genetically engineered bacterium of the invention is Escherichia coli, and the ROS-sensing transcription factor is RosR, e.g., from Corynebacterium glutamicum, wherein the Escherichia coli does not comprise binding sites for said RosR. In some embodiments, the heterologous transcription factor minimizes or eliminates off-target effects on endogenous regulatory regions and genes in the genetically engineered bacteria.

In some embodiments, the tunable regulatory region is a ROS-repressible regulatory region, and binding of a corresponding transcription factor represses downstream gene expression; in the presence of ROS, the transcription factor senses ROS and binds to the ROS-repressible regulatory region, thereby repressing expression of the operatively linked gene or gene cassette. In some embodiments, the ROS-sensing transcription factor is capable of binding to a regulatory region that overlaps with part of the promoter sequence. In alternate embodiments, the ROS-sensing transcription factor is capable of binding to a regulatory region that is upstream or downstream of the promoter sequence.

In some embodiments, the tunable regulatory region is a ROS-repressible regulatory region, and the transcription factor that senses ROS is PerR. In Bacillus subtilis, PerR “when bound to DNA, represses the genes coding for proteins involved in the oxidative stress response (katA, ahpC, and mrgA), metal homeostasis (hemAXCDBL, fur, and zoaA) and its own synthesis (perR)” (Marinho et al., 2014). PerR is a “global regulator that responds primarily to H2O2” (Dubbs et al., 2012) and “interacts with DNA at the per box, a specific palindromic consensus sequence (TTATAATNATTATAA (SEQ ID NO: 164)) residing within and near the promoter sequences of PerR-controlled genes” (Marinho et al., 2014). PerR is capable of binding a regulatory region that “overlaps part of the promoter or is immediately downstream from it” (Dubbs et al., 2012). The genetically engineered bacteria of the invention may comprise any suitable ROS-responsive regulatory region from a gene that is repressed by PerR. Genes that are capable of being repressed by PerR are known in the art (see, e.g., Dubbs et al., 2012).

In these embodiments, the genetically engineered bacteria may comprise a two repressor activation regulatory circuit, which is used to express an amino acid catabolism enzyme. The two repressor activation regulatory circuit comprises a first ROS-sensing repressor, e.g., PerR, and a second repressor, e.g., TetR, which is operatively linked to a gene or gene cassette, e.g., an amino acid catabolism enzyme. In one aspect of these embodiments, the ROS-sensing repressor inhibits transcription of the second repressor, which inhibits the transcription of the gene or gene cassette. Examples of second repressors useful in these embodiments include, but are not limited to, TetR, C1, and LexA. In some embodiments, the ROS-sensing repressor is PerR. In some embodiments, the second repressor is TetR. In this embodiment, a PerR-repressible regulatory region drives expression of TetR, and a TetR-repressible regulatory region drives expression of the gene or gene cassette, e.g., an amino acid catabolism enzyme. In the absence of PerR binding (which occurs in the absence of ROS), tetR is transcribed, and TetR represses expression of the gene or gene cassette, e.g., an amino acid catabolism enzyme. In the presence of PerR binding (which occurs in the presence of ROS), tetR expression is repressed, and the gene or gene cassette, e.g., an amino acid catabolism enzyme, BCAA transporter, and/or BCAA binding protein is expressed.

A ROS-responsive transcription factor may induce, derepress, or repress gene expression depending upon the regulatory region sequence used in the genetically engineered bacteria. For example, although “OxyR is primarily thought of as a transcriptional activator under oxidizing conditions . . . OxyR can function as either a repressor or activator under both oxidizing and reducing conditions” (Dubbs et al., 2012), and OxyR “has been shown to be a repressor of its own expression as well as that of fhuF (encoding a ferric ion reductase) and flu (encoding the antigen 43 outer membrane protein)” (Zheng et al., 2001). The genetically engineered bacteria of the invention may comprise any suitable ROS-responsive regulatory region from a gene that is repressed by OxyR. In some embodiments, OxyR is used in a two repressor activation regulatory circuit, as described above. Genes that are capable of being repressed by OxyR are known in the art (see, e.g., Zheng et al., 2001; Table 1). Or, for example, although RosR is capable of repressing a number of genes, it is also capable of activating certain genes, e.g., the narKGHJI operon. In some embodiments, the genetically engineered bacteria comprise any suitable ROS-responsive regulatory region from a gene that is activated by RosR. In addition, “PerR-mediated positive regulation has also been observed . . . and appears to involve PerR binding to distant upstream sites” (Dubbs et al., 2012). In some embodiments, the genetically engineered bacteria comprise any suitable ROS-responsive regulatory region from a gene that is activated by PerR.

One or more types of ROS-sensing transcription factors and corresponding regulatory region sequences may be present in genetically engineered bacteria. For example, “OhrR is found in both Gram-positive and Gram-negative bacteria and can coreside with either OxyR or PerR or both” (Dubbs et al., 2012). In some embodiments, the genetically engineered bacteria comprise one type of ROS-sensing transcription factor, e.g., OxyR, and one corresponding regulatory region sequence, e.g., from oxyS. In some embodiments, the genetically engineered bacteria comprise one type of ROS-sensing transcription factor, e.g., OxyR, and two or more different corresponding regulatory region sequences, e.g., from oxyS and katG. In some embodiments, the genetically engineered bacteria comprise two or more types of ROS-sensing transcription factors, e.g., OxyR and PerR, and two or more corresponding regulatory region sequences, e.g., from oxyS and katA, respectively. One ROS-responsive regulatory region may be capable of binding more than one transcription factor. In some embodiments, the genetically engineered bacteria comprise two or more types of ROS-sensing transcription factors and one corresponding regulatory region sequence.

In some embodiments, genetically engineered bacteria comprise a nucleic acid sequence that is at least about 80%, at least about 85%, at least about 90%, at least about 95%, or at least about 99% homologous to the DNA sequence of an exemplary OxyR-regulated regulatory region, or a functional fragment thereof. Such sequences include, but are not limited to aktG, dps, ahpC, and oxyS.

In some embodiments, the genetically engineered bacteria of the invention comprise a gene encoding a ROS-sensing transcription factor, e.g., the oxyR gene, that is controlled by its native promoter, an inducible promoter, a promoter that is stronger than the native promoter, e.g., the GlnRS promoter or the P(Bla) promoter, or a constitutive promoter. In some instances, it may be advantageous to express the ROS-sensing transcription factor under the control of an inducible promoter in order to enhance expression stability. In some embodiments, expression of the ROS-sensing transcription factor is controlled by a different promoter than the promoter that controls expression of the therapeutic molecule. In some embodiments, expression of the ROS-sensing transcription factor is controlled by the same promoter that controls expression of the therapeutic molecule. In some embodiments, the ROS-sensing transcription factor and therapeutic molecule are divergently transcribed from a promoter region.

In some embodiments, the genetically engineered bacteria of the invention comprise a gene for a ROS-sensing transcription factor from a different species, strain, or substrain of bacteria. In some embodiments, the genetically engineered bacteria comprise a ROS-responsive regulatory region from a different species, strain, or substrain of bacteria. In some embodiments, the genetically engineered bacteria comprise a ROS-sensing transcription factor and corresponding ROS-responsive regulatory region from a different species, strain, or substrain of bacteria. The heterologous ROS-sensing transcription factor and regulatory region may increase the transcription of genes operatively linked to said regulatory region in the presence of ROS, as compared to the native transcription factor and regulatory region from bacteria of the same subtype under the same conditions.

In some embodiments, the genetically engineered bacteria comprise a ROS-sensing transcription factor, OxyR, and corresponding regulatory region, oxyS, from Escherichia coli. In some embodiments, the native ROS-sensing transcription factor, e.g., OxyR, is left intact and retains wild-type activity. In alternate embodiments, the native ROS-sensing transcription factor, e.g., OxyR, is deleted or mutated to reduce or eliminate wild-type activity.

In some embodiments, the genetically engineered bacteria of the invention comprise multiple copies of the endogenous gene encoding the ROS-sensing transcription factor, e.g., the oxyR gene. In some embodiments, the gene encoding the ROS-sensing transcription factor is present on a plasmid. In some embodiments, the gene encoding the ROS-sensing transcription factor and the gene or gene cassette for producing the therapeutic molecule are present on different plasmids. In some embodiments, the gene encoding the ROS-sensing transcription factor and the gene or gene cassette for producing the therapeutic molecule are present on the same. In some embodiments, the gene encoding the ROS-sensing transcription factor is present on a chromosome. In some embodiments, the gene encoding the ROS-sensing transcription factor and the gene or gene cassette for producing the therapeutic molecule are present on different chromosomes. In some embodiments, the gene encoding the ROS-sensing transcription factor and the gene or gene cassette for producing the therapeutic molecule are present on the same chromosome.

In some embodiments, the genetically engineered bacteria comprise a wild-type gene encoding a ROS-sensing transcription factor, e.g., the soxR gene, and a corresponding regulatory region, e.g., a soxS regulatory region, that is mutated relative to the wild-type regulatory region from bacteria of the same subtype. The mutated regulatory region increases the expression of the branched chain amino acid catabolism enzyme, BCAA transporter, and/or BCAA binding protein in the presence of ROS, as compared to the wild-type regulatory region under the same conditions. In some embodiments, the genetically engineered bacteria comprise a wild-type ROS-responsive regulatory region, e.g., the oxyS regulatory region, and a corresponding transcription factor, e.g., OxyR, that is mutated relative to the wild-type transcription factor from bacteria of the same subtype. The mutant transcription factor increases the expression of the branched chain amino acid catabolism enzyme, BCAA transporter, and/or BCAA binding protein in the presence of ROS, as compared to the wild-type transcription factor under the same conditions. In some embodiments, both the ROS-sensing transcription factor and corresponding regulatory region are mutated relative to the wild-type sequences from bacteria of the same subtype in order to increase expression of the branched chain amino acid catabolism enzymein the presence of ROS.

In some embodiments, the gene or gene cassette for producing the branched chain amino acid catabolism enzyme is present on a plasmid and operably linked to a promoter that is induced by ROS. In some embodiments, the gene or gene cassette for producing the branched chain amino acid catabolism enzyme is present in the chromosome and operably linked to a promoter that is induced by ROS. In some embodiments, the gene or gene cassette for producing the branched chain amino acid catabolism enzyme is present on a chromosome and operably linked to a promoter that is induced by exposure to tetracycline. In some embodiments, the gene or gene cassette for producing the branched chain amino acid catabolism enzyme, BCAA transporter, and/or BCAA binding protein is present on a plasmid and operably linked to a promoter that is induced by exposure to tetracycline. In some embodiments, expression is further optimized by methods known in the art, e.g., by optimizing ribosomal binding sites, manipulating transcriptional regulators, and/or increasing mRNA stability.

In some embodiments, the genetically engineered bacteria may comprise multiple copies of the gene(s) capable of producing an amino acid catabolism enzyme(s), BCAA transporter(s), and/or BCAA binding protein(s). In some embodiments, the gene(s) capable of producing an amino acid catabolism enzyme(s), BCAA transporter(s), and/or BCAA binding protein(s) is present on a plasmid and operatively linked to a ROS-responsive regulatory region. In some embodiments, the gene(s) capable of producing a branched chain amino acid catabolism enzyme, BCAA transporter, and/or BCAA binding protein is present in a chromosome and operatively linked to a ROS-responsive regulatory region.

Thus, in some embodiments, the genetically engineered bacteria or genetically engineered yeast or virus produce one or more amino acid catabolism enzymes under the control of an oxygen level-dependent promoter, a reactive oxygen species (ROS)-dependent promoter, or a reactive nitrogen species (RNS)-dependent promoter, and a corresponding transcription factor.

In some embodiments, the genetically engineered bacteria comprise a stably maintained plasmid or chromosome carrying a gene for producing an amino acid catabolism enzyme, BCAA transporter, and/or BCAA binding protein such that the branched chain amino acid catabolism enzyme, BCAA transporter, and/or BCAA binding protein can be expressed in the host cell, and the host cell is capable of survival and/or growth in vitro, e.g., in medium, and/or in vivo. In some embodiments, a bacterium may comprise multiple copies of the gene encoding the amino acid catabolism enzyme, BCAA transporter, and/or BCAA binding protein. In some embodiments, the gene encoding the branched chain amino acid catabolism enzyme, BCAA transporter, and/or BCAA binding protein is expressed on a low-copy plasmid. In some embodiments, the low-copy plasmid may be useful for increasing stability of expression. In some embodiments, the low-copy plasmid may be useful for decreasing leaky expression under non-inducing conditions. In some embodiments, the gene encoding the branched chain amino acid catabolism enzyme, BCAA transporter, and/or BCAA binding protein is expressed on a high-copy plasmid. In some embodiments, the high-copy plasmid may be useful for increasing expression of the amino acid catabolism enzyme, BCAA transporter, and/or BCAA binding protein. In some embodiments, the gene encoding the branched chain amino acid catabolism enzyme, BCAA transporter, and/or BCAA binding protein is expressed on a chromosome.

In some embodiments, the bacteria are genetically engineered to include multiple mechanisms of action (MOAs), e.g., circuits producing multiple copies of the same product (e.g., to enhance copy number) or circuits performing multiple different functions. For example, the genetically engineered bacteria may include four copies of the gene encoding a particular branched chain amino acid catabolism enzyme, BCAA transporter, and/or BCAA binding protein inserted at four different insertion sites. Alternatively, the genetically engineered bacteria may include three copies of the gene encoding a particular branched chain amino acid catabolism enzyme, BCAA transporter, and/or BCAA binding protein inserted at three different insertion sites and three copies of the gene encoding a different branched chain amino acid catabolism enzyme, BCAA transporter, and/or BCAA binding protein inserted at three different insertion sites.

In some embodiments, under conditions where the branched chain amino acid catabolism enzyme, BCAA transporter, and/or BCAA binding protein is expressed, the genetically engineered bacteria of the disclosure produce at least about 1.5-fold, at least about 2-fold, at least about 10-fold, at least about 15-fold, at least about 20-fold, at least about 30-fold, at least about 50-fold, at least about 100-fold, at least about 200-fold, at least about 300-fold, at least about 400-fold, at least about 500-fold, at least about 600-fold, at least about 700-fold, at least about 800-fold, at least about 900-fold, at least about 1,000-fold, or at least about 1,500-fold more of the amino acid catabolism enzyme, BCAA transporter, and/or BCAA binding protein and/or transcript of the gene(s) in the operon as compared to unmodified bacteria of the same subtype under the same conditions.

In some embodiments, quantitative PCR (qPCR) is used to amplify, detect, and/or quantify mRNA expression levels of the branched chain amino acid catabolism enzyme, BCAA transporter, and/or BCAA binding protein gene(s). Primers specific for branched chain amino acid catabolism enzyme, BCAA transporter, and/or BCAA binding protein gene(s) may be designed and used to detect mRNA in a sample according to methods known in the art. In some embodiments, a fluorophore is added to a sample reaction mixture that may contain branched chain amino acid catabolism enzyme mRNA, and a thermal cycler is used to illuminate the sample reaction mixture with a specific wavelength of light and detect the subsequent emission by the fluorophore. The reaction mixture is heated and cooled to predetermined temperatures for predetermined time periods. In certain embodiments, the heating and cooling is repeated for a predetermined number of cycles. In some embodiments, the reaction mixture is heated and cooled to 90-100° C., 60-70° C., and 30-50° C. for a predetermined number of cycles. In a certain embodiment, the reaction mixture is heated and cooled to 93-97° C., 55-65° C., and 35-45° C. for a predetermined number of cycles. In some embodiments, the accumulating amplicon is quantified after each cycle of the qPCR. The number of cycles at which fluorescence exceeds the threshold is the threshold cycle (CT). At least one CT result for each sample is generated, and the CT result(s) may be used to determine mRNA expression levels of the branched chain amino acid catabolism enzyme, BCAA transporter, and/or BCAA binding protein gene(s).

In some embodiments, quantitative PCR (qPCR) is used to amplify, detect, and/or quantify mRNA expression levels of the branched chain amino acid catabolism enzyme, BCAA transporter, and/or BCAA binding protein gene(s). Primers specific for branched chain amino acid catabolism enzyme, BCAA transporter, and/or BCAA binding protein gene(s) may be designed and used to detect mRNA in a sample according to methods known in the art. In some embodiments, a fluorophore is added to a sample reaction mixture that may contain branched chain amino acid catabolism enzyme, BCAA transporter, and/or BCAA binding protein mRNA, and a thermal cycler is used to illuminate the sample reaction mixture with a specific wavelength of light and detect the subsequent emission by the fluorophore. The reaction mixture is heated and cooled to predetermined temperatures for predetermined time periods. In certain embodiments, the heating and cooling is repeated for a predetermined number of cycles. In some embodiments, the reaction mixture is heated and cooled to 90-100° C., 60-70° C., and 30-50° C. for a predetermined number of cycles. In a certain embodiment, the reaction mixture is heated and cooled to 93-97° C., 55-65° C., and 35-45° C. for a predetermined number of cycles. In some embodiments, the accumulating amplicon is quantified after each cycle of the qPCR. The number of cycles at which fluorescence exceeds the threshold is the threshold cycle (CT). At least one CT result for each sample is generated, and the CT result(s) may be used to determine mRNA expression levels of the branched chain amino acid catabolism enzyme, BCAA transporter, and/or BCAA binding protein gene(s).

In other embodiments, the inducible promoter is a propionate responsive promoter. For example, the prpR promoter is a propionate responsive promoter.

Inducible Promoters (Nutritional and or Chemical Inducer(s) and or Metabolite(s))

In some embodiments, one or more optimized gene sequence(s), e.g., kivD, leuDH, adh2, and brnQ, is present on a plasmid and operably linked to promoter a directly or indirectly inducible by one or more nutritional and/or chemical inducer(s) and/or metabolite(s). In some embodiments, the bacterial cell comprises a stably maintained plasmid or chromosome carrying the gene encoding the branched chain amino acid catabolism enzyme, which is induced by one or more nutritional and/or chemical inducer(s) and/or metabolite(s), such that the branched chain amino acid catabolism enzyme can be expressed in the host cell, and the host cell is capable of survival and/or growth in vitro, e.g., under culture conditions, and/or in vivo, e.g., in the gut. In some embodiments, bacterial cell comprises two or more distinct branched chain amino acid catabolism cassette(s), one or more of which are induced by one or more nutritional and/or chemical inducer(s) and/or metabolite(s). In some embodiments, the genetically engineered bacteria comprise multiple copies of the same branched chain amino acid catabolism enzyme gene(s) and/or gene cassette(s) which are induced by one or more nutritional and/or chemical inducer(s) and/or metabolite(s). In some embodiments, the genetically engineered bacteria comprise multiple copies of different branched chain amino acid catabolism enzyme genes or gene cassette(s), one or more of which are induced by one or more nutritional and/or chemical inducer(s) and/or metabolite(s).

In some embodiments, the gene encoding the branched chain amino acid catabolism enzyme is present on a plasmid and operably linked to a promoter that is induced by one or more nutritional and/or chemical inducer(s) and/or metabolite(s). In some embodiments, the gene encoding the branched chain amino acid catabolism enzyme is present in the chromosome and operably linked to a promoter that is induced by one or more nutritional and/or chemical inducer(s) and/or metabolite(s).

In some embodiments, the bacterial cell comprises a stably maintained plasmid or chromosome carrying the one or more gene sequences(s), inducible by one or more nutritional and/or chemical inducer(s) and/or metabolite(s), encoding a transporter of branched chain amino acid(s) and/or one or more metabolites thereof, e.g., livKHMGF and/or brnQ, such that the transporter can be expressed in the host cell, and the host cell is capable of survival and/or growth in vitro, e.g., in medium, and/or in vivo, e.g., in the gut. In some embodiments, bacterial cell comprises two or more distinct copies of the one or more gene sequences(s) encoding a branched chain amino acid transporter, which is controlled by a promoter inducible one or more nutritional and/or chemical inducer(s) and/or metabolite(s). In some embodiments, the genetically engineered bacteria comprise multiple copies of the same one or more gene sequences(s) encoding a branched chain amino acid transporter, which is controlled by a promoter inducible one or more nutritional and/or chemical inducer(s) and/or metabolite(s). In some embodiments, the one or more gene sequences(s) encoding a transporter of branched chain amino acid(s), is present on a plasmid and operably linked to a directly or indirectly inducible promoter inducible by one or more nutritional and/or chemical inducer(s) and/or metabolite(s). In some embodiments, the one or more gene sequences(s) encoding a branched chain amino acid transporter, is present on a chromosome and operably linked to a directly or indirectly inducible by one or more nutritional and/or chemical inducer(s) and/or metabolite(s).

In some embodiments, the gene encoding branched chain amino acid binding protein is present on a plasmid and operably linked to a promoter that is induced by one or more nutritional and/or chemical inducer(s) and/or metabolite(s). In some embodiments, the gene encoding branched chain amino acid binding protein is present in the chromosome and operably linked to a promoter that is induced by one or more nutritional and/or chemical inducer(s) and/or metabolite(s).

In some embodiments, the promoter that is operably linked to the gene encoding the branched chain amino acid catabolism enzyme and the promoter that is operably linked to the gene encoding the branched chain amino acid transporter and/or BCAA binding protein and/or BCAA exporter, is directly or indirectly induced by one or more nutritional and/or chemical inducer(s) and/or metabolite(s).

In some embodiments, one or more inducible promoter(s) are useful for or induced during in vivo expression of the one or more protein(s) of interest. In some embodiments, the promoters are induced during in vivo expression of one or more branched chain amino acid catabolism enzymes and/or branched chain amino acid transporter(s) and/or BCAA binding protein(s) and/or BCAA exporter(s). In some embodiments, expression of one or more branched chain amino acid catabolism enzyme(s) and/or branched chain amino acid transporter(s) and/or BCAA binding protein(s) and/or BCAA exporter(s) is driven directly or indirectly by one or more arabinose inducible promoter(s) in vivo. In some embodiments, the promoter is directly or indirectly induced by a chemical and/or nutritional inducer and/or metabolite which is co-administered with the genetically engineered bacteria of the invention.

In some embodiments, expression of one or more branched chain amino acid catabolism enzyme gene(s) and/or branched chain amino acid transporter(s) and/or BCAA binding protein(s) and/or BCAA exporter(s), is driven directly or indirectly by one or more promoter(s) induced by a chemical and/or nutritional inducer and/or metabolite during in vitro growth, preparation, or manufacturing of the strain prior to in vivo administration. In some embodiments, the promoter(s) induced by a chemical and/or nutritional inducer and/or metabolite are induced in culture, e.g., grown in a flask, fermenter or other appropriate culture vessel, e.g., used during cell growth, cell expansion, fermentation, recovery, purification, formulation, and/or manufacture. In some embodiments, the promoter is directly or indirectly induced by a molecule that is added to in the bacterial culture to induce expression and pre-load the bacterium with branched chain amino acid catabolism enzyme(s) and/or branched chain amino acid transporter(s) and/or BCAA binding protein(s) and/or BCAA exporter(s) prior to administration. In some embodiments, the cultures, which are induced by a chemical and/or nutritional inducer and/or metabolite, are grown aerobically. In some embodiments, the cultures, which are induced by a chemical and/or nutritional inducer and/or metabolite, are grown anaerobically.

In some embodiments, the genetically engineered bacteria encode one or more gene sequence(s) which are inducible through an arabinose inducible system.

The genes of arabinose metabolism are organized in one operon, AraBAD, which is controlled by the PAraBAD promoter. The PAraBAD (or Para) promoter suitably fulfills the criteria of inducible expression systems. PAraBAD displays tighter control of payload gene expression than many other systems, likely due to the dual regulatory role of AraC, which functions both as an inducer and as a repressor. Additionally, the level of ParaBAD-based expression can be modulated over a wide range of L-arabinose concentrations to fine-tune levels of expression of the payload. However, the cell population exposed to sub-saturating L-arabinose concentrations is divided into two subpopulations of induced and uninduced cells, which is determined by the differences between individual cells in the availability of L-arabinose transporter (Zhang et al., Development and Application of an Arabinose-Inducible Expression System by Facilitating Inducer Uptake in Corynebacterium glutamicum; Appl. Environ. Microbiol. August 2012 vol. 78 no. 16 5831-5838). Alternatively, inducible expression from the ParaBad can be controlled or fine-tuned through the optimization of the ribosome binding site (RBS), as described herein.

In one embodiment, expression of one or more optimized enzyme(s), e.g., kivD, leuDH, adh2, and/or brnQ, e.g., as described herein, is driven directly or indirectly by one or more arabinose inducible promoter(s). In one embodiment, the genetically engineered bacteria encode one or more branched chain amino acid transporter(s) e.g., livKHMGF and/or brnQ, described herein, whose expression is driven directly or indirectly by one or more arabinose inducible promoter(s). In one embodiment, expression of one or more branched chain amino acid exporter(s), e.g., as described herein, is driven directly or indirectly by one or more arabinose inducible promoter(s). In some embodiments, the arabinose inducible promoter is useful for or induced during in vivo expression of the one or more protein(s) of interest. In some embodiments, expression of one or more branched chain amino acid catabolism enzyme(s) and/or branched chain amino acid transporter(s) and/or BCAA binding protein(s) and/or BCAA exporter(s) is driven directly or indirectly by one or more arabinose inducible promoter(s) in vivo. In some embodiments, the promoter is directly or indirectly induced by a molecule (e.g., arabinose) that is co-administered with the genetically engineered bacteria of the invention.

In some embodiments, expression of one or more branched chain amino acid catabolism enzyme(s) and/or branched chain amino acid transporter(s) and/or BCAA binding protein(s) and/or BCAA exporter(s), is driven directly or indirectly by one or more arabinose inducible promoter(s) during in vitro growth, preparation, or manufacturing of the strain prior to in vivo administration. In some embodiments, the arabinose inducible promoter(s) are induced in culture, e.g., grown in a flask, fermenter or other appropriate culture vessel, e.g., used during cell growth, cell expansion, fermentation, recovery, purification, formulation, and/or manufacture. In some embodiments, the promoter is directly or indirectly induced by a molecule, e.g., arabinose, that is added to in the bacterial culture to induce expression and pre-load the bacterium with branched chain amino acid catabolism enzyme(s) and/or branched chain amino acid transporter(s) and/or BCAA binding protein(s) and/or BCAA exporter(s) prior to administration. In some embodiments, the cultures, which are induced by arabinose, are grown aerobically. In some embodiments, the cultures, which are induced by arabinose, are grown anaerobically.

In some embodiments, bacterial cell comprises two or more distinct branched chain amino acid catabolism cassette(s) or transporter(s) or binding protein(s) or exporter(s), one or more of which are induced by arabinose. In some embodiments, the genetically engineered bacteria comprise multiple copies of the same branched chain amino acid catabolism enzyme gene sequence(s) and/or transporter gene sequence(s), e.g., as described herein, which are induced by one or more nutritional and/or chemical inducer(s) and/or metabolite(s). In some embodiments, the genetically engineered bacteria comprise multiple copies of different branched chain amino acid catabolism enzyme genes sequence(s) and/or transporter gene sequence(s) and/or BCAA binding protein gene sequence(s) and/or BCAA exporter gene sequence(s), e.g., as described herein, one or more of which are induced by one or more nutritional and/or chemical inducer(s) and/or metabolite(s).

In a first example, the arabinose inducible promoter drives the expression of a construct comprising one or more polypeptides of interest described herein jointly with a second promoter, e.g., a second constitutive or inducible promoter. In some embodiments, two promoters are positioned proximally to the construct and drive its expression, wherein the arabinose inducible promoter drives expression under a first set of exogenous conditions, and the second promoter drives the expression under a second set of exogenous conditions. In second example, the arabinose promoter drives the expression of one or more gene cassette(s) under a first inducing condition and another inducible promoter drives the expression of one or more of the same or different gene cassette(s) expressing one or more polypeptides of interest, under a second inducing condition. In both examples, the first and second conditions can be two sequential inducing culture conditions (i.e., during preparation of the culture in a flask, fermenter or other appropriate culture vessel, e.g., arabinose and IPTG). In another non-limiting example, the first inducing conditions are culture conditions, e.g., the presence of arabinose, and the second inducing conditions are in vivo conditions. Such in vivo conditions include low-oxygen, microaerobic, or anaerobic conditions, presence of gut metabolites, and/or nutritional and/or chemical inducers and/or metabolites administered in combination with the bacterial strain. In some embodiments, the one or more arabinose promoters drive expression of one or more protein(s) of interest, in combination with the FNR promoter driving the expression of the same gene sequence(s).

In some embodiments, the gene sequence(s) encoding the branched chain amino acid catabolism enzyme(s) or other polypeptide(s) of interest, are present on a plasmid and operably linked to a promoter that is induced by arabinose. In some embodiments, the gene sequence(s) encoding the branched chain amino acid catabolism enzyme(s) or branched chain amino acid transporter(s) is present in the chromosome and operably linked to a promoter that is induced by arabinose.

In some embodiments, the arabinose inducible construct further comprises a gene encoding AraC, which is divergently transcribed from the same promoter as the one or more one or more branched chain amino acid catabolism enzyme(s).

In some embodiments, the genetically engineered bacteria comprise one or more gene sequence(s) which are inducible through a rhamnose inducible system. The genes rhaBAD are organized in one operon which is controlled by the rhaP BAD promoter. The rhaP BAD promoter is regulated by two activators, RhaS and RhaR, and the corresponding genes belong to one transcription unit which divergently transcribed in the opposite direction of rhaBAD. In the presence of L-rhamnose, RhaR binds to the rhaP RS promoter and activates the production of RhaR and RhaS. RhaS together with L-rhamnose then bind to the rhaP BAD and the rhaP T promoter and activate the transcription of the structural genes. In contrast to the arabinose system, in which AraC is provided and divergently transcribed in the gene sequence(s), it is not necessary to express the regulatory proteins in larger quantities in the rhamnose expression system because the amounts expressed from the chromosome are sufficient to activate transcription even on multi-copy plasmids. Therefore, only the rhaP BAD promoter is cloned upstream of the gene that is to be expressed. Full induction of rhaBAD transcription also requires binding of the CRP-cAMP complex, which is a key regulator of catabolite repression. Alternatively, inducible expression from the rhaBAD can be controlled or fine-tuned through the optimization of the ribosome binding site (RBS), as described herein.

In one embodiment, expression of one or more branched chain amino acid catabolism enzyme(s), e.g., kivD, leuDH, adh2, and/or brnQ, e.g., as described herein, is driven directly or indirectly by one or more rhamnose inducible promoter(s). In one embodiment, the genetically engineered bacteria encode one or more branched chain amino acid transporter(s), e.g., livKHMGF and/or brnQ, described herein, whose expression is driven directly or indirectly by one or more rhamnose inducible promoter(s). In one embodiment, the genetically engineered bacteria encode one or more branched chain amino acid exporter(s), described herein, whose expression is driven directly or indirectly by one or more rhamnose inducible promoter(s).

In some embodiments, the rhamnose inducible promoter is useful for or induced during in vivo expression of the one or more protein(s) of interest. In some embodiments, expression of one or more branched chain amino acid catabolism enzyme(s) and/or branched chain amino acid transporter(s) and/or BCAA binding protein(s) and/or BCAA exporter(s) is driven directly or indirectly by one or more rhamnose inducible promoter(s) in vivo. In some embodiments, the promoter is directly or indirectly induced by a molecule (e.g., rhamnose) that is co-administered with the genetically engineered bacteria of the invention.

In some embodiments, expression of one or more branched chain amino acid catabolism enzyme(s) and/or branched chain amino acid transporter(s) and/or BCAA binding protein(s) and/or BCAA exporter(s), is driven directly or indirectly by one or more rhamnose inducible promoter(s) during in vitro growth, preparation, or manufacturing of the strain prior to in vivo administration. In some embodiments, the rhamnose inducible promoter(s) are induced in culture, e.g., grown in a flask, fermenter or other appropriate culture vessel, e.g., used during cell growth, cell expansion, fermentation, recovery, purification, formulation, and/or manufacture. In some embodiments, the promoter is directly or indirectly induced by a molecule, e.g., rhamnose, that is added to in the bacterial culture to induce expression and pre-load the bacterium with branched chain amino acid catabolism enzyme(s) and/or BCAA transporter(s) and/or BCAA binding protein(s) and/or BCAA exporter(s) prior to administration. In some embodiments, the cultures, which are induced by rhamnose, are grown aerobically. In some embodiments, the cultures, which are induced by rhamnose, are grown anaerobically.

In some embodiments, bacterial cell comprises two or more distinct branched chain amino acid catabolism cassette(s) or other polypeptide(s) of interest, one or more of which are induced by rhamnose. In some embodiments, the genetically engineered bacteria comprise multiple copies of the same branched chain amino acid catabolism enzyme gene sequence(s) and/or transporter gene sequence(s) and/or BCAA binding protein gene sequence(s) and/or BCAA exporter gene sequence(s), e.g., as described herein, which are induced by rhamnose. In some embodiments, the genetically engineered bacteria comprise multiple copies of different branched chain amino acid catabolism enzyme genes sequence(s) and/or transporter gene sequence(s) and/or BCAA binding protein gene sequence(s) and/or BCAA exporter gene sequence(s), e.g., as described herein, one or more of which are induced by rhamnose.

In a first example, the rhamnose inducible promoter drives the expression of a construct comprising one or more polypeptides of interest described herein jointly with a second promoter, e.g., a second constitutive or inducible promoter. In some embodiments, two promoters are positioned proximally to the construct and drive its expression, wherein the rhamnose inducible promoter drives expression under a first set of exogenous conditions, and the second promoter drives the expression under a second set of exogenous conditions. In second example, the rhamnose promoter drives the expression of one or more gene cassette(s) under a first inducing condition and another inducible promoter drives the expression of one or more of the same or different gene cassette(s) expressing one or more polypeptides of interest, under a second inducing condition. In both examples, the first and second conditions can be two sequential inducing culture conditions (i.e., during preparation of the culture in a flask, fermenter or other appropriate culture vessel, e.g., rhamnose and IPTG). In another non-limiting example, the first inducing conditions are culture conditions, e.g., the presence of rhamnose, and the second inducing conditions are in vivo conditions. Such in vivo conditions include low-oxygen, microaerobic, or anaerobic conditions, presence of gut metabolites, and/or nutritional and/or chemical inducers and/or metabolites administered in combination with the bacterial strain. In some embodiments, the one or more rhamnose promoters drive expression of one or more protein(s) of interest, in combination with the FNR promoter driving the expression of the same gene sequence(s).

In some embodiments, the gene sequence(s) encoding the branched chain amino acid catabolism enzyme(s) or other polypeptide(s) of interest, are present on a plasmid and operably linked to a promoter that is induced by rhamnose. In some embodiments, the gene sequence(s) encoding the branched chain amino acid catabolism enzyme(s) or branched chain amino acid transporter(s) and/or BCAA binding protein(s) and/or BCAA exporter(s) is present in the chromosome and operably linked to a promoter that is induced by rhamnose.

In some embodiments, the genetically engineered bacteria comprise one or more gene sequence(s) which are inducible through an Isopropyl β-D-1-thiogalactopyranoside (IPTG) inducible system or other compound which induced transcription from the Lac Promoter. IPTG is a molecular mimic of allolactose, a lactose metabolite that activates transcription of the lac operon. In contrast to allolactose, the sulfur atom in IPTG creates a non-hydrolyzable chemical blond, which prevents the degradation of IPTG, allowing the concentration to remain constant. IPTG binds to the lac repressor and releases the tetrameric repressor (lacI) from the lac operator in an allosteric manner, thereby allowing the transcription of genes in the lac operon. Since IPTG is not metabolized by E. coli, its concentration stays constant and the rate of expression of Lac promoter-controlled is tightly controlled, both in vivo and in vitro. IPTG intake is independent on the action of lactose permease, since other transport pathways are also involved. Inducible expression from the PLac can be controlled or fine-tuned through the optimization of the ribosome binding site (RBS), as described herein. Other compounds which inactivate LacI, can be used instead of IPTG in a similar manner.

In one embodiment, expression of the optimized enzyme(s), e.g., kivD, leuDH, adh2, and brnQ, e.g., as described herein, is driven directly or indirectly by one or more IPTG inducible promoter(s). In one embodiment, the genetically engineered bacteria encode one or more branched chain amino acid transporter(s), e.g., livKHMGF and/or brnQ, described herein, whose expression is driven directly or indirectly by one or more IPTG inducible promoter(s). In one embodiment, the genetically engineered bacteria encode one or more branched chain amino acid exporter(s) described herein, whose expression is driven directly or indirectly by one or more IPTG inducible promoter(s).

In some embodiments, the IPTG inducible promoter is useful for or induced during in vivo expression of the one or more protein(s) of interest. In some embodiments, expression of one or more branched chain amino acid catabolism enzyme(s) and/or branched chain amino acid transporter(s) is driven directly or indirectly by one or more IPTG inducible promoter(s) in vivo. In some embodiments, the promoter is directly or indirectly induced by a molecule (e.g., IPTG) that is co-administered with the genetically engineered bacteria of the invention.

In some embodiments, expression of one or more branched chain amino acid catabolism enzyme(s) and/or branched chain amino acid transporter(s) and/or BCAA binding protein(s) and/or BCAA exporter(s), is driven directly or indirectly by one or more IPTG inducible promoter(s) during in vitro growth, preparation, or manufacturing of the strain prior to in vivo administration. In some embodiments, the IPTG inducible promoter(s) are induced in culture, e.g., grown in a flask, fermenter or other appropriate culture vessel, e.g., used during cell growth, cell expansion, fermentation, recovery, purification, formulation, and/or manufacture. In some embodiments, the promoter is directly or indirectly induced by a molecule, e.g., IPTG, that is added to in the bacterial culture to induce expression and pre-load the bacterium with branched chain amino acid catabolism enzyme(s) prior to administration. In some embodiments, the cultures, which are induced by IPTG, are grown aerobically. In some embodiments, the cultures, which are induced by IPTG, are grown anaerobically.

In some embodiments, bacterial cell comprises two or more distinct branched chain amino acid catabolism cassette(s) or other polypeptide(s) of interest, one or more of which are induced by IPTG. In some embodiments, the genetically engineered bacteria comprise multiple copies of the same branched chain amino acid catabolism enzyme gene sequence(s) and/or transporter gene sequence(s), e.g., as described herein, which are induced IPTG. In some embodiments, the genetically engineered bacteria comprise multiple copies of different branched chain amino acid catabolism enzyme genes sequence(s) and/or transporter gene sequence(s) and/or other gene sequence(s) of interest, as described herein, one or more of which are induced by IPTG.

In a first example, the IPTG inducible promoter drives the expression of a construct comprising one or more polypeptides of interest described herein jointly with a second promoter, e.g., a second constitutive or inducible promoter. In some embodiments, two promoters are positioned proximally to the construct and drive its expression, wherein the IPTG inducible promoter drives expression under a first set of exogenous conditions, and the second promoter drives the expression under a second set of exogenous conditions. In second example, the IPTG promoter drives the expression of one or more gene cassette(s) under a first inducing condition and another inducible promoter drives the expression of one or more of the same or different gene cassette(s) expressing one or more polypeptides of interest, under a second inducing condition. In both examples, the first and second conditions can be two sequential inducing culture conditions (i.e., during preparation of the culture in a flask, fermenter or other appropriate culture vessel, e.g., IPTG and IPTG). In another non-limiting example, the first inducing conditions are culture conditions, e.g., the presence of IPTG, and the second inducing conditions are in vivo conditions. Such in vivo conditions include low-oxygen, microaerobic, or anaerobic conditions, presence of gut metabolites, and/or nutritional and/or chemical inducers and/or metabolites administered in combination with the bacterial strain. In some embodiments, the one or more IPTG promoters drive expression of one or more protein(s) of interest, in combination with the FNR promoter driving the expression of the same gene sequence(s).

In some embodiments, the gene sequence(s) encoding the branched chain amino acid catabolism enzyme(s) or other polypeptide(s) of interest, are present on a plasmid and operably linked to a promoter that is induced by IPTG. In some embodiments, the gene sequence(s) encoding the branched chain amino acid catabolism enzyme(s) or branched chain amino acid transporter(s) is present in the chromosome and operably linked to a promoter that is induced by IPTG.

In some embodiments, the IPTG inducible construct further comprises a gene encoding lacI, which is divergently transcribed from the same promoter as the one or more one or more branched chain amino acid catabolism enzyme(s) and/or transporters described herein.

In some embodiments, the genetically engineered bacteria comprise one or more gene sequence(s) which are inducible through a tetracycline inducible system. The initial system Gossen and Bujard (Gossen M & Bujard H. PNAS, 1992 Jun. 15; 89(12):5547-51) developed is known as tetracycline off: in the presence of tetracycline, expression from a tet-inducible promoter is reduced. Tetracycline-controlled transactivator (tTA) was created by fusing tetR with the C-terminal domain of VP16 (virion protein 16) from herpes simplex virus. In the absence of tetracycline, the tetR portion of tTA will bind tetO sequences in the tet promoter, and the activation domain promotes expression. In the presence of tetracycline, tetracycline binds to tetR, precluding tTA from binding to the tetO sequences. Next, a reverse Tet repressor (rTetR), was developed which created a reliance on the presence of tetracycline for induction, rather than repression. The new transactivator rtTA (reverse tetracycline-controlled transactivator) was created by fusing rTetR with VP16. The tetracycline on system is also known as the rtTA-dependent system.

In one embodiment, expression of the optimized enzyme(s), e.g., kivD, leuDH, adh2, and brnQ, e.g., as described herein, is driven directly or indirectly by one or more tetracycline inducible promoter(s). In one embodiment, the genetically engineered bacteria encode one or more branched chain amino acid transporter(s), e.g., livKHMGF and/or brnQ, described herein, whose expression is driven directly or indirectly by one or more tetracycline inducible promoter(s).

In one embodiment, the genetically engineered bacteria encode one or more branched chain amino acid exporter(s).

In some embodiments, the tetracycline inducible promoter is useful for or induced during in vivo expression of the one or more protein(s) of interest. In some embodiments, expression of one or more branched chain amino acid catabolism enzyme(s) and/or branched chain amino acid transporter(s) and or other polypeptide(s) of interest is driven directly or indirectly by one or more tetracycline inducible promoter(s) in vivo. In some embodiments, the promoter is directly or indirectly induced by a molecule (e.g., tetracycline) that is co-administered with the genetically engineered bacteria of the invention.

In some embodiments, expression of one or more branched chain amino acid catabolism enzyme(s) and/or branched chain amino acid transporter(s) and or other polypeptide(s) of interest, is driven directly or indirectly by one or more tetracycline inducible promoter(s) during in vitro growth, preparation, or manufacturing of the strain prior to in vivo administration. In some embodiments, the tetracycline inducible promoter(s) are induced in culture, e.g., grown in a flask, fermenter or other appropriate culture vessel, e.g., used during cell growth, cell expansion, fermentation, recovery, purification, formulation, and/or manufacture. In some embodiments, the promoter is directly or indirectly induced by a molecule, e.g., tetracycline, that is added to in the bacterial culture to induce expression and pre-load the bacterium with branched chain amino acid catabolism enzyme(s) prior to administration. In some embodiments, the cultures, which are induced by tetracycline, are grown aerobically. In some embodiments, the cultures, which are induced by tetracycline, are grown anaerobically.

In some embodiments, bacterial cell comprises two or more distinct branched chain amino acid catabolism cassette(s) and/or other polypeptide(s) of interest, one or more of which are induced by tetracycline. In some embodiments, the genetically engineered bacteria comprise multiple copies of the same branched chain amino acid catabolism enzyme gene sequence(s) and/or transporter gene sequence(s) and/or gene sequence(s) for the expression of other polypeptide(s) of interest, e.g., as described herein, which are induced by tetracycline. In some embodiments, the genetically engineered bacteria comprise multiple copies of different branched chain amino acid catabolism enzyme genes sequence(s) and/or transporter gene sequence(s) and gene sequence(s) for the expression of other polypeptide(s) of interest, e.g., as described herein, one or more of which are induced by tetracycline.

In a first example, the tetracycline inducible promoter drives the expression of a construct comprising one or more polypeptides of interest described herein jointly with a second promoter, e.g., a second constitutive or inducible promoter. In some embodiments, two promoters are positioned proximally to the construct and drive its expression, wherein the tetracycline inducible promoter drives expression under a first set of exogenous conditions, and the second promoter drives the expression under a second set of exogenous conditions. In second example, the tetracycline promoter drives the expression of one or more gene cassette(s) under a first inducing condition and another inducible promoter drives the expression of one or more of the same or different gene cassette(s) expressing one or more polypeptides of interest, under a second inducing condition. In both examples, the first and second conditions can be two sequential inducing culture conditions (i.e., during preparation of the culture in a flask, fermenter or other appropriate culture vessel, e.g., tetracycline and IPTG). In another non-limiting example, the first inducing conditions are culture conditions, e.g., the presence of tetracycline, and the second inducing conditions are in vivo conditions. Such in vivo conditions include low-oxygen, microaerobic, or anaerobic conditions, presence of gut metabolites, and/or nutritional and/or chemical inducers and/or metabolites administered in combination with the bacterial strain. In some embodiments, the one or more tetracycline promoters drive expression of one or more protein(s) of interest, in combination with the FNR promoter driving the expression of the same gene sequence(s).

In some embodiments, the gene sequence(s) encoding the branched chain amino acid catabolism enzyme(s) or other polypeptide(s) of interest, are present on a plasmid and operably linked to a promoter that is induced by tetracycline. In some embodiments, the gene sequence(s) encoding the branched chain amino acid catabolism enzyme(s) or branched chain amino acid transporter(s) is present in the chromosome and operably linked to a promoter that is induced by tetracycline.

In some embodiments, the tetracycline inducible construct further comprises a gene encoding AraC, which is divergently transcribed from the same promoter as the one or more one or more branched chain amino acid catabolism enzyme(s) and/or transporters described herein.

In some embodiments, the genetically engineered bacteria comprise one or more gene sequence(s) whose expression is controlled by a temperature sensitive mechanism. Thermoregulators are advantageous because of strong transcriptional control without the use of external chemicals or specialized media (see, e.g., Nemani et al., Magnetic nanoparticle hyperthermia induced cytosine deaminase expression in microencapsulated E. coli for enzyme-prodrug therapy; J Biotechnol. 2015 Jun. 10; 203: 32-40, and references therein). Thermoregulated protein expression using the mutant cI857 repressor and the pL and/or pR phage λ promoters have been used to engineer recombinant bacterial strains. The gene of interest cloned downstream of the a promoters can then be efficiently regulated by the mutant thermolabile cI857 repressor of bacteriophage λ. At temperatures below 37° C., cI857 binds to the oL or regions of the pR promoter and blocks transcription by RNA polymerase. At higher temperatures, the functional cI857 dimer is destabilized, binding to the oL or oR DNA sequences is abrogated, and mRNA transcription is initiated. Inducible expression from the thermoregulated promoter can be controlled or further fine-tuned through the optimization of the ribosome binding site (RBS), as described herein.

In one embodiment, expression of one or more protein(s) of interest is driven directly or indirectly by one or more thermoregulated promoter(s). In one embodiment, expression of the optimized enzyme(s), e.g., kivD, leuDH, adh2, and brnQ, e.g., as described herein, is driven directly or indirectly by one or more thermoregulated inducible promoter(s). In one embodiment, the genetically engineered bacteria encode one or more branched chain amino acid transporter(s), e.g., livKHMGF and/or brnQ, described herein, whose expression is driven directly or indirectly by one or more thermoregulated inducible promoter(s). In one embodiment, the genetically engineered bacteria encode one or more branched chain amino acid exporter(s) described herein, whose expression is driven directly or indirectly by one or more thermoregulated inducible promoter(s).

In some embodiments, the thermoregulated promoter is useful for or induced during in vivo expression of the one or more protein(s) of interest. In some embodiments, expression of one or more branched chain amino acid catabolism enzyme(s) and/or branched chain amino acid transporter(s) and/or other protein(s) of interest is driven directly or indirectly by one or more thermoregulated promoter(s) in vivo.

In some embodiments, expression of one or more protein(s) of interest is driven directly or indirectly by one or more thermoregulated promoter(s) during in vitro growth, preparation, or manufacturing of the strain prior to in vivo administration. In some embodiments, it may be advantageous to shut off production of the one or more branched chain amino acid catabolism enzyme(s) and/or branched chain amino acid transporter(s). This can be done in a thermoregulated system by growing the strain at lower temperatures, e.g., 30° C. Expression can then be induced by elevating the temperature to 37° C. and/or 42° C. In some embodiments, the thermoregulated promoter(s) are induced in culture, e.g., grown in a flask, fermenter or other appropriate culture vessel, e.g., used during cell growth, cell expansion, fermentation, recovery, purification, formulation, and/or manufacture. In some embodiments, the cultures, which are induced by temperatures between 37° C. and 42° C., are grown aerobically. In some embodiments, the cultures, which are induced by induced by temperatures between 37° C. and 42° C., are grown anaerobically.

In some embodiments, bacterial cell comprises two or more distinct branched chain amino acid catabolism cassette(s) or branched chain amino acid transporter(s) and/or cassette(s) for the expression of other protein(s) of interest, one or more of which are induced by temperature. In some embodiments, the genetically engineered bacteria comprise multiple copies of the same branched chain amino acid catabolism enzyme gene sequence(s) and/or transporter gene sequence(s) and/or gene sequence(s) for the expression of other proteins of interest, e.g., as described herein, which are induced by temperature. In some embodiments, the genetically engineered bacteria comprise multiple copies of different branched chain amino acid catabolism enzyme genes sequence(s) and/or transporter gene sequence(s) or other gene sequence(s) of interest, e.g., as described herein, one or more of which are induced by temperature.

In a first example, the temperature inducible promoter drives the expression of a construct comprising one or more polypeptides of interest described herein jointly with a second promoter, e.g., a second constitutive or inducible promoter. In some embodiments, two promoters are positioned proximally to the construct and drive its expression, wherein the temperature inducible promoter drives expression under a first set of exogenous conditions, and the second promoter drives the expression under a second set of exogenous conditions. In second example, the temperature promoter drives the expression of one or more gene cassette(s) under a first inducing condition and another inducible promoter drives the expression of one or more of the same or different gene cassette(s) expressing one or more polypeptides of interest, under a second inducing condition. In both examples, the first and second conditions can be two sequential inducing culture conditions (i.e., during preparation of the culture in a flask, fermenter or other appropriate culture vessel, e.g., temperature regulation and IPTG). In another non-limiting example, the first inducing conditions are culture conditions, e.g., the permissive temperature, and the second inducing conditions are in vivo conditions. Such in vivo conditions include low-oxygen, microaerobic, or anaerobic conditions, presence of gut metabolites, and/or nutritional and/or chemical inducers and/or metabolites administered in combination with the bacterial strain. In some embodiments, the one or more temperature regulated promoters drive expression of one or more protein(s) of interest, in combination with the FNR promoter driving the expression of the same gene sequence(s).

In some embodiments, the gene sequence(s) encoding the branched chain amino acid catabolism enzyme(s) or other polypeptide(s) of interest, are present on a plasmid and operably linked to a promoter that is induced by temperature. In some embodiments, the gene sequence(s) encoding the branched chain amino acid catabolism enzyme(s) or branched chain amino acid transporter(s) is present in the chromosome and operably linked to a promoter that is induced by temperature.

In some embodiments, the thermoregulated construct further comprises a gene encoding mutant cI857 repressor, which is divergently transcribed from the same promoter as the one or more one or more branched chain amino acid catabolism enzyme(s) and/or transporters described herein. In some embodiments, the genetically engineered bacteria comprise one or more gene sequence(s) which are indirectly inducible through a system driven by the PssB promoter. The Pssb promoter is active under aerobic conditions, and shuts off under anaerobic conditions.

This promoter can be used to express a gene of interest under aerobic conditions. This promoter can also be used to tightly control the expression of a gene product such that it is only expressed under anaerobic conditions. In this case, the oxygen induced PssB promoter induces the expression of a repressor, which represses the expression of a gene of interest. As a result, the gene of interest is only expressed in the absence of the repressor, i.e., under anaerobic conditions. This strategy has the advantage of an additional level of control for improved fine-tuning and tighter control.

In one embodiment, expression of the optimized enzyme(s), e.g., kivD, leuDH, adh2, and brnQ, e.g., as described herein, is driven directly or indirectly by one or more PssB promoter(s). In one embodiment, the genetically engineered bacteria encode one or more branched chain amino acid transporter(s), e.g., livKHMGF and/or brnQ, described herein, whose expression is driven directly or indirectly by one or more PssB promoter(s). In one embodiment, the genetically engineered bacteria encode one or more branched chain amino acid exporter(s), described herein, whose expression is driven directly or indirectly by one or more PssB promoter(s).

In some embodiments, the PssB promoter is useful for or induced during in vivo expression of the one or more protein(s) of interest. In some embodiments, expression of one or more branched chain amino acid catabolism enzyme(s) and/or branched chain amino acid transporter(s) and/or other protein(s) of interest is driven directly or indirectly by one or more PssB promoter(s) in vivo. In some embodiments, the promoter is directly or indirectly induced by a molecule (e.g., arabinose) that is co-administered with the genetically engineered bacteria of the invention.

In some embodiments, expression of one or more branched chain amino acid catabolism enzyme(s) and/or branched chain amino acid transporter(s) and/or other protein(s) of interest, is driven directly or indirectly by one or more PssB promoter(s) during in vitro growth, preparation, or manufacturing of the strain prior to in vivo administration. In some embodiments, the PssB promoter(s) are induced in culture, e.g., grown in a flask, fermenter or other appropriate culture vessel, e.g., used during cell growth, cell expansion, fermentation, recovery, purification, formulation, and/or manufacture. In some embodiments, the promoter is directly or indirectly induced by a molecule, e.g., arabinose, that is added to in the bacterial culture to induce expression and pre-load the bacterium with branched chain amino acid catabolism enzyme(s) prior to administration. In some embodiments, the cultures, which are induced by arabinose, are grown aerobically. In some embodiments, the cultures, which are induced by arabinose, are grown anaerobically.

In some embodiments, bacterial cell comprises two or more distinct branched chain amino acid catabolism cassette(s) or other polypeptide(s) of interest, one or more of which are induced by arabinose. In some embodiments, the genetically engineered bacteria comprise multiple copies of the same branched chain amino acid catabolism enzyme gene sequence(s) and/or transporter gene sequence(s) and/or other gene sequence(s) of interest, e.g., as described herein, which are induced by one or more nutritional and/or chemical inducer(s) and/or metabolite(s). In some embodiments, the genetically engineered bacteria comprise multiple copies of different branched chain amino acid catabolism enzyme genes sequence(s) and/or transporter gene sequence(s), e.g., as described herein, one or more of which are induced by one or more nutritional and/or chemical inducer(s) and/or metabolite(s).

In a first example, the PssB promoter drives the expression of a construct comprising one or more polypeptides of interest described herein jointly with a second promoter, e.g., a second constitutive or inducible promoter. In some embodiments, two promoters are positioned proximally to the construct and drive its expression, wherein the PssB promoter drives expression under a first set of exogenous conditions, and the second promoter drives the expression under a second set of exogenous conditions. In second example, the PssB promoter drives the expression of one or more gene cassette(s) under a first inducing condition and another inducible promoter drives the expression of one or more of the same or different gene cassette(s) expressing one or more polypeptides of interest, under a second inducing condition. In both examples, the first and second conditions can be two sequential inducing culture conditions (i.e., during preparation of the culture in a flask, fermenter or other appropriate culture vessel, e.g., PssB and IPTG). In another non-limiting example, the first inducing conditions are culture conditions, e.g., the presence of arabinose, and the second inducing conditions are in vivo conditions. Such in vivo conditions include low-oxygen, microaerobic, or anaerobic conditions, presence of gut metabolites, and/or nutritional and/or chemical inducers and/or metabolites administered in combination with the bacterial strain. In some embodiments, the one or more PssB promoters drive expression of one or more protein(s) of interest, in combination with the FNR promoter driving the expression of the same gene sequence(s).

In some embodiments, the gene sequence(s) encoding the branched chain amino acid catabolism enzyme(s) or other polypeptide(s) of interest, are present on a plasmid and operably linked to a promoter that is induced by arabinose. In some embodiments, the gene sequence(s) encoding the branched chain amino acid catabolism enzyme(s) or branched chain amino acid transporter(s) is present in the chromosome and operably linked to a promoter that is induced by arabinose.

In another non-limiting example, this strategy can be used to control expression of thyA and/or dapA, e.g., to make a conditional auxotroph. The chromosomal copy of dapA or ThyA is knocked out. Under anaerobic conditions, dapA or thyA—as the case may be—are expressed, and the strain can grow in the absence of dap or thymidine. Under aerobic conditions, dapA or thyA expression is shut off, and the strain cannot grow in the absence of dap or thymidine. Such a strategy can, for example be employed to allow survival of bacteria under anaerobic conditions, e.g., the gut, but prevent survival under aerobic conditions (biosafety switch).

Essential Genes and Auxotrophs

As used herein, the term “essential gene” refers to a gene which is necessary to for cell growth and/or survival. Bacterial essential genes are well known to one of ordinary skill in the art, and can be identified by directed deletion of genes and/or random mutagenesis and screening (see, for example, Zhang and Lin, 2009, DEG 5.0, a database of essential genes in both prokaryotes and eukaryotes, Nucl. Acids Res., 37:D455-D458 and Gerdes et al., Essential genes on metabolic maps, Curr. Opin. Biotechnol., 17(5):448-456, the entire contents of each of which are expressly incorporated herein by reference).

An “essential gene” may be dependent on the circumstances and environment in which an organism lives. For example, a mutation of, modification of, or excision of an essential gene may result in the recombinant bacteria of the disclosure becoming an auxotroph. An auxotrophic modification is intended to cause bacteria to die in the absence of an exogenously added nutrient essential for survival or growth because they lack the gene(s) necessary to produce that essential nutrient.

An auxotrophic modification is intended to cause bacteria to die in the absence of an exogenously added nutrient essential for survival or growth because they lack the gene(s) necessary to produce that essential nutrient. In some embodiments, any of the genetically engineered bacteria described herein also comprise a deletion or mutation in one or more gene(s) required for cell survival and/or growth.

In some embodiments, the bacterial cell comprises a genetic mutation in one or more endogenous gene(s) encoding a branched chain amino acid biosynthesis gene, wherein the genetic mutation reduces biosynthesis of one or more branched chain amino acids in the bacterial cell. In some embodiments, the endogenous gene encoding a branched chain amino acid biosynthesis gene is a keto acid reductoisomerase gene. Keto acid reductoisomerase gene is required for branched chain amino acid synthesis. Knock-out of this gene creates an auxotroph and requires the cell to import leucine to survive. In some embodiments, the bacterial cell comprises a genetic mutation in ilvC gene.

In one embodiment, the essential gene is an oligonucleotide synthesis gene, for example, thyA. In another embodiment, the essential gene is a cell wall synthesis gene, for example, dapA. In yet another embodiment, the essential gene is an amino acid gene, for example, serA or MetA. Any gene required for cell survival and/or growth may be targeted, including but not limited to, cysE, glnA, ilvD, leuB, lysA, serA, metA, glyA, hisB, ilvA, pheA, proA, thrC, trpC, tyrA, thyA, uraA, dapA, dapB, dapD, dapE, dapF, flhD, metB, metC, proAB, and thi1, as long as the corresponding wild-type gene product is not produced in the bacteria.

Table 6 lists depicts exemplary bacterial genes which may be disrupted or deleted to produce an auxotrophic strain. These include, but are not limited to, genes required for oligonucleotide synthesis, amino acid synthesis, and cell wall synthesis.

TABLE 6
Non-limiting Examples of Bacterial Genes
Useful for Generation of an Auxotroph

| Amino Acid | Oligonucleotide | Cell Wall |
|---|---|---|
| cysE | thyA | dapA |
| glnA | uraA | dapB |
| ilvD | | dapD |
| leuB | | dapE |
| lysA | | dapF |
| serA | | |
| metA | | |
| glyA | | |
| hisB | | |
| ilvA | | |
| pheA | | |
| proA | | |
| thrC | | |
| trpC | | |
| tyrA | | |

Table 7 shows the survival of various amino acid auxotrophs in the mouse gut, as detected 24 hrs and 48 hrs post-gavage. These auxotrophs were generated using BW25113, a non-Nissle strain of E. coli.

TABLE 7
Survival of amino acid auxotrophs in the mouse gut

| Gene | AA Auxotroph | Pre-Gavage | 24 hours | 48 hours |
|---|---|---|---|---|
| argA | Arginine | Present | Present | Absent |
| cysE | Cysteine | Present | Present | Absent |
| glnA | Glutamine | Present | Present | Absent |
| glyA | Glycine | Present | Present | Absent |
| hisB | Histidine | Present | Present | Present |
| ilvA | Isoleucine | Present | Present | Absent |
| leuB | Leucine | Present | Present | Absent |
| lysA | Lysine | Present | Present | Absent |
| metA | Methionine | Present | Present | Present |
| pheA | Phenylalanine | Present | Present | Present |
| proA | Proline | Present | Present | Absent |
| serA | Serine | Present | Present | Present |
| thrC | Threonine | Present | Present | Present |
| trpC | Tryptophan | Present | Present | Present |
| tyrA | Tyrosine | Present | Present | Present |
| ilvD | Valine/Isoleucine/ | Present | Present | Absent |
| | Leucine | | | |
| thyA | Thiamine | Present | Absent | Absent |
| uraA | Uracil | Present | Absent | Absent |
| flhD | FlhD | Present | Present | Present |

For example, thymine is a nucleic acid that is required for bacterial cell growth; in its absence, bacteria undergo cell death. The thyA gene encodes thimidylate synthetase, an enzyme that catalyzes the first step in thymine synthesis by converting dUMP to dTMP (Sat et al., 2003). In some embodiments, the bacterial cell of the disclosure is a thyA auxotroph in which the thyA gene is deleted and/or replaced with an unrelated gene. A thyA auxotroph can grow only when sufficient amounts of thymine are present, e.g., by adding thymine to growth media in vitro, or in the presence of high thymine levels found naturally in the human gut in vivo. In some embodiments, the bacterial cell of the disclosure is auxotrophic in a gene that is complemented when the bacterium is present in the mammalian gut. Without sufficient amounts of thymine, the thyA auxotroph dies. In some embodiments, the auxotrophic modification is used to ensure that the bacterial cell does not survive in the absence of the auxotrophic gene product (e.g., outside of the gut).

Diaminopimelic acid (DAP) is an amino acid synthetized within the lysine biosynthetic pathway and is required for bacterial cell wall growth (Meadow et al., 1959; Clarkson et al., 1971). In some embodiments, any of the genetically engineered bacteria described herein is a dapD auxotroph in which dapD is deleted and/or replaced with an unrelated gene. A dapD auxotroph can grow only when sufficient amounts of DAP are present, e.g., by adding DAP to growth media in vitro. Without sufficient amounts of DAP, the dapD auxotroph dies. In some embodiments, the auxotrophic modification is used to ensure that the bacterial cell does not survive in the absence of the auxotrophic gene product (e.g., outside of the gut).

In other embodiments, the genetically engineered bacterium of the present disclosure is a uraA auxotroph in which uraA is deleted and/or replaced with an unrelated gene. The uraA gene codes for UraA, a membrane-bound transporter that facilitates the uptake and subsequent metabolism of the pyrimidine uracil (Andersen et al., 1995). A uraA auxotroph can grow only when sufficient amounts of uracil are present, e.g., by adding uracil to growth media in vitro. Without sufficient amounts of uracil, the uraA auxotroph dies. In some embodiments, auxotrophic modifications are used to ensure that the bacteria do not survive in the absence of the auxotrophic gene product (e.g., outside of the gut).

In complex communities, it is possible for bacteria to share DNA. In very rare circumstances, an auxotrophic bacterial strain may receive DNA from a non-auxotrophic strain, which repairs the genomic deletion and permanently rescues the auxotroph. Therefore, engineering a bacterial strain with more than one auxotroph may greatly decrease the probability that DNA transfer will occur enough times to rescue the auxotrophy. In some embodiments, the genetically engineered bacteria comprise a deletion or mutation in two or more genes required for cell survival and/or growth.

Other examples of essential genes include, but are not limited to yhbV, yagG, hemB, secD, secF, ribD, ribE, thiL, dxs, ispA, dnaX adk, hemH, lpxH, cysS, fold, rplT, infC, thrS, nadE, gapA, yeaZ, aspS, argS, pgsA, yejM, metG, folE, yejM, gyrA, nrdA, nrdB, folC, accD, fabB, gltX ligA, zipA, dapE, dapA, der, hisS, ispG, suhB, tadA, acpS, era, rnc, ftsB, eno, pyrG, chpR, lgt, jbaA, pgk, yqgD, metK, yqgF, plsC, ygiT, pare, ribB, cca, ygjD, tdcF, yraL, yihA, ftsN, murI, murB, birA, secE, nusG, rplJ, rplL, rpoB, rpoC, ubiA, plsB, lexA, dnaB, ssb, alsK, groS, psd, orn, yjeE, rpsR, chpS, ppa, valS, yjgP, yjgQ, dnaC, ribF, lspA, ispH, dapB, folA, imp, yabQ, ftsL, ftsI, murE, murF, mraY, murD, ftsW, murG, murC, ftsQ, ftsA, ftsZ, lpxC, secM, secA, can, folK, hemL, yadR, dapD, map, rpsB, infB, nusA, ftsH, obgE, rpmA, rplU, ispB, murA, yrbB, yrbK, yhbN, rpsl, rplM, degS, mreD, mreC, mreB, accB, accC, yrdC, def fmt, rplQ, rpoA, rpsD, rpsK, rpsM, entD, mrdB, mrdA, nadD, hlepB, rpoE, pssA, yfiO, rplS, trmD, rpsP, fjh, grpE, yfjB, csrA, ispF, ispD, rplW, rplD, rplC, rpsJ, fusA, rpsG, rpsL, trpS, yrfF, asd, rpoH, ftsX, ftsE, ftsY, frr, dxr, ispU, rfaK, kdtA, coaD, rpmB, dfp, dut, gmk, spot, gyrB, dnaN, dnaA, rpmH, rnpA, yidC, tnaB, glmS, glmU, wzyE, hemD, hemC, yigP, ubiB, ubiD, hemG, secY, rplO, rpmD, rpsE, rplR, rplF, rpsH, rpsN, rplE, rplX rplN, rpsQ, rpmC, rplP, rpsC, rplV, rpsS, rplB, cdsA, yaeL, yaeT, lpxD, fabZ, lpxA, lpxB, dnaE, accA, tilS, proS, yafF, tsf, pyrH, olA, rlpB, leuS, lnt, glnS, fldA, cydA, infA, cydC, ftsK, lolA, serS, rpsA, msbA, lpxK, kdsB, mukF, mukE, mukB, asnS, fabA, mviN, rne, yceQ, fabD, fabG, acpP, tmk, holB, lolC, lolD, lolE, purB, ymfK, minE, mind, pth, rsA, ispE, lolB, hemA, prfA, prmC, kdsA, topA, ribA, fabI, racR, dicA, ydfB, tyrS, ribC, ydiL, pheT, pheS, yhhQ, bcsB, glyQ, yibJ, and gpsA. Other essential genes are known to those of ordinary skill in the art.

In some embodiments, the genetically engineered bacterium of the present disclosure is a synthetic ligand-dependent essential gene (SLiDE) bacterial cell. SLiDE bacterial cells are synthetic auxotrophs with a mutation in one or more essential genes that only grow in the presence of a particular ligand (see Lopez and Anderson “Synthetic Auxotrophs with Ligand-Dependent Essential Genes for a BL21 (DE3 Biosafety Strain,” ACS Synthetic Biology (2015) DOI: 10.1021/acssynbio.5b00085, the entire contents of which are expressly incorporated herein by reference).

In some embodiments, the SLiDE bacterial cell comprises a mutation in an essential gene. In some embodiments, the essential gene is selected from the group consisting of pheS, dnaN, tyrS, metG and adk. In some embodiments, the essential gene is dnaN comprising one or more of the following mutations: H191N, R240C, I317S, F319V, L340T, V347I, and S345C. In some embodiments, the essential gene is dnaN comprising the mutations H191N, R240C, I317S, F319V, L340T, V347I, and S345C. In some embodiments, the essential gene is pheS comprising one or more of the following mutations: F125G, P183T, P184A, R186A, and I188L. In some embodiments, the essential gene is pheS comprising the mutations F125G, P183T, P184A, R186A, and I188L. In some embodiments, the essential gene is tyrS comprising one or more of the following mutations: L36V, C38A and F40G. In some embodiments, the essential gene is tyrS comprising the mutations L36V, C38A and F40G. In some embodiments, the essential gene is metG comprising one or more of the following mutations: E45Q, N47R, I49G, and A51C. In some embodiments, the essential gene is metG comprising the mutations E45Q, N47R, I49G, and A51C. In some embodiments, the essential gene is adk comprising one or more of the following mutations: I4L, L5I and L6G. In some embodiments, the essential gene is adk comprising the mutations I4L, L5I and L6G.

In some embodiments, the genetically engineered bacterium is complemented by a ligand. In some embodiments, the ligand is selected from the group consisting of benzothiazole, indole, 2-aminobenzothiazole, indole-3-butyric acid, indole-3-acetic acid, and L-histidine methyl ester. For example, bacterial cells comprising mutations in metG (E45Q, N47R, I49G, and A51C) are complemented by benzothiazole, indole, 2-aminobenzothiazole, indole-3-butyric acid, indole-3-acetic acid or L-histidine methyl ester. Bacterial cells comprising mutations in dnaN (H191N, R240C, I317S, F319V, L340T, V347I, and S345C) are complemented by benzothiazole, indole or 2-aminobenzothiazole. Bacterial cells comprising mutations in pheS (F125G, P183T, P184A, R186A, and I188L) are complemented by benzothiazole or 2-aminobenzothiazole. Bacterial cells comprising mutations in tyrS (L36V, C38A, and F40G) are complemented by benzothiazole or 2-aminobenzothiazole. Bacterial cells comprising mutations in adk (I4L, L5I and L6G) are complemented by benzothiazole or indole.

In some embodiments, the genetically engineered bacterium comprises more than one mutant essential gene that renders it auxotrophic to a ligand. In some embodiments, the bacterial cell comprises mutations in two essential genes. For example, in some embodiments, the bacterial cell comprises mutations in tyrS (L36V, C38A, and F40G) and metG (E45Q, N47R, 149G, and A51C). In other embodiments, the bacterial cell comprises mutations in three essential genes. For example, in some embodiments, the bacterial cell comprises mutations in tyrS (L36V, C38A, and F40G), metG (E45Q, N47R, 149G, and A51C), and pheS (F125G, P183T, P184A, R186A, and I188L).

In some embodiments, the genetically engineered bacterium is a conditional auxotroph whose essential gene(s) is replaced using the arabinose system described herein.

Genetic Regulatory Circuits

In some embodiments, the genetically engineered bacteria comprise multi-layered genetic regulatory circuits for expressing the constructs described herein (see, e.g., U.S. Provisional Application No. 62/184,811, incorporated herein by reference in its entirety). The genetic regulatory circuits are useful to screen for mutant bacteria that produce a branched chain amino acid catabolism enzyme, BCAA transporter, and/or BCAA binding protein or rescue an auxotroph. In certain embodiments, the invention provides methods for selecting genetically engineered bacteria that produce one or more genes of interest.

In some embodiments, the invention provides genetically engineered bacteria comprising a gene or gene cassette for producing a payload and a T7 polymerase-regulated genetic regulatory circuit. For example, the genetically engineered bacteria comprise a first gene encoding a T7 polymerase, wherein the first gene is operably linked to a fumarate and nitrate reductase regulator (FNR)-responsive promoter; a second gene or gene cassette for producing a payload, wherein the second gene or gene cassette is operably linked to a T7 promoter that is induced by the T7 polymerase; and a third gene encoding an inhibitory factor, lysY, that is capable of inhibiting the T7 polymerase. In the presence of oxygen, FNR does not bind the FNR-responsive promoter, and the payload is not expressed. LysY is expressed constitutively (β-lac constitutive) and further inhibits T7 polymerase. In the absence of oxygen, FNR dimerizes and binds to the FNR-responsive promoter, T7 polymerase is expressed at a level sufficient to overcome lysY inhibition, and the payload is expressed. In some embodiments, the lysY gene is operably linked to an additional FNR binding site. In the absence of oxygen, FNR dimerizes to activate T7 polymerase expression as described above, and also inhibits lysY expression.

In some embodiments, the invention provides genetically engineered bacteria comprising a gene or gene cassette for producing a payload and a protease-regulated genetic regulatory circuit. For example, the genetically engineered bacteria comprise a first gene encoding an mf-lon protease, wherein the first gene is operably linked to a FNR-responsive promoter; a second gene or gene cassette for producing a payload operably linked to a tet regulatory region (tetO); and a third gene encoding an mf-lon degradation signal linked to a tet repressor (tetR), wherein the tetR is capable of binding to the tet regulatory region and repressing expression of the second gene or gene cassette. The mf-lon protease is capable of recognizing the mf-lon degradation signal and degrading the tetR. In the presence of oxygen, FNR does not bind the FNR-responsive promoter, the repressor is not degraded, and the payload is not expressed. In the absence of oxygen, FNR dimerizes and binds the FNR-responsive promoter, thereby inducing expression of mf-lon protease. The mf-lon protease recognizes the mf-lon degradation signal and degrades the tetR, and the payload is expressed.

In some embodiments, the invention provides genetically engineered bacteria comprising a gene or gene cassette for producing a payload and a repressor-regulated genetic regulatory circuit. For example, the genetically engineered bacteria comprise a first gene encoding a first repressor, wherein the first gene is operably linked to a FNR-responsive promoter; a second gene or gene cassette for producing a payload operably linked to a first regulatory region comprising a constitutive promoter; and a third gene encoding a second repressor, wherein the second repressor is capable of binding to the first regulatory region and repressing expression of the second gene or gene cassette. The third gene is operably linked to a second regulatory region comprising a constitutive promoter, wherein the first repressor is capable of binding to the second regulatory region and inhibiting expression of the second repressor. In the presence of oxygen, FNR does not bind the FNR-responsive promoter, the first repressor is not expressed, the second repressor is expressed, and the payload is not expressed. In the absence of oxygen, FNR dimerizes and binds the FNR-responsive promoter, the first repressor is expressed, the second repressor is not expressed, and the payload is expressed.

Examples of repressors useful in these embodiments include, but are not limited to, ArgR, TetR, ArsR, AscG, LacI, CscR, DeoR, DgoR, FruR, GalR, GatR, CI, LexA, RafR, QacR, and PtxS (US20030166191).

In some embodiments, the invention provides genetically engineered bacteria comprising a gene or gene cassette for producing a payload and a regulatory RNA-regulated genetic regulatory circuit. For example, the genetically engineered bacteria comprise a first gene encoding a regulatory RNA, wherein the first gene is operably linked to a FNR-responsive promoter, and a second gene or gene cassette for producing a payload. The second gene or gene cassette is operably linked to a constitutive promoter and further linked to a nucleotide sequence capable of producing an mRNA hairpin that inhibits translation of the payload. The regulatory RNA is capable of eliminating the mRNA hairpin and inducing payload translation via the ribosomal binding site. In the presence of oxygen, FNR does not bind the FNR-responsive promoter, the regulatory RNA is not expressed, and the mRNA hairpin prevents the payload from being translated. In the absence of oxygen, FNR dimerizes and binds the FNR-responsive promoter, the regulatory RNA is expressed, the mRNA hairpin is eliminated, and the payload is expressed.

In some embodiments, the invention provides genetically engineered bacteria comprising a gene or gene cassette for producing a payload and a CRISPR-regulated genetic regulatory circuit. For example, the genetically engineered bacteria comprise a Cas9 protein; a first gene encoding a CRISPR guide RNA, wherein the first gene is operably linked to a FNR-responsive promoter; a second gene or gene cassette for producing a payload, wherein the second gene or gene cassette is operably linked to a regulatory region comprising a constitutive promoter; and a third gene encoding a repressor operably linked to a constitutive promoter, wherein the repressor is capable of binding to the regulatory region and repressing expression of the second gene or gene cassette. The third gene is further linked to a CRISPR target sequence that is capable of binding to the CRISPR guide RNA, wherein said binding to the CRISPR guide RNA induces cleavage by the Cas9 protein and inhibits expression of the repressor. In the presence of oxygen, FNR does not bind the FNR-responsive promoter, the guide RNA is not expressed, the repressor is expressed, and the payload is not expressed. In the absence of oxygen, FNR dimerizes and binds the FNR-responsive promoter, the guide RNA is expressed, the repressor is not expressed, and the payload is expressed.

In some embodiments, the invention provides genetically engineered bacteria comprising a gene or gene cassette for producing a payload and a recombinase-regulated genetic regulatory circuit. For example, the genetically engineered bacteria comprise a first gene encoding a recombinase, wherein the first gene is operably linked to a FNR-responsive promoter, and a second gene or gene cassette for producing a payload operably linked to a constitutive promoter. The second gene or gene cassette is inverted in orientation (3′ to 5′) and flanked by recombinase binding sites, and the recombinase is capable of binding to the recombinase binding sites to induce expression of the second gene or gene cassette by reverting its orientation (5′ to 3′). In the presence of oxygen, FNR does not bind the FNR-responsive promoter, the recombinase is not expressed, the payload remains in the 3′ to 5′ orientation, and no functional payload is produced. In the absence of oxygen, FNR dimerizes and binds the FNR-responsive promoter, the recombinase is expressed, the payload is reverted to the 5′ to 3′ orientation, and functional payload is produced.

In some embodiments, the invention provides genetically engineered bacteria comprising a gene or gene cassette for producing a payload and a polymerase- and recombinase-regulated genetic regulatory circuit. For example, the genetically engineered bacteria comprise a first gene encoding a recombinase, wherein the first gene is operably linked to a FNR-responsive promoter; a second gene or gene cassette for producing a payload operably linked to a T7 promoter; a third gene encoding a T7 polymerase, wherein the T7 polymerase is capable of binding to the T7 promoter and inducing expression of the payload. The third gene encoding the T7 polymerase is inverted in orientation (3′ to 5′) and flanked by recombinase binding sites, and the recombinase is capable of binding to the recombinase binding sites to induce expression of the T7 polymerase gene by reverting its orientation (5′ to 3′). In the presence of oxygen, FNR does not bind the FNR-responsive promoter, the recombinase is not expressed, the T7 polymerase gene remains in the 3′ to 5′ orientation, and the payload is not expressed. In the absence of oxygen, FNR dimerizes and binds the FNR-responsive promoter, the recombinase is expressed, the T7 polymerase gene is reverted to the 5′ to 3′ orientation, and the payload is expressed.

Isolated Plasmids

In other embodiments, the disclosure provides an isolated plasmid comprising a first nucleic acid encoding a first payload operably linked to a first inducible promoter, and a second nucleic acid encoding a second payload operably linked to a second inducible promoter. In other embodiments, the disclosure provides an isolated plasmid further comprising a third nucleic acid encoding a third payload operably linked to a third inducible promoter. In other embodiments, the disclosure provides a plasmid comprising four, five, six, or more nucleic acids encoding four, five, six, or more payloads operably linked to inducible promoters. In any of the embodiments described here, the first, second, third, fourth, fifth, sixth, etc “payload(s)” can be a branched chain amino acid catabolism enzyme, a transporter of branched chain amino acids, a binding protein of branched chain amino acids, or other sequence described herein. In one embodiment, the nucleic acid encoding the first payload and the nucleic acid encoding the second payload are operably linked to the first inducible promoter. In one embodiment, the nucleic acid encoding the first payload is operably linked to a first inducible promoter and the nucleic acid encoding the second payload is operably linked to a second inducible promoter. In one embodiment, the first inducible promoter and the second inducible promoter are separate copies of the same inducible promoter. In another embodiment, the first inducible promoter and the second inducible promoter are different inducible promoters. In other embodiments comprising a third nucleic acid, the nucleic acid encoding the third payload and the nucleic acid encoding the first and second payloads are all operably linked to the same inducible promoter. In other embodiments, the nucleic acid encoding the first payload is operably linked to a first inducible promoter, the nucleic acid encoding the second payload is operably linked to a second inducible promoter, and the nucleic acid encoding the third payload is operably linked to a third inducible promoter. In some embodiments, the first, second, and third inducible promoters are separate copies of the same inducible promoter. In other embodiments, the first inducible promoter, the second inducible promoter, and the third inducible promoter are different inducible promoters. In some embodiments, the first promoter, the second promoter, and the optional third promoter, or the first promoter and the second promoter and the optional third promoter, are each directly or indirectly induced by low-oxygen or anaerobic conditions. In other embodiments, the first promoter, the second promoter, and the optional third promoter, or the first promoter and the second promoter and the optional third promoter, are each a fumarate and nitrate reduction regulator (FNR) responsive promoter. In other embodiments, the first promoter, the second promoter, and the optional third promoter, or the first promoter and the second promoter and the optional third promoter are each a ROS-inducible regulatory region. In other embodiments, the first promoter, the second promoter, and the optional third promoter, or the first promoter and the second promoter and the optional third promoter are each a RNS-inducible regulatory region.

In some embodiments, the heterologous gene encoding a branched chain amino acid catabolism enzyme is operably linked to a constitutive promoter. In one embodiment, the constitutive promoter is a lac promoter. In another embodiment, the constitutive promoter is a tet promoter. In another embodiment, the constitutive promoter is a constitutive Escherichia coli σ³² promoter. In another embodiment, the constitutive promoter is a constitutive Escherichia coli σ⁷⁰ promoter. In another embodiment, the constitutive promoter is a constitutive Bacillus subtilis σ^{A} promoter. In another embodiment, the constitutive promoter is a constitutive Bacillus subtilis σ^{B} promoter. In another embodiment, the constitutive promoter is a Salmonella promoter. In other embodiments, the constitutive promoter is a bacteriophage T7 promoter. In other embodiments, the constitutive promoter is and a bacteriophage SP6 promoter. In any of the above-described embodiments, the plasmid further comprises a heterologous gene encoding a transporter of a branched chain amino acid, a BCAA binding protein, and/or a kill switch construct, which may be operably linked to a constitutive promoter or an inducible promoter.

In some embodiments, the isolated plasmid comprises at least one heterologous branched chain amino acid catabolism enzyme gene operably linked to a first inducible promoter; a heterologous gene encoding a TetR protein operably linked to a P_{araBAD} promoter, a heterologous gene encoding AraC operably linked to a P_{araC} promoter, a heterologous gene encoding an antitoxin operably linked to a constitutive promoter, and a heterologous gene encoding a toxin operably linked to a P_{TetR} promoter. In another embodiment, the isolated plasmid comprises at least one heterologous gene encoding a branched chain amino acid catabolism enzyme operably linked to a first inducible promoter; a heterologous gene encoding a TetR protein and an anti-toxin operably linked to a P_{araBAD} promoter, a heterologous gene encoding AraC operably linked to a P_{araC} promoter, and a heterologous gene encoding a toxin operably linked to a P_{TetR} promoter.

In one embodiment, the plasmid is a high-copy plasmid. In another embodiment, the plasmid is a low-copy plasmid.

In another aspect, the disclosure provides a recombinant bacterial cell comprising an isolated plasmid described herein. In another embodiment, the disclosure provides a pharmaceutical composition comprising the recombinant bacterial cell.

Integration

In some embodiments, any of the gene(s) or gene cassette(s) of the present disclosure may be integrated into the bacterial chromosome at one or more integration sites. One or more copies of the gene (for example, an amino acid catabolism gene, BCAA transporter gene, and/or BCAA binding protein gene) or gene cassette (for example, a gene cassette comprising an amino acid catabolism gene, an amino acid transporter gene, a BCAA binding protein gene) may be integrated into the bacterial chromosome. Having multiple copies of the gene or gene cassette integrated into the chromosome allows for greater production of the payload, e.g., amino acid catabolism enzyme, BCAA transporter gene, and/or BCAA binding protein gene and other enzymes of a gene cassette, and also permits fine-tuning of the level of expression. Alternatively, different circuits described herein, such as any of the kill-switch circuits, in addition to the therapeutic gene(s) or gene cassette(s) could be integrated into the bacterial chromosome at one or more different integration sites to perform multiple different functions.

Pharmaceutical Compositions and Formulations

Pharmaceutical compositions comprising the genetically engineered microorganisms of the invention may be used to treat, manage, ameliorate, and/or prevent a disorder associated with branched chain amino acid catabolism or symptom(s) associated with diseases or disorders associated with branched chain amino acid catabolism. Pharmaceutical compositions of the invention comprising one or more genetically engineered bacteria, and/or one or more genetically engineered yeast or virus, alone or in combination with prophylactic agents, therapeutic agents, and/or pharmaceutically acceptable carriers are provided.

In certain embodiments, the pharmaceutical composition comprises one species, strain, or subtype of bacteria that are engineered to comprise one or more of the genetic modifications described herein, e.g., selected from expression of at least one branched chain amino acid catabolism enzyme, BCAA transporter, BCAA binding protein, auxotrophy, kill-switch, exporter knock-out, etc. In alternate embodiments, the pharmaceutical composition comprises two or more species, strains, and/or subtypes of bacteria that are each engineered to comprise the genetic modifications described herein.

The pharmaceutical compositions of the invention described herein may be formulated in a conventional manner using one or more physiologically acceptable carriers comprising excipients and auxiliaries, which facilitate processing of the active ingredients into compositions for pharmaceutical use. Methods of formulating pharmaceutical compositions are known in the art (see, e.g., “Remington's Pharmaceutical Sciences,” Mack Publishing Co., Easton, Pa.). In some embodiments, the pharmaceutical compositions are subjected to tabletting, lyophilizing, direct compression, conventional mixing, dissolving, granulating, levigating, emulsifying, encapsulating, entrapping, or spray drying to form tablets, granulates, nanoparticles, nanocapsules, microcapsules, microtablets, pellets, or powders, which may be enterically coated or uncoated. Appropriate formulation depends on the route of administration.

The genetically engineered microorganisms may be formulated into pharmaceutical compositions in any suitable dosage form (e.g., liquids, capsules, sachet, hard capsules, soft capsules, tablets, enteric coated tablets, suspension powders, granules, or matrix sustained release formations for oral administration) and for any suitable type of administration (e.g., oral, topical, injectable, intravenous, sub-cutaneous, immediate-release, pulsatile-release, delayed-release, or sustained release). Suitable dosage amounts for the genetically engineered bacteria may range from about 104 to 1012 bacteria. The composition may be administered once or more daily, weekly, or monthly. The composition may be administered before, during, or following a meal. In one embodiment, the pharmaceutical composition is administered before the subject eats a meal. In one embodiment, the pharmaceutical composition is administered currently with a meal. In on embodiment, the pharmaceutical composition is administered after the subject eats a meal

The genetically engineered bacteria or genetically engineered yeast or virus may be formulated into pharmaceutical compositions comprising one or more pharmaceutically acceptable carriers, thickeners, diluents, buffers, buffering agents, surface active agents, neutral or cationic lipids, lipid complexes, liposomes, penetration enhancers, carrier compounds, and other pharmaceutically acceptable carriers or agents. For example, the pharmaceutical composition may include, but is not limited to, the addition of calcium bicarbonate, sodium bicarbonate, calcium phosphate, various sugars and types of starch, cellulose derivatives, gelatin, vegetable oils, polyethylene glycols, and surfactants, including, for example, polysorbate 20. In some embodiments, the genetically engineered bacteria of the invention may be formulated in a solution of sodium bicarbonate, e.g., 1 molar solution of sodium bicarbonate (to buffer an acidic cellular environment, such as the stomach, for example). The genetically engineered bacteria may be administered and formulated as neutral or salt forms. Pharmaceutically acceptable salts include those formed with anions such as those derived from hydrochloric, phosphoric, acetic, oxalic, tartaric acids, etc., and those formed with cations such as those derived from sodium, potassium, ammonium, calcium, ferric hydroxides, isopropylamine, triethylamine, 2-ethylamino ethanol, histidine, procaine, etc.

The genetically engineered microorganisms may be administered intravenously, e.g., by infusion or injection.

The genetically engineered microorganisms of the disclosure may be administered intrathecally. In some embodiments, the genetically engineered microorganisms of the invention may be administered orally. The genetically engineered microorganisms disclosed herein may be administered topically and formulated in the form of an ointment, cream, transdermal patch, lotion, gel, shampoo, spray, aerosol, solution, emulsion, or other form well known to one of skill in the art. See, e.g., “Remington's Pharmaceutical Sciences,” Mack Publishing Co., Easton, Pa. In an embodiment, for non-sprayable topical dosage forms, viscous to semi-solid or solid forms comprising a carrier or one or more excipients compatible with topical application and having a dynamic viscosity greater than water are employed. Suitable formulations include, but are not limited to, solutions, suspensions, emulsions, creams, ointments, powders, liniments, salves, etc., which may be sterilized or mixed with auxiliary agents (e.g., preservatives, stabilizers, wetting agents, buffers, or salts) for influencing various properties, e.g., osmotic pressure. Other suitable topical dosage forms include sprayable aerosol preparations wherein the active ingredient in combination with a solid or liquid inert carrier, is packaged in a mixture with a pressurized volatile (e.g., a gaseous propellant, such as freon) or in a squeeze bottle. Moisturizers or humectants can also be added to pharmaceutical compositions and dosage forms. Examples of such additional ingredients are well known in the art. In one embodiment, the pharmaceutical composition comprising the recombinant bacteria of the invention may be formulated as a hygiene product. For example, the hygiene product may be an antibacterial formulation, or a fermentation product such as a fermentation broth. Hygiene products may be, for example, shampoos, conditioners, creams, pastes, lotions, and lip balms.

The genetically engineered microorganisms disclosed herein may be administered orally and formulated as tablets, pills, dragees, capsules, liquids, gels, syrups, slurries, suspensions, etc. Pharmacological compositions for oral use can be made using a solid excipient, optionally grinding the resulting mixture, and processing the mixture of granules, after adding suitable auxiliaries if desired, to obtain tablets or dragee cores. Suitable excipients include, but are not limited to, fillers such as sugars, including lactose, sucrose, mannitol, or sorbitol; cellulose compositions such as maize starch, wheat starch, rice starch, potato starch, gelatin, gum tragacanth, methyl cellulose, hydroxypropylmethyl-cellulose, sodium carbomethylcellulose; and/or physiologically acceptable polymers such as polyvinylpyrrolidone (PVP) or polyethylene glycol (PEG). Disintegrating agents may also be added, such as cross-linked polyvinylpyrrolidone, agar, alginic acid or a salt thereof such as sodium alginate.

Tablets or capsules can be prepared by conventional means with pharmaceutically acceptable excipients such as binding agents (e.g., pregelatinised maize starch, polyvinylpyrrolidone, hydroxypropyl methylcellulose, carboxymethylcellulose, polyethylene glycol, sucrose, glucose, sorbitol, starch, gum, kaolin, and tragacanth); fillers (e.g., lactose, microcrystalline cellulose, or calcium hydrogen phosphate); lubricants (e.g., calcium, aluminum, zinc, stearic acid, polyethylene glycol, sodium lauryl sulfate, starch, sodium benzoate, L-leucine, magnesium stearate, talc, or silica); disintegrants (e.g., starch, potato starch, sodium starch glycolate, sugars, cellulose derivatives, silica powders); or wetting agents (e.g., sodium lauryl sulphate). The tablets may be coated by methods well known in the art. A coating shell may be present, and common membranes include, but are not limited to, polylactide, polyglycolic acid, polyanhydride, other biodegradable polymers, alginate-polylysine-alginate (APA), alginate-polymethylene-co-guanidine-alginate (A-PMCG-A), hydroymethylacrylate-methyl methacrylate (HEMA-MMA), multilayered HEMA-MMA-MAA, polyacrylonitrilevinylchloride (PAN-PVC), acrylonitrile/sodium methallylsulfonate (AN-69), polyethylene glycol/poly pentamethylcyclopentasiloxane/polydimethylsiloxane (PEG/PD5/PDMS), poly N,N-dimethyl acrylamide (PDMAAm), siliceous encapsulates, cellulose sulphate/sodium alginate/polymethylene-co-guanidine (CS/A/PMCG), cellulose acetate phthalate, calcium alginate, k-carrageenan-locust bean gum gel beads, gellan-xanthan beads, poly(lactide-co-glycolides), carrageenan, starch poly-anhydrides, starch polymethacrylates, polyamino acids, and enteric coating polymers.

In some embodiments, the genetically engineered microorganisms are enterically coated for release into the gut or a particular region of the gut, for example, the large intestine. The typical pH profile from the stomach to the colon is about 1-4 (stomach), 5.5-6 (duodenum), 7.3-8.0 (ileum), and 5.5-6.5 (colon). In some diseases, the pH profile may be modified. In some embodiments, the coating is degraded in specific pH environments in order to specify the site of release. In some embodiments, at least two coatings are used. In some embodiments, the outside coating and the inside coating are degraded at different pH levels.

Liquid preparations for oral administration may take the form of solutions, syrups, suspensions, or a dry product for constitution with water or other suitable vehicle before use. Such liquid preparations may be prepared by conventional means with pharmaceutically acceptable agents such as suspending agents (e.g., sorbitol syrup, cellulose derivatives, or hydrogenated edible fats); emulsifying agents (e.g., lecithin or acacia); non-aqueous vehicles (e.g., almond oil, oily esters, ethyl alcohol, or fractionated vegetable oils); and preservatives (e.g., methyl or propyl-p-hydroxybenzoates or sorbic acid). The preparations may also contain buffer salts, flavoring, coloring, and sweetening agents as appropriate. Preparations for oral administration may be suitably formulated for slow release, controlled release, or sustained release of the genetically engineered microorganisms described herein.

In one embodiment, the genetically engineered microorganisms of the disclosure may be formulated in a composition suitable for administration to pediatric subjects. As is well known in the art, children differ from adults in many aspects, including different rates of gastric emptying, pH, gastrointestinal permeability, etc. (Ivanovska et al., Pediatrics, 134(2):361-372, 2014). Moreover, pediatric formulation acceptability and preferences, such as route of administration and taste attributes, are critical for achieving acceptable pediatric compliance. Thus, in one embodiment, the composition suitable for administration to pediatric subjects may include easy-to-swallow or dissolvable dosage forms, or more palatable compositions, such as compositions with added flavors, sweeteners, or taste blockers. In one embodiment, a composition suitable for administration to pediatric subjects may also be suitable for administration to adults.

In one embodiment, the composition suitable for administration to pediatric subjects may include a solution, syrup, suspension, elixir, powder for reconstitution as suspension or solution, dispersible/effervescent tablet, chewable tablet, gummy candy, lollipop, freezer pop, troche, chewing gum, oral thin strip, orally disintegrating tablet, sachet, soft gelatin capsule, sprinkle oral powder, or granules. In one embodiment, the composition is a gummy candy, which is made from a gelatin base, giving the candy elasticity, desired chewy consistency, and longer shelf-life. In some embodiments, the gummy candy may also comprise sweeteners or flavors.

In one embodiment, the composition suitable for administration to pediatric subjects may include a flavor. As used herein, “flavor” is a substance (liquid or solid) that provides a distinct taste and aroma to the formulation. Flavors also help to improve the palatability of the formulation. Flavors include, but are not limited to, strawberry, vanilla, lemon, grape, bubble gum, and cherry.

In certain embodiments, the genetically engineered microorganisms may be orally administered, for example, with an inert diluent or an assimilable edible carrier. The compound may also be enclosed in a hard or soft shell gelatin capsule, compressed into tablets, or incorporated directly into the subject's diet. For oral therapeutic administration, the compounds may be incorporated with excipients and used in the form of ingestible tablets, buccal tablets, troches, capsules, elixirs, suspensions, syrups, wafers, and the like. To administer a compound by other than parenteral administration, it may be necessary to coat the compound with, or co-administer the compound with, a material to prevent its inactivation.

In another embodiment, the pharmaceutical composition comprising the recombinant bacteria of the invention may be a comestible product, for example, a food product. In one embodiment, the food product is milk, concentrated milk, fermented milk (yogurt, sour milk, frozen yogurt, lactic acid bacteria-fermented beverages), milk powder, ice cream, cream cheeses, dry cheeses, soybean milk, fermented soybean milk, vegetable-fruit juices, fruit juices, sports drinks, confectionery, candies, infant foods (such as infant cakes), nutritional food products, animal feeds, or dietary supplements. In one embodiment, the food product is a fermented food, such as a fermented dairy product. In one embodiment, the fermented dairy product is yogurt. In another embodiment, the fermented dairy product is cheese, milk, cream, ice cream, milk shake, or kefir. In another embodiment, the recombinant bacteria of the invention are combined in a preparation containing other live bacterial cells intended to serve as probiotics. In another embodiment, the food product is a beverage. In one embodiment, the beverage is a fruit juice-based beverage or a beverage containing plant or herbal extracts. In another embodiment, the food product is a jelly or a pudding. Other food products suitable for administration of the recombinant bacteria of the invention are well known in the art. For example, see U.S. 2015/0359894 and US 2015/0238545, the entire contents of each of which are expressly incorporated herein by reference. In yet another embodiment, the pharmaceutical composition of the invention is injected into, sprayed onto, or sprinkled onto a food product, such as bread, yogurt, or cheese.

In some embodiments, the composition is formulated for intraintestinal administration, intrajejunal administration, intraduodenal administration, intraileal administration, gastric shunt administration, or intracolic administration, via nanoparticles, nanocapsules, microcapsules, or microtablets, which are enterically coated or uncoated. The pharmaceutical compositions may also be formulated in rectal compositions such as suppositories or retention enemas, using, e.g., conventional suppository bases such as cocoa butter or other glycerides. The compositions may be suspensions, solutions, or emulsions in oily or aqueous vehicles, and may contain suspending, stabilizing and/or dispersing agents.

The genetically engineered microorganisms described herein may be administered intranasally, formulated in an aerosol form, spray, mist, or in the form of drops, and conveniently delivered in the form of an aerosol spray presentation from pressurized packs or a nebuliser, with the use of a suitable propellant (e.g., dichlorodifluoromethane, trichlorofluoromethane, dichlorotetrafluoroethane, carbon dioxide or other suitable gas). Pressurized aerosol dosage units may be determined by providing a valve to deliver a metered amount. Capsules and cartridges (e.g., of gelatin) for use in an inhaler or insufflator may be formulated containing a powder mix of the compound and a suitable powder base such as lactose or starch.

The genetically engineered microorganisms may be administered and formulated as depot preparations. Such long acting formulations may be administered by implantation or by injection, including intravenous injection, subcutaneous injection, local injection, direct injection, or infusion. For example, the compositions may be formulated with suitable polymeric or hydrophobic materials (e.g., as an emulsion in an acceptable oil) or ion exchange resins, or as sparingly soluble derivatives (e.g., as a sparingly soluble salt).

In some embodiments, disclosed herein are pharmaceutically acceptable compositions in single dosage forms. Single dosage forms may be in a liquid or a solid form. Single dosage forms may be administered directly to a patient without modification or may be diluted or reconstituted prior to administration. In certain embodiments, a single dosage form may be administered in bolus form, e.g., single injection, single oral dose, including an oral dose that comprises multiple tablets, capsule, pills, etc. In alternate embodiments, a single dosage form may be administered over a period of time, e.g., by infusion.

Single dosage forms of the pharmaceutical composition may be prepared by portioning the pharmaceutical composition into smaller aliquots, single dose containers, single dose liquid forms, or single dose solid forms, such as tablets, granulates, nanoparticles, nanocapsules, microcapsules, microtablets, pellets, or powders, which may be enterically coated or uncoated. A single dose in a solid form may be reconstituted by adding liquid, typically sterile water or saline solution, prior to administration to a patient.

In other embodiments, the composition can be delivered in a controlled release or sustained release system. In one embodiment, a pump may be used to achieve controlled or sustained release. In another embodiment, polymeric materials can be used to achieve controlled or sustained release of the therapies of the present disclosure (see e.g., U.S. Pat. No. 5,989,463). Examples of polymers used in sustained release formulations include, but are not limited to, poly(2-hydroxy ethyl methacrylate), poly(methyl methacrylate), poly(acrylic acid), poly(ethylene-co-vinyl acetate), poly(methacrylic acid), polyglycolides (PLG), polyanhydrides, poly(N-vinyl pyrrolidone), poly(vinyl alcohol), polyacrylamide, poly(ethylene glycol), polylactides (PLA), poly(lactide-co-glycolides) (PLGA), and polyorthoesters. The polymer used in a sustained release formulation may be inert, free of leachable impurities, stable on storage, sterile, and biodegradable. In some embodiments, a controlled or sustained release system can be placed in proximity of the prophylactic or therapeutic target, thus requiring only a fraction of the systemic dose. Any suitable technique known to one of skill in the art may be used.

Dosage regimens may be adjusted to provide a therapeutic response. Dosing can depend on several factors, including severity and responsiveness of the disease, route of administration, time course of treatment (days to months to years), and time to amelioration of the disease. For example, a single bolus may be administered at one time, several divided doses may be administered over a predetermined period of time, or the dose may be reduced or increased as indicated by the therapeutic situation. The specification for the dosage is dictated by the unique characteristics of the active compound and the particular therapeutic effect to be achieved. Dosage values may vary with the type and severity of the condition to be alleviated. For any particular subject, specific dosage regimens may be adjusted overtime according to the individual need and the professional judgment of the treating clinician. Toxicity and therapeutic efficacy of compounds provided herein can be determined by standard pharmaceutical procedures in cell culture or animal models. For example, LD50, ED50, EC50, and IC50 may be determined, and the dose ratio between toxic and therapeutic effects (LD50/ED50) may be calculated as the therapeutic index. Compositions that exhibit toxic side effects may be used, with careful modifications to minimize potential damage to reduce side effects. Dosing may be estimated initially from cell culture assays and animal models. The data obtained from in vitro and in vivo assays and animal studies can be used in formulating a range of dosage for use in humans.

The ingredients are supplied either separately or mixed together in unit dosage form, for example, as a dry lyophilized powder or water-free concentrate in a hermetically sealed container such as an ampoule or sachet indicating the quantity of active agent. If the mode of administration is by injection, an ampoule of sterile water for injection or saline can be provided so that the ingredients may be mixed prior to administration.

The pharmaceutical compositions may be packaged in a hermetically sealed container such as an ampoule or sachet indicating the quantity of the agent. In one embodiment, one or more of the pharmaceutical compositions is supplied as a dry sterilized lyophilized powder or water-free concentrate in a hermetically sealed container and can be reconstituted (e.g., with water or saline) to the appropriate concentration for administration to a subject. In an embodiment, one or more of the prophylactic or therapeutic agents or pharmaceutical compositions is supplied as a dry sterile lyophilized powder in a hermetically sealed container stored between 2° C. and 8° C. and administered within 1 hour, within 3 hours, within 5 hours, within 6 hours, within 12 hours, within 24 hours, within 48 hours, within 72 hours, or within one week after being reconstituted. Cryoprotectants can be included for a lyophilized dosage form, principally 0-10% sucrose (optimally 0.5-1.0%). Other suitable cryoprotectants include trehalose and lactose. Other suitable bulking agents include glycine and arginine, either of which can be included at a concentration of 0-0.05%, and polysorbate-80 (optimally included at a concentration of 0.005-0.01%). Additional surfactants include but are not limited to polysorbate 20 and BRIJ surfactants. The pharmaceutical composition may be prepared as an injectable solution and can further comprise an agent useful as an adjuvant, such as those used to increase absorption or dispersion, e.g., hyaluronidase.

In some embodiments, the genetically engineered viruses are prepared for delivery, taking into consideration the need for efficient delivery and for overcoming the host antiviral immune response. Approaches to evade antiviral response include the administration of different viral serotypes as part of the treatment regimen (serotype switching), formulation, such as polymer coating to mask the virus from antibody recognition and the use of cells as delivery vehicles.

In another embodiment, the composition can be delivered in a controlled release or sustained release system. In one embodiment, a pump may be used to achieve controlled or sustained release. In another embodiment, polymeric materials can be used to achieve controlled or sustained release of the therapies of the present disclosure (see e.g., U.S. Pat. No. 5,989,463). Examples of polymers used in sustained release formulations include, but are not limited to, poly(2-hydroxy ethyl methacrylate), poly(methyl methacrylate), poly(acrylic acid), poly(ethylene-co-vinyl acetate), poly(methacrylic acid), polyglycolides (PLG), polyanhydrides, poly(N-vinyl pyrrolidone), poly(vinyl alcohol), polyacrylamide, poly(ethylene glycol), polylactides (PLA), poly(lactide-co-glycolides) (PLGA), and polyorthoesters. The polymer used in a sustained release formulation may be inert, free of leachable impurities, stable on storage, sterile, and biodegradable. In some embodiments, a controlled or sustained release system can be placed in proximity of the prophylactic or therapeutic target, thus requiring only a fraction of the systemic dose. Any suitable technique known to one of skill in the art may be used.

The genetically engineered bacteria of the invention may be administered and formulated as neutral or salt forms. Pharmaceutically acceptable salts include those formed with anions such as those derived from hydrochloric, phosphoric, acetic, oxalic, tartaric acids, etc., and those formed with cations such as those derived from sodium, potassium, ammonium, calcium, ferric hydroxides, isopropylamine, triethylamine, 2-ethylamino ethanol, histidine, procaine, etc. In vivo Methods

The recombinant bacteria disclosed herein may be evaluated in vivo, e.g., in an animal model. Any suitable animal model of a disease or condition associated with catabolism of a branched chain amino acid may be used (see, e.g., Skvorak, J. Inherit. Metab. Dis., 2009, 32:229-246 and Homanics et al., BMC Med. Genet., 2006, 7(33):1-13), including the Dbt−/− model (E2 subunit of BCKDH, which has a 3-fold increase in blood and urine BCAA levels and results in neonatal lethalthy) (serves as classic MSUD model). This model is partially rescued by two transgenes (LAP-tTA and TRE-E2), allowing 5-6% of normal BCKDH activity and an increase in mice survival to three or four weeks (serves as an intermediate MSUD model) (as described in Homanics et al., 2006, the contents of which is herein incorporated by reference in its entirety). In addition, intermediate MSUD mice can be used to show development of neuropathology with striking similarity to human MSUD. In this model, branched-chain amino acid accumulation was associated with neurotransmitter deficiency, behavioral changes and limited survival, and providing intermediate MSUD mice with a choice between normal and branched-chain amino acid free diet prevented brain injury and dramatically improved survival (Zinnanti et al., Dual mechanism of brain injury and novel treatment strategy in maple syrup urine disease; Brain 2009: 132; 903-918, the contents of which is herein incorporated by reference in its entirety). In some embodiments, the animal model is a mouse model of Maple Syrup Urine Disease. In one embodiment, the mouse model of MSUD is a branched-chain amino transferase knockout mouse (Wu et al., J. Clin. Invest, 113:434-440, 2004 or She et al., Cell Metabol., 6:181-194, 2007). In another embodiment, the mouse model of MSUD is a dihydrolipoamine dehydrogenase (E3) subunit knock-out mouse (Johnson et al., Proc. Natl. Acad. Sci. U.S.A., 94:14512-14517, 1997). In another embodiment, the mouse model of classic MSUD is a deletion of exon 5 and part of exon 4 of the E2 subunit of the branched-chain alpha-keto acid dehydrogenase (Homanics et al., BMC Med. Genet., 7:33, 2006) or the mouse model of intermediate MSUD (Homanics et al., BMC Med. Genet., 7:33, 2006). In another embodiment, the model is a Polled Shorthorn calf model of disease or a Polled Hereford calf model of disease (Harper et al., Aus. Vet. J., 66(2):46-49, 1988). Other relevant animal models include those described in She et al., Cell Metab. 2007 September; 6(3): 181-194; Wu et al., J. Clin. Invest. 113:434-440 (2004); Bridi, et al., J. Neurosci Methods, 2006 Sep. 15; 155(2):224-30.

The recombinant bacterial cells disclosed herein may administered to the animal, e.g., by oral gavage, and treatment efficacy is determined, e.g., by measuring blood leucine levels before and after treatment. The animal may be sacrificed, and tissue samples may be collected and analyzed.

Methods of Screening

Generation of Bacterial Strains with Enhance Ability to Transport Amino Acids

Due to their ease of culture, short generation times, very high population densities and small genomes, microbes can be evolved to unique phenotypes in abbreviated timescales. Adaptive laboratory evolution (ALE) is the process of passaging microbes under selective pressure to evolve a strain with a preferred phenotype. Most commonly, this is applied to increase utilization of carbon/energy sources or adapting a strain to environmental stresses (e.g., temperature, pH), whereby mutant strains more capable of growth on the carbon substrate or under stress will outcompete the less adapted strains in the population and will eventually come to dominate the population.

This same process can be extended to any essential metabolite by creating an auxotroph. An auxotroph is a strain incapable of synthesizing an essential metabolite and must therefore have the metabolite provided in the media to grow. In this scenario, by making an auxotroph and passaging it on decreasing amounts of the metabolite, the resulting dominant strains should be more capable of obtaining and incorporating this essential metabolite.

For example, if the biosynthetic pathway for producing an amino acid is disrupted a strain capable of high-affinity capture of said amino acid can be evolved via ALE. First, the strain is grown in varying concentrations of the auxotrophic amino acid, until a minimum concentration to support growth is established. The strain is then passaged at that concentration, and diluted into lowering concentrations of the amino acid at regular intervals. Over time, cells that are most competitive for the amino acid—at growth-limiting concentrations—will come to dominate the population. These strains will likely have mutations in their amino acid-transporters resulting in increased ability to import the essential and limiting amino acid.

Similarly, by using an auxotroph that cannot use an upstream metabolite to form an amino acid, a strain can be evolved that not only can more efficiently import the upstream metabolite, but also convert the metabolite into the essential downstream metabolite. These strains will also evolve mutations to increase import of the upstream metabolite, but may also contain mutations which increase expression or reaction kinetics of downstream enzymes, or that reduce competitive substrate utilization pathways.

A metabolite innate to the microbe can be made essential via mutational auxotrophy and selection applied with growth-limiting supplementation of the endogenous metabolite. However, phenotypes capable of consuming non-native compounds can be evolved by tying their consumption to the production of an essential compound. For example, if a gene from a different organism is isolated which can produce an essential compound or a precursor to an essential compound this gene can be recombinantly introduced and expressed in the heterologous host. This new host strain will now have the ability to synthesize an essential nutrient from a previously non-metabolizable substrate.

Hereby, a similar ALE process can be applied by creating an auxotroph incapable of converting an immediately downstream metabolite and selecting in growth-limiting amounts of the non-native compound with concurrent expression of the recombinant enzyme. This will result in mutations in the transport of the non-native substrate, expression and activity of the heterologous enzyme and expression and activity of downstream native enzymes. It should be emphasized that the key requirement in this process is the ability to tether the consumption of the non-native metabolite to the production of a metabolite essential to growth.

Once the basis of the selection mechanism is established and minimum levels of supplementation have been established, the actual ALE experimentation can proceed. Throughout this process several parameters must be vigilantly monitored. It is important that the cultures are maintained in an exponential growth phase and not allowed to reach saturation/stationary phase. This means that growth rates must be check during each passaging and subsequent dilutions adjusted accordingly. If growth rate improves to such a degree that dilutions become large, then the concentration of auxotrophic supplementation should be decreased such that growth rate is slowed, selection pressure is increased and dilutions are not so severe as to heavily bias subpopulations during passaging. In addition, at regular intervals cells should be diluted, grown on solid media and individual clones tested to confirm growth rate phenotypes observed in the ALE cultures.

Predicting when to halt the stop the ALE experiment also requires vigilance. As the success of directing evolution is tied directly to the number of mutations “screened” throughout the experiment and mutations are generally a function of errors during DNA replication, the cumulative cell divisions (CCD) acts as a proxy for total mutants which have been screened. Previous studies have shown that beneficial phenotypes for growth on different carbon sources can be isolated in about 10^{11.2} CCD¹. This rate can be accelerated by the addition of chemical mutagens to the cultures—such as N-methyl-N-nitro-N-nitrosoguanidine (NTG)—which causes increased DNA replication errors. However, when continued passaging leads to marginal or no improvement in growth rate the population has converged to some fitness maximum and the ALE experiment can be halted.

At the conclusion of the ALE experiment, the cells should be diluted, isolated on solid media and assayed for growth phenotypes matching that of the culture flask. Best performers from those selected are then prepped for genomic DNA and sent for whole genome sequencing. Sequencing with reveal mutations occurring around the genome capable of providing improved phenotypes, but will also contain silent mutations (those which provide no benefit but do not detract from desired phenotype). In cultures evolved in the presence of NTG or other chemical mutagen, there will be significantly more silent, background mutations. If satisfied with the best performing strain in its current state, the user can proceed to application with that strain. Otherwise the contributing mutations can be deconvoluted from the evolved strain by reintroducing the mutations to the parent strain by genome engineering techniques. See Lee, D.-H., Feist, A. M., Barrett, C. L. & Palsson, B. O. Cumulative Number of Cell Divisions as a Meaningful Timescale for Adaptive Laboratory Evolution of Escherichia coli. PLoS ONE 6, e26172 (2011).

Similar methods can be used to generate E. coli Nissle mutants that consume or import branched chain amino acids, e.g., leucine, valine, and/or isoleucine.

Specific Screen to Identify Strains with Improved BCAA Degradation Enzyme Activity

Screens using genetic selection are conducted to improve BCAA consumption in the genetically engineered bacteria. Toxic BCAA analogs exert their mechanism of action (MOA) by being incorporated into cellular protein, causing cell death. These compounds, e.g., fluoro-leucine and/or aza-leucine, have utility in an untargeted approach to select BCAA enzymes with increased activity. Assuming that these toxic compounds can be metabolized by BCAA enzymes into a non-toxic metabolite, rather than being incorporated into cellular protein, genetically engineered bacteria which have improved BCAA degradation activity can tolerate higher levels of these compounds, and can be screened for and selected on this basis.

Use of Valine and Leucine Sensitivity to Identify Strains with Improved BCAA Degradation Enzyme Activity

Valine and Leucine sensitivity can be used as a genetic screening tool using the E. coli K12 strain. There are three AHAS (acetohydroxybutanoate synthase) isozymes in E. coli (AHAS I: ilvBN, AHAS II: ilvGM, and AHAS III: ilvIH). Valine and leucine exert feedback inhibition on AHAS I and AHAS III; AHAS II is resistant to Val and Leu inhibition. E. coli K12 has a frameshift mutation in ilvG (AHAS II) and is unable to produce BCAA endogenously in the presence of valine and leucine. In contrast, E. coli Nissle has a functional ilvG and is insensitive to valine and leucine and therefore cannot be used for this screen. A genetically engineered strain derived from E. coli K12, which more efficiently degrades leucine, has a greater reduction in sensitivity to leucine (through relieving the feedback inhibition on AHAS I and III). As a result, this pathway can be used as a tool to select and identify a strain with improved resistance to leucine.

Use of Leucine Auxotrophy and D-Leucine as a Method to Identify Strains with Improved BCAA Uptake Ability.

Bacterial mutants with increased leucine transport into the bacterial cell may be identified using a leucine auxotroph and providing D-leucine instead of L-leucine in the media, as D-leucine can be imported through the same transporters. The bacteria can grow in the presence of D-leucine, because the bacterial stain has a racemase, which can convert D-leucine to L-leucine. However, the uptake of D-leucine through LivKHMGF is less efficient than the uptake of L-leucine. The leucine auxotroph can still grow if high concentrations of D-Leucine are provided, even though the D-leucine uptake is less efficient than L-leucine uptake. When concentrations of D-leucine in the media are lowered, the cells can no longer grow, unless transport efficiency is increased, ergo, mutants with increased D-leucine uptake can be selected.

Methods of Treatment

Further disclosed herein are methods of treating a disease or disorder associated with the catabolism of a branched chain amino acid. In some embodiments, disclosed herein are methods for reducing, ameliorating, or eliminating one or more symptom(s) associated with these diseases or disorders. In one embodiment, the disorder involving the catabolism of a branched chain amino acid is a metabolic disorder involving the abnormal catabolism of a branched chain amino acid. Metabolic diseases associated with abnormal catabolism of a branched chain amino acid include maple syrup urine disease (MSUD), isovaleric acidemia, propionic acidemia, methylmalonic acidemia, diabetes ketoacidosis, MCC Deficiency, 3-Methylglutaconyl-CoA hydratase Deficiency, HMG-CoA Lyase Deficiency, Acetyl-CoA Carboxylase Deficiency, Malonyl-CoA Decarboxylase Deficiency, short-branched chain acylCoA dehydrogenase deficiency, 2-methyl-3-hydroxybutyric acidemia, beta-ketothiolase deficiency, isobutyryl-CoA dehydrogenase deficiency, HIBCH deficiency), and 3-Hydroxyisobutyric aciduria.

In one embodiment, the disease associated with abnormal catabolism of a branched chain amino acid is isovaleric acidemia. In one embodiment, the disease associated with abnormal catabolism of a branched chain amino acid is propionic acidemia. In one embodiment, the disease associated with abnormal catabolism of a branched chain amino acid is methylmalonic acidemia. In another embodiment, the disease associated with abnormal catabolism of a branched chain amino acid is diabetes.

In one embodiment, the disease is maple syrup urine disease (MSUD). Maple syrup urine disease (MSUD), also known as branched-chain ketoaciduria, is an autosomal recessive metabolic disorder caused by impaired activity of the branched-chain α-keto acid dehydrogenase (BCKDH) complex (Skvorak 2009). The overall incidence for MSUD is 1:185,000, although it is higher in certain populations, such as Mennonites, where the incidence is 1:176. The BCKDH complex is responsible for the oxidative decarboxylation of branched-chain keto acids (BCKAs) derived from branched chain amino acids (BCAAs) (Homanics et al. 2006). Patients with MSUD are unable to properly process BCKAs, which can lead to the toxic accumulation of BCAAs and their derivatives in the blood, cerebrospinal fluid and tissues (Skvorak 2009). Specifically, deficiencies of the BCKDH complex in MSUD patients results in accumulation of the BCAAs isoleucine, leucine, and valine, as well as their corresponding branched-chain α-keto acids (BCKAs) α-keto-β-methylvalerate, α-ketoisocaproate, and α-ketoisovalerate) in the tissues in plasma. Clinical manifestations of the disease vary depending on the degree of enzyme deficiency and include poor feeding, vomiting, dehydration, lethargy, hypotonia, seizures, hypoglycemia, ketoacidosis, pancreatitis, coma, and neurological decline (Homanics et al. 2009).

The BCKDH complex is composed of three catalytic components: a dehydrogenase/decarboxylase (E1), which is a heterotetramer composed of two E1α and two E1β subunits, a dihydrolipoyl transacylase (E2), and a dihydrolipoamide dehydrogenase (E3) (Skvorak 2009). Additionally, the complex is associated with two regulatory enzymes, a BCKDH kinase and a BCKDH phosphatase, which control its activity through reversible phosphorylation-dephosphorylation of E1α (Chuang 1998, Homanics et al. 2006). To date, MSUD has been associated with mutations in the E1, E2 and E3 subunits of the BCKDH complex (Cheung 1998, Homanics et al. 2006).

MSUD is a very complex, genetically heterogeneous disease. At least 150 mutations in genes encoding BCKDH complex components have been identified that result in MSUD (Skvorak 2009). For example, see Table 8 below, adapted from Chuang, J. Pediatrics, 132(3), Part 2, S17-S23, 1998. As indicated below, E2 mutants are the most prevalent in human disease.

TABLE 8
MSUD Phenotypes

| | | | Number of |
|---|---|---|---|
| Molecular | Affected | | Mutations |
| Phenotype | Gene | Clinical Phenotype | Identified |
| IA | E1α | Classic, Intermediate MSUD | 15 |
| IB | E1β | Classic MSUD | 4 |
| II | E2 | Classic, thiamine-responsive | 26 |
| | | MSUD | |
| III | E3 | E3-deficient | 4 |
| IV | Kinase | None reported | None reported |
| V | Phosphatase | None reported | None reported |

Currently available treatments for MSUD are inadequate for the long term management of the disease and have severe limitations (Svkvorak 2009). A low protein/BCAA-restricted diet, with micronutrient and vitamin supplementation, as necessary, is the widely accepted long-term disease management strategy for MSUD (Homanics et al. 2006). However, BCAA-intake restrictions can be particularly problematic since BCAAs can only be acquired through diet and are necessary for several metabolic activities (Skvorak 2009). Even with proper monitoring and patient compliance, BCAA dietary restrictions result in a high incidence of mental retardation and mortality (Skvorak 2009, Homanics et al 2009). Further, a few cases of MSUD have been treated by liver transplantation (Popescu and Dima 2012) or treatment with phenylbutyrate. However, the limited availability of donor organs, the costs associated with the transplantation itself, and the undesirable effects associated with continued immunosuppressant therapy limit the practicality of liver transplantation for treatment of disease (Homanics et al. 2012, Popescu and Dima 2012). Therefore, there is significant unmet need for effective, reliable, and/or long-term treatment for MSUD.

The present disclosure surprisingly demonstrates that pharmaceutical compositions comprising the recombinant bacterial cells disclosed herein may be used to treat metabolic diseases involving the abnormal catabolism of a branched chain amino acid, such as MSUD. In one embodiment, the metabolic disease is selected from the group consisting of classic MSUD, intermediate MSUD, intermittent MSUD, E3-Deficient MSUD, and thiamine-responsive MSUD. In one embodiment, the disease is classic MSUD. In another embodiment, the disease is intermediate MSUD. In another embodiment, the disease is intermittent MSUD. In another embodiment, the disease is E3-deficient MSUD. In another embodiment, the disease is thiamine-responsive MSUD.

In one embodiment, the subject having MSUD has a mutation in an E1α gene. In another embodiment, the subject having MSUD has a mutation in the E1β gene. In another embodiment, the subject having MSUD has a mutation in the E2 gene. In another embodiment, the subject having MSUD has a mutation in the E3 gene.

In one embodiment, the target degradation rates of branched chain amino acids from food intake in breastfed infants and adults is as indicated in the Table 9, below.

TABLE 9
Target Degradation rates for BCAA.

| Age (year) | <1 | | | 1-3 | | | 4-8 | | | 8-12 | | | >12 | | |
|---|---|---|---|---|---|---|---|---|---|---|---|---|---|---|---|
| Amino acid: Leu (L) Val (V) Ile (I) | L | V | I | L | V | I | L | V | I | L | V | I | L | V | I |
| MSUD patient daily tolerance (mg/kg) | 40 | 30 | 20 | 20 | 10 | 5 | 5 | 10 | 5 | 5 | 10 | 5 | 5 | 10 | 5 |
| Recommended Dietary Allowance | 93 | 58 | 43 | 63 | 37 | 28 | 49 | 28 | 22 | 46 | 26 | 20 | 46 | 26 | 20 |
| (RDA) (mg/kg) | | | | | | | | | | | | | | | |
| Target reduction (mg/kg) | 53 | 28 | 23 | 43 | 27 | 23 | 44 | 18 | 17 | 41 | 16 | 15 | 41 | 16 | 15 |
| Target reduction (mg); | 530 | 280 | 230 | 602 | 378 | 322 | 1144 | 468 | 442 | 1681 | 656 | 615 | 2870 | 1120 | 1050 |
| (based on 10, 14, 26, 41 and 70 kg | | | | | | | | | | | | | | | |
| weight for the different age groups) | | | | | | | | | | | | | | | |
| Target reduction (mmol) | 4.04 | 2.39 | 1.75 | 4.59 | 3.23 | 2.45 | 8.72 | 3.99 | 3.37 | 12.81 | 5.60 | 4.69 | 21.88 | 9.56 | 8.00 |
| Target degradation rate | 0.56 | 0.33 | 0.24 | 0.64 | 0.45 | 0.34 | 1.21 | 0.55 | 0.47 | 1.78 | 0.78 | 0.65 | 3.04 | 1.33 | 1.11 |
| (μmol/10⁹ CFUs/hr); | | | | | | | | | | | | | | | |
| (based on 3.10¹¹ CFUs/day dose) | | | | | | | | | | | | | | | |

| | | | | | |
|---|---|---|---|---|---|
| Combined BCAA target degradation | 1.14 | 1.43 | 2.23 | 3.21 | 5.48 |
| rate (mol/10⁹ CFUs/hr) | | | | | |

In one embodiment, the target degradation rates of branched chain amino acids from food intake in breastfed infants and adults is as indicated in the charts, below.

The leucine consumption kinetics and dosing are set forth herein. Food intake is based on adult recommended daily allowance of 40 mg/kg/day. MSUD patients are primarily children with restricted protein intake.

In another embodiment, the disorder involving the catabolism of a branched chain amino acid is a disorder caused by the activation of mTor (mammalian target of rapamycin). mTor is a senne-threonine kinase and has been implicated in a wide range of biological processes including transcription, translation, autophagy, actin organization and ribosome biogenesis, cell growth, cell proliferation, cell motility, and survival. mTOR exists in two complexes, mTORC1 and mTORC2. mTORC1 contains the raptor subunit and mTORC2 contains rictor. These complexes are differentially regulated, and have distinct substrate specificities and rapamycin sensitivity. For example, mTORCl phosphorylates S6 kinase (S6K) and 4EBP1, promoting increased translation and ribosome biogenesis to facilitate cell growth and cell cycle progression. S6K also acts in a feedback pathway to attenuate PI3K/Akt activation. mTORC2 is generally insensitive to rapamycin and is thought to modulate growth factor signaling by phosphorylating the C-terminal hydrophobic motif of some AGC kinases, such as Akt.

It is known in the art that mTor activation is caused by branched chain amino acids or alpha keto acids in the subject (see, for example, Harlan et al., Cell Metabolism, 17:599-606, 2013). Specifically, activation of mTorC1 (mTor complex 1) is caused by leucine (see Han et al., Cell, 149:410-424, 2012 and Lynch, J. Nutr., 131(3):861S-865S, 2001). Thus, in one embodiment, the disclosure provides methods of treating disorders involving the catabolism of leucine, caused by the activation of mTor by leucine in the subject. In one embodiment, the leucine levels in the subject are normal, and lowering leucine levels in the subject leads to the decreased activity of mTor and, thus, treatment of the disease. In another embodiment, the leucine levels in the subject are increased, and lowering leucine levels in the subject leads to the decreased activity of mTor and, thus, treatment of the disease. In one embodiment, the activation of mTor is increased as compared to the normal level of activation of mTor in a healthy subject, and lowering leucine levels in the subject leads to the decreased activation of mTor and, thus, treatment of the disease. In one embodiment, the level of activity of mTor is increased as compared to the normal level of activity of mTor in a healthy subject, and lowering leucine levels in the subject leads to the decreased activity of mTor and, thus, treatment of the disease. In another embodiment, the expression of mTor is increased as compared to the normal level of expression of mTor in a healthy subject, and lowering leucine levels in the subject leads to the decreased activity of mTor and, thus, treatment of the disease. In one embodiment, the activation of mTor is an abnormal activation of mTor.

Diseases caused by the activation of mTor are known in the art. See, for example, Laplante and Sabatini, Cell, 149(2):74-293, 2012. As used herein, the term “disease caused by the activation of mTor” includes cancer, obesity, type 2 diabetes, neurodegeneration, autism, Alzheimer's disease, Lymphangioleiomyomatosis (LAM), transplant rejection, glycogen storage disease, obesity, tuberous sclerosis, hypertension, cardiovascular disease, hypothalamic activation, musculoskeletal disease, Parkinson's disease, Huntington's disease, psoriasis, rheumatoid arthritis, lupus, multiple sclerosis, Leigh's syndrome, and Friedrich's ataxia.

In another aspect, the disclosure provides methods for decreasing the plasma level of at least one branched chain amino acid or branched chain α-keto acid in a subject by administering a pharmaceutical composition comprising a bacterial cell disclosed herein to the subject, thereby decreasing the plasma level of the at least one branched chain amino acid or branched chain alpha-keto acid or other BCAA metabolite in the subject. In one embodiment, the subject has a disease or disorder involving the catabolism of a branched chain amino acid. In one embodiment, the disorder involving the catabolism of a branched chain amino acid is a metabolic disorder involving the abnormal catabolism of a branched chain amino acid. In another embodiment, the disorder involving the catabolism of a branched chain amino acid is a disorder caused by the activation of mTor. In one embodiment, the disease or disorder is a maple syrup urine disorder (MSUD). In one embodiment, the branched chain amino acid is leucine, isoleucine, or valine. In one embodiment, the branched chain amino acid is leucine. In another embodiment, the branched chain amino acid is isoleucine. In another embodiment, the branched chain amino acid is valine. In another embodiment, the branched chain α-keto acid is α-ketoisocaproic acid (KIC). In another embodiment, the branched chain α-keto acid is α-ketoisovaleric acid (KIV). In another embodiment, the branched chain α-keto acid is α-keto-β-methylvaleric acid (KMV).

In some embodiments, the disclosure provides methods for reducing, ameliorating, or eliminating one or more symptom(s) associated with these diseases, including but not limited to neurological deficits, mental retardation, brain damage, brain oedema, blindness, branched chain α-keto acid acidosis, myelinization failure, hyperammonaemia, coma, developmental delay, neurological impairment, failure to thrive, ketoacidosis, seizure, ataxia, neurodegeneration, hypotonia, lactic acidosis, recurrent myoglobinuria, and/or liver failure. In some embodiments, the disease is secondary to other conditions, e.g., liver disease.

In certain embodiments, the bacterial cells disclosed herein are capable of catabolizing branched chain amino acid(s), e.g., leucine, in the diet of the subject in order to treat a disease associated with catabolism of a branched chain amino acid, e.g., MSUD. In these embodiments, the bacterial cells are delivered simultaneously with dietary protein. In another embodiment, the bacterial cells are delivered simultaneously with phenylbutyrate. In another embodiment, the bacterial cells are delivered simultaneously with a thiamine supplement. In some embodiments, the bacterial cells and dietary protein are delivered after a period of fasting or leucine-restricted dieting. In these embodiments, a patient suffering from a disorder involving the catabolism of a branched chain amino acid, e.g., MSUD, may be able to resume a substantially normal diet, or a diet that is less restrictive than a leucine-free diet. In some embodiments, the bacterial cells may be capable of catabolizing leucine from additional sources, e.g., the blood, in order to treat a disease associated with the catabolism of a branched chain amino acid, e.g., MSUD. In these embodiments, the bacterial cells need not be delivered simultaneously with dietary protein, and a leucine gradient is generated, e.g., from blood to gut, and the recombinant bacteria catabolize the branched chain amino acid, e.g., leucine, and reduce plasma levels of the branched chain amino acid, e.g., leucine.

The method may comprise preparing a pharmaceutical composition with at least one genetically engineered species, strain, or subtype of bacteria described herein, and administering the pharmaceutical composition to a subject in a therapeutically effective amount. In some embodiments, the bacterial cells disclosed herein are administered orally, e.g., in a liquid suspension. In some embodiments, the bacterial cells disclosed herein are lyophilized in a gel cap and administered orally. In some embodiments, the bacterial cells disclosed herein are administered via a feeding tube or gastric shunt. In some embodiments, the bacterial cells disclosed herein are administered rectally, e.g., by enema. In some embodiments, the genetically engineered bacteria are administered topically, intraintestinally, intrajejunally, intraduodenally, intraileally, and/or intracolically.

In certain embodiments, the administering the pharmaceutical composition described herein reduces branched chain amino acid levels in a subject. In some embodiments, the methods of the present disclosure reduce the branched chain amino acid levels, e.g., leucine levels, in a subject by at least about 10%, 20%, 25%, 30%, 40%, 50%, 60%, 70%, 75%, 80%, 85%, 90%, 95%, or more. In another embodiment, the methods of the present disclosure reduce the branched chain amino acid levels, e.g., leucine levels, in a subject by at least two-fold, three-fold, four-fold, five-fold, six-fold, seven-fold, eight-fold, nine-fold, or ten-fold. In some embodiments, reduction is measured by comparing the branched chain amino acid level in a subject before and after administration of the pharmaceutical composition. In one embodiment, the branched chain amino acid level is reduced in the gut of the subject. In another embodiment, the branched chain amino acid level is reduced in the blood of the subject. In another embodiment, the branched chain amino acid level is reduced in the plasma of the subject. In another embodiment, the branched chain amino acid level is reduced in the brain of the subject.

In one embodiment, the pharmaceutical composition described herein is administered to reduce branched chain amino acid levels in a subject to normal levels. In another embodiment, the pharmaceutical composition described herein is administered to reduce branched chain amino acid levels in a subject below normal levels to, for example, decrease the activation of mTor.

In certain embodiments, the pharmaceutical composition described herein is administered to reduce branched chain α-keto-acid levels in a subject. In some embodiments, the methods of the present disclosure reduce the branched chain α-keto-acid levels, in a subject by at least about 10%, 20%, 25%, 30%, 40%, 50%, 60%, 70%, 75%, 80%, 85%, 90%, 95%, or more as compared to levels in an untreated or control subject. In another embodiment, the methods of the present disclosure reduce the branched chain α-keto-acid levels, in a subject by at least two-fold, three-fold, four-fold, five-fold, six-fold, seven-fold, eight-fold, nine-fold, or ten-fold. In some embodiments, reduction is measured by comparing the branched chain α-keto-acid levels in a subject before and after administration of the pharmaceutical composition. In one embodiment, the branched chain α-keto-acid level is reduced in the gut of the subject. In another embodiment, the branched chain α-keto-acid level is reduced in the blood of the subject. In another embodiment, the branched chain α-keto-acid level is reduced in the plasma of the subject. In another embodiment, the branched chain α-keto-acid level is reduced in the brain of the subject.

In one embodiment, the pharmaceutical composition described herein is administered to reduce the branched chain α-keto-acid level in a subject to a normal level. In another embodiment, the pharmaceutical composition described herein is administered to reduce the branched chain α-keto-acid level in a subject below a normal level to, for example, decrease the activation of mTor.

In some embodiments, the method of treating the disorder involving the catabolism of a branched chain amino acid, e.g., MSUD, allows one or more symptoms of the condition or disorder to improve by at least about 10%, 20%, 30%, 40%, 50%, 60%, 70%, 80%, 90%, 95%, or more. In some embodiments, the method of treating the disorder involving the catabolism of a branched chain amino acid, e.g., MSUD, allows one or more symptoms of the condition or disorder to improve by at least about two-fold, three-fold, four-fold, five-fold, six-fold, seven-fold, eight-fold, nine-fold, or ten-fold.

Before, during, and after the administration of the pharmaceutical composition, branched chain amino acid levels, e.g., leucine levels, in the subject may be measured in a biological sample, such as blood, serum, plasma, urine, peritoneal fluid, cerebrospinal fluid, fecal matter, intestinal mucosal scrapings, a sample collected from a tissue, and/or a sample collected from the contents of one or more of the following: the stomach, duodenum, jejunum, ileum, cecum, colon, rectum, and anal canal. In some embodiments, the methods may include administration of the compositions disclosed herein to reduce levels of the branched chain amino acid, e.g., leucine. In some embodiments, the methods may include administration of the compositions disclosed herein to reduce the branched chain amino acid, e.g., leucine, to undetectable levels in a subject. In some embodiments, the methods may include administration of the compositions disclosed herein to reduce the branched chain amino acid, e.g., leucine, concentrations to undetectable levels, or to less than about 1%, 2%, 5%10%, 20%, 25%, 30%, 40%, 50%, 60%, 70%, 75%, 80%, 85%, 90%, or 95% of the subject's branched chain amino acid levels prior to treatment.

In some embodiments, the recombinant bacterial cells disclosed herein produce a branched chain amino acid catabolism enzyme, e.g., KivD, BCKD and/or other BCAA catabolism enzyme, BCAA transporter, BCAA binding protein, etc, under exogenous environmental conditions, such as the low-oxygen environment of the mammalian gut, to reduce levels of branched chain amino acids in the blood or plasma by at least about 1.5-fold, at least about 2-fold, at least about 3-fold, at least about 4-fold, at least about 5-fold, at least about 6-fold, at least about 7-fold, at least about 8-fold, at least about 9-fold, at least about 10-fold, at least about 15-fold, at least about 20-fold, at least about 30-fold, at least about 40-fold, or at least about 50-fold as compared to unmodified bacteria of the same subtype under the same conditions.

In one embodiment, the bacteria disclosed herein reduce plasma levels of the branched chain amino acid, e.g., leucine, will be reduced to less than 4 mg/dL. In one embodiment, the bacteria disclosed herein reduce plasma levels of the branched chain amino acid, e.g., leucine, will be reduced to less than 3.9 mg/dL. In one embodiment, the bacteria disclosed herein reduce plasma levels of the branched chain amino acid, e.g., leucine, will be reduced to less than 3.8 mg/dL, 3.7 mg/dL, 3.6 mg/dL, 3.5 mg/dL, 3.4 mg/dL, 3.3 mg/dL, 3.2 mg/dL, 3.1 mg/dL, 3.0 mg/dL, 2.9 mg/dL, 2.8 mg/dL, 2.7 mg/dL, 2.6 mg/dL, 2.5 mg/dL, 2.0 mg/dL, 1.75 mg/dL, 1.5 mg/dL, 1.0 mg/dL, or 0.5 mg/dL.

In one embodiment, the subject has plasma levels of at least 4 mg/dL prior to administration of the pharmaceutical composition disclosed herein. In another embodiment, the subject has plasma levels of at least 4.1 mg/dL, 4.2 mg/dL, 4.3 mg/dL, 4.4 mg/dL, 4.5 mg/dL, 4.75 mg/dL, 5.0 mg/dL, 5.5 mg/dL, 6 mg/dL, 7 mg/dL, 8 mg/dL, 9 mg/dL, or 10 mg/dL prior to administration of the pharmaceutical composition disclosed herein.

Certain unmodified bacteria will not have appreciable levels of branched chain amino acid, e.g., leucine, processing. In embodiments using genetically modified forms of these bacteria, processing of branched chain amino acids, e.g., leucine, will be appreciable under exogenous environmental conditions.

Branched chain amino acid levels, e.g., leucine levels, may be measured by methods known in the art, e.g., blood sampling and mass spectrometry. In some embodiments, branched chain amino acid catabolism enzyme expression is measured by methods known in the art. In another embodiment, branched chain amino acid catabolism enzyme activity is measured by methods known in the art to assess BCAA activity.

In certain embodiments, the recombinant bacteria are E. coli Nissle. The recombinant bacteria may be destroyed, e.g., by defense factors in the gut or blood serum (Sonnenborn et al., 2009) or by activation of a kill switch, several hours or days after administration. Thus, the pharmaceutical composition comprising the recombinant bacteria may be re-administered at a therapeutically effective dose and frequency. In alternate embodiments, the recombinant bacteria are not destroyed within hours or days after administration and may propagate and colonize the gut.

In one embodiments, the bacterial cells disclosed herein are administered to a subject once daily. In another embodiment, the bacterial cells disclosed herein are administered to a subject twice daily. In another embodiment, the bacterial cells disclosed herein are administered to a subject in combination with a meal. In another embodiment, the bacterial cells disclosed herein are administered to a subject prior to a meal. In another embodiment, the bacterial cells disclosed herein are administered to a subject after a meal. The dosage of the pharmaceutical composition and the frequency of administration may be selected based on the severity of the symptoms and the progression of the disease. The appropriate therapeutically effective dose and/or frequency of administration can be selected by a treating clinician.

The methods disclosed herein may comprise administration of a composition disclosed herein alone or in combination with one or more additional therapies, e.g., the phenylbutyrate, thiamine supplementation, and/or a low-branched chain amino acid, e.g., a low-leucine, diet. An important consideration in the selection of the one or more additional therapeutic agents is that the agent(s) should be compatible with the bacteria disclosed herein, e.g., the agent(s) must not interfere with or kill the bacteria. In some embodiments, the genetically engineered bacteria are administered in combination with a low protein diet. In some embodiments, administration of the genetically engineered bacteria provides increased tolerance, so that the patient can consume more protein.

The methods disclosed herein may further comprise isolating a plasma sample from the subject prior to administration of a composition disclosed herein and determining the level of the branched chain amino acid, e.g., leucine, or branched chain alpha-keto-acid in the sample. In some embodiments, the methods disclosed herein may further comprise isolating a plasma sample from the subject after to administration of a composition disclosed herein and determining the level of the branched chain amino acid, e.g., leucine, or branched chain alpha-keto-acid in the sample.

In one embodiment, the methods disclosed herein further comprise comparing the level of the branched chain amino acid or branched chain α-keto-acid in the plasma sample from the subject after administration of a composition disclosed herein to the subject to the plasma sample from the subject before administration of a composition disclosed herein to the subject. In one embodiment, a reduced level of the branched chain amino acid or branched chain alpha-keto-acid in the plasma sample from the subject after administration of a composition disclosed herein indicates that the plasma levels of the branched chain amino acid or branched chain alpha-keto-acid are decreased, thereby treating the disorder involving the catabolism of the branched chain amino acid in the subject. In one embodiment, the plasma level of the branched chain amino acid or branched chain α-keto-acid is decreased at least 10%, 20%, 30%, 40$, 50%, 60%, 70%, 80%, 90%, or 100% in the sample after administration of the pharmaceutical composition as compared to the plasma level in the sample before administration of the pharmaceutical composition. In another embodiment, the plasma level of the branched chain amino acid or branched chain α-keto-acid is decreased at least two-fold, three-fold, four-fold, or five-fold in the sample after administration of the pharmaceutical composition as compared to the plasma level in the sample before administration of the pharmaceutical composition.

In one embodiment, the methods disclosed herein further comprise comparing the level of the branched chain amino acid or branched chain α-keto-acid in the plasma sample from the subject after administration of a composition disclosed herein to a control level of the branched chain amino acid or branched chain alpha-keto-acid. In one embodiment, the control level of the branched chain amino acid is 4 mg/dL. In one embodiment, the subject is considered treated if the level of branched chain amino acid, e.g., leucine, in the plasma sample from the subject after administration of the pharmaceutical composition disclosed herein, is less than 4 mg/dL. In one embodiment, the subject is considered treated if the level of branched chain amino acid, e.g., leucine, in the plasma sample from the subject after administration of the pharmaceutical composition disclosed herein is less than 3.9, 3.8, 3.7, 3.6, 3.5, 3.4, 3.3, 3.2, 3.1, 3.0, 2.5, 2.0, 1.5, 1.0 or 0.5 mg/dL.

### EXEMPLIFICATION

The present disclosure is further illustrated by the following examples which should not be construed as limiting in any way. The contents of all cited references, including literature references, issued patents, and published patent applications, as cited throughout this application are hereby expressly incorporated herein by reference. It should further be understood that the contents of all the figures and tables attached hereto are also expressly incorporated herein by reference.

### Experimental Methods for all Examples

### 1-1. Design and Construction of LeuDH, KivD, and Adh Enzyme Libraries

Metagenomic Enzyme Discovery.

Machine-learning-based bioinformatics tools were used to identify enzyme candidates for each of the three desired activities (leucine dehydrogenase, 1.4.1.9; ketoisovalerate decarboxylase, 4.1.1.1; and alcohol dehydrogenase 1.1.1.1) in public sequence databases (SwissProt and TrEMBL, together known as UniProt). For LeuDH and Adh, sequence diversity was maximized using proprietary algorithms developed at Ginkgo. For KivD, a stratified sampling approach was used.

Rational Enzyme Design.

For LeuDH and Adh, molecular models of the enzyme-transition state complex were built using Rosetta software, and systematic mutations of the active site residues to each of the 20 amino acids were designed.

Library Synthesis.

DNA sequences for all LeuDH, KivD, and Adh enzymes were codon-optimized for expression in E. coli via Ginkgo-developed sequence design tools. Coding sequences were synthesized in an inducible E. coli expression vector under the control of the T7 promoter at Twist Bioscience.

### 1-2. High-Throughput In Vitro Enzyme Assays

Cell growth and enzyme preparation. Strains harboring library plasmids were transformed into an E. coli T7 expression host. 5 μL/well of thawed glycerol stocks were stamped into 500 μL/well of LB+100 ug/mL Carbenicillin (LB-Carb100) in half-height deepwell plates, which were sealed with AeraSeals. Samples were incubated at 37° C. and shaken at 1000 RPM in 80% humidity overnight. 50 μL/well of the resulting precultures were stamped into 450 μL/well of LB-Carb100+1 mM IPTG in half-height deepwell plates, which were sealed with AeraSeals. Samples were incubated at 30° C. and shaken at 1000 RPM in 80% humidity overnight. 250 μL/well of the resulting production cultures were stamped into deepwell plates containing 500 uL of phosphate buffered saline (PBS) and centrifuged for 10 minutes at 4000*G. Supernatant was removed and the resulting cell pellet was resuspended in 200 uL of BugBuster Protein Extraction Reagent+1 uL/mL purified Benzonase+1 uL/6 mL purified Lysozyme. Samples were incubated for 10 minutes at room temperature to generate the cell lysates used in in vitro enzyme assays.

LeuDH Activity Assay.

10 uL of lysate was transferred to a half-area flat-bottom plate containing 90 uL/well assay buffer (20 mM amino acid [L-Leucine, L-Valine, or L-Isoleucine], 200 mM Glycine, 200 mM KCl, 0.4 mM NAD, pH 10.5). Optical measurements were taken on a plate reader, with absorbance readings taken at 340 nm for 10 minutes. The resulting kinetic data was used to resolve the rate of NAD+ reduction, a proxy for LeuDH activity.

KivD Activity Assay.

10 uL of lysate was transferred to a half-area flat-bottom plate containing 90 uL/well assay buffer (100 mM PIPES-KOH, 100 mM Potassium glutamate, 1 mM Dithiothreitol, 0.4 mM NAD, 1.5 mM Thiamine pyrophosphate, 10 mM Magnesium glutamate, 20 mM ketoisocaproate (KIC), pH 7.5). A coupling enzyme was used to indirectly measure KivD activity on KIC. Optical absorbance measurements were taken over 10 minutes. The resulting kinetic data was used to determine KivD activity.

Adh Activity Assay.

10 uL of lysate was transferred to a half-area flat-bottom plate containing 90 uL/well assay buffer (50 mM MOPS buffer, 0.4 mM NADH, and 30 mM isovaleraldehyde, pH 7.0). Optical absorbance measurements were taken on a plate reader at 340 nm for 10 minutes. The resulting kinetic data was used to resolve the rate of NADH oxidation, a proxy for ADH activity.

LeuDH Selectivity Assay.

To measure LeuDH selectivity (specific deamination of L-Leu in the presence of L-Ile and L-Val), lysate was diluted four-fold in lysis buffer, and 10 uL/well of the newly diluted lysate was stamped into 90 uL/well of a modified assay buffer from above, featuring 0.5 mM of each amino acid (L-leucine, L-isoleucine, L-valine), 200 mM Glycine, 200 mM Potassium chloride, and 4 mM NAD. The reaction was quenched at different timepoints and submitted for LC-MS quantification of leucine, isoleucine, and valine.

### 1-3. Design and Construction of Optimized BCAA Pathways

Based on their performance in the in vitro assay, six (6) LeuDH enzymes, three (3) KivD enzymes, and three (3) Adh enzymes were selected to build candidate optimized BCAA operons. For translational control of each enzyme, we generated 3 ribosome binding sites of differing strengths. These RBS-gene pairs were then assembled into a partial combinatorial library in Synlogic's pathway plasmid. All pathway operons maintained the gene order of Synlogic's prototype pathway (LeuDH-KivD-Adh) and the final BmQ gene from the parent pathway.

### 1-4. High-Throughput BCAA Pathway Screening

Cell Preparation.

BCAA pathway plasmids were transformed into the SYN469 chassis (ΔilvC, ΔleuE, lacZ::Ptet-livKHMGF) and plated on LB agar plates supplemented with 100 μg/ml carbenicillin to select for strains harboring operon plasmids. Up to 6 colonies were picked from each transformation, grown in LB media supplemented with 100 μg/ml carbenicillin and cryopreserved in 20% glycerol prior to screening. For screening, strains were inoculated from cryostocks into FM1 medium supplemented with 100 μg/ml carbenicillin and grown overnight at 37° C. to generate pre-cultures. Pre-cultures were back-diluted in to FM1 media supplemented with 100 μg/ml carbenicillin and grown at 37° C. for approximately 2-2.5 hours to OD600 of ˜1.5-2. Cultures were then induced at a final concentration of 100 μg/ml aTc and allowed to grow for 2.5 hours at 37° C. Cultures were harvested and cryopreserved in Cryopreservation Buffer in 96-well plates.

Pathway Screening.

Cells were thawed on ice and the OD₆₀₀ value of each culture plate was measured. The OD₆₀₀ value was averaged across each 96-well plate. The average OD₆₀₀ for each plate was then used to determine the dilution necessary to achieve a final OD₆₀₀ of 2 in 0.8 ml Assay Buffer without leucine. Following this dilution, the samples were centrifuged, and the supernatant was discarded. The resulting cell pellets were resuspended in 0.8 ml Assay Buffer without leucine. An equal volume of 2× Assay Buffer (8 mM leucine) was added to the samples for a final volume of 1.6 ml at an OD600 value of 1 to initiate the reaction. Plates were sealed with foil heat seals and incubated at 37° C. without shaking for 4 hours. Samples were collected at 0, 2 and 4 h intervals. Upon collection, samples were centrifuged to remove cells and supernatant was stored at 4° C. prior to analysis by LC-MS, LC-MS-UV and/or GC-MS.

### 1-5. Metabolite Analysis

Leu, Val, and Ile were measured using LC-MS in a multiplexed QQQ method with linearity from 1 μg/ml-1000 μg/ml. Keto isocaproate (KIC) and isovaleraldehyde was detected by derivatizing assay samples with 2-hydrazinoquinoline. The KIC and isovaleraldehyde derivatives were detected using LC-MS-UV. IPA was measured in assay samples using GC-MS.

### 1-6. In Vitro Assay

Materials prepared from flasks or fermenters were thawed on ice, and cell density was measured by light absorption at 600 nm (OD₆₀₀). OD₆₀₀ of 1.0 was assumed to be equal to 10⁹ cells/mL in this method. A volume was calculated to target 1 mL of 2×10⁹ cells/mL cell resuspension and the cells were transferred into a 96-deep well plate and washed once with cold PBS. After centrifugation (4000 rpm, 4° C., 10 min), the PBS was discarded, and the cell pellets were then resuspended in 1 mL of 1×M9+50 mM MOPS+0.5% glucose (MMG) buffer. Eight hundred (800) μL of each sample was transferred into a new 96-deep well plate and 800 μL of MMG containing 16 mM leucine was added, mixed well by pipetting. A sample (200 μL) assigned as time zero was collected at this moment. The plate was then covered by a breathable membrane and moved to anaerobic chamber to incubate at 37° C. Samples were also collected at 2 h and 4 h during incubation in the anaerobic chamber. The samples were centrifuged for 10 min at 4000 rpm at 4° C. immediately after collection. 100 μL of the supernatant was transferred into a new 96-well plate and stored at −80° C. for future analysis. For different testing purposes, various amounts of branched chain amino acid(s) could be added to the reaction.

### 1-7. Flask Cell Preparation

The culture was inoculated into 4 mL of 2×YT medium containing proper antibiotics in a culture tube incubated at 37° C. with shaking (250 RPM) overnight. The next day, 500 μL of the overnight culture was transferred into 50 mL of FM1 medium containing proper antibiotics in a 125 mL baffled flask. The flasks were incubated for 2 hours at 37° C. with shaking at 250 RPM. Inducer was added after this 2-hour incubation for induction of pathway enzymes. The flasks remained shaking for 4 hours after induction. Cultures were then transferred into a 50 mL tube and centrifuged for 16 min at 5000 rpm at 4° C. The cell pellet was washed once with cold PBS and then resuspended into 800 μL of cold buffer (1×PBS+15% glycerol) and aliquoted into PCR tubes to store at −80° C.

### 1-8. Fermentation Cell Preparation

A frozen master cell bank scrape was used to inoculate 50 mL fermentation media in a 500 mL ultrayield flask, and grown to stationary phase overnight at 350 RPM, 37° C., resulting in a final density of 30-40 OD₆₀₀. AMBR250 vessels were prepared containing 200 mL (unless volume of feed was too high, then reduced to 100 mL) of media, with either 25 g/L glucose or 40 g/L glycerol and appropriate antibiotics, and equilibrated at 37° C., 1500 RPM, 100 mL/min air flow, and pH 7 (pH control by ammonium hydroxide). They were inoculated to approximately 0.15 OD₆₀₀ from the overnight seed culture and grown to varying OD₆₀₀ levels before addition of varying levels of inducing agent, tetracycline, and other supplementations (see experimental section below). Cultures were then grown to a final density target of 30 OD₆₀₀ where possible, maintaining dissolved oxygen levels at 60% using agitation up to 3500 RPM and then up to 80% substituted oxygen in the gas (100 mL/min fixed total gas flow) via cascade control. This material was spun down at 2500×g at 4° C. in 1 L centrifuge bottles, then resuspended in 100 mM K-phosphate pH 7.5 buffer with 15% (vol/vol) glycerol to ⅕th the volume, or approximately 1×10¹¹ cells/mL, and then frozen for storage at −80° C.

### 1-9. In Vitro Gastrointestinal Simulation of Strain Activity

To characterize the viability and metabolic activity of engineered bacterial strains, and to predict their function in vivo, an in vitro gastrointestinal simulation (IVS) model was designed to simulate key aspects of the human gastrointestinal tract, including oxygen concentration, gastric and pancreatic enzymes, and bile. The IVS model is comprised of a series of incubations in 96-well microplate format designed to simulate stomach, small intestine, and colonic conditions. The stomach and small intestinal portions of the IVS model were adapted from Minekus et al., 2014 (3). To study the activity of engineered strains designed for the treatment of MSUD, only the simulated stomach was considered.

Briefly, frozen aliquots of bacterial cells were first thawed at room temperature and resuspended in 0.077 M sodium bicarbonate buffer at 5.0×10⁹ CFU/mL. This solution was then mixed with equal parts of simulated gastric fluid (SGF; Minekus et al., 2014) containing 5 mM leucine and incubated for 2 hours at 37° C. with shaking in a Coy microaerobic chamber. The atmosphere within the microaerobic chamber was initially calibrated to 7% oxygen and gradually decreased to 2% oxygen over 2 hours. The cell density in SGF is 2.5×10⁹ CFU/mL. For testing the mixture of BCAA, 5 mM of each BCAA was added into the system.

To determine strain activity over time, SGF aliquots were collected periodically and centrifuged at 4000 rpm for 5 mins using a tabletop centrifuge. Cell free supernatants were collected and stored at −20° C. prior to mass spectrometry analysis of leucine and isopentanol concentration.

1-10. Efficacy of Engineered E. coli Nissle in iMSUD Mice

The iMSUD mouse model was generated as previously described (3) and breeder pairs sent to Charles River Laboratories' (CRL) Genetically Engineered Models (GEMS) for colony maintenance. Male and female mice aged 21-45 days were shipped to the Synlogic facility and allowed to acclimate for a minimum of 4 days on a mixture of branched chain amino acid free diet (Dyets, 510081) and 18% protein diet (Envigo, TD.2018.15) diet before study initiation. Mice were assigned to study groups according to date of birth and body weight and orally administered vehicle (15% glycerol in PBS), SYN1980 (8.9×10⁹ CFU) or SYN5941(8.9×10⁹ CFU) twice a day (BID) for a total of 4 days. Male and female mice were switched to a 70% high-protein diet and provided hydrogel (Envigo, TD.06723) on days 2 and 3, respectively, while receiving vehicle or bacteria BID. On the morning of day 5, mice received 66.7 mg peptone followed by vehicle or bacteria 30 minutes later. Two hours post bacteria dose, submandibular bleedings were performed, and plasma isolated for measurement of branch-chain amino acids (BCAA) leucine, isoleucine and valine levels by mass spectrometry.

1-11. Efficacy of Engineered E. coli Nissle in Healthy Non-Human Primates

Twelve monkeys were studied. Each study included 2 conditions (vehicle, SYN1980 or SYN5941). A total of 3 studies were performed and combined so each group would include n=10-12. Animals were allowed a 1 week break between dosing to allow strains to clear from the intestine between studies.

On the morning of the study, each monkey was removed from its cage and an oral gavage tube was inserted before 7.8 mL of vehicle (15% glycerol in PBS) or bacteria (1×1012 CFUs) was administered to each animal, followed by 5 mL of 0.36 M sodium bicarbonate, 14 mL of 500 g/L peptone and 5 mL water flush. Animals were then returned to their cages and blood was collected at 0, 0.5, 1, 2, 4 and 6 hours post dosing. Plasma was isolated and stored at −70° C. for analysis of BCAAs by mass spectrometry.

### 1-12. Analysis of Branched Chain Amino Acids by LC-MS-MS

Leucine, Isoleucine, and Valine were quantitated in bacterial supernatant, plasma, and urine by liquid chromatography coupled to tandem mass spectrometry (LC-MS/MS) using either an Ultimate 3000 UHPLC-TSQ Quantum or a Vanquish UHPLC-TSQ Altis system. Samples were extracted with 9 parts 2:1 acetonitrile: water containing 1 ug/mL leucine-d3 as an internal standard, vortexed, and centrifuged. Supernatants were diluted with 9 parts 0.1% formic acid and analyzed concurrently with standards processed as above from 0.8 to 1000 ug/mL. Samples were separated on a Phenominex Synergi 4 um Hydro-RP 80A, 75×2 mm using a 0.1% formic acid (A), 0.1% formic acid/acetonitrile (B) at 0.3 mL/min and 50° C. After a 2 uL injection and an initial 5% B hold from 0 to 0.5 minutes, analytes were gradient eluted from 5 to 90% B over 0.5 to 1.5 minutes followed by high organic wash and aqueous equilibration steps. Analytes were detected using Selected Reacting Monitoring (SRM) of compound specific collision induced fragments in electrospray positive ion mode (leucine: 132>86, isoleucine: 132>69, Valine: 118>72, leucine-d3: 135>89). SRM chromatograms were integrated and the unknown/internal standard peak area ratios were used to calculate concentrations against the standard curve.

### 1-13. Analysis of Isopentanol by GC-MS

Isopentanol was quantitated in bacterial supernatant by gas chromatography—mass spectrometry (GC-MS) using an Agilent 7890B-5977B system. Samples were extracted with 2 parts ethyl acetate containing 100 ug/mL isopentanol-d4 as an internal standard, vortexed, and centrifuged. The upper organic layer was removed and analyzed concurrently with standards from 4 to 1000 mg/mL processed as above. Samples or standards, 1 uL injection (split 10:1, inlet at 250 C), were separated on a RESTEK Rxi-35Sil MS, 30 m×0.25 mmID, 0.25 umdf at 1 mL/min helium. After a 5-minute hold at 35° C., a temperature gradient from 35 to 310° C. over 5 minutes was used to elute the analytes. Compound were detected by the Selected Ion Monitoring (SIM) of specific positive fragment ions produced by electron ionization (isopentanol: 55.1, isopentanol-d4: 59.1). SIM chromatograms were integrated, and the unknown/internal standard peak area ratios were used to calculate concentrations against the standard curve.

### Example 1. Generation of Various Recombinant Bacterial Strains

Table 10 lists all the bacterial strains used in this application. Escherichia coli Nissle 1917 (EcN), designated as SYN001 here, was purchased from the German Collection of Microorganisms and Cell Cultures (DSMZ Braunschweig, E. coli DSM 6601). The procedures of generating recombinant bacteria (including SYN094, SYN469 and SYN1980) starting from the wild type strain SYN001 were fully disclosed in Examples 1-4 and others of International Application No. PCT/US2016/032565, the entire contents of which are incorporated herein by reference. Existing vectors described below including the operon pSC101-Pₜₑₜ-leuDH-kivd-adh2-brnQ-ampR was used to generate SYN1980 and disclosed in Example 19 and others of International Application No. PCT/US2016/032565. For ease of reference, pSC101-Pₜₑₜ-leuDH-kivd-adh2-brnQ-ampR is referred herein as “Original Operon”. Recombinant strains corresponding to ID 5-14 of Table 10 were newly generated by the present application.

SYN001 with streptomycin resistance was obtained by streaking ˜10¹¹ cells of SYN001 on LB plate containing 300 ng/mL streptomycin and taking the single colony formed, designated as SYN094. The deletion of the ilvC and leuE gene was done by P1 transduction from the corresponding BW25113 strains and cured of antibiotic cassette afterwards. The correct colonies were verified by PCR and stored for future usage. For insertion of the lacZ::Ptet-livKHPMF cassette, the proper DNA fragment was generated by overlapping PCR and integrated into the chromosome by lambda red recombineering method. Colonies were selected on LB agar containing proper antibiotics (30 μg/mL chloramphenicol) and correct recombination events were verified by PCR. A verified colony was saved for future operations. Chromosomal insertions of extra brnQ cassette into the EcN genome were carried out using the well-characterized lambda red recombineering approach as well. For this insertion, (1) a pKD3- or pKD4-based plasmid containing ˜1,000 bp of 5′ and 3′ EcN genome homology for recombination was built, followed by (2) insertion of the target fragment into the plasmid by isothermal assembly (HiFI DNA Assembly Master Mix, NEB), (3) amplification of the insertion fragment from the plasmid by PCR (including EcN homology regions and antibiotic resistance cassette, Q5 High-Fidelity Master Mix, NEB), (4) recombineering of the insertion fragment by electroporation via pKD46 and subsequent pKD46 removal, and (5) the removal of antibiotic resistance cassettes via pCP20 and subsequent pCP20 removal.

A plasmid harboring the BCAA-consuming operon under the control of various promoters was either synthesized or PCR amplified from corresponding vectors containing an ampicillin resistance gene, a low copy number origin of replication (pSC101) and under the regulatory control of the Pₜₑₜ promoter. Various promoter systems such as P_{cl857}, P_{lacI/fnr}, P_{BAD} or P_{cmt} promoters were PCR-amplified from existing vectors and subcloned into target plasmids by Gibson assembly method. These plasmids were used to electroporate SYN001, SYN469 or other parental host strains with extra integrated brnQ cassette into the malE/K or malP/T locus. After the electroporation (Eporator, Eppendorf, 1.8-kV pulse, 1-mm gap length electro-cuvettes) the transformed cells were selected as colonies on LB agar (Sigma, L2897) containing proper antibiotics.

TABLE 10

| | | | | Induction |
|---|---|---|---|---|
| ID | Strain # | Genotype | Description* | Activity |
| 1 | SYN001 | Control bacterium | N/A | N/A |
| 2 | SYN094 | SYN001 with strep | Control bacterium with strep resistance | N/A |
| | | resistance | | |
| 3 | SYN469 | SYN001, ΔilvC, ΔleuE, | Bacterium with deleted BCAA | N/A |
| | | lacZ::Pₜₑₜ-livKHPMF | synthesis enzyme IlvC, leucine | |
| | | | exporter LeuE, and insertion of BCAA | |
| | | | importer LivKHPMF under the control | |
| | | | of Pₜₑₜ promoter | |
| 4 | SYN1980 | SYN001, ΔilvC, ΔleuE, | Bacterium with plasmid containing | aTc |
| | | lacZ::Pₜₑₜ-livKHPMF, | original BCAA consumption operon | |
| | | pSC101-Pₜₑₜ-leuDH- | under the control of Pₜₑₜ promoter | |
| | | kivd-adh2-brnQ-ampR | | |
| 5 | SYN5721- | SYN001, ΔilvC, ΔleuE, | Bacterium with plasmid containing | aTc |
| | SYN5744 | lacZ::Pₜₑₜ-livKHPMF, | optimized BCAA consumption operon | |
| | | pSC101-Pₜₑₜ-optimized | under the control of Ptet promoter | |
| | | operon-brnQ-ampR | | |
| 6 | SYN5941- | SYN001, pSC101-Pₜₑₜ- | Bacterium with plasmid containing | aTc |
| | SYN5943 | optimized operon- | optimized BCAA consumption operon | |
| | | brnQ-ampR | under the control of Ptet promoter | |
| 7 | SYN5944- | SYN001, ΔilvC, ΔleuE, | Bacterium with plasmid containing | aTc, |
| | SYN5946 | lacZ::Pₜₑₜ-livKHPMF, | optimized BCAA consumption operon | anaerobic, |
| | | malP/T::P_{lacI/fnr}-brnQ, | under the control of Ptet promoter and | IPTG |
| | | pSC101-Pₜₑₜ-optimized | extra integrated BCAA importer BrnQ | |
| | | operon-brnQ-ampR | under the control of P_{lacI/fnr} promoter | |
| 8 | SYN5947- | SYN001, ΔilvC, ΔleuE, | Bacterium with plasmid containing | aTc, |
| | SYN5949 | lacZ::Pₜₑₜ-livKHPMF, | optimized BCAA consumption operon | cumate |
| | | malE/K::P_{cmt}-brnQ, | under the control of Ptet promoter and | |
| | | pSC101-Pₜₑₜ-optmized | extra integrated BCAA importer BrnQ | |
| | | operon-brnQ-ampR | under the control of P_{cmt} promoter | |
| 9 | SYN5950- | SYN001, ΔilvC, ΔleuE, | Bacterium with plasmid containing | aTc, |
| | SYN5952 | lacZ::Pₜₑₜ-livKHPMF, | optimized BCAA consumption operon | cumate |
| | | malE/K::P_{cmt}-brnQ, | under the control of Ptet promoter and | |
| | | malP/T::P_{cmt}-brnQ, | two extra integrated BCAA importer | |
| | | pSC101-Pₜₑₜ-optimized | BrnQ under the control of P_{cmt} promoter | |
| | | operon-brnQ-ampR | | |
| 10 | SYN6034 | SYN001, pSC101-Pₜₑₜ- | Bacterium with plasmid containing | aTc |
| | | leuDH-kivd-adh2- | original BCAA consumption operon | |
| | | brnQ-ampR | under the control of Pₜₑₜ promoter | |
| 11 | SYN6035 | SYN001, pSC101-P_{cmt}- | Bacterium with plasmid containing | cumate |
| | | optimized operon- | optimized BCAA consumption operon | |
| | | brnQ-ampR | under the control of P_{cmt} promoter | |
| 12 | SYN6036 | SYN001, pSC101- | Bacterium with plasmid containing | IPTG, |
| | | P_{lacI/fnr}-optimized | optimized BCAA consumption operon | anaerobic |
| | | operon-brnQ-ampR | under the control of P_{lacI/fnr} promoter | |
| 13 | SYN6037 | SYN001, pSC101- | Bacterium with plasmid containing | heat |
| | | P_{cI857}-optimized | optimized BCAA consumption operon | |
| | | operon-brnQ-ampR | under the control of P_{cI857} promoter | |
| 14 | SYN6038 | SYN001, pSC101- | Bacterium with plasmid containing | arabinose |
| | | P_{BAD}-optimized operon- | optimized BCAA consumption operon | |
| | | brnQ-ampR | under the control of P_{BAD} promoter | |

*Original BCAA consumption operon refers to pSC101-Pₜₑₜ-leuDH-kivd-adh2-brnQ-ampR. Optimized BCAA consumption operon refers to the operons generated in the present application.

### Example 2. Design and Synthesis of LeuDH, KivD, and Adh Enzyme Libraries

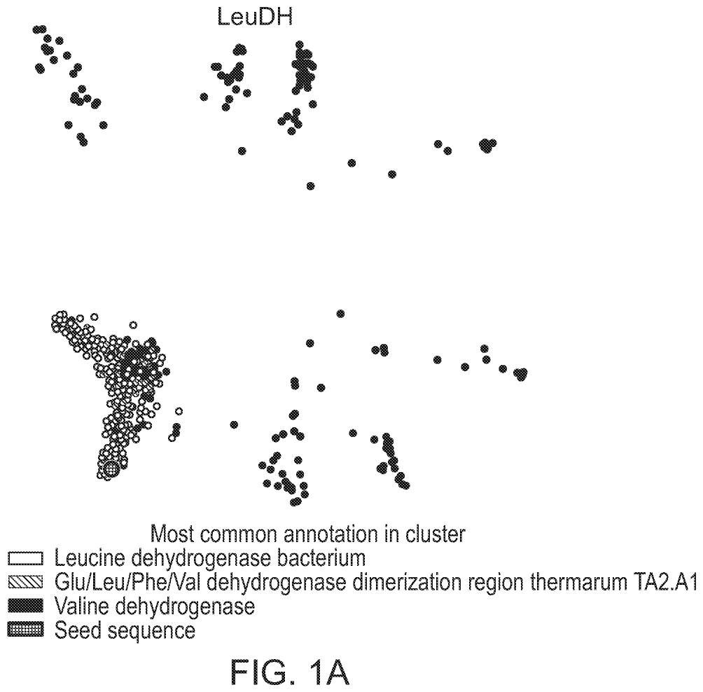

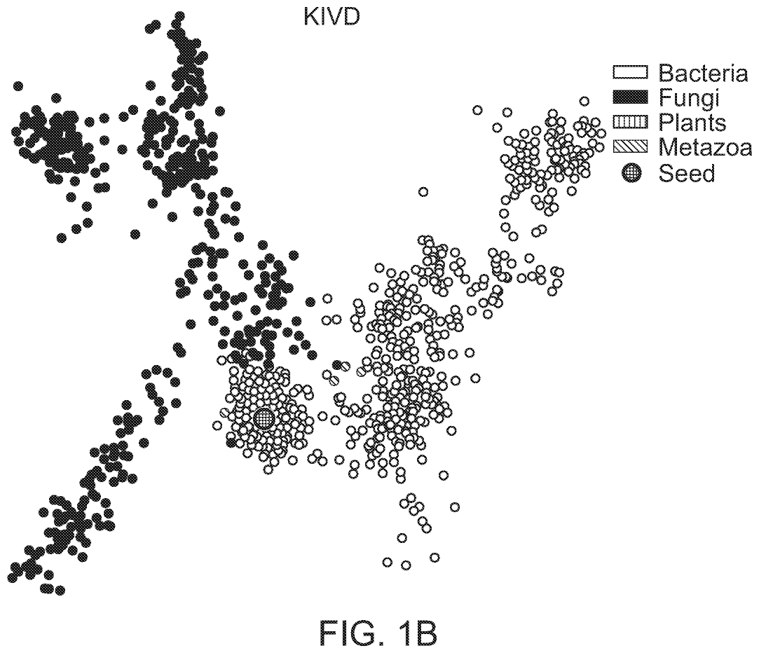

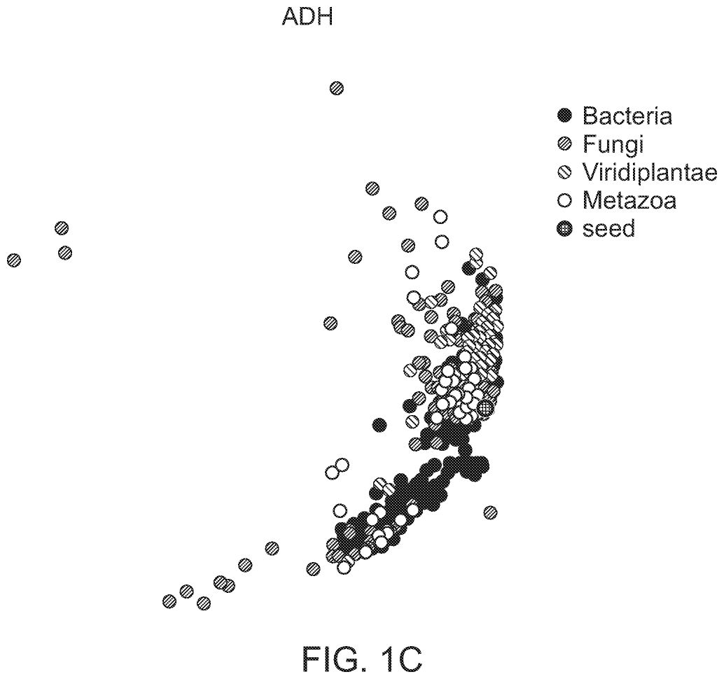

The metagenomic enzyme discovery and rational enzyme design described above were used to design a library for each enzyme family (LeuDH, KivD, and Adh) to identify LeuDH, KivD, and Adh pathway enzymes (i) with increased activity than the parent pathway enzymes from SYN1980: Bacillus cereus LeuDH, Lactococcus lactis KivD, and Saccharomyces cerevisiae ADH2, respectively; and (ii) with increased specificity for leucine (Leu) relative to valine (Val) and isoleucine (Ile). Table 11 summarizes the enzyme library composition. For each enzyme, a metagenomic library of >1000 enzymes was designed to sample the full metagenomic sequence space available in sequence databases and depicted in FIG. 1. For LeuDH and Adh, additional designs were constructed using rational design based on available structural data for the B. cereus LeuDH and S. cerevisiae Adh enzymes. Enzyme sequences for all libraries were optimized for expression in E. coli, synthesized in an inducible E. coli expression vector, and transformed into E. coli for high throughput screening.

TABLE 11

| | | | | | | Total |
|---|---|---|---|---|---|---|
| Library | Bacteria | Fungi | Animal | Plant | Rational | Designs |
| LeuDH | 1129 | 11 | 23 | 12 | 270 | 1445 |
| KivD | 783 | 508 | 1 | 4 | 0 | 1296 |
| Adh | 654 | 273 | 128 | 122 | 140 | 1317 |

### Example 3. Characterization of Pathway Enzyme Libraries

To screen the 3×˜1300-member enzyme libraries, high-throughput methods were developed to screen for LeuDH, KivD, and Adh enzyme activities in E. coli cell lysates. In brief, strains were cultivated in 96-deepwell plates to induce protein production, with positive and negative control strains included in each plate. Cells were lysed, and enzyme activity was measured in cell lysates using enzyme-specific spectrophotometric assays described above in the method section. Enzyme assays were executed on the fully automated robotic workcell known as ATLAS.

For each enzyme family, the full library (˜1300 members each) was measured in biological duplicate, and hits were selected based on the highest activity in a single replicate for each enzyme. This method of hit selection reduced the impact of false negative activity measurements for a given replicate. For each library, 55-220 enzymes were selected as primary hits. To increase the confidence in the hit selected from the primary screen and establish enzyme ranking based on activity, the primary hits were re-screened in a secondary screen with additional replication (8 technical replicates).

Leucine Dehydrogenase (leuDH)

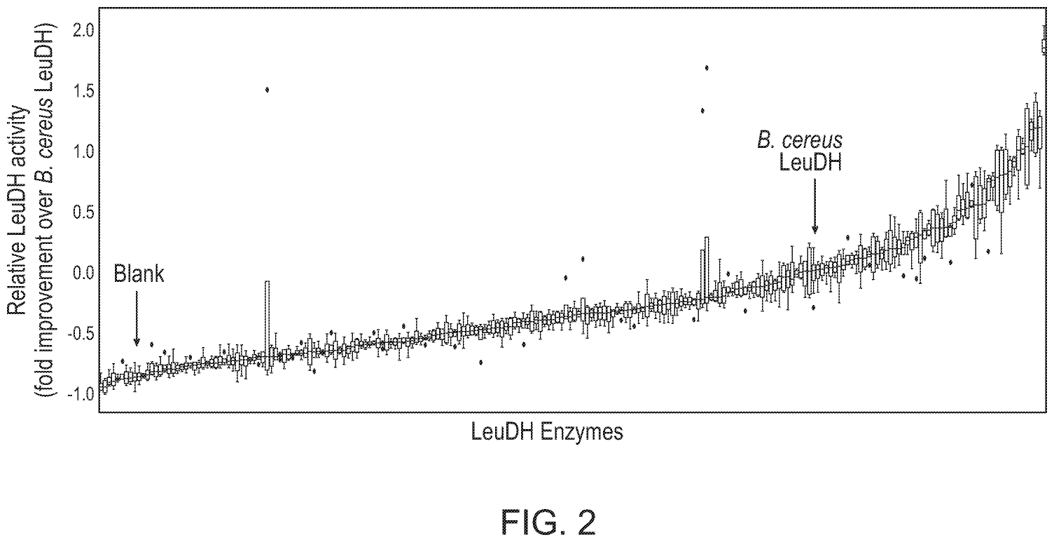

A total of 1378 LeuDH enzymes were first screened for the ability to deaminate leucine. An initial round of screening identified 220 enzymes with activity similar to or better than the parent LeuDH enzyme from B. subtilis. These primary hits were further analyzed in a secondary screen for validation of LeuDH enzyme hits (FIG. 2). In the secondary screen, LeuDH enzymes with up to a 1.8-fold increase in LeuDH activity on Leu were validated.

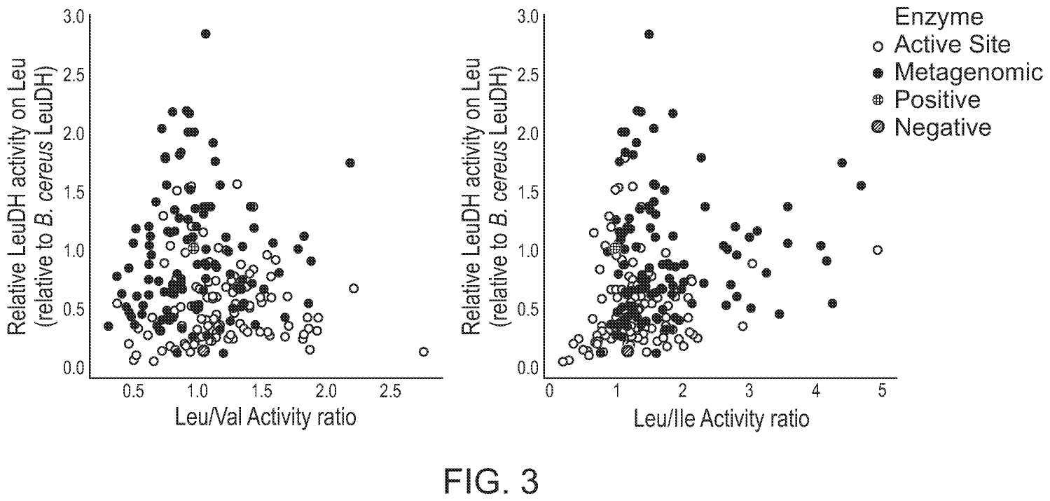

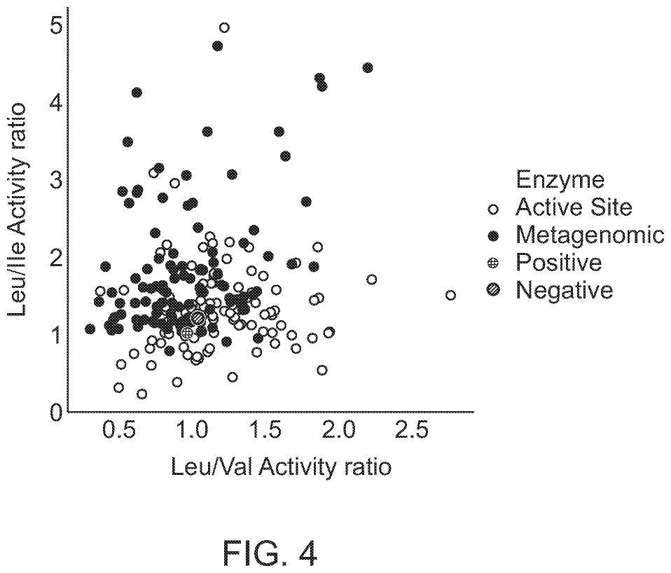

To determine if any of the primary LeuDH hits exhibited increased specificity for Leu over Val and Ile, all 220 primary hits were also screened for activity on Val and Ile. Specificity was measured as the ratio of activity on Leu to the activity on Ile or Val. As shown in FIG. 3, enzymes exhibited up to ˜2.7-fold preference for Leu over Val, and up to a 5-fold preference for Leu over Ile. By comparing specificity for Leu/Ile to Leu/Val, hits were identified with increased specificity for Leu relative to both Leu and Val (FIG. 4), as compared with the control B. cereus LeuDH from the prototype pathway SYN1980.

Ketoisovalerate Decarboxylase (kivD)

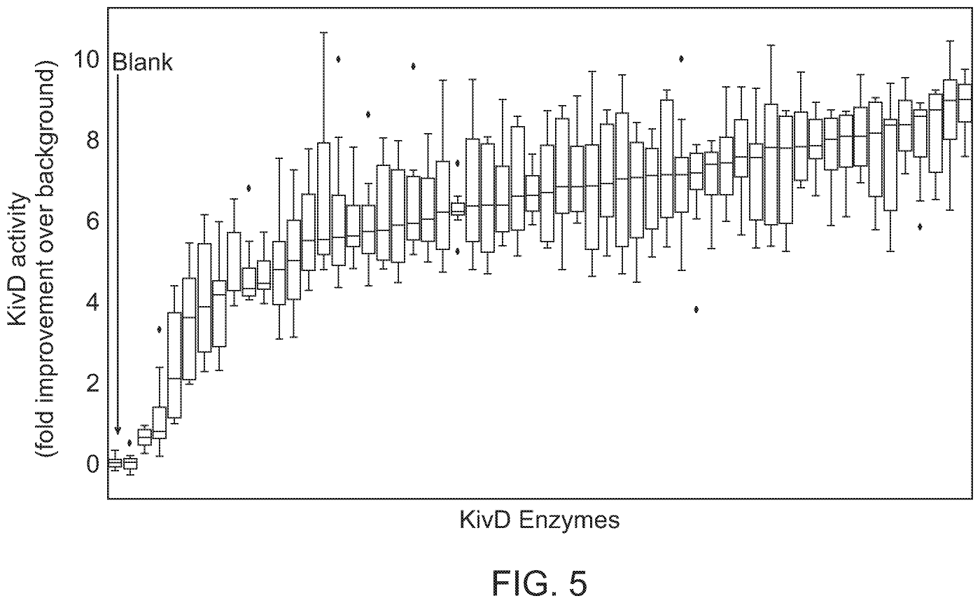

A total of 1248 KivD enzymes were screened for the decarboxylase activity on ketoisocaproate. An initial round of screening identified 55 enzymes with higher activity than the control KivD enzyme from S. aureus, which did not exhibit activity above greater than the background lysate decarboxylase activity in this assay and was equated to the non-zero measurable background activity. These primary KivD hits were further analyzed in a secondary screen (FIG. 5). In the secondary screen, more than 40 KivD enzymes were identified with at least 6- to 8-fold increase in KivD activity relative to the background lysate activity.

Alcohol Dehydrogenase (adh)

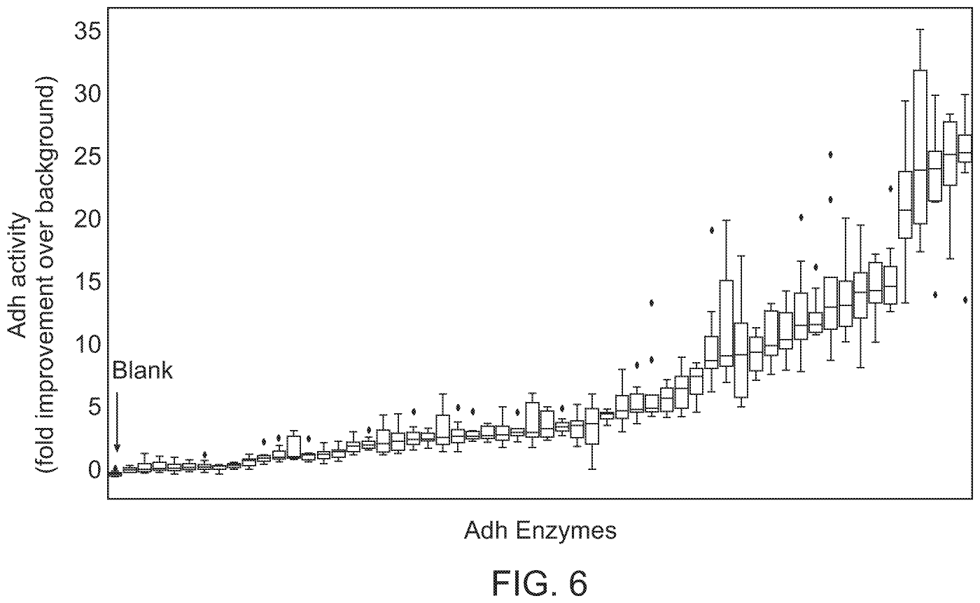

A total of 1215 Adh enzymes were screened for the ability to reduce isovaleraldehyde to isopentanol. An initial round of screening identified 55 enzymes with higher activity than the parent ADH2 enzyme from S. cerevisiae, which did not exhibit activity greater than the background lysate alcohol dehydrogenase activity in this assay and was equated to the non-zero measurable background activity. Because activity of the ADH2 enzyme for S. cerevisiae was indistinguishable from the background activity of the lysate, an Equus caballus Adh was used as a positive control for the screen. These primary Adh hits were further analyzed in a secondary screen (FIG. 6). In the secondary screen, 5 Adh enzymes were identified with at least 20-fold increase in Adh activity relative to the background lysate activity.

### Example 4. Selectivity of Top LeuDH Candidate Enzymes for Optimized BCAA Consumption

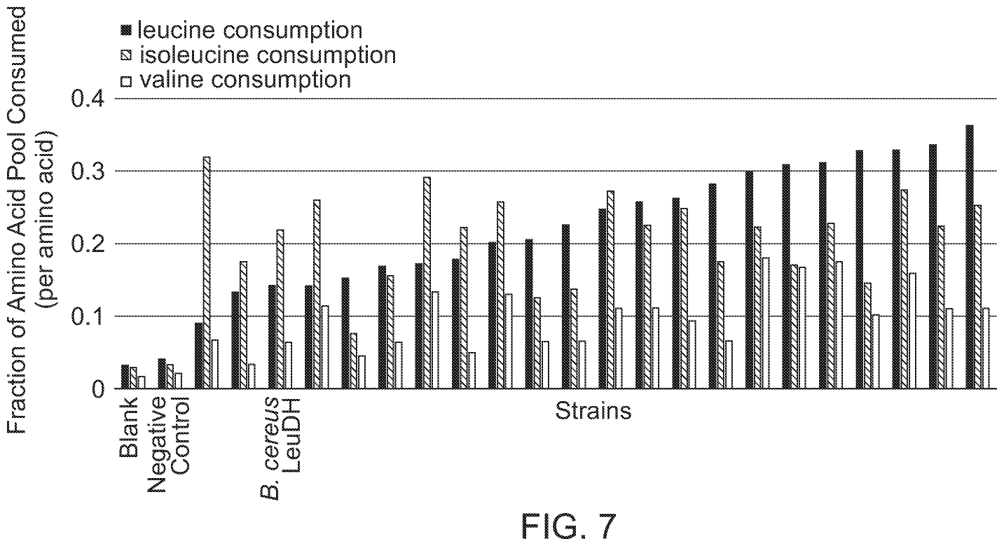

In an attempt to better predict the performance of the top LeuDH hits with regard to mixed-substrates pools, the selectivity of LeuDH enzymes for Leu (i.e. the preference of LeuDH for Leu when Leu, Val, and Ile are all present in the reaction mixture) were measured. A total of 21 LeuDH enzymes were screened in cell lysate assays similar to the HTP screen, except that the reaction mixture contained Leu, Val, and Ile at 1:1:1 molar ratio. Rate of Leu, Val, and Ile disappearance was monitored in the reaction mixture. FIG. 7 reports consumption of Leu, Ile, and Val within the reaction mixture for each LeuDH enzyme. At least 10 LeuDH enzymes showed improved preference for Leu over Val and Ile when compared to the parent B. subtilis LeuDH. For nearly all LeuDH enzymes, least preference was shown for valine.

### Example 5. Pathway Enzyme Hit Selection, Operon Assembly and Operon Screening for Optimized BCAA Consumption

### Materials and Methods

### Cell Preparation

Branched-chain amino acid (BCAA) pathway operon plasmids were transformed into E. coli Nissle strain 1917, which was purchased from the German Collection of Microorganisms and Cell Cultures (DSMZ Braunschweig, E. coli DSM 6601). Transformed cells were thawed on ice and cell density was measured by light absorption at 600 nm (OD600). OD600 of 1.0 was assumed to be equal to 109 cells/mL in this method. A volume was calculated to target 1 mL of 2×109 cells/mL cell resuspension, and the cells were transferred into a 96-deep well plate and washed once with cold PBS. After centrifugation (4000 rpm, 4° C., 10 min), the PBS was discarded, and the cell pellets were then resuspended in 1 mL of 1×M9+50 mM MOPS+0.5% glucose (MMG) buffer. Eight hundred (800) μL of each sample was transferred into a new 96-deep well plate and 800 μL of MMG containing 16 mM leucine was added, mixed well by pipetting. A sample (200 μL) assigned as time zero was collected at this moment. The plate was then covered by a breathable membrane and moved to an anaerobic chamber to incubate at 37° C. Samples were also collected at 2 hours and 4 hours during incubation in the anaerobic chamber. The samples were centrifuged for 10 minutes at 4000 rpm at 4° C. immediately after collection. 100 μL of the supernatant was transferred into a new 96-well plate and stored at −80° C. for future analysis.

### Leucine Activity Assay

Leucine was quantitated in bacterial supernatant by liquid chromatography coupled to tandem mass spectrometry (LC-MS/MS) using either an Ultimate 3000 UHPLC-TSQ Quantum or a Vanquish UHPLC-TSQ Altis system. Samples were extracted with 9 parts 2:1 acetonitrile:water containing 1 pug/mL leucine-d3 as an internal standard, vortexed, and centrifuged. Supernatants were diluted with 9 parts 0.1% formic acid and analyzed concurrently with standards processed as above from 0.8 to 1000 pug/mL. Samples were separated on a Phenominex Synergi 4 um Hydro-RP 80A, 75×2 mm using a 0.1% formic acid (A), 0.1% formic acid/acetonitrile (B) at 0.3 mL/min and 50 degrees C. After a 2 uμL injection and an initial 5% B hold from 0 to 0.5 minutes, analytes were gradient eluted from 5 to 90% B over 0.5 to 1.5 minutes followed by high organic wash and aqueous equilibration steps. Analytes were detected using Selected Reacting Monitoring (SRM) of compound specific collision induced fragments in electrospray positive ion mode (leucine: 132>86, isoleucine: leucine-d3: 135>89). SRM chromatograms were integrated, and the unknown/internal standard peak area ratios were used to calculate concentrations against the standard curve.

To improve the overall Leu consumption of the BCAA pathway, multiple enzymes were selected for each step that demonstrated superior performance relative to the B. cereus LeuDH enzyme. For LeuDH, 6 hits were selected based on two criteria: (1) enzyme activity on Leu and (2) specificity for Leu relative to Val and Ile. Because LeuDH selectivity analysis was conducted in parallel to operon assembly, the selectivity data set did not factor into LeuDH selection. For KivD and ADH, 3 hits were selected for each enzyme family based on in vitro enzyme activity. In total, 12 enzymes were advanced to the final operon design.

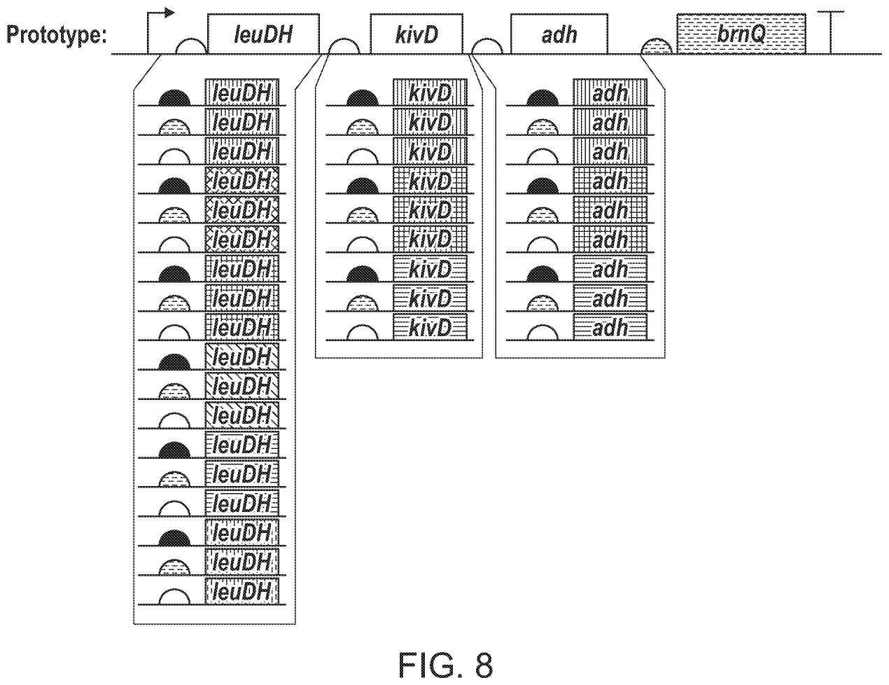

To balance the flux of the Leu-consuming pathway, enzyme identity and enzyme level for each enzyme in the Leu-consuming pathway were varied. Three (3) ribosome binding sites (RBSs) were selected for each pathway enzyme candidate. These RBS-enzyme pairs were then assembled into a partial combinatorial library of 462 pathways, and the gene order was held constant (LeuDH-KivD-Adh), as shown in FIG. 8. Pathways were synthesized and cloned into a MSUD pathway backbone encoding a branched chain amino acid transporter (BrnQ). The final pathways were composed of four-gene operons (LeuDH-KivD-Adh-BrnQ) in which the LeuDH, KivD, and Adh enzymes and their RBSs were varied while the BrnQ RBS and coding sequence is held constant.

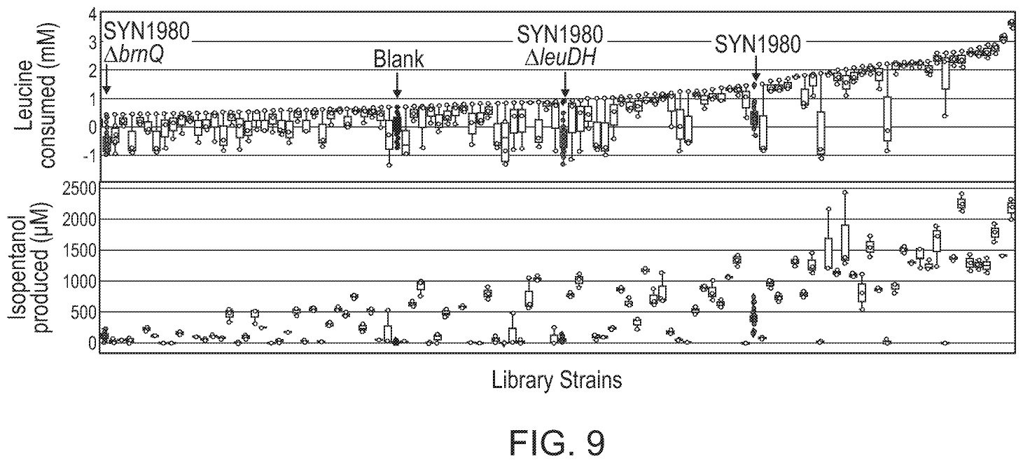

The operon library was screened in HTP to identify optimal pathway designs for Leu consumption in SYN469 (ΔilvC, ΔleuE, lacZ::Ptet-livKHMGF). Of the 462 operons designed, 383 were successfully synthesized and 254 were successfully transformed into SYN469 for screening. All pathway strains were cultured and screened for Leu consumption in 96-well plates. For plate-to-plate standardization, each plate included control strains which harbored the prototype pathway SYN1980 or versions lacking LeuDH (SYN1980 ΔLeuDH) or BrnQ (SYN1980 ΔBrnQ). As shown in FIG. 9, multiple pathways were identified that Leu consumption and isopentanol production each occurred at a faster rate than the prototype pathway SYN1980. Strains were ranked based on Leu consumption, and 24 strains were validated in the flask-based model described above in the method section. Analysis of the enzyme composition of the top 24 pathway operons showed that 4 of 6 LeuDHs and all 3 Adh enzymes were represented in the top 24 operons. However, 23 of the 24 operons contained the same KivD, supporting a correlation between this KivD enzyme and the overall pathway activity.

### Results

The top Leu consuming operons identified through HTP screening were transformed into E. coli Nissle 1917 (and labeled as strain 5941, 5942 and 5943) and compared to the prototype strain 1980. Strain 5941 contains the LeuDH enzyme of Cetobacterium ceti, the KivD enzyme of Erwinia iniecta, and the Adh enzyme of Alcanivoriax dieselolei. Strain 5942 has the LeuDH enzyme of Cetobacterium ceti, the KivD enzyme of Erwinia iniecta, and the Adh enzyme of Rhizobiales bacterium NRL2. Strain 5943 has LeuDH enzyme of Cetobacterium ceti, the KivD enzyme of Erwinia iniecta, and the Adh enzyme of Rhizobiales bacterium NRL2. The operons further contain BrnQ of E. coli. The prototype strain contains Bacillus cereus LeuDH, Lactococcus lactis KivD, Saccharomyces cerevisiae ADH2, as well as E. coli BrnQ.

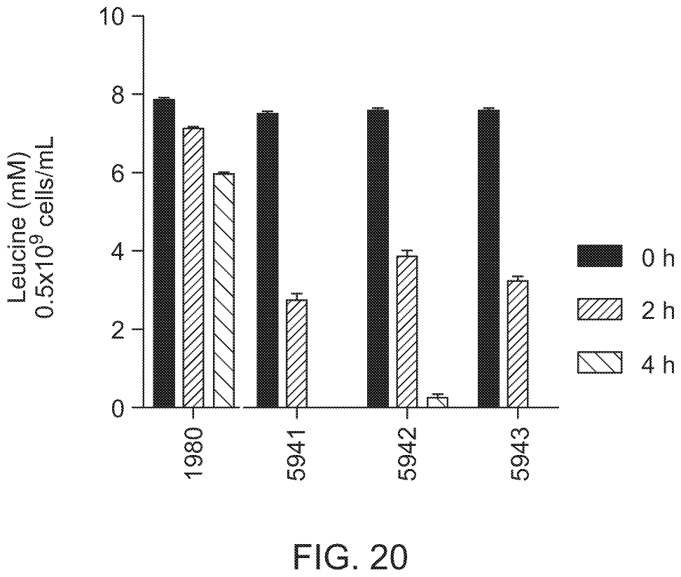

Samples from the top Leu consuming operons and the prototype strain were analyzed for Leu consumption (FIG. 20). The top Leu consuming operon-containing strains (5941, 5942 and 5943) were found to consume Leu at a significantly faster rate than the prototype strain (1980).

### Example 6. Metabolomics Analysis of Top Pathways for Optimized BCAA Consumption

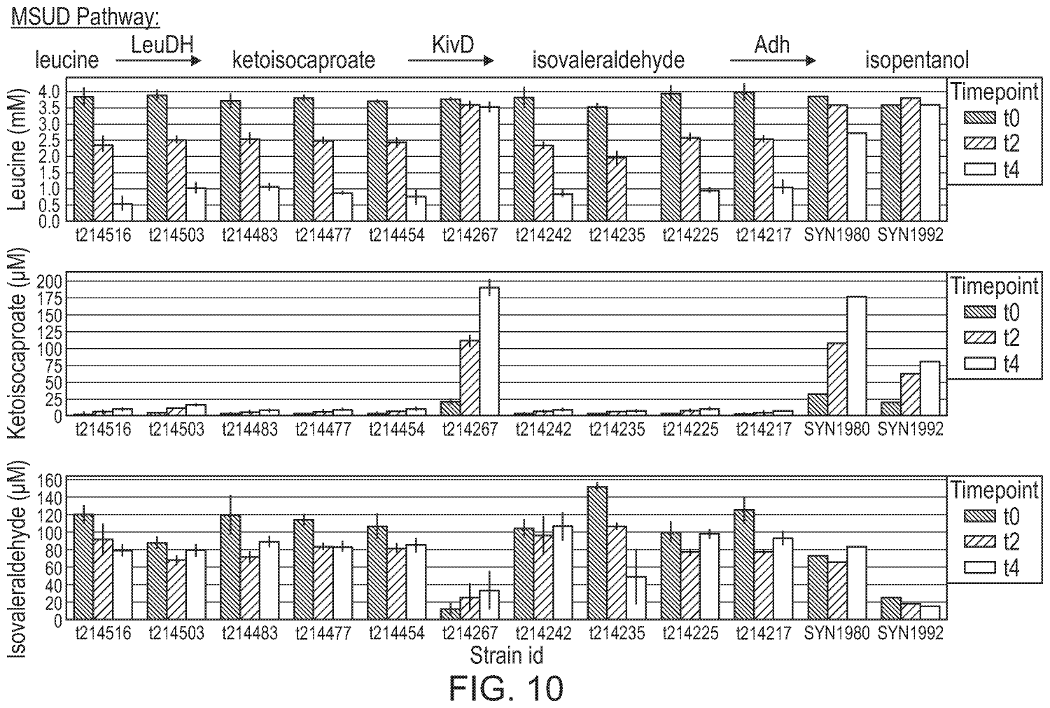

The top 10 Leu consuming strains identified through HTP screening described above in the method section shown in FIG. 9 were selected for targeted metabolomic analysis of pathway intermediates. In brief, strains harboring optimized or controlled plasmids were grown and induced in a flask-based model in biological triplicate. Cells were harvested, normalized individually by OD₆₀₀, and prepared for the Leu-consumption assay. The assay was initiated by addition of cells to the Leu assay buffer, and reactions were sampled over time and preserved for analysis. Samples were analyzed for Leu, ketoisocaproate (KIC), and isovaleraldehyde (FIG. 10). Isopentanol measurements were not collected for this experiment. Of the 10 selected pathways, 9 consumed Leu at a faster rate than the prototype strain (SYN1980) and the SYN1980 ΔBrnQ variant (i.e. SYN1992). The 9 better performing strains had drastically reduced levels of KIC when compared the control strains, indicating that a KivD bottleneck was relieved through the pathway optimization campaign, providing further evidence that KivD is a bottle neck in the overall pathway. Of the top 9 strains, t214235 exhibited the fastest rate of Leu consumption and a downward IVA trend over time.

### Example 7. In Vitro Activity Validation for BCAA Consuming Strains Generated from Host Strain SYN469

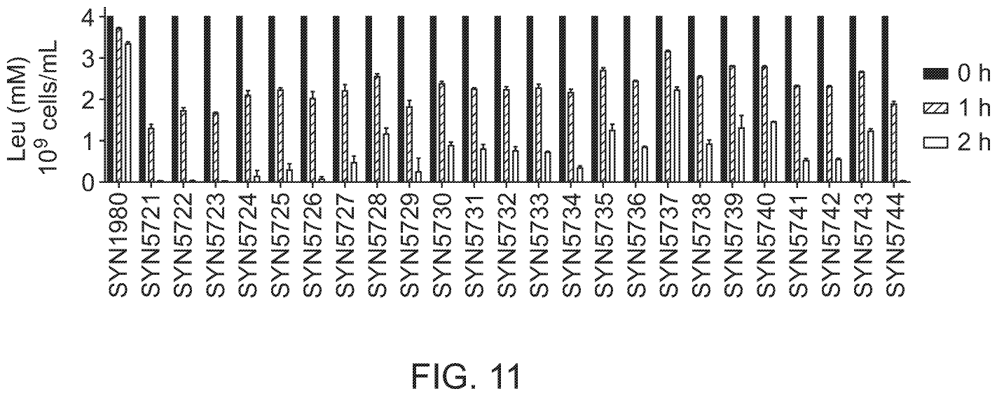

Plasmids containing optimized operons were transformed into SYN469, resulting in strains SYN5721 to SYN5744. The in vitro leucine consumption assay was performed to assess their activity using SYN1980 as a control. As shown in FIG. 11, all new strains demonstrated improved activity in vitro as compared to SYN1980. Top hits such as SYN5721, SYN5722 and SYN5723 were selected for further testing.

These selected strains were tested using BCAA mixture as substrates in vitro. The goal here was to understand the substrate specificity when all three branched chain amino acids were present simultaneously. The leucine:isoleucine:valine ratio was 2:1:1, corresponding to the ratio analyzed previously for in vivo GI content. Strains showed some specificity advantage towards leucine versus valine, but not isoleucine (Table 12).

TABLE 12

| | SYN- | SYN- | SYN- | SYN- | SYN- | SYN- |
|---|---|---|---|---|---|---|
| | 1980 | 5721 | 5722 | 5723 | 5725 | 5729 |
| Val | 0.27 | 0.49 | 0.46 | 0.59 | 0.64 | 0.40 |
| (2 mM) | | | | | | |
| Ile | 0.31 | 0.82 | 0.78 | 0.59 | 0.69 | 0.67 |
| (2 mM) | | | | | | |
| Leu | 0.59 | 1.51 | 1.43 | 1.40 | 1.18 | 1.25 |
| (4 mM) | | | | | | |
| BCAA | 1.17 | 2.82 | 2.66 | 2.58 | 2.51 | 2.32 |

### Example 8. Host Strain Screening Study and Promoter Screening Study to Screen for Optimized BCAA Consuming Strains

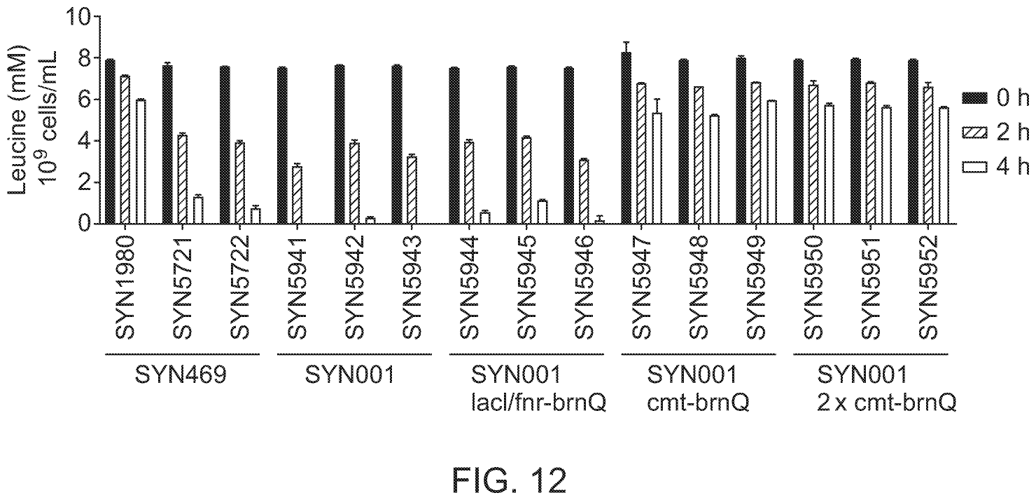

In addition to various modifications existed in SYN469 (ΔilvC, ΔleuE, lacZ::Ptet-livKHMGF), which was used for the construction of SYN1980 and SYN5721-SYN5744. A panel of new strains was built using the most active operons and different host strains. In these set of host strains, either modifications were removed such as using the wild-type strain SYN001 or extra modifications were introduced such as extra copy(s) of BCAA transporter BmQ. The in vitro leucine consumption activity was measured. SYN5941 demonstrated the highest leucine consumption rate under this test condition (FIG. 12).

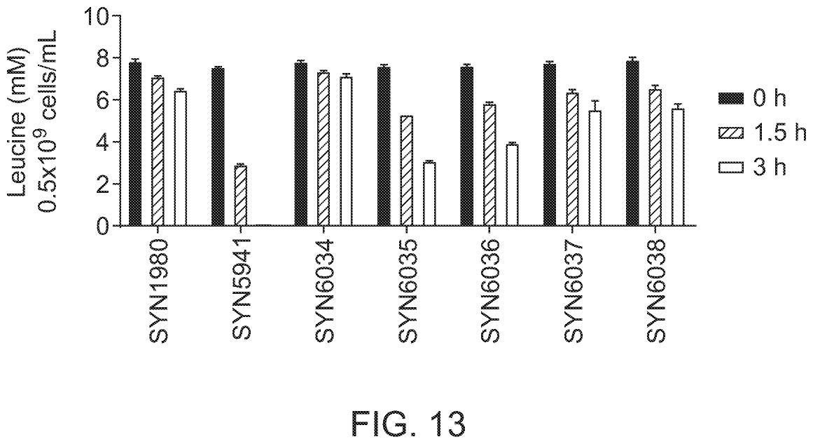

A panel of selected promoters were chosen to drive the optimized operon and tested for in vitro leucine consumption. The operon in SYN1980 was used as a control. The host strain was SYN001 for testing the new promoters. All the clinical candidate strain compatible promoters-FNRS, P_{cmt}, P_{cl857}, and P_{BAD}-showed reasonable in vitro leucine consumption activity, though lower than SYN5941 harvesting the Pₜₑₜ driven operon (FIG. 13 and Table 13).

TABLE 13

| | | | | | IPTG | Heat | |
|---|---|---|---|---|---|---|---|
| Inducer | aTc | aTc | aTc | Cumate | (fnr) | (37° C.) | arabinose |
| Final Strain # | SYN1980 | SYN6034 | SYN5941 | SYN6035 | SYN6036 | SYN6037 | SYN6038 |
| Activity (Leu) | 0.48 | 0.29 | 3.08 | 1.54 | 1.20 | 0.91 | 0.89 |
| (umol/10⁹ cells/h) | | | | | | | |
| Host | SYN469 | SYN001 | SYN001 | SYN001 | SYN001 | SYN001 | SYN001 |
| Operon source | 1980 | 1980 | 5721 | 5721 | 5721 | 5721 | 5721 |

### Example 9. In Vitro Activity Study for Optimized BCAA Consuming Strain SYN 5941 in a Fermentation Scale-up Process

Various parameters were tested such as, but not limited to, carbon source, feeding rate, temperature, time of induction and inducer concentration. Using SYN5941, FM1 media with glucose and 6× anhydrotetracycline (aTc) concentration (600 μg/mL) was chosen as the growth condition and cells were harvested 4 hours post induction from the fermenter for cryopreservation. A greater-than-ten-fold improvement in in vitro leucine consumption was observed using the optimized strain SYN5941 with newly developed fermentation process (Table 14). As shown in Table 15, SYN5941 exhibited a much higher leucine consumption rate than SYN1980.

TABLE 14

| | Carbon | Induction | aTc (1x = | | |
|---|---|---|---|---|---|
| Media | source | OD | 100 μg/mL) | Feed | Temp |
| FM1 | 25 g/L glucose | 1.5, 10, 20 | 0.1, 1, 3, 6, 10X | BCAAs (hi/lo rates) | Shift to 30° C. |
| | | | | | mid-induction |
| FM5 | None | 1.5, 10 | 1X | YE + glycerol, | NA |
| | | | | YE + glucose, | |
| | | | | BCAA + glycerol | |
| FM2 | 40 g/L glycerol, | 1.5, 10 | 1X | NA | Shift to 30° C. |
| | 25 g/L glucose | | | | mid-induction |
| FM4 | 25 g/L | 1.5 | 1X | BCAAs (hi/lo rates) | NA |
| | 5glucose | | | | |

FM1: 25 g/L YE, K-Phosphate buffer, MgSO₄, trace metals, ammonium phosphate, iron chloride.
FM2: 24 g/L YE, 12 g/L soytone, K-Phosphate buffer.
FM5: 8 g/L YE, K-Phosphate buffer, MgSO₄, trace metals, ammonium phosphate, iron chloride.
FM4: K-phosphate buffer, MgSO₄, trace metals, citric acid, ammonium sulfate, iron citrate.

TABLE 15

| STRAIN# | SYN5941 | SYN1980 |
|---|---|---|
| Final Batch# | SYN5941BR12-13 | SYN1980BR16-17 |
| Activity (μmol/10\hat{ }9 CFU/h) | 2.69 | 0.17 |

### Example 10. In Vitro Activity Study for Optimized BCAA Consuming Strain SYN 5941 in a Gastrointestinal Simulation System

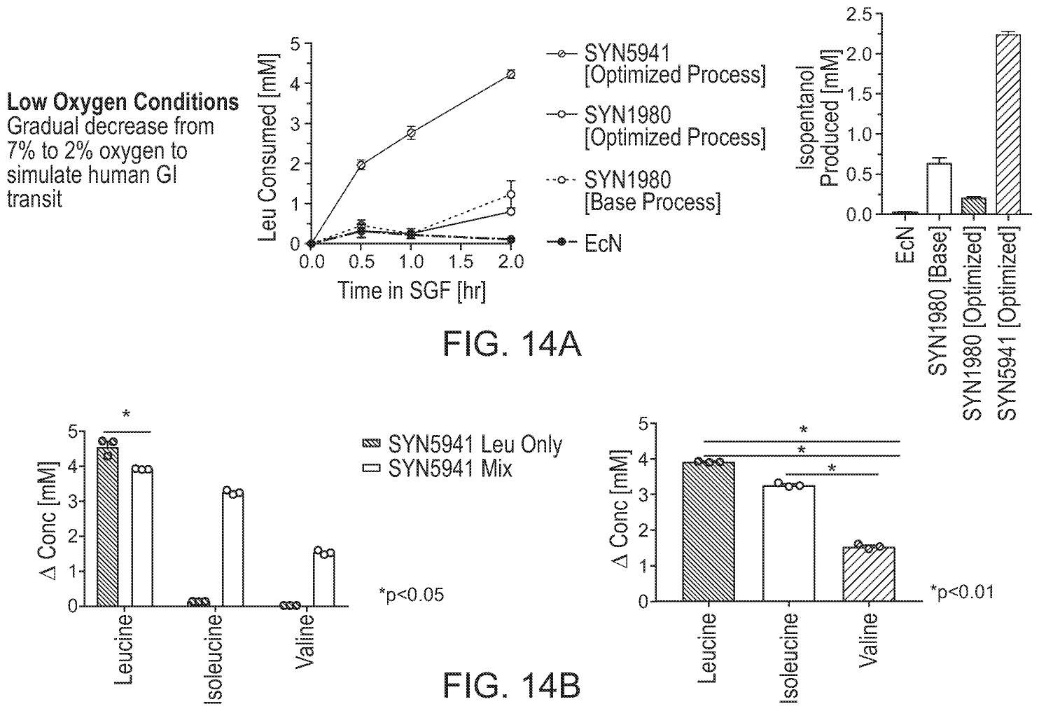

The in vitro activity of the optimized strain, SYN5941, was compared to the that of the original variant, SYN1980, using the in vitro gastrointestinal simulation (IVS) model described above in the method section. FIG. 14A displays representative data concerning leucine consumption (left) and isopentanol production (right) by these two strains during two hours' incubation in simulated gastric fluid with bicarbonate buffering. The oxygen concentration in this assay was decreased gradually from 7% to 4% oxygen, mimicking the low oxygen conditions of the human upper gastrointestinal tract. SYN5941 displayed increased leucine consumption and isopentanol production in IVS, as compared to SYN1980. Notably, this increased leucine consumption was not attributable to fermentation process changes, as SYN1980 prepared with the optimized process demonstrated comparable activity to the SYN1980 prepared with the base process. The consumption specificity towards leucine only or leucine, isoleucine and valine mixture as the substrate(s) were tested in the IVS system as well. As demonstrated in FIG. 14B, SYN5941 had the highest activity towards leucine when leucine, isoleucine and valine were present in equal amount of 5 mM. The overall leucine consumption was also similar to the reaction system when only leucine was present.

Leucine consumption rates and isopentanol production rates were calculated based on the data points collected between time zero and the first 2 hours. As shown in Table 16, the rate of leucine consumption was greater for SYN5941 than for SYN1980 (0.85±0.04 versus 0.25±0.12 μmol/10⁹ CFU/hr). The same trend can be said for isopentanol production (0.45±0.01 versus 0.13±0.02 μmol/10⁹ CFU/hr).

TABLE 16

| | | Leu | | Isopentanol | |
|---|---|---|---|---|---|
| | MSUD | Consumption Rate | | Production Rate | |
| | Fermentation | (μmol/10⁹ CFU/h) | | (μmol/10⁹ CFU/hr) | |
| Strain | Process | Mean | StDev | Mean | StDev |
| SYN5941 | Optimized | 0.85 | 0.04 | 0.45 | 0.01 |
| SYN1980 | Optimized | 0.16 | 0.03 | 0.04 | 0.00 |
| SYN1980 | Original | 0.25 | 0.12 | 0.13 | 0.02 |
| EcN | NA | 0.02 | 0.03 | 0.00 | 0.00 |

### Example 11. In Vitro Characterization of SYN5941

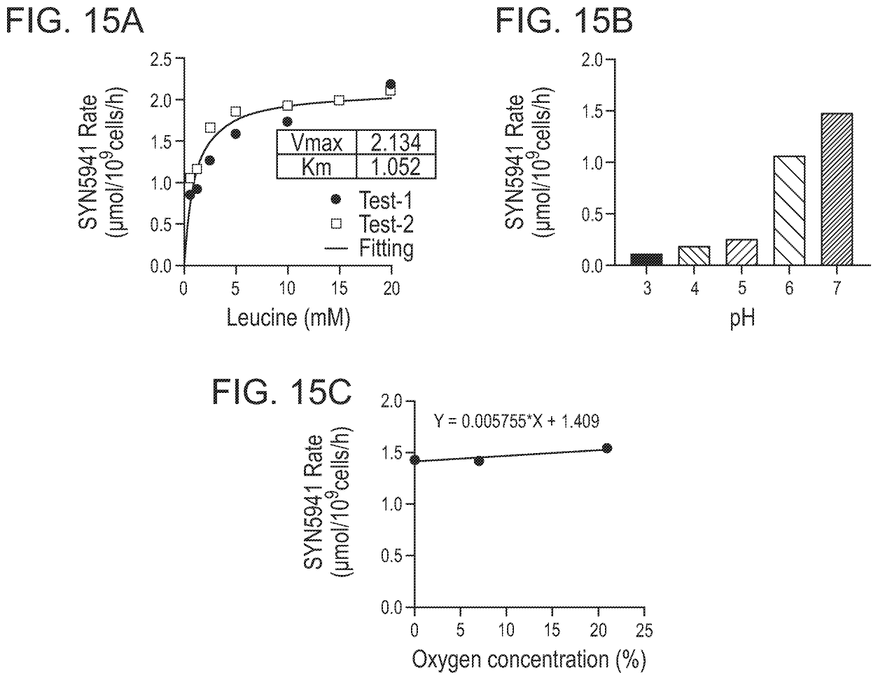

The in vitro activity of the optimized strain, SYN5941, was characterized in various conditions. FIG. 15 depicts leucine consumption rate of SYN5941 in increase leucine concentration (FIG. 15A), increasing pH (FIG. 15B), and increasing oxygen concentration (FIG. 15C). Various substrate concentrations (0, 0.63, 1.25, 5, 10, 15, or 20 mM leucine) were added into the reaction media and the leucine consumption rate was measured for each concentration, respectively. The leucine concentration was measured by HPLC at different time points to obtain the rates. The leucine consumption rates were plotted against the substrate concentration and fitted by a hyperbolic curve. SYN5941 was considered as one enzyme molecule, then the corresponding values of the Ymax and the X value to reach ½ Ymax can be obtained to use as Vmax and Km for SYN5941 during catalysis of leucine degradation. Similar assays were done a fixed substrate concentration (10 mM) but varied the media pH (3, 4, 5, 6, or 7) and oxygen level (0%, 7%, or 21%) to investigate the pH and oxygen dependence. The optimal strain activity is at pH 7. However, it is relatively insensitive to oxygen level which is consistent with the original reason to choose this redox neutral pathway.

### Example 12. In Vivo Efficacy Study for Optimized BCAA Consuming Strain SYN5941 in iMSUD Mice

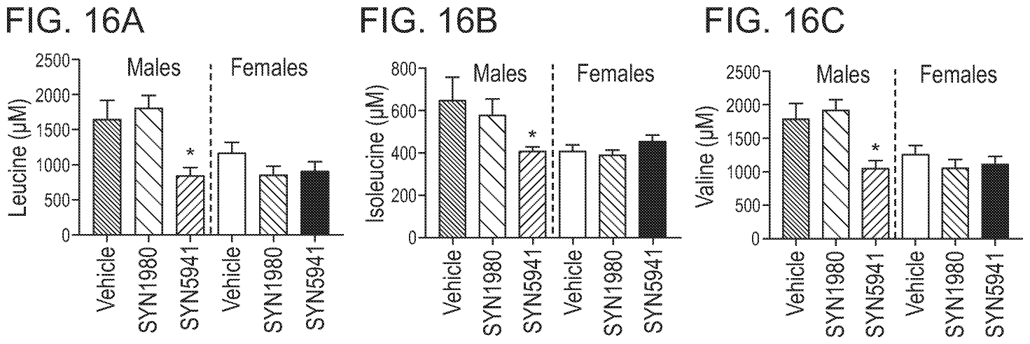

Two independent studies were performed to compare the efficacy of SYN5941 against SYN1980 in a mouse model of MSUD (iMSUD) provided with a high-protein diet. In the first study (FIG. 16), the leucine levels in the high-protein diet were increased to 1647±265.8 μM in iMSUD males and 1165±151.9 μM in iMSUD females. SYN5941 lowered leucine in males to 843.4±113.4 μM, which was more significant than SYN1980 (FIG. 16A). The high protein diet also increased the levels of isoleucine and valine in males and females but similar to leucine, a more significant effect was observed with SYN5941 in males (FIG. 16B-C).

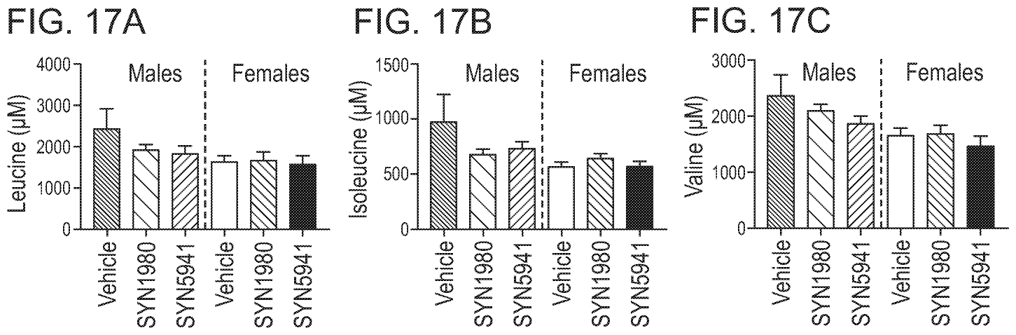

In the second study (FIG. 17), the leucine levels in the high-protein diet were further increased to 2428±485.1 μM in iMSUD males and 1626±150.1 μM in iMSUD females. In contrast to the first study, neither SYN5941 nor SYN1980 significantly lowered leucine levels in either males or females (FIG. 17A). The high protein diet also increased the levels of isoleucine and valine in both males and females but similar to leucine, the bacteria had no significant effect on the levels of these amino acids (FIG. 17B-C).

### Example 13. In Vivo Efficacy Study for Optimized BCAA Consuming Strain SYN5941 in Healthy Non-Human Primates

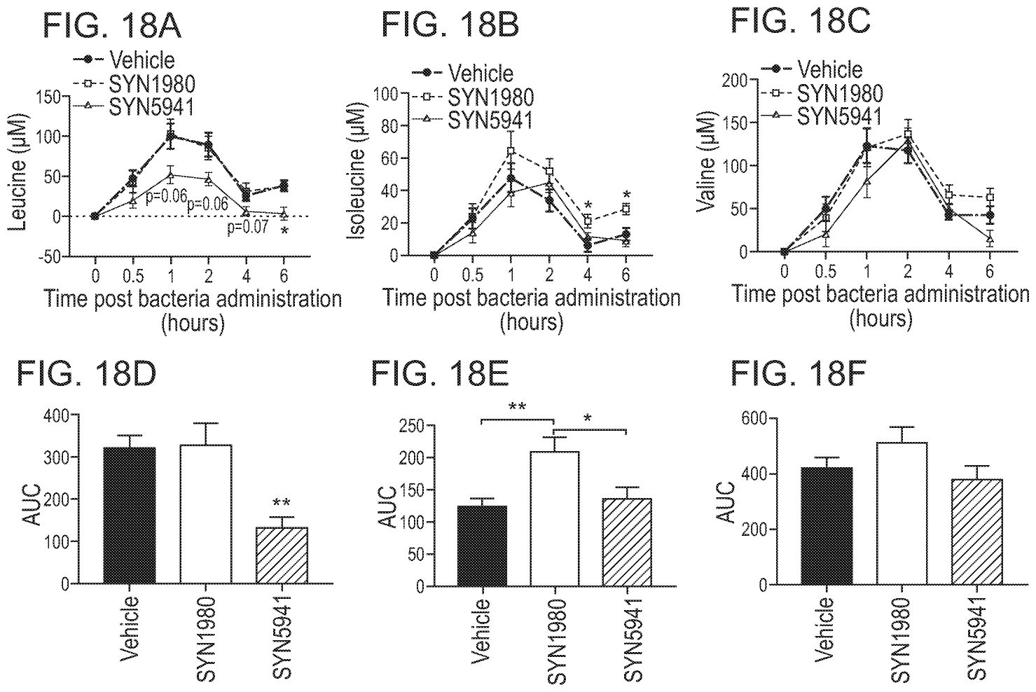

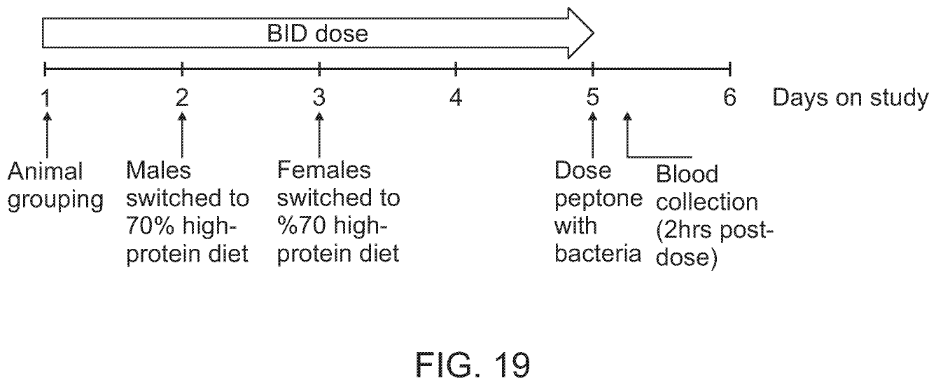

To assess the efficacy of SYN5941 and SYN1980 in healthy non-human primates, monkeys received a single dose of vehicle or bacteria concomitantly with the meat digest peptone. Branched chain amino acid levels leucine, isoleucine and valine peaked approximately 1 hour post-peptone administration in vehicle-treated animals to 99.7±16.5 μM, 47.7±9.3 μM and 123.2±21.4 μM, respectively (FIG. 18A-18C). SYN5941 lowered the levels of leucine more significantly than SYN1908 (FIG. 18).

Table 17 shows that SYN5941 exhibited significantly higher leucine consumption activities than SYN1980 in all three systems demonstrated above in Examples 9, 10 and 13.

TABLE 17

| Strain | In vitro activity | IVS | NHP |
|---|---|---|---|
| SYN1980* | 0.17 | 0.16 | 327.3 |
| SYN5941** | 2.69 | 0.85 | 133.5 |
| UNIT | (μmol/10⁹ CFU/h) | (μmol/10⁹ CFU/h) | (area under curve) |
| Fold of | 15.8 | 5.3 | 59.2% decrease |
| improvement | | | |

*Original operon in strain SYN1980: leuDH (Bacillus cereus) - kivD (Lactococcus lactis) - adh (Saccharomyces cerevisiae) Original chassis for strain SYN1980: SYN469 = SYN001, ΔilvC, ΔleuE, lacZ::Ptet-livKHMGF
**Optimized operon in new strain SYN5941: leuDH (Cetobacterium Ceti) - kivD (Erwinia iniecta) - adh (Alcanivoriax dieselolei) Optimized chassis for new strain SYN5941: SYN094 = SYN001, strpR

### Example 14. Molar Balance Closure of the Isopentanol Pathway

The performance and molar balance closure of the isopentanol pathway in SYN5941 was assessed in Ambr15 bioreactors. The reactors were filled to 17 mL with M9 media with 0.5% glucose, 10 mM Leu, 10 mM Val, and 5 mM Ile. Conditions were controlled with 0% dissolved oxygen and pH at 7.0. Activated biomass was inoculated to an OD600 of 1, and samples of the supernatant were taken over time to monitor metabolite concentrations.

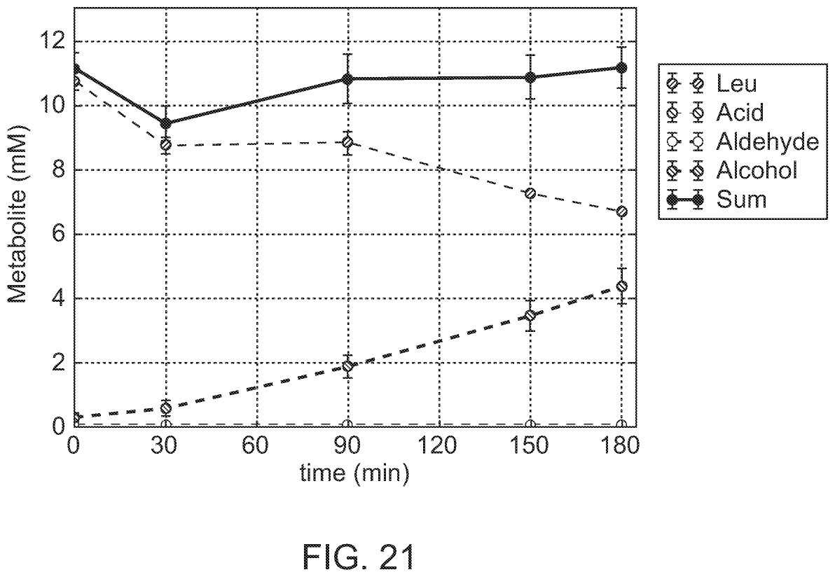

The extracellular concentration profiles of pathway intermediates are shown in the Figure below. Over the course of 180 minutes, 4.1±0.3 mM of Leucine was consumed and 4.4±0.5 mM of isopentanol accumulated in the media. The keto-acid (2-oxoisocaproate) and aldehyde (isovaleraldehyde) were not observed in the supernatant. Thus, the flux through the pathway is balanced and accounted for. This is also shown by the conservation of total moles of the pathway intermediates (FIG. 21).

### Methods—Fermentation

The assay was performed in an AMBR15f, microbioreactor system from Sartorius. The vessels were filled with 17 mls of 1×m9 media salts, supplemented with 2.0 mm MgSO4, 0.1 mM CaCl, 5% glucose, 10 mM L-leucine, 5 mM L-isoleucine, and 10 mM valine. The vessels were filled 18 hrs prior to inoculation, to enable both the pH and DO optodes to hydrate. The temperature in the reactors was kept at 37c, the pH was maintained at 7 using 2N NaOH, and the dissolved oxygen was kept at 0 using a 0.14 vvm N2 flow rate. The agitation was set to 500 RPM to enable good mixing throughout the experiment. The bioreactors were inoculated to an OD600 of 1, from activated biomass supplied by Synlogic. The bioreactors were sampled at 0, 30, 90, 150, and 180 minutes post inoculation. Samples were immediately centrifuged at 15000×g for 30 secs in a microcentrifuge and the supernatant was removed for analysis. Supernatants were stored at −20° C. until ready for analysis.

### Methods—Analytics

Analytics were developed for two methods, one method towards liquid chromatography mass spectrometry (LCMS) for the quantification of leucine (Leu), ketoisocaproate acid (Leu acid), and isovaleraldehyde (Leu aldehyde). The method (data not shown), was also validated and used for quantification of valine and isoleucine (and there respected acid and aldehyde products). While the second method developed for gas chromatography mass spectrometry (GCMS) for the quantification of isopentanol (Leu alcohol). Thus, completing the pathway for mass balance for SYN5941. The GCMS method (data not shown), was also validated and used for quantification of valine and isoleucine alcohol products.

LCMS analysis was performed on a Thermo Ultimate 3000 UPLC system with a Thermo Q-Exactive quadrupole-orbitrap mass detector and a Thermo Accucore PFP column (2.1×100 mm, 2.6 um packing) using the following elution solvents: A=0.1% formic acid and 0.1% TFA in water; B=0.1% formic acid in acetonitrile. The gradient was at 0.5 mL/min of 1% B in A for 60 seconds, followed by a linear ramp from 1% to 40% B in A over 270 seconds. The column is then flushed with 95% B in A for 60 seconds, and re-equilibrated with 1% B in A for 180 seconds. MS acquisition was from 0.8 to 5.3 minutes.

Column effluent is introduced into the mass spectrometer via a standard Thermo ESI source with positive mode ionization at +3800V, vaporizer temperature of 400° C., and ion transfer tube temperature of 375° C. Thermo reports gas flow rates in arbitrary units probably approximating L/min at STP. Set points were: sheath gas, 60; aux gas, 30; sweep gas, 1. To increase data acquisition rate, orbitrap resolution was set to 17,500. Quadrupole resolution was 1 m/z.

This method also derivatizes both aldehydes and keto acids, improving the stability of those analytes. Numerous derivatizing agents were explored, and it was found that 2-(Dimethylamino)ethylhydrazine in methanol resulted in the best sensitivity in positive mode. A buffer of 0.5M acetic acid and 0.5M sodium acetate in methanol was used for the quantification of LEU ACID and LEU ALDEHYDE, while also measuring non-derivatized LEU.

GC-MS analysis was performed on an Agilent GCMS/MSD with a Gerstel autosampler, using a J&W DB-WAX GC Column (15 m) and chloroform as the extraction solvent. Front injector was set at 250° C. and a flow rate of 1 mL/min. The oven temperature held at 40° C. for 1 minute, followed by a ramp to 130° C. (15° C./min), and then ramped up to 200° C. (65° C./min). Ms acquisition scan window was at 40-150 mz, with the MS source and MS quad at 250 C and 200° C. respectively.

Finally to facilitate high throughput and automation, a Gerstel autosampler was used to inject the extracted bottom chloroform layer in a 96 well plate format with the aqueous ambr15 culture matrix on top acting as an overlay to prevent product evaporation. To account for any other potential alcohol product evaporation, 2-heptanol was added to the chloroform as an internal.

1. A recombinant bacterium capable of consuming leucine at a rate of at least about 0.5 μmol/10⁹ CFU/h in vitro.

2. The recombinant bacterium of claim 1, wherein the bacterium is capable of consuming leucine at a rate of at least about 0.75, 1, 1.25, 1.5, 1.75, 2, 2.25, 2.5, or 2.75 μmol/109 CFU/h.

3. The recombinant bacterium of claim 1, wherein the bacterium is capable of consuming leucine at a rate of about 2.5 to about 2.75 μmol/10⁹ CFU/h.

4. The recombinant bacterium of any one of the previous claims, wherein the bacterium comprises at least one gene sequence encoding at least one leucine catabolism enzyme operably linked to at least one directly or indirectly inducible promoter that is not associated with a gene encoding the at least one leucine catabolism enzyme in nature.

5. The recombinant bacterium of claim 4, wherein the promoter is selected from the group consisting of an FNRS promoter, a Pₜₑₜ promoter, a P_{cmt} promoter, an FNRS promoter, a P_{cl857} promoter, and a P_{BAD} promoter.

6. The recombinant bacterium of claim 4, wherein the at least one gene sequence encoding the at least one leucine catabolism enzyme is leuDH, kivD, and/or adh2.

7. The recombinant bacterium of claim 6, wherein the bacterium further comprises at least one gene sequence encoding at least one transporter capable of transporting leucine.

8. The recombinant bacterium of claim 7, wherein the at least one gene sequence encoding the at least one transporter is brnQ.

9. The recombinant bacterium of claim 8, wherein the bacterium comprises an operon, wherein the operon comprises leuDH, kivD, adh2, and brnQ.

10. The recombinant bacterium of claim 9, wherein the operon comprises a sequence having at least 90% identity to a sequence of any one of SEQ ID NOs: 25-48.

11. The recombinant bacterium of any one of claims 8-10,
wherein the leuDH gene encodes a LeuDH protein which comprises a sequence having at least 90% identity to a sequence of any one of SEQ ID NOs: 49-72;
wherein the kivD gene encodes a KivD protein which comprises a sequence having at least 90% identity to a sequence of any one of SEQ ID NOs: 73-96;
wherein the adh2 gene encodes an Adh2 protein which comprises a sequence having at least 90% identity to a sequence of any one of SEQ ID NOs: 97-120; and/or
wherein the brnQ gene encodes a BrnQ protein which comprises a sequence having at least 90% identity to a sequence of any one of SEQ ID NOs: 121-144.

12. A recombinant bacterium comprising an operon,
wherein the operon comprises an optimized leuDH gene which encodes a LeuDH protein, an optimized kivD gene which encodes a KivD protein, and an optimized adh2 gene which encodes an Adh2 protein, and an optimized brnQ gene which encodes a BrnQ protein,
wherein the operon comprises a sequence having at least 90% identity to a sequence of any one of SEQ ID NOs: 25-48, and
wherein the operon is operably linked to an inducible promoter that is not associated with a leuDH gene, a kivD gene, a adh2 gene, or a brnQ gene in nature.

13. The recombinant bacterium of claim 12, wherein the LeuDH protein comprises a sequence having at least 90% identity to a sequence of any one of SEQ ID NOs: 49-72.

14. The recombinant bacterium of claim 12 or claim 13, wherein the KivD protein comprises a sequence having at least 90% identity to a sequence of any one of SEQ ID NOs: 73-96.

15. The recombinant bacterium of any one of claims 12-14, wherein the Adh2 protein comprises a sequence having at least 90% identity to a sequence of any one of SEQ ID NOs: 97-120.

16. The recombinant bacterium of any one of claims 12-15, wherein the BrnQ protein comprises a sequence having at least 90% identity to a sequence of any one of SEQ ID NOs: 121-144.

17. The recombinant bacterium of any one of claims 4-8, wherein the at least one gene sequence encoding the at least one leucine catabolism enzyme and the inducible promoter, are integrated into the chromosome of the bacterium.

18. The recombinant bacterium of any one of claims 9-16, wherein the operon and the inducible promoter are integrated into the chromosome of the bacterium.

19. The recombinant bacterium of any one of claims 8-18, wherein the bacterium further comprises a second brnQ gene which encodes a second BmQ protein, wherein the second brnQ gene is operably linked to an inducible promoter that is not associated with a brnQ gene in nature, and wherein the second brnQ gene is inserted into the malE K or malP T locus on a chromosome of the bacterium.

20. The recombinant bacterium of any one of the previous claims, wherein the bacterium comprises a genetic modification in leuE that reduces leucine export from the bacterium.

21. The recombinant bacterium of any one of the previous claims, wherein the bacterium comprises a genetic modification in ilvC that reduces endogenous biosynthesis of leucine in the bacterium.

22. The recombinant bacterium of any one of the previous claims, wherein the bacterium further comprises a livKHMGF gene sequence encoding at least one transporter capable of transporting leucine.

23. The recombinant bacterium of claim 22, wherein the livKHMGF gene sequence is operably linked to at least one promoter that is not associated with a livKHMGF gene in nature.

24. The recombinant bacterium of any one of claims 12-23, wherein the bacterium is capable of consuming leucine at a rate of at least about 0.5 μmol/10⁹ CFU/h in vitro.

25. The recombinant bacterium of any one of the previous claims, wherein the bacterium is capable of producing isopentanol at a rate of at least about 0.2 μmol/10⁹ CFU/h in vitro.

26. The recombinant bacterium of claim 25, wherein the bacterium is capable of producing isopentanol at a rate of at least about 0.2 to 0.5 μmol/10⁹ CFU/h in vitro

27. The recombinant bacterium of any one of the previous claims, wherein the bacterium exhibits preferentially consumes leucine over valine and isoleucine.

28. The recombinant bacterium of claim 27, wherein the bacterium exhibits a leucine/valine activity ratio of at least about 1.1 to at least about 2.75.

29. The recombinant bacterium of claim 27 or claim 28, wherein the bacterium exhibits a leucine/isoleucine activity ratio of at least about 1.1 to at least about 5.

30. The recombinant bacterium of any one of the previous claims, wherein the bacterium is a probiotic bacterium selected from the group consisting of Bacteroides, Bifidobacterium, Clostridium, Escherichia, Lactobacillus, and Lactococcus.

31. The recombinant bacterium of claim 30, wherein the bacterium is Escherichia coli strain Nissle.

32. A pharmaceutically acceptable composition comprising the recombinant bacterium of any one of the previous claims and a pharmaceutically acceptable carrier.

33. A method of reducing the level of leucine in a subject, the method comprising a step of administering to the subject the pharmaceutically acceptable composition of claim 32.

34. A method of treating a disease associated with excess leucine and/or a metabolic disorder involving the abnormal catabolism of leucine in a subject, the method comprising a step of administering to the subject the pharmaceutically acceptable composition of claim 32.

35. The method of claim 33 or claim 34, wherein the subject has, or is suspected of having maple syrup urine disease (MSUD).
